(19) United States
(12) Patent Application Publication
(10) Pub. No.: US 2025/0304957 A1
(43) Pub. Date: Oct. 2, 2025

(54) SINGLE-STRANDED LOOP OLIGONUCLEOTIDES

(71) Applicant: ALNYLAM PHARMACEUTICALS, INC., Cambridge, MA (US)

(72) Inventors: Muthiah MANOHARAN, Cambridge, MA (US); Ivan ZLATEV, Cambridge, MA (US)

(21) Appl. No.: 18/864,172

(22) PCT Filed: May 12, 2023
(86) PCT No.: PCT/US2023/066971

§ 371 Date: Nov. 8, 2024

Related U.S. Application Data
(60) Provisional application No. 63/364,715, filed on May 13, 2022.
(60) Provisional application No. 63/401,946, filed on Aug. 29, 2022.

(51) Int. Cl. C12N 15/113

(52) U.S. Cl. CPC C12N 15/113; C12N 2310/14; C12N 2310/321; C12N 2310/334; C12N 2310/335; C12N 2310/336; C12N 2310/531

(57) ABSTRACT

One aspect of the present invention relates to a single-stranded oligonucleotide having a having a sequence represented by formula (I): (5′-Z¹-3′)-Q¹-L-Q²-(5′-Z²-3′) (I). In formula (I), Z¹ is a first oligonucleotide, comprising 15-100 optionally modified nucleotides that is substantially complementary to a target gene; Z² is a second oligonucleotide, comprising 15-100 optionally modified nucleotides that is substantially complementary to Z¹; and Z¹ and Z² are capable of forming an intra-strand duplexed region comprising 3 or more consecutive base pairs. L is a linking group. Q¹ and Q² each independently represent 0 to 12 optionally modified nucleotides. At least one nucleotide in formula (I) is a modified nucleotide. Other aspects of the invention relate to a pharmaceutical composition and a method for inhibiting the expression of one or more target gene in a subject.

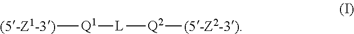

### CROSS-REFERENCE TO RELATED APPLICATIONS

This application claims benefit of priority to U.S. Provisional Application No. 63/364,715, filed May 13, 2022, and U.S. Provisional Application No. 63/401,946 filed Aug. 29, 2022, both of which are herein incorporated by reference in their entirety.

### SEQUENCE LISTING

The instant application contains a Sequence Listing which has been submitted in XML format and is hereby incorporated by reference in its entirety. Said XML copy, created on May 12, 2023, is named 29520_1509-PCT__ALN-464__SL.xml and is 1,482,842 bytes in size.

### FIELD OF INVENTION

This invention generally relates to the field of RNA interference technology with single-stranded loop oligonucleotides.

### BACKGROUND

Chemical modifications of the nucleobases, ribose sugar, and phosphate backbone have been used in double-stranded RNAi agents to improve drug-like properties of these therapeutic oligonucleotides and to confer favorable pharmacological properties to GalNAc-oligonucleotide conjugates in preclinical and clinical development.

Various siRNA designs have been developed to achieve better stability and potency. The current studies addressed the stability and duration-related challenges by incorporating chemical modifications, but overlooked process-related challenges in synthesizing the double-stranded siRNAs. Sense and antisense strands are typically synthesized separately, go through a tedious multistep purification as single strands, and then annealed into a duplex which further undergoes another round of purification and quality control. This process is complex, time-taking, expensive, and raises environmental sustainability concerns.

However, there is a continuing need for an improved design for the RNAi agent to involve simplified manufacturing and purification processes, yet at the same time preserving or improving the efficacy of the RNAi agent.

### SUMMARY

One aspect of the invention relates to a single-stranded oligonucleotide capable of inhibiting the expression of a target gene, having a sequence represented by formula (I):

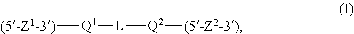

wherein:
Z¹ is a first oligonucleotide, comprising 10-100 optionally modified nucleotides (e.g., 15-100) that is substantially complementary to a target gene;
Z² is a second oligonucleotide, comprising 10-100 optionally modified nucleotides (e.g., 15-100) that is substantially complementary to Z¹;
Z¹ and Z² are capable of forming an intra-strand duplexed region comprising 3 or more consecutive base pairs;
L is a linking group;
Q¹ and Q² each independently represent 0 to 12 optionally modified nucleotides; and
at least one nucleotide in formula (I) is a modified nucleotide.

The first oligonucleotide Z¹ and second oligonucleotide Z² each may independently comprise 15-100 optionally modified nucleotides. For instance, Z¹ and Z² each may independently comprise 15-40, 15-25, or 19-23 optionally modified nucleotides. In some embodiments, the first oligonucleotide Z¹ and second oligonucleotide Z² each may independently comprise at least 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. Z¹ and Z² each may independently have about 10 to about 50 nucleotides, about 10 to about 40 nucleotides, about 15 to about 40 nucleotides, about 10 to about 35 nucleotides, about 10 to about 30 nucleotides, about 10 to about 25 nucleotides, about 10 to about 20 nucleotides, about 15 to about 50 nucleotides, about 15 to about 40 nucleotides, about 15 to about 35 nucleotides, about 15 to about 30 nucleotides, about 15 to about 25 nucleotides, about 15 to about 20 nucleotides, about 19 to about 23 nucleotides, about 19 to about 21 nucleotides, or about 18 to about 20 nucleotides in length. Each of the nucleotides in first oligonucleotide Z¹ and second oligonucleotide Z² may be independently and optionally modified. In some embodiments, Z¹ and Z² each contain the same number of optionally modified nucleotides.

Q¹ and Q² each may independently comprise 0 to 12 optionally modified nucleotides. For instance, Q¹ and Q² each may independently comprise 0 to 10, 0 to 6, 0 to 4, 0 to 3, 0 to 2, 1 to 6, 1 to 4, 1 to 3, or 2 to 3 optionally modified nucleotides. In some embodiments, Q¹ and Q² each are 0. In some embodiments, one of Q¹ and Q² is 0. In some embodiments, Q¹ and Q² have the same number of optionally modified nucleotides.

In some embodiments, the single-stranded oligonucleotide can be cleaved at the linking group L. The first oligonucleotide Z¹ can be cleaved into an antisense strand that is substantially complementary to a target gene (e.g., a target mRNA or DNA), and the second oligonucleotide Z² can be cleaved into a sense strand that is substantially complementary to Z¹.

The first oligonucleotide Z¹ and second oligonucleotide Z² can form an intramolecular double-stranded region comprising 3 or more consecutive base pairs (e.g., a duplex region of 3, 4, 5, 6, 7, 8, 9, 10, 11, 12, 13, 14, 15, 16, 17, 18, 19, 20, 21, 22, 23, 24, or 25 base pairs). For instance, the duplex region may comprise 10-25, 15-25, 19-23, 19, 20, 21, 22, or 23 base pairs. The intra-strand duplexed region formed by Z¹ and Z² may contain all consecutive base pairs, or may contain no more than 3 (e.g., 0, 1, 2, or 3) mismatch based pairs.

In some embodiments, the single-stranded oligonucleotide comprises at least one chemical modification. In some embodiments, each of the first oligonucleotide Z¹ and second oligonucleotide Z² comprise at least one chemical modification. In some embodiments, all the nucleotides in Z² are modified nucleotides. In some embodiments, all the nucleotides in Z¹ are modified nucleotides. In some embodiments, all the nucleotides of the single-stranded oligonucleotide are modified.

The chemical modification to the nucleotide(s) may include an internucleoside linkage modification, a nucleobase modification, a sugar modification, or combinations thereof.

In certain embodiments, the chemical modification is selected from the group consisting of LNA, ENA, HNA, CeNA, 2′-O-methoxyalkyl (e.g., 2′-O-methoxymethyl, 2′-O-methoxyethyl, or 2′-O-2-methoxypropanyl), 2′-O-alkyl (e.g., 2′-OMethyl), 2′-O-allyl, 2′-C-allyl, 2′-fluoro, 2′-deoxy, 2′-O—N-methylacetamido (2′-O-NMA), 2′-O-dimethylaminoethoxyethyl (2′-O-DMAEOE), 2′-O-aminopropyl (2′-O-AP), 2′-ara-F, L-nucleoside modification (such as 2′-modified L-nucleoside, e.g., 2′-deoxy-L-nucleoside), BNA abasic sugar, abasic cyclic and open-chain alkyl, and combinations thereof.

In certain embodiments, the chemical modification is selected from the group consisting of at least one of the modified nucleotides is a deoxy-nucleotide, a 3′-terminal deoxythimidine (dT) nucleotide, a 2′-O-methyl modified nucleotide, a 2′-fluoro modified nucleotide, a 2′-deoxy-modified nucleotide, a locked nucleotide (e.g., LNA), an unlocked nucleotide (e.g., UNA), a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxy-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a 5′-phosphorothioate group, a nucleotide comprising a 5′-methylphosphonate group, a nucleotide comprising a 5′ phosphate or 5′ phosphate mimic, a nucleotide comprising vinyl phosphonate, a nucleotide comprising glycol nucleic acid (GNA), a nucleotide comprising glycol nucleic acid (GNA) S-Isomer (S-GNA), a nucleotide comprising 2-hydroxymethyl-tetrahydrofuran-5-phosphate, a nucleotide comprising 2′-deoxythymidine-3′phosphate, a nucleotide comprising 2′-deoxyguanosine-3′-phosphate, a 2′-5′-linked nucleotide (“3′-RNA”), or a terminal nucleotide linked to a cholesteryl derivative or a dodecanoic acid bisdecylamide group.

In certain embodiments, the chemical modification is a 2′-modification selected from the group consisting of 2′-O-methyl, 2′-O-allyl, 2′-O-methoxyalkyl (e.g., 2′-O-methoxymethyl, 2′-O-methoxyethyl, or 2′-O-2-methoxypropanyl), 2′-deoxy, 2′-fluoro, and combinations thereof.

In some embodiments, about 100%, 95%, 90%, 85%, 80%, 75%, 70%, 65%, 60%, 55%, 50%, 45%, 40%, 35% or 30% of Z¹ are modified. In some embodiments, about 100%, 95%, 90%, 85%, 80%, 75%, 70%, 65%, 60%, 55%, 50%, 45%, 40%, 35% or 30% of Z² are modified. In some embodiments, about 100%, 95%, 90%, 85%, 80%, 75%, 70%, 65%, 60%, 55%, 50%, 45%, 40%, 35% or 30% of all the nucleotides in the single-stranded oligonucleotide are modified. For example, when 50% of all the nucleotides are modified, 50% of all nucleotides present in the single-stranded oligonucleotide contain at least one modification as described herein.

In one embodiment, at least 50% of the nucleotides of the single-stranded oligonucleotide are independently modified with 2′-O-methyl, 2′-O-allyl, 2′-O-methoxyalkyl (e.g., 2′-O-methoxymethyl, 2′-O-methoxyethyl, or 2′-O-2-methoxypropanyl), 2′-deoxy, or 2′-fluoro.

In some embodiments, one or more of the five internucleotide linkages among the six 3′-terminal nucleotides is a modified internucleotide linkage. In some embodiments, one or more of the five internucleotide linkages among the six 5′-terminal nucleotides is a modified internucleotide linkage.

In some embodiments, one or more of the five internucleotide linkages among the six 5′-terminal nucleotides of Z² is a modified internucleotide linkage. In some embodiments, one or more of the five internucleotide linkages among the six 5′-terminal nucleotides of Z¹ is a modified internucleotide linkage.

In some embodiments, the single-stranded oligonucleotide further comprises one or more modified internucleotide linkage between the 3′-terminal nucleotide of Z¹ and the first nucleotide of Q¹. In some embodiments, the single-stranded oligonucleotide further comprises one or more modified internucleotide linkages between the nucleotides of Q¹.

In some embodiments, the single-stranded oligonucleotide further comprises a phosphate or phosphate mimic at the 5′-end of a nucleotide sequence (e.g., Z¹ and/or Z²). In some embodiments, the single-stranded oligonucleotide comprises a phosphate mimic at the 5′-end of a nucleotide sequence (e.g., Z¹ and/or Z²). In one embodiment, at least one phosphate mimic is at the 5′ end of Z¹. In one embodiment, the phosphate mimic is a 5′-vinyl phosphonate (VP). In one embodiment, the phosphate mimic is a 5′-cyclopropyl phosphonate. In one embodiment, the phosphate mimic is a 5′-vinyl phosphate.

In some embodiments, the 5′-end or 3′-end nucleotide in the single-stranded oligonucleotide of formula (I) comprise a 2′-5′-linked nucleotide modification; or the 5′-end or 3′-end nucleotide is conjugated to an abasic nucleotide, an inverted nucleotide, or an inverted abasic nucleotide (e.g., ribonucleotide), optionally via a phosphodiester, phosphorothioate, or phosphodithioate linkage.

In some embodiments, the 5′-end or 3′-end nucleotide in the single-stranded oligonucleotide of formula (I) is modified to comprise a linking moiety containing a mono-, di-, tri-, tetra-, penta- or polyprolinol, or mono-, di-, tri-, tetra-, penta- or polyhydroxyprolinol.

In some embodiments, the single-stranded oligonucleotide further comprises at least one terminal, chiral modification (such as a terminal, chiral phosphorus atom).

A site specific, chiral modification to the internucleotide linkage may occur at the 5′ end, 3′ end, or both the 5′ end and 3′ end of a nucleotide sequence. This is being referred to herein as a “terminal, chiral” modification. The terminal modification may occur at a 3′ or 5′ terminal position in a terminal region, e.g., at a position on a terminal nucleotide or within the last 2, 3, 4, 5, 6, 7, 8, 9 or 10 nucleotides of a nucleotide sequence. Each of the chiral pure phosphorus atoms may be in either Rp configuration or Sp configuration, and combination thereof. More details regarding chiral modifications and chirally-modified RNA agents can be found in WO 2019/126651A1, which is incorporated herein by reference in its entirety.

In some embodiments, the single-stranded oligonucleotide comprises at least two consecutive phosphorothioate or methylphosphonate internucleotide linkage modifications. In some embodiments, the single-stranded oligonucleotide comprises at least two blocks of two consecutive phosphorothioate or methylphosphonate internucleotide linkage modifications. In some embodiments, the single-stranded oligonucleotide comprises at least three blocks of two consecutive phosphorothioate or methylphosphonate internucleotide linkage modifications.

In some embodiments, the single-stranded oligonucleotide has at least two phosphorothioate internucleotide linkages at the first five nucleotides on a nucleotide sequence (counting from the 5′ end) (e.g., Z¹ and/or Z²).

In some embodiments, a nucleotide sequence of the single-stranded oligonucleotide (e.g., Z¹ and/or Z²) comprises two blocks of one, two, or three phosphorothioate internucleotide linkages separated by 1, 2, 3, 4, 5, 6, 7, 8, 9, 10, 11, 12, 13, 14, 15, 16, 17, or 18 phosphate internucleotide linkages.

In one embodiment, a nucleotide sequence of the single-stranded oligonucleotide (e.g., Z¹ and/or Z²) comprises at least two consecutive phosphorothioate internucleotide linkage modifications within positions 18-23 of the nucleotide sequence, counting from the 5′-end of the nucleotide sequence. In one embodiment, a nucleotide sequence of the single-stranded oligonucleotide (e.g., Z¹ and/or Z²) comprises at least two consecutive phosphorothioate internucleotide linkage modifications within position 1-5 of the nucleotide sequence, counting from the 5′-end of the nucleotide sequence.

In some embodiments, each of Z¹ and Z² of the single-stranded oligonucleotide comprises at least two consecutive phosphorothioate internucleotide linkage modifications. In one embodiment, each of Z¹ and Z² of the single-stranded oligonucleotide comprises: at least two consecutive phosphorothioate internucleotide linkage modifications within positions 18-23 of the nucleotide sequence, and at least two consecutive phosphorothioate internucleotide linkage modifications within position 1-5 of the nucleotide sequence, counting from the 5′-end of the nucleotide sequence.

In all the above embodiments, the target gene may be a mRNA, pre-mRNA, microRNA, pre-miRNA, long non-coding RNA (lncRNA), or DNA.

In all the above embodiments, the single-stranded oligonucleotide may be an inhibitory single-stranded oligonucleotide, such as an antisense oligonucleotide (ASO), an antimiR (antagomir) oligonucleotide, microRNA mimic, supermir, aptamer, U1 adaptor, triplex-forming oligonucleotide, RNA activator, immuno-stimulatory oligonucleotide, decoy oligonucleotide, heteroduplex-forming oligonucleotide, or a single-stranded siRNA (ss-siRNA) oligonucleotide.

In some embodiments, at least one, two, three, four, or each of the five terminal phosphorous-containing linkages of the 5′-end or the 3′-end of the single-stranded oligonucleotide (e.g., Z¹ and/or Z²) are not phosphorothioate linkages. In one embodiment, the at least one, two, three, four, or each of the five terminal phosphorous-containing linkages of the 5′-end or the 3′-end of the single-stranded oligonucleotide (e.g., Z¹ and/or Z²) are each independently a natural phosphate group or a phosphodiester linkage, or a nitrogen-modified phosphorous-containing linkage (PN-linkage).

In some embodiments, the PN-linkage can have the formula of —N(R)P(═X)(OH)O— or —OP(═X)(OH)N(R)—, —O—P(NR)(═X)O—, —N(SO₂R)P(═X)(OH)O— or —OP(═X)(OH)N(SO₂R)—, or —O—P(NSO₂R)(═X)O—, wherein X is O or S; R may be optionally substituted alkyl, aryl, heteroaryl, or heterocyclyl; or NR may be an optionally substituted cyclic guanidine moiety, an optionally substituted triazolyl group, or a Tmg group

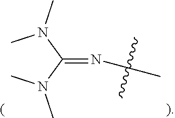 [SMILES: *N=C(N(C)C)N(C)C]

In some embodiments, the PN-linkage comprises an optionally substituted cyclic guanidine moiety. For instance, the PN-linkage can have the structure of

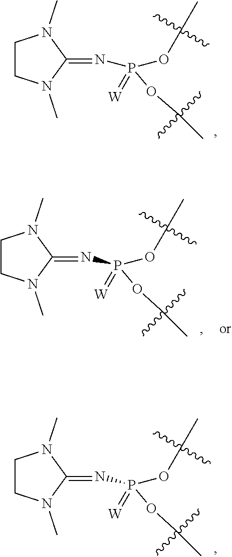

wherein W is O or S. In some embodiments, W is O. In some embodiments, W is S. In some embodiments, the PN-linkage is stereochemically controlled.

In some embodiments, the PN-linkage comprises a triazole moiety (e.g., an optionally substituted triazolyl group). For instance, the PN-linkage can have the structure of

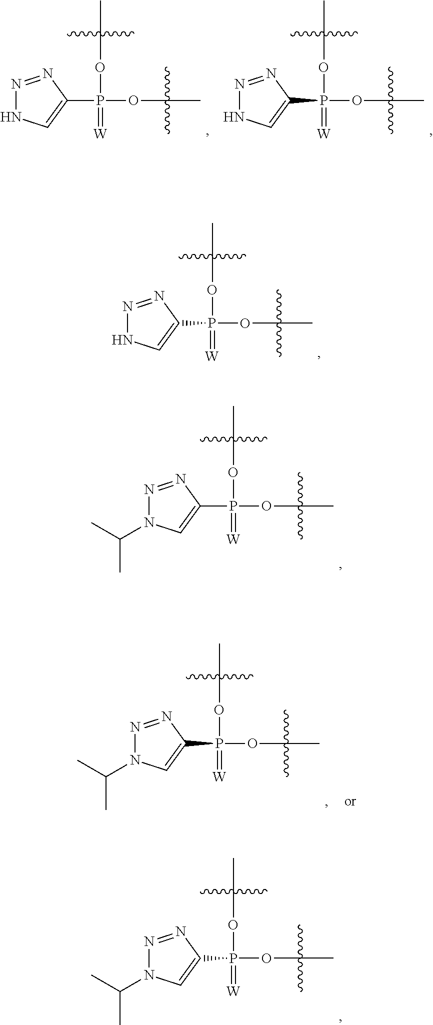

wherein W is O or S. In some embodiments, W is O. In some embodiments, W is S. In some embodiments, the PN-linkage is stereochemically controlled.

In some embodiments, the PN-linkage comprises an alkyne moiety (e.g., an optionally substituted alkynyl group). For instance, the PN-linkage can have the structure of

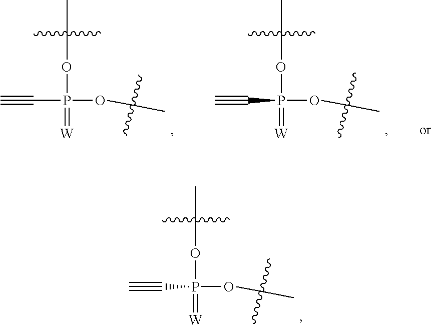

wherein W is O or S. In some embodiments, W is O.
In some embodiments, W is S. In some embodiments, the PN-linkage is stereochemically controlled.

In some embodiments, the PN-linkage comprises a Tmg group

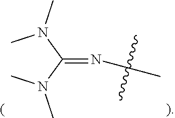 [SMILES: *N=C(N(C)C)N(C)C]

For instance, the PN-linkage can have the structure of

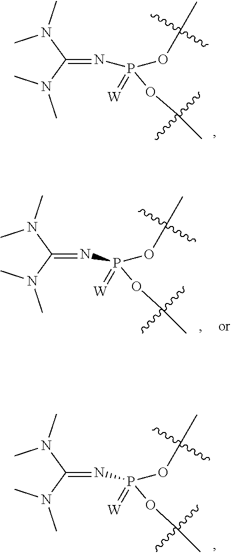

wherein W is O or S. In some embodiments, W is O. In some embodiments, W is S. In some embodiments, the PN-linkage is stereochemically controlled.

Additional suitable PN-linkages may include those described in WO 2019/032612 and WO2021/030778, which are incorporated herein by reference in their entirety.

In some embodiments, L of formula (I) is a cleavable linking group. In some embodiments, the cleavable linking group is cleavable in a homogenate, tritosome, cytosol, or endosome of any types of cells. For instance, the cleavable linking group may be cleavable in liver homogenates, liver tritosomes, liver lysosomes, liver cytosol, liver endosome, brain homogenates, brain tritosomes, brain lysosomes, brain cytosol, or brain endosome. In certain embodiments, the cleavable linking group is a redox cleavable linker (such as a reductively cleavable linker; e.g., a disulfide group), an acid cleavable linker (e.g., a hydrazone group, an ester group, an acetal group, or a ketal group), an esterase cleavable linker (e.g., an ester group), a phosphatase cleavable linker (e.g., an ester group), a peptidase cleavable linker (e.g., an ester group), or endosomal cleavable linker (or a protease cleavable linker, e.g., a carbohydrate linker).

In some embodiments, the cleavable linking group (tether) is an endosomal cleavable linker or a protease cleavable linker, for instance, a carbohydrate linker, wherein the linker is cleaved at least 1.25 times faster in the cell (or under in vitro conditions selected to mimic intracellular conditions) as compared to blood or serum (or under in vitro conditions selected to mimic extracellular conditions).

In some embodiments, L of formula (I) contains a linking moiety represented by a formula: #—(N)ₙ—**. In this formula, # is the bond to Q¹ and ** is the bond to Q²; n is 3 to 12; and each N is independently a linking moiety. For instance, each N may be independently a linking monomer having a chain length of 3 or more atoms.

Herein, the term “chain length” refers to the number of atoms in the shortest linear chain formed by the linking monomer. For instance, for a PEG/PEO, having a structure of

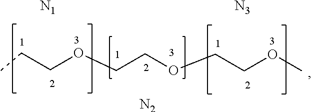

the chain length of the linking monomer is 3 (triethylene glycol). As another example, for a peptide linking monomer, the chain length of the linking monomer having a formula of

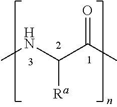

is 3. In one embodiment, the chain length of the linking monomer having a formula of

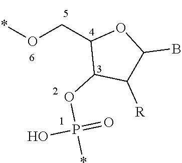

is 6. In one embodiment, the chain length of the linking monomer having a formula of

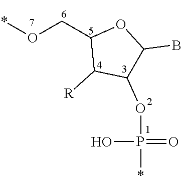

is 7. In one embodiment, the chain length of the linking monomer having a formula of

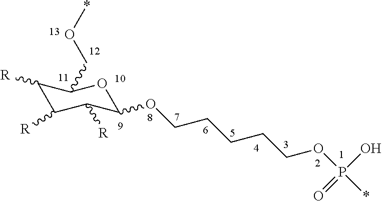

is 13.

In some embodiments, one or more linking moieties (N) in L of formula (I) may be an optionally modified nucleotide.

In some embodiments, one or more linking moieties (N) in L of formula (I) may be independently selected from the group consisting of a 2′-deoxynucleotide (dN), a 2′-deoxy-2′-fluoro nucleotide (fN), a ribonucleotide (rN), 2′-O-methylnucleotide (mN), and 2′-ara nucleotide (aN) (e.g., 2′-ara-2′-deoxy, 2′-ara-2′-F, 2′-ara-2′-OMe, or 2′-ara ribonucleotide). Ara-nucleotides feature an opposite stereochemistry at the 2′ carbon atom compared to ribo-nucleotides.

In certain embodiments, one or more linking moieties (N) in L of formula (I) may contain a modified internucleotide linkage selected from the group consisting of a phosphodiester, phosphotriester (optionally comprising the linkage phosphorus atom in either Rp configuration or Sp configuration), hydrogen phosphonate, alkyl or aryl phosphonate, phosphoramidate (optionally comprising the linkage phosphorus atom in either Rp configuration or Sp configuration), phosphorothioate (optionally comprising the linkage phosphorus atom in either Rp configuration or Sp configuration), methylenemethylimino, a nitrogen-modified phosphorous-containing linkage (PN-linkage) (optionally comprising the linkage phosphorus atom in either Rp configuration or Sp configuration), thiodiester, thionocarbamate, N,N′-dimethylhydrazine, phosphoroselenate, borano phosphate, borano phosphate ester, amide, hydroxylamino, siloxane, dialkylsiloxane, carboxamide, carbonate, carboxymethyl, carbamate, carboxylate ester, thioether, ethylene oxide linker, sulfide, sulfonate, sulfonamide, sulfonate ester, thioformacetal, formacetal, oxime, methyleneimino, methylenecarbonylamino, methylenehydrazo, methylenedimethylhydrazo, methyleneoxymethylimino, ether, thioacetamido, and combinations thereof.

In certain embodiments, one or more linking moieties (N) in L of formula (I) may contain a moiety selected from the group consisting of an aliphatic saturated or unsaturated alkyl chain; a phosphorous-containing linkage, including a phosphate, a phosphonate, a phosphoramidate (optionally comprising the linkage phosphorus atom in either Rp configuration or Sp configuration), phosphodiester, phosphotriester (optionally comprising the linkage phosphorus atom in either Rp configuration or Sp configuration), phosphorothioate (optionally comprising the linkage phosphorus atom in either Rp configuration or Sp configuration), and a nitrogen-modified phosphorous-containing linkage (PN-linkage) (optionally comprising the linkage phosphorus atom in either Rp configuration or Sp configuration); a (poly)ethylene glycol chain, including diethylene glycol, triethylene glycol, tetra, penta, hexa, hepta, octa, nona, or deca ethylene glycol; glycerol or glycerol ester; an aminoalkyl ether; and combinations thereof.

In some embodiments, one or more linking moieties (N) in L of formula (I) may contain a moiety selected from the group consisting of DNA, RNA, disulfide, amide, functionalized monosaccharides or oligosaccharides of galactosamine, glucosamine, glucose, galactose, mannose, and combinations thereof.

In some embodiments, one or more linking moieties (N) in L of formula (I) may be independently selected from the group consisting of:

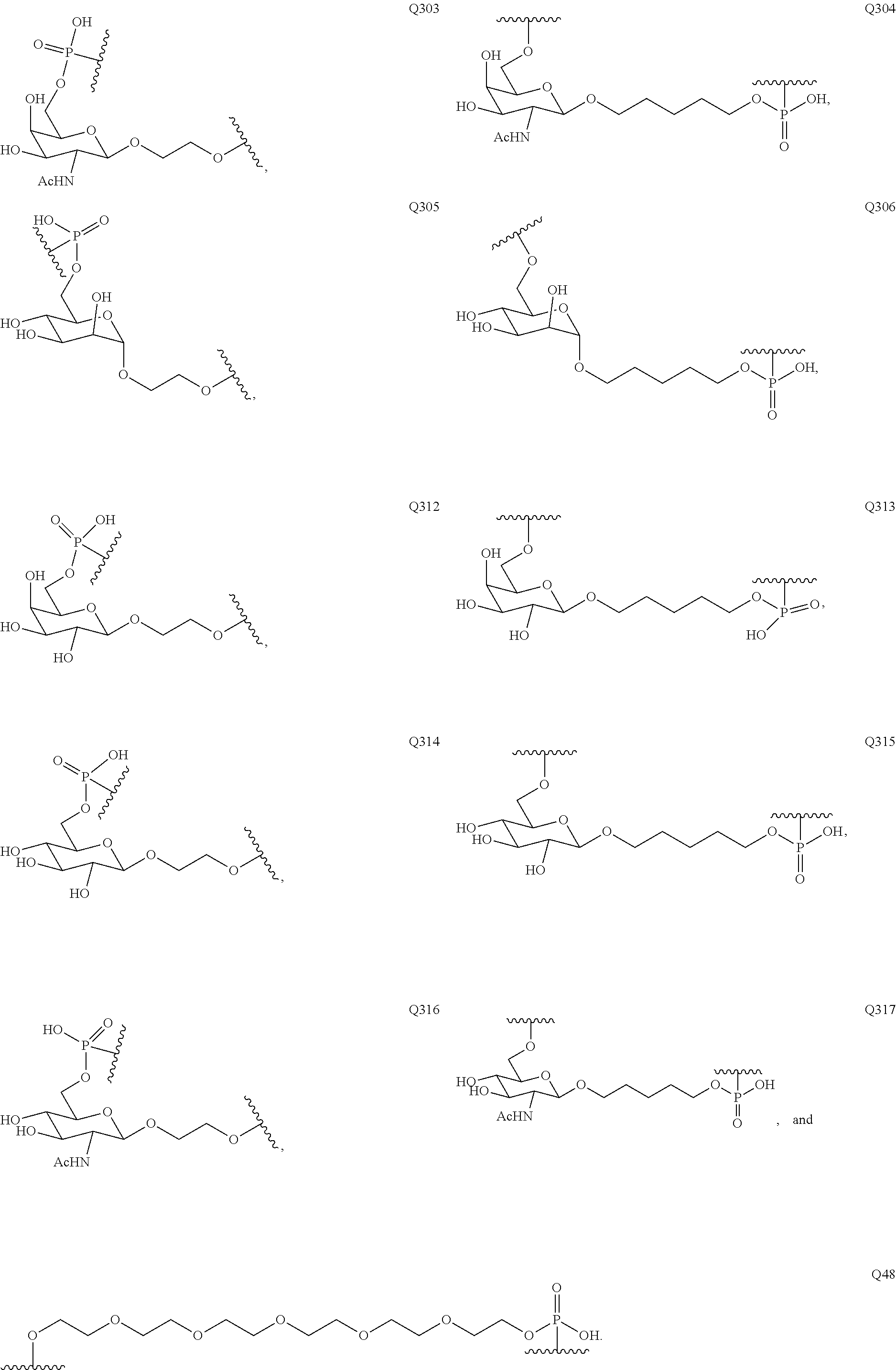

In some embodiments, one or more linking moieties (N) in L of formula (I) may be independently selected from the group consisting of:

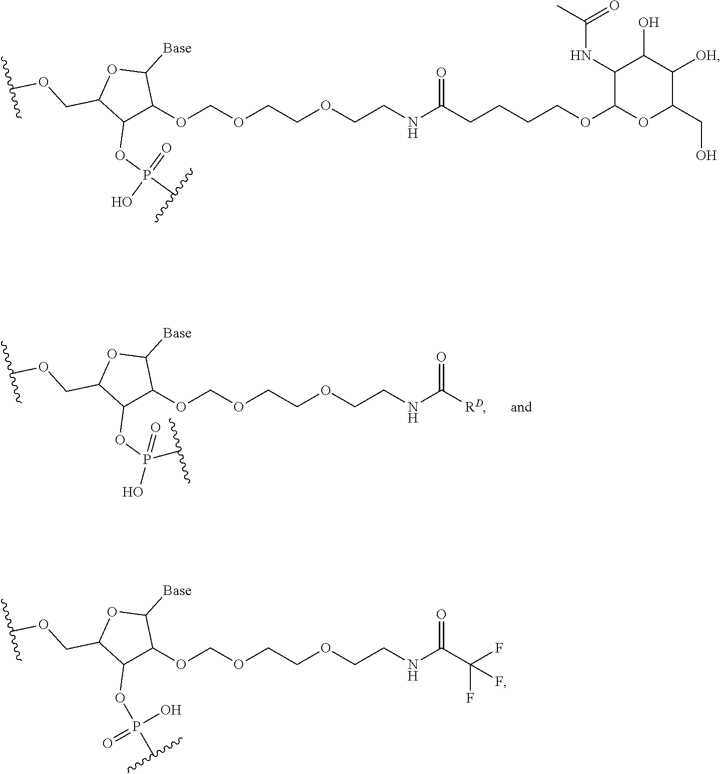

wherein:
Base is an optionally modified nucleobase, and
R^{D} is a C₄₋₃₀ alkyl, C₄₋₃₀ alkyenyl, or C₄₋₃₀ alkynyl.

In some embodiments, one or more linking moieties (N) in L of formula (I) comprise a mono-, di-, tri-, tetra-, penta- or poly-prolinol, optionally conjugated with a ligand; a mono-, di-, tri-, tetra-, penta- or poly-hydroxyprolinol, optionally conjugated with a ligand; an optionally modified nucleotide; or combinations thereof.

In some embodiments, L of formula (I) contains one or more of a mono-, di-, tri-, tetra-, penta- or poly-prolinol, optionally conjugated with a ligand; and one or more optionally modified nucleotides.

In some embodiments, L of formula (I) contains one or more of a mono-, di-, tri-, tetra-, penta- or poly-hydroxyprolinol, optionally conjugated with a ligand; and one or more optionally modified nucleotides.

In some embodiments, one or more linking moieties (N) in L of formula (I) comprises a moiety selected from the group consisting of:

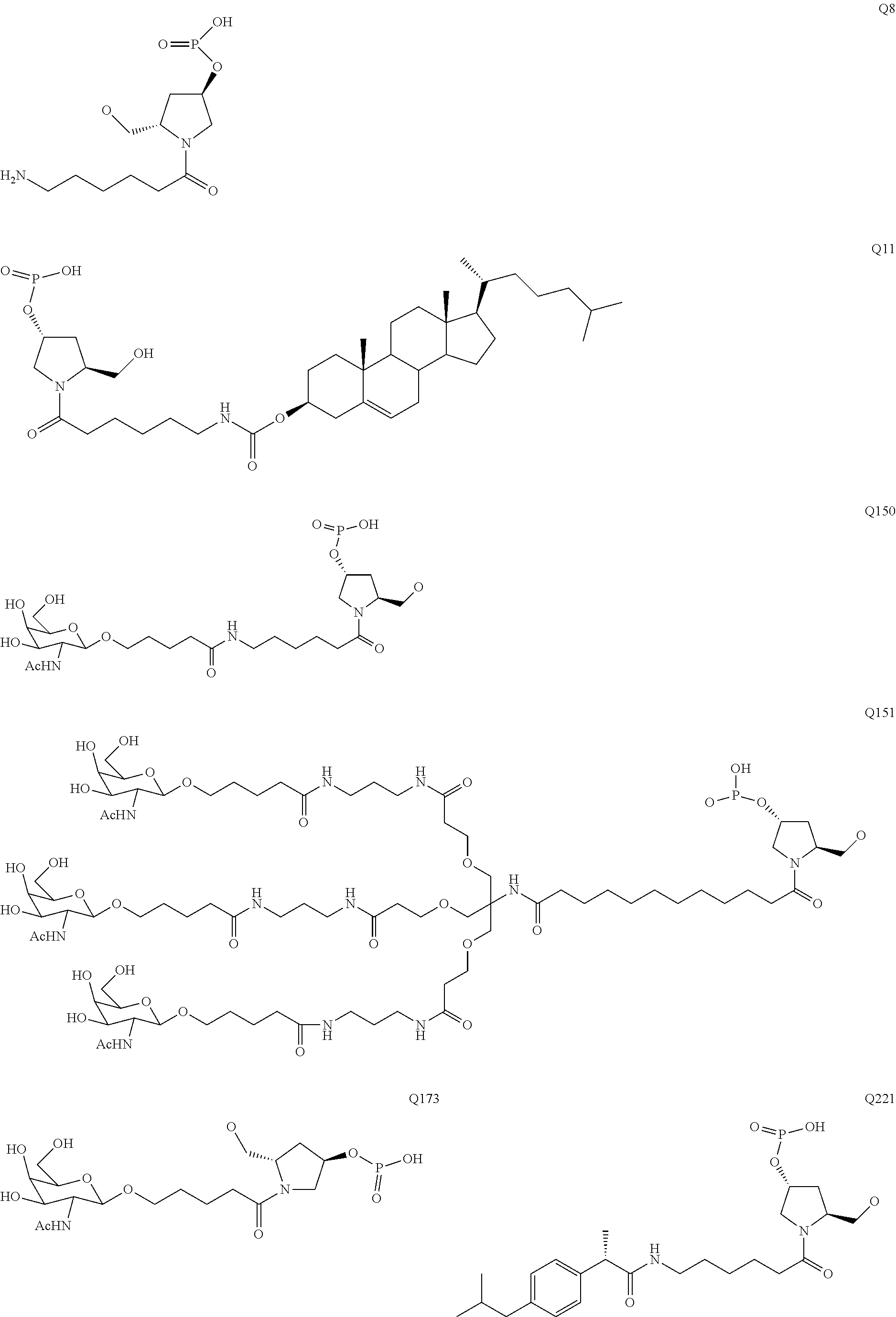

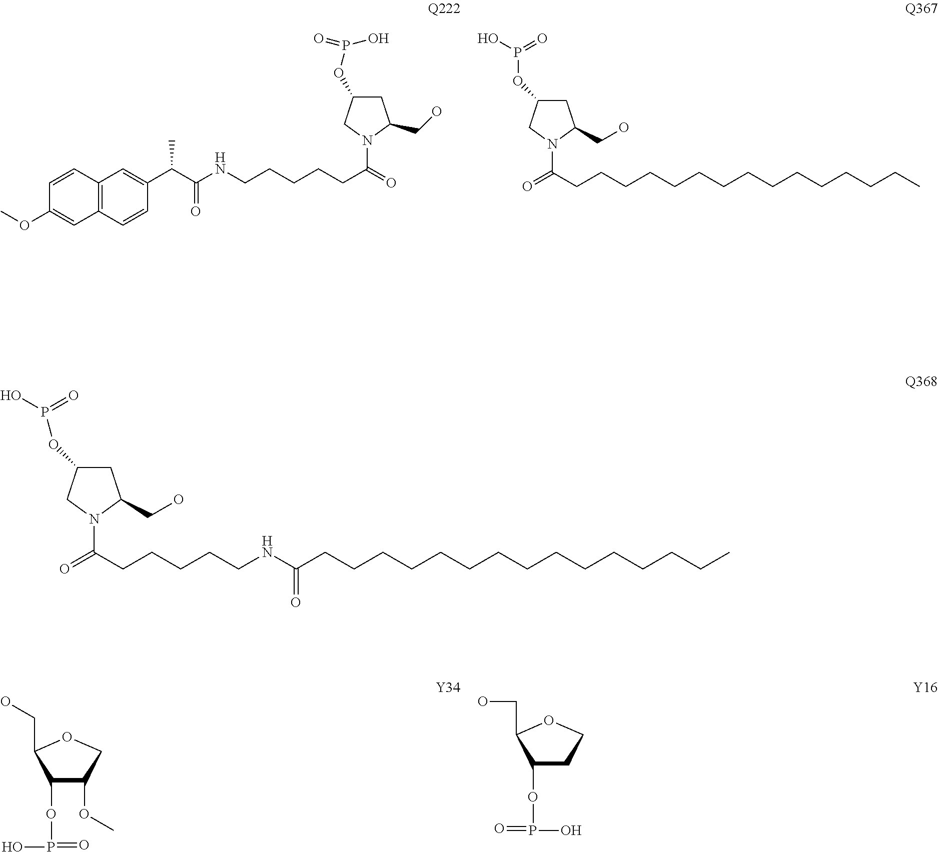

In some embodiments, L of formula (I) contains a linking moiety represented by a formula: #—(N)ₙ—**. In this formula, # is the bond to Q¹ and ** is the bond to Q²; n is 3 to 12; and each N is independently an optionally modified nucleotide, Y34, Y16, Q48, Q303, Q304, Q305, Q306, Q312, Q313, Q314, Q315, Q316, Q317, Q8, Q11, Q150, Q151, Q173, Q221, Q222, Q367, or Q368. In some embodiments, n is 3 to 8, 4 to 8, 3 to 7, 4 to 7, 3 to 6, 4 to 6, or 3 to 5. In one embodiment, n is 5.

In some embodiments, L of formula (I) contains 3-5 of 2′-deoxy nucleotides, a triplet of 2′-deoxy-2′-fluoro nucleotides, a triplet of ribonucleotides, a triplet of 2′-O-methyl nucleotides, or a triplet of Q304.

In some embodiments, L of formula (I) contains one of the followings:
#-mN-mN-mN-mN-mN-**,
#-rN-rN-rN-rN-rN-**,
#-rN-rN-fN-fN-fN-**,
#-dN-rN-rN-rN-dN-**,
#-mN-mN-dN-dN-dN-**,
#-mN-mN-rN-dN-dN-**,
#-mN-mN-rN-rN-rN-**, and
#-mN-mN-fN-fN-fN-**,
wherein:
dN represents a 2′-deoxy nucleotide,
fN represents a 2′-deoxy-2′-fluoro nucleotide,
rN represents a ribonucleotide, and
mN represents a 2′-O-methyl nucleotide.

In some embodiments, L of formula (I) contains one of the followings:
#---mN-mN-Q304-Q304-Q304---**
#---dN-dN-Q304-Q304-Q304---**
#---rN-rN-Q304-Q304-Q304---**
#---rN-dN-Q304-Q304-Q304---** and
#---dN-rN-Q304-Q304-Q304---**
wherein:
dN represents a 2′-deoxy nucleotide,
fN represents a 2′-deoxy-2′-fluoro nucleotide,
rN represents a ribonucleotide, and
mN represents a 2′-O-methyl nucleotide.

In the above embodiments, one or more internucleotide linkages between the nucleotides in L may be modified internucleotide linkages independently selected from the group consisting of a phosphodiester, phosphotriester (optionally comprising the linkage phosphorus atom in either Rp configuration or Sp configuration), hydrogen phosphonate, alkyl or aryl phosphonate, phosphoramidate (optionally comprising the linkage phosphorus atom in either Rp configuration or Sp configuration), phosphorothioate (optionally comprising the linkage phosphorus atom in either Rp configuration or Sp configuration), and a nitrogen-modified phosphorous-containing linkage (PN-linkage) (optionally comprising the linkage phosphorus atom in either Rp configuration or Sp configuration).

In certain embodiments, L of formula (I) may contain one or more linking moiety selected from the group consisting of a triazole linkage, an amide linkage, a sulfide or disulfide linkage, a phosphate linkage, an oxime linkage, a hydrazo linkage, a N,N′-dialkylenehydrazo linkage, a methyleneimino linkage, a methylenecarbonylamino linkage, a methylenemethylimino linkage, a methylenehydrazo linkage, a methylenedimethylhydrazo linkage, a methyleneoxymethylimino linkage, a hydroxylamino linkage, a formacetal linkage, an alkyl or aryl linkage, a PEG linkage, an ether linkage, a thioether linkage, a thiodiester linkage, a thionocarbamate linkage, a thioacetamido linkage, a sulfonate linkage, a sulfonamide linkage, a sulfonate ester linkage, a thioformacetal linkage, an urea linkage, a carbonate linkage, an amine linkage, a maleimide-thioether linkage, a phosphodiester linkage, a phosphotriester linkage, a hydrogen phosphonate linkage, an alkyl or aryl phosphonate linkage, a phosphoramidate linkage, a phosphorothioate linkage, a nitrogen-modified phosphorous-containing linkage (PN-linkage), a phosphoroselenate linkage, a borano phosphate linkage, a borano phosphate ester linkage, a sulfonamide linkage, a carbamate linkage, a carboxamide linkage, a carboxymethyl linkage, a carboxylate ester linkage, a siloxane linkage, a dialkylsiloxane linkage, an ethylene oxide linkage, and combinations thereof.

In certain embodiments, L of formula (I) may contain one or more cyclic groups selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuranyl, and decalinyl.

In some embodiments, L of formula (I) contains a nucleotide-based linker (tether). In some embodiments, L contains a non-nucleotide-based linker (tether).

In certain embodiments, the nucleotide-based or non-nucleotide-based linker (tether) contained in L is a stable linker (tether) that is stable in a biological fluid. For instance, the nucleotide-based or non-nucleotide based stable linker (tether) is stable in plasma or artificial cerebrospinal fluid.

In certain embodiments, the cleavable linking group (tether) comprises a moiety selected from the group consisting of

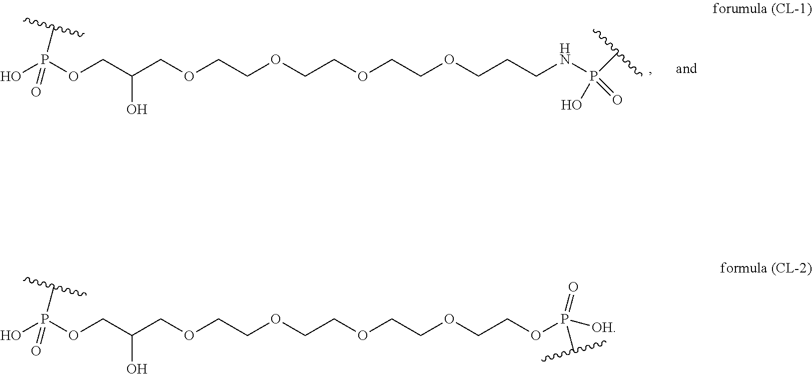 [SMILES: *P(=O)(O)NCCCOCCOCCOCCOCC(O)COP(*)(=O)O ; *P(=O)(O)OCCOCCOCCOCCOCC(O)COP(*)(=O)O]

In certain embodiments, the cleavable linking group (tether) comprises a moiety selected from the following:
—(CH₂)₁₂— (C12 linker or Q50),
—(CH₂)₆—S—S—(CH₂)₆— (C6-S—S—C6 linker or Q51),

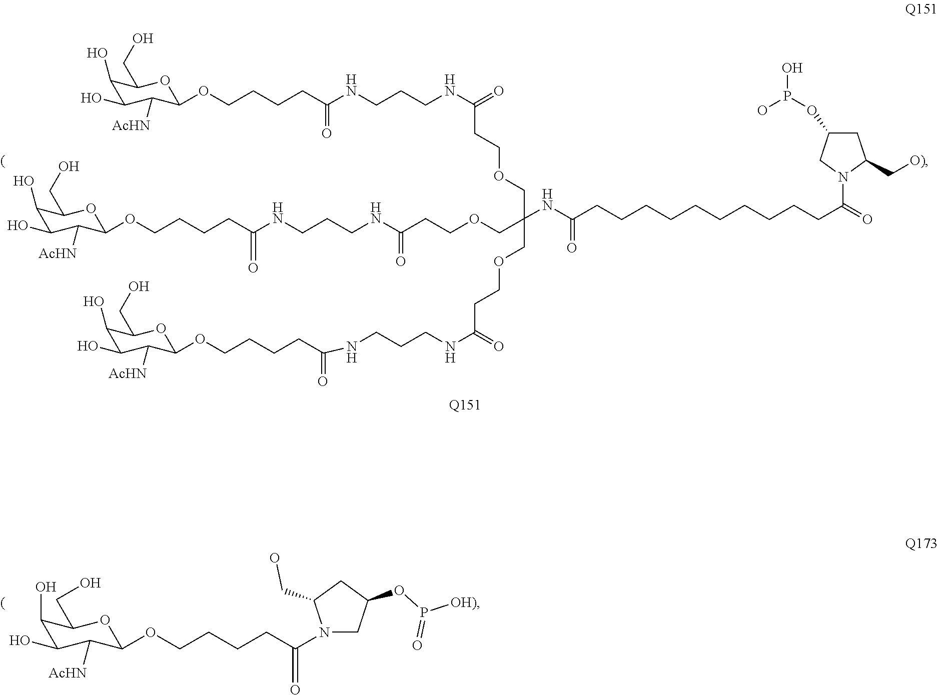

—CH₂CH₂O—(CH₂CH₂)ₙ—CH₂CH₂O—CH₂CH₂O—, wherein n is 0 or 1-20;
—(CH₂)₉(CH₂)ₙ—CH₂—, wherein n is 0 or 1-20;
mono-, di-, tri-, tetra-, penta- or polyprolinol, optionally conjugated with a ligand;
mono-, di-, tri-, tetra-, penta- or polyhydroxyprolinol, optionally conjugated with a ligand.

In certain embodiments, the cleavable linking group (tether) comprises a nucleic acid linker of 1 to 15 nucleotides in length. For instance, the nucleic acid linker may be 2 to 7, 5 to 7, 2 to 5, or 3, 4, or 5 optionally modified nucleotides in length.

In certain embodiments, the cleavable linking group (tether) comprises a nucleic acid linker comprising one or more nucleotides selected from the group consisting of 2′-O-methyl nucleotides, 2′-fluoro nucleotides, deoxyribonucleotides, and ribonucleotides. In one embodiment, all nucleic acid linker nucleotides are the same type of nucleotide. In one embodiment, the nucleic acid linker entirely comprises 2′-O-methyl nucleotides, entirely comprises 2′-fluoro nucleotides, or entirely comprises deoxyribonucleotides.

In certain embodiments, the cleavable linking group (tether) comprises a polynucleotide comprising a modified ribonucleotide sequence, optionally a polynucleotide comprising one or more modifications selected from the group consisting of a 2′-O-methyl ribonucleotide modification, a 2′-fluoro-ribonucleotide modification, a 2′-5′-linked nucleotide with different 3′-modification (3′-ribo, 3′-O-methyl, 3′-deoxy, 3′-fluoro), a glycol nucleic acid (GNA) modification, a locked nucleic acid (LNA) modification, a hexanol nucleic acid (HNA) modification, an abasic ribose modification, an abasic deoxyribose modification, and an abasic hydroxyprolinol modification.

In some embodiments, the linking group L in the single-stranded oligonucleotide of formula (I) comprises a nucleotide-based cleavable linking group (tether) that is cleavable by DICER. In some embodiments, the single-stranded oligonucleotide comprises a substrate cleavable by DICER.

In certain embodiments, the single-stranded oligonucleotide contains a cleavable linking group (nucleotide-based or non-nucleotide-based) capable of generating a metabolite of a 5′-monophosphate at at least one nucleotide sequence (e.g., Z¹ and/or Z²) of the single-stranded oligonucleotide.

In some embodiments, the single-stranded oligonucleotide may further comprise one or more ligands (e.g., targeting ligands). In one embodiment, Z¹ comprises at least one ligand (e.g., a targeting ligand), at the 5′ or 3′ end of the sequence. In one embodiment, Z² comprises at least one ligand (e.g., a targeting ligand), at the 5′ or 3′ end of the sequence. In one embodiment, each of Z¹ and Z² comprises at least one ligand (e.g., a targeting ligand), at the 5′ or 3′ end of the sequence.

In certain embodiments, at least one of the ligands is a lipophilic moiety.

In one embodiment, the lipophilic moiety is lipid, cholesterol, retinoic acid, cholic acid, adamantane acetic acid, 1-pyrene butyric acid, docosanoic acid (DCA), dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, lithocholic acid, O3-(oleoyl)lithocholic acid, O3-(oleoyl)cholenic acid, dimethoxytrityl, or phenoxazine. In certain embodiments, the lipid is a fatty acid (an omega-3 fatty acid, for example), selected from the group consisting of eicosapentaenoic acid (EPA) and docosahexaenoic acid (DHA).

In some embodiments, the lipophilic moiety contains a saturated or unsaturated C₄-C₃₀ hydrocarbon chain (e.g., C₄-C₃₀ alkyl or alkenyl), and an optional functional group selected from the group consisting of hydroxyl, amine, carboxylic acid, sulfonate, phosphate, thiol, azide, and alkyne.

In some embodiments, the lipophilic moiety contains a saturated or unsaturated C₆-C₁₈ hydrocarbon chain (e.g., a linear C₆-C₁₈ alkyl or alkenyl), e.g., a saturated or unsaturated C₁₆ hydrocarbon chain (e.g., a linear C₁₆ alkyl or alkenyl). In some embodiments, the lipophilic moiety contains a saturated or unsaturated C₁₄-C₂₄ hydrocarbon chain (e.g., a linear C₁₄-C₂₄ alkyl or alkenyl), e.g., a saturated or unsaturated C₂₂ hydrocarbon chain (e.g., a linear C₂₂ alkyl or alkenyl). For example, one or more non-terminal positions of the single-stranded oligonucleotide may have the following structure:

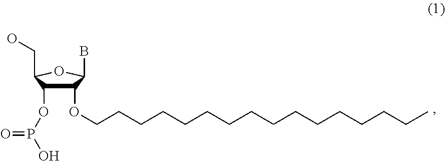

wherein B is a natural or modified nucleotide base (e.g., adenine, guanine, cytosine, thymine or uracil, or their modified derivatives), and the n-hexadecyl chain is the lipophilic moiety. The modification shown in formula (1) is referred to herein as “2′-C₁₆”. In another example, one or more non-terminal positions of the single-stranded oligonucleotide may have the following structure:

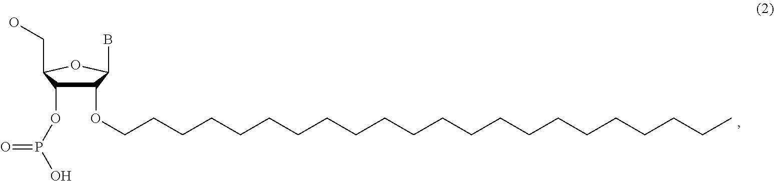

wherein B is a natural or modified nucleotide base (e.g., adenine, guanine, cytosine, thymine or uracil, or their modified derivatives), and the n-docosanyl chain is the lipophilic moiety. The modification shown in formula (2) is referred to herein as “2′-C₂₂”.

Similar modifications replacing the n-hexadecyl chain or the n-docosanyl chain with C₄-C₃₀ hydrocarbon chain is referred to as “2′-C₄-C₃₀ hydrocarbon chain” (or replacing with C₆-C₁₈ hydrocarbon chain or C₁₄-C₂₄ hydrocarbon chain is referred to as “2′-C₆-C₁₈ hydrocarbon chain” or “2′-C₁₄-C₂₄ hydrocarbon chain”).

In a related embodiment, one or more non-terminal nucleotide positions of at least one of Z¹ and Z² have the 2′-C₄-C₃₀ hydrocarbon chain structure, 2′-C₆-C₁₈ hydrocarbon chain structure, 2′-C₁₄-C₂₄ hydrocarbon chain structure, 2′-C₁₆ structure of formula (1), or 2′-C₂₂ structure of formula (2).

In one embodiment, one or more non-terminal nucleotide positions of both Z¹ and Z² have the 2′-C₄-C₃₀ hydrocarbon chain structure, 2′-C₆-C₁₈ hydrocarbon chain structure, 2′-C₁₄-C₂₄ hydrocarbon chain structure, 2′-C16 structure of formula (1), or 2′-C22 structure of formula (2).

In some embodiments, the lipophilic moiety contains one or more phospholipids.

In some embodiments, the lipophilic moiety contains one or more lipids or lipophilic ligands disclosed in International PCT Application Publication Nos. WO 2019/232255A1 and WO 2021/108662A1, and U.S. Pat. No. 10,184,124; all of which are herein incorporated by reference in their entirety.

In some embodiments, the ligands include one or more of the following formulas:

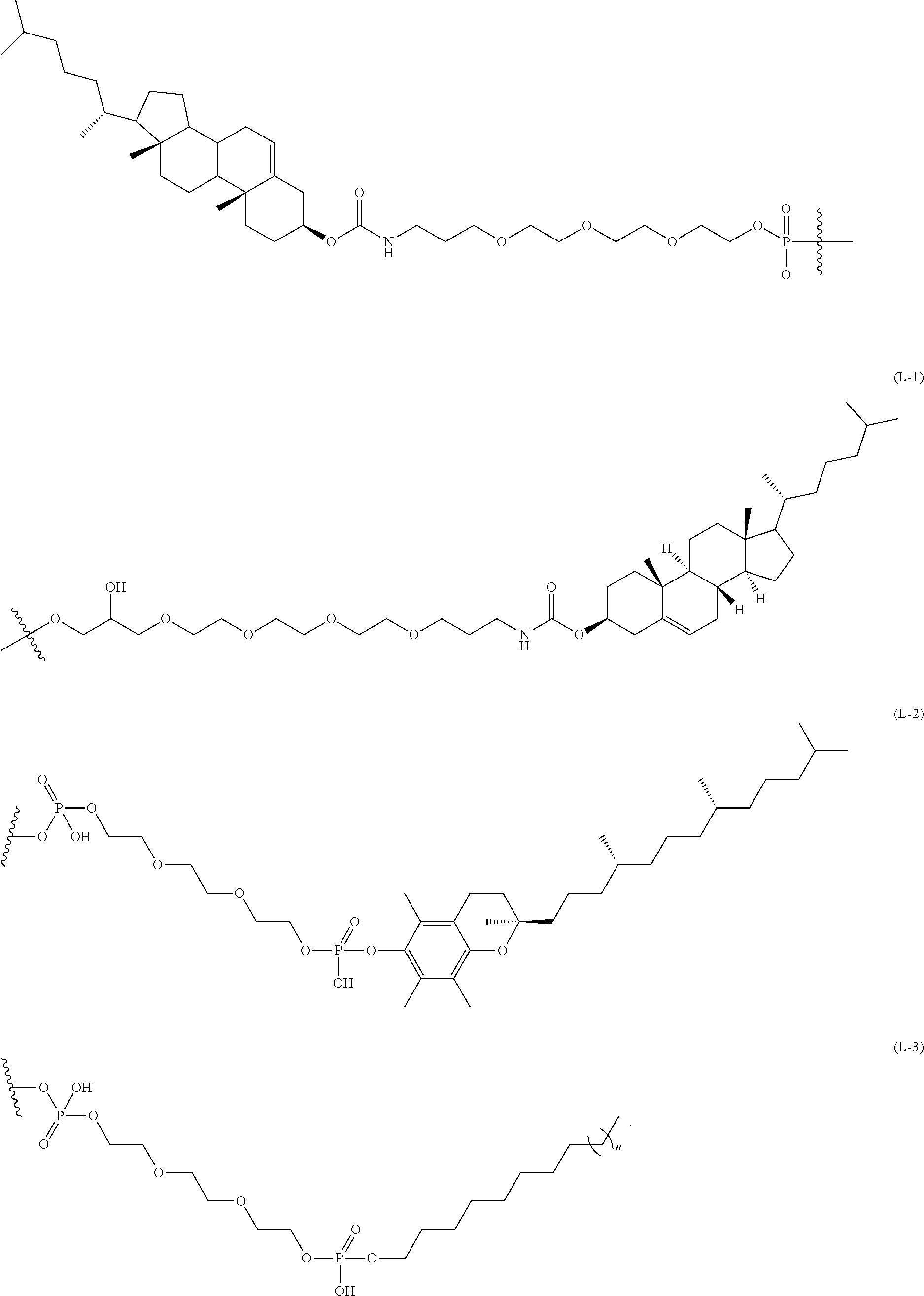

wherein n is 1 in “C10-TEG-”, and n is 7 in “C16-TEG-”.

In some embodiments, the ligands include those disclosed in International PCT Application Publication Nos. WO2017/053999, WO2019/118916, WO2022/031433, WO2022/056269, WO2022/056273, and WO2022/056277; all of which are herein incorporated by reference in their entirety.

In some embodiments, at least one of Z¹ and Z² comprises one or more lipophilic moieties conjugated independently to one or more of the internal positions (i.e., non-terminal positions) excluding positions 9-12 on a nucleotide sequence; for instance, positions 4-8 and 13-18 on a nucleotide sequence; positions 5, 6, 7, 15, and 17 on a nucleotide sequence; or positions 4, 6, 7, and 8 on a nucleotide sequence, each counting from the 5′-end of the nucleotide sequence as position 1.

In some embodiments, at least one of Z¹ and Z² comprises one or more lipophilic moieties conjugated independently to position 6 of the nucleotide sequence, counting from the 5′-end of the nucleotide sequence. In one embodiment, each of Z¹ and Z² comprises a lipophilic moiety conjugated to position 6 of the nucleotide sequence; optionally the lipophilic moiety comprises a saturated or unsaturated C₆-C₁₈ hydrocarbon chain, or a saturated or unsaturated C₁₄-C₂₄ hydrocarbon chain; optionally the lipophilic moiety comprises a saturated or unsaturated C₁₆ hydrocarbon chain or a saturated or unsaturated C₂₂ hydrocarbon chain.

In some embodiments, at least one of Z¹ and Z² comprises one or more lipophilic moieties conjugated independently to one or more of internal positions (i.e., non-terminal positions) on a nucleotide sequence; for instance, positions 6-10 and 15-18 on a nucleotide sequence; and positions 15 and 17 on a nucleotide sequence, each counting from the 5′-end of the nucleotide sequence as position 1.

In some embodiments, at least one of the ligands is a targeting ligand selected from the group consisting of an antibody, antigen, folate, receptor ligand, carbohydrate, aptamer, integrin receptor ligand, chemokine receptor ligand, transferrin, biotin, serotonin receptor ligand, PSMA, endothelin, GCPII, somatostatin, LDL and HDL ligand. In one embodiment, at least one of the ligands is an integrin receptor ligand.

The targeting ligand may be conjugated to an internal position of a nucleotide sequence (e.g., Z¹¹ and Z¹²), optionally via a linker or carrier. Alternatively, the targeting ligand may be conjugated to the 3′-end or 5′-end of Z¹¹ or Z¹², optionally via a linker or carrier.

In certain embodiments, at least one of the ligands is a carbohydrate-based ligand. The carbohydrate-based ligand may be D-galactose, multivalent galactose, N-acetyl-D-galactosamine (GalNAc), multivalent GalNAc, D-mannose, multivalent mannose, multivalent lactose, N-acetyl-glucosamine, glucose, multivalent glucose, multivalent fucose, glycosylated polyaminoacids, or lectins.

In certain embodiments, the carbohydrate-based ligand is an ASGPR ligand. For example, the ASGPR ligand is one or more GalNAc derivatives attached through a bivalent or trivalent branched linker, such as:

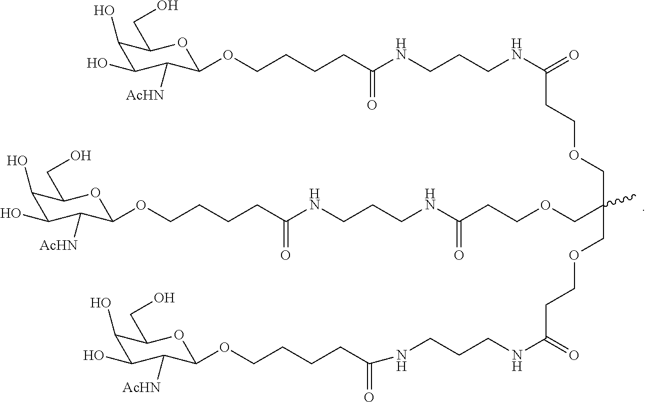 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In certain embodiments, at least one of the ligands may be conjugated at the 3′-end, 5′-end, or an internal position of a nucleotide sequence (e.g., Z¹ and Z²).

In some embodiments, at least one of the ligands may be conjugated to the single-stranded oligonucleotide via a direct attachment to the ribosugar of the oligonucleotide. Alternatively, the ligand may be conjugated to the single-stranded oligonucleotide via one or more linkers (tethers), and/or a carrier.

In some embodiments, the ligand may be conjugated to the single-stranded oligonucleotide via a monovalent or branched bivalent or trivalent linker.

In some embodiments, the ligand may be conjugated to the single-stranded oligonucleotide via a carrier that replaces one or more nucleotide(s). The carrier can be a cyclic group or an acyclic group. In one embodiment, the cyclic group is selected from the group consisting of cyclohexyl, pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuranyl, and decalinyl. In one embodiment, the acyclic group is a moiety based on a serinol backbone or a diethanolamine backbone.

In some embodiments, the single-stranded oligonucleotide may be characterized by one or more of:
(a) Z¹ and Z² each independently contain 19-23 optionally modified nucleotides;
(b) Q¹ and Q² each independently contain 0 to 2 optionally modified nucleotides;
(c) the duplexed region formed by Z¹ and Z² contains no more than 3 mismatched base pairs;
(d) the duplexed region formed by Z¹ and Z² forms a blunt end;
(e) at least one nucleotide in Z² is a modified nucleotide;
(f) at least one nucleotide in Z¹ is a modified nucleotide;
(g) Z² comprises at least one modified internucleotide linkage;
(h) Z¹ comprises at least one modified internucleotide linkage;
(i) the 5′-terminal nucleotide comprises a 5′-phosphate or 5′-phosphate mimic modification;
(j) the 3′-terminal nucleotide is conjugated to a ligand, optionally through a linker;
(k) Z¹ contains no more than 3 mismatches to the target gene;
(l) Z¹ and Z² each independently contain 19-23 optionally modified nucleotides; and
(m) L contains a linking moiety represented by a formula: #—(N)ₙ—**, wherein n is 3 to 5; and each N is independently an optionally modified nucleotide, Y16, Y34, Q48, Q303, Q304, Q305, Q306, Q312, Q313, Q314, Q315, Q316, Q317, Q8, Q11, Q150, Q151, Q173, Q221, Q222, Q367, or Q368.

In some embodiments, the single-stranded oligonucleotide may be characterized by two or more, three or more, four or more, five or more, six or more, seven or more, eight or more, nine or more, ten or more, eleven or more, twelve or more, or all of the above features.

In some embodiments, the single-stranded oligonucleotide may be characterized by one or more of:
(a) Z¹ and Z² each independently contain 21 optionally modified nucleotides;
(b) Q¹ and Q² each independently contain 2 optionally modified nucleotides;
(c) the duplexed region formed by Z¹ and Z² contains no more than 3 mismatched base pairs;
(d) the duplexed region formed by Z¹ and Z² forms a blunt end;
(e) all the nucleotides in Z² are modified nucleotides;
(f) all the nucleotides in Z¹ are modified nucleotides;
(g) Z² comprises at least two consecutive modified internucleotide linkages;
(h) Z¹ comprises at least two consecutive modified internucleotide linkages;
(i) the 5′-terminal nucleotide of Z¹ comprises a 5′-phosphate or 5′-phosphate mimic modification;
(j) the 3′-terminal nucleotide of Z² is conjugated to a ligand, optionally through a linker;
(k) Z¹ contains no more than 3 mismatches to the target gene; and
(l) L contains a linking moiety represented by a formula: #—(N)ₙ—**, wherein n is 5; and each N is independently an optionally modified nucleotide, or Q304.

In some embodiments, the single-stranded oligonucleotide may be characterized by two or more, three or more, four or more, five or more, six or more, seven or more, eight or more, nine or more, ten or more, eleven or more, twelve or more, or all of the above features.

Another aspect of the invention relates to a single-stranded oligonucleotide according to formula (II) or (III):

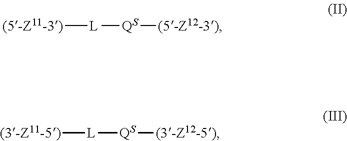

wherein:
Z¹¹ is a first oligonucleotide, comprising 15-100 optionally modified nucleotides that is substantially complementary to a target gene;
Z¹² is a second oligonucleotide, comprising 10-100 optionally modified nucleotides that is substantially complementary to Z¹¹;
Z¹¹ and Z¹² are capable of forming an intra-strand duplexed region comprising 7 or more consecutive base pairs;
Q^{S} represents 0 to 12 optionally modified nucleotides;
L is an optional linking group;
at least one nucleotide in formula (II) is a modified nucleotide; and
at least one nucleotide in formula (III) is a modified nucleotide, wherein
   at least one nucleotide at the 3′ end of Z¹¹, for formula (II), at least one nucleotide at the 5′ end of Z¹¹, for formula (III), in either case together with L and
Q^{S} form a loop region connecting Z¹¹ and Z¹².

In some embodiments, the single-stranded oligonucleotide may be an inhibitory single-stranded oligonucleotide, such as an antisense oligonucleotide (ASO), an antimiR (antagomir) oligonucleotide, microRNA mimic, supermir, aptamer, U1 adaptor, triplex-forming oligonucleotide, RNA activator, immuno-stimulatory oligonucleotide, decoy oligonucleotide, heteroduplex-forming oligonucleotide, or a single-stranded siRNA (ss-siRNA) oligonucleotide.

The first oligonucleotide Z¹¹ and second oligonucleotide Z¹² each may independently comprise 10-100 optionally modified nucleotides. For instance, Z¹¹ and Z¹² each may independently comprise 10-40, 10-30, 12-26, 12-23, 12-21, 15-26, 15-23, 15-21, 19-26, 19-23, or 19-21 optionally modified nucleotides. In some embodiments, the first oligonucleotide Z¹¹ and second oligonucleotide Z¹² each may independently comprise at least 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. Z¹¹ and Z¹² each may independently have about 10 to about 50 nucleotides, about 10 to about 40 nucleotides, about 10 to about 35 nucleotides, about 10 to about 30 nucleotides, about 10 to about 26 nucleotides, about 10 to about 23 nucleotides, about 10 to about 21 nucleotides, about 12 to about 50 nucleotides, about 12 to about 40 nucleotides, about 12 to about 35 nucleotides, about 12 to about 30 nucleotides, about 12 to about 26 nucleotides, about 12 to about 23 nucleotides, about 12 to about 21 nucleotides, about 15 to about 50 nucleotides, about 15 to about 40 nucleotides, about 15 to about 35 nucleotides, about 15 to about 30 nucleotides, about 15 to about 26 nucleotides, about 15 to about 23 nucleotides, about 15 to about 21 nucleotides, about 19 to about 50 nucleotides, about 19 to about 40 nucleotides, about 19 to about 35 nucleotides, about 19 to about 30 nucleotides, about 19 to about 26 nucleotides, about 19 to about 23 nucleotides, about 19 to about 21 nucleotides, or about 18 to about 20 nucleotides in length.

In some embodiments, Z¹¹ and Z¹² each independently comprise 10-40 optionally modified nucleotides. In some embodiments, Z¹¹ and Z¹² each independently comprise 12-26 optionally modified nucleotides.

In some embodiments, Z¹¹ and Z¹² each contain the same number of optionally modified nucleotides. In some embodiments, Z¹¹ contain a larger number of optionally modified nucleotides than Z¹². In some embodiments, Z¹¹ comprises 19-26 optionally modified nucleotides, and Z¹² comprises 12-21 optionally modified nucleotides.

In some embodiments, the single-stranded oligonucleotide can be cleaved at the linking group L. The first oligonucleotide Z¹¹ can be cleaved into an antisense strand that is substantially complementary to a target gene (e.g., a target mRNA or DNA), and the second oligonucleotide Z¹² can be cleaved into a sense strand that is substantially complementary to Z¹¹.

Q^{S} may comprise 0 to 12 optionally modified nucleotides. For instance, Q^{S} may comprise 0 to 10, 0 to 6, 0 to 4, 0 to 3, 0 to 2, 1 to 6, 1 to 4, 1 to 3, 1 to 2, or 2 to 3 optionally modified nucleotides. In some embodiments, Q^{S} is 0. In some embodiments, Q^{S} is 1 to 6 optionally modified nucleotides. In some embodiments, Q^{S} is 2 optionally modified nucleotides. In some embodiments, Q^{S} is 1 optionally modified nucleotide.

In some embodiments, one or more nucleotides of Q^{S} form a mismatched base pair with the opposite nucleotide in Z¹¹. In some embodiments, Q^{S} is 2 optionally modified nucleotides, and is characterized by one of the followings:
both nucleotides of Q^{S} form mismatched base pairs with their opposite nucleotides in Z¹¹,
one nucleotide of Q^{S} forms a mismatched base pair with the opposite nucleotide in Z¹¹ (e.g., the nucleotide of Q^{S} next to Z¹² forms a mismatched base pair with the opposite nucleotide in Z¹¹), or
both nucleotides of Q^{S} form base pairs with their opposite nucleotides in Z¹¹.

The first oligonucleotide Z¹¹ is substantially complementary to a target gene, i.e., Z¹¹ contains no more than 3 (e.g., 0, 1, 2, or 3) mismatches to the target gene. In some embodiments, the target gene may be a mRNA, pre-mRNA, microRNA, pre-miRNA, long non-coding RNA (lncRNA), or DNA.

The first oligonucleotide Z¹¹ and the second oligonucleotide Z¹² are capable of forming an intra-strand duplexed region, e.g., comprising 7 or more consecutive base pairs. In some embodiments, Z¹¹ and Z¹² are capable of forming an intra-strand duplexed region comprising 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs. In some embodiments, Z¹¹ and Z¹² are capable of forming an intra-strand duplexed region having base pairs with all the nucleotides of Z¹². The intra-strand duplexed region formed by Z¹¹ and Z¹² may contain all consecutive base pairs, or may contain up to 3 mismatch based pairs (e.g., 0, 1, 2, or 3). In some embodiments, the intra-strand duplexed region formed by Z¹¹ and Z¹² contain 1 mismatch based pair.

In some embodiments, the first oligonucleotide Z¹¹ and the second oligonucleotide Z¹² are capable of forming an intra-strand duplexed region at the seed region of Z¹¹ (e.g., the seed region of an antisense strand; e.g., at positions 2-8 of the 5′-end of the antisense strand).

In some embodiments, the first oligonucleotide Z¹¹ contains a loop at the 3′-end or 5′-end. In some embodiments, the first oligonucleotide Z¹¹ comprises W-LP, wherein W is capable of forming an intra-strand duplexed region of at least 7 base pairs with Z¹², and LP, optionally together with L, forms the loop between W and Z¹² at the 3′-end or 5′-end. In some embodiments, W and Z¹² are capable of forming an intra-strand duplexed region comprising 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs. In some embodiments, W and Z¹² are capable of forming an intra-strand duplexed region having base pairs with all the nucleotides of Z². The intra-strand duplexed region formed by W and Z¹² may contain all consecutive base pairs, or may contain no more than 3 (e.g., 0, 1, 2, or 3) mismatch based pairs.

In some embodiments, the single-stranded oligonucleotide is represented by formula (IIa) or formula (IIIa):

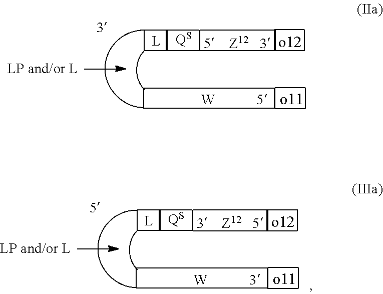

wherein:
Z¹¹ comprises W-LP,
W forms an intra-strand duplexed region at least 7 base pairs with Z¹²,
LP, optionally together with L, forms a loop between W and Z¹² at the 3′-end or 5′-end,
[symbol] represents an optional presence of L,
[symbol] represents an optional presence of Q^{S},
[symbol] represents an optional overhang at 5′-end or 3′-end of Z¹¹, and
[symbol] represents an optional overhang at 5′-end or 3′-end of Z¹².

In some embodiments, the duplexed region formed by Z¹¹ and Z¹² at the non-loop terminal has a blunt end. In some embodiments, the duplexed region formed by W and Z¹² at the non-loop terminal has a blunt end.

In some embodiments, Z¹¹ at the non-loop terminal has an overhang of 1-3 nucleotides in length. In some embodiments, W at the non-loop terminal has an overhang of 1-3 nucleotides in length. In some embodiments, [symbol] is present and is 1-3 nucleotides in length.

In some embodiments, Z¹² has an overhang of 1-3 nucleotides in length. In some embodiments, [symbol] is present and is 1-3 nucleotides in length.

In some embodiments, the overhang is 1 nucleotide in length. In some embodiments, the overhang is 2 nucleotides in length. In some embodiments, the overhang is 3 nucleotides in length.

Each of the nucleotides in the single-stranded oligonucleotide may be independently and optionally modified. Each of the nucleotides in first oligonucleotide Z¹¹ and second oligonucleotide Z¹² may be independently and optionally modified.

In some embodiments, the single-stranded oligonucleotide comprises at least one chemical modification. In some embodiments, each of the first oligonucleotide Z¹¹ and second oligonucleotide Z¹² comprise at least one chemical modification. In some embodiments, W comprises at least one chemical modification. In some embodiments, all the nucleotides in Z¹¹ are modified nucleotides. In some embodiments, all the nucleotides in W are modified nucleotides. In some embodiments, all the nucleotides in Z¹² are modified nucleotides. In some embodiments, all the nucleotides of the single-stranded oligonucleotide are modified.

The chemical modification to the nucleotide(s) may include an internucleoside linkage modification, a nucleobase modification, a sugar modification, or combinations thereof.

In certain embodiments, the chemical modification is selected from the group consisting of LNA, ENA, HNA, CeNA, 2′-O-methoxyalkyl (e.g., 2′-O-methoxymethyl, 2′-O-methoxyethyl, or 2′-O-2-methoxypropanyl), 2′-O-alkyl, 2′-O-allyl, 2′-C-allyl, 2′-fluoro, 2′-deoxy, 2′-O—N-methylacetamido (2′-O-NMA), 2′-O-dimethylaminoethoxyethyl (2′-O-DMAEOE), 2′-O-aminopropyl (2′-O-AP), 2′-ara-F, L-nucleoside modification (such as 2′-modified L-nucleoside, e.g., 2′-deoxy-L-nucleoside), BNA abasic sugar, abasic cyclic and open-chain alkyl, and combinations thereof.

In certain embodiments, the chemical modification is selected from the group consisting of at least one of the modified nucleotides is a deoxy-nucleotide, a 3′-terminal deoxythimidine (dT) nucleotide, a 2′-O-methyl modified nucleotide, a 2′-fluoro modified nucleotide, a 2′-deoxy-modified nucleotide, a locked nucleotide (LNA), an unlocked nucleotide (UNA), a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxy-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a 5′-phosphorothioate group, a nucleotide comprising a 5′-methylphosphonate group, a nucleotide comprising a 5′ phosphate or 5′ phosphate mimic, a nucleotide comprising vinyl phosphonate, a nucleotide comprising glycol nucleic acid (GNA), a nucleotide comprising glycol nucleic acid (GNA) S-Isomer (S-GNA), a nucleotide comprising 2-hydroxymethyl-tetrahydrofuran-5-phosphate, a nucleotide comprising 2′-deoxythymidine-3′phosphate, a nucleotide comprising 2′-deoxyguanosine-3′-phosphate, a 2′-5′-linked nucleotide (“3′-RNA”), or a terminal nucleotide linked to a cholesteryl derivative or a dodecanoic acid bisdecylamide group.

In certain embodiments, the chemical modification is a 2′-modification selected from the group consisting of 2′-O-methyl, 2′-O-allyl, 2′-O-methoxyalkyl (e.g., 2′-O-methoxymethyl, 2′-O-methoxyethyl, or 2′-O-2-methoxypropanyl), 2′-deoxy, 2′-fluoro, and combinations thereof.

In some embodiments, about 100%, 95%, 90%, 85%, 80%, 75%, 70%, 65%, 60%, 55%, 50%, 45%, 40%, 35% or 30% of Z¹¹ are modified. In some embodiments, about 100%, 95%, 90%, 85%, 80%, 75%, 70%, 65%, 60%, 55%, 50%, 45%, 40%, 35% or 30% of Z¹² are modified. In some embodiments, about 100%, 95%, 90%, 85%, 80%, 75%, 70%, 65%, 60%, 55%, 50%, 45%, 40%, 35% or 30% of all the nucleotides in the single-stranded oligonucleotide are modified. For example, when 50% of all the nucleotides are modified, 50% of all nucleotides present in the single-stranded oligonucleotide contain at least one modification as described herein.

In one embodiment, at least 50% of the nucleotides of the single-stranded oligonucleotide are independently modified with 2′-O-methyl, 2′-O-allyl, 2′-O-methoxyalkyl (e.g., 2′-O-methoxymethyl, 2′-O-methoxyethyl, or 2′-O-2-methoxypropanyl), 2′-deoxy, or 2′-fluoro.

In some embodiments, the single-stranded oligonucleotide of formula (II) (or IIa) or formula (III) (or (IIIa) further comprises one or more of the following internucleotide linkage modifications;
(i) one or more internucleotide linkages among the six 3′-terminal nucleotides is a modified internucleotide linkage; and
(ii) one or more internucleotide linkages among the six 5′-terminal nucleotides is a modified internucleotide linkage.

In some embodiments, the single-stranded oligonucleotide of formula (II) (or IIa) or formula (III) (or (IIIa) further comprises one or more internucleotide linkages among the eight 3′-terminal nucleotides of Z¹¹ for formula (II), or one or more internucleotide linkages among the eight 5′-terminal nucleotides of Z¹¹ for formula (III), is a modified internucleotide linkage.

In some embodiments, the single-stranded oligonucleotide of formula (II) (or IIa) or formula (III) (or (IIIa) further comprises one or more of the following internucleotide linkage modifications:
(i) two consecutive internucleotide linkages among the six 3′-terminal nucleotides are modified internucleotide linkages; and
(ii) two consecutive internucleotide linkages among the six 5′-terminal nucleotides are modified internucleotide linkages.

In some embodiments, when the single-stranded oligonucleotide contains a terminal conjugation of a ligand to the 5′-end or 3′-end nucleotide, or contains a terminal conjugation of an abasic nucleotide, an inverted nucleotide, or an inverted abasic nucleotide to the 5′-end or 3′-end nucleotide, then at that terminus, the above internucleotide linkage modifications to the terminal nucleotide can be omitted.

In some embodiments, the single-stranded oligonucleotide of formula (II) (or IIa) or formula (III) (or (IIIa) further comprises one of the following internucleotide linkage modifications:
(i) one or more internucleotide linkages among the eight 3′-terminal nucleotides of Z¹¹ for formula (II) (or IIa), or one or more internucleotide linkages among the eight 5′-terminal nucleotides of Z¹¹ for formula (III) (or (IIIa)), is a modified internucleotide linkage;
(ii) one or more internucleotide linkages among the six 5′-terminal nucleotides of Z¹¹ for formula (II) (or IIa), or one or more internucleotide linkages among the six 3′-terminal nucleotides of Z¹¹ for formula (III) (or (IIIa)), is a modified internucleotide linkage;
(iii) one or more internucleotide linkages among the six 5′-terminal nucleotides of Z² for formula (II) (or (IIa)), or one or more internucleotide linkages among the six 3′-terminal nucleotides of Z¹² for formula (III) (or (IIIa)), is a modified internucleotide linkage; and
(iv) one or more internucleotide linkages among the six 3′-terminal nucleotides of Z² for formula (II) (or (IIa)), or one or more internucleotide linkages among the six 5′-terminal nucleotides of Z¹² for formula (III) (or (IIIa)), is a modified internucleotide linkage.

In some embodiments, the single-stranded oligonucleotide of formula (II) (or IIa) or formula (III) (or (IIIa)) further comprises one or more of the following internucleotide linkage modifications:
(i) two consecutive internucleotide linkages among the three 5′-terminal nucleotides of Z² for formula (II) (or (IIa)), or two consecutive internucleotide linkages among the three 3′-terminal nucleotides of Z² for formula (III) (or (IIIa)), are modified internucleotide linkages;
(ii) two consecutive internucleotide linkages among the three 3′-terminal nucleotides of Z² for formula (II) (or (IIa)), or two consecutive internucleotide linkages among the three 5′-terminal nucleotides of Z² for formula (III) (or (IIIa)), are modified internucleotide linkages; and
(iii) two consecutive internucleotide linkages among the three or four 5′-terminal nucleotides of Z¹¹ for formula (II) (or (IIa)), or two consecutive internucleotide linkages among the three or four 3′-terminal nucleotides of Z¹¹ for formula (III) (or (IIIa)), are modified internucleotide linkages.

In some embodiments, the single-stranded oligonucleotide of formula (II) (or IIa) further comprises one or more modified internucleotide linkage between the 5′-end nucleotide of Z² and the first nucleotide of Q^{S}. In some embodiments, the single-stranded oligonucleotide of formula (III) (or (IIIa)) further comprises one or more modified internucleotide linkage between the 3′-end nucleotide of Z² and the first nucleotide of Q^{S}. In some embodiments, the single-stranded oligonucleotide of formula (II) (or IIa) or formula (III) (or (IIIa)) further comprises one or more modified internucleotide linkages between the nucleotides of Q^{S}.

In some embodiments, in all above embodiments, the modified internucleotide linkage is phosphorothioate linkage.

In some embodiments, the single-stranded oligonucleotide of formula (II) (or IIa) or formula (III) (or (IIIa) comprises at least two consecutive phosphorothioate or methylphosphonate internucleotide linkage modifications. In some embodiments, the single-stranded oligonucleotide comprises at least two blocks of two consecutive phosphorothioate or methylphosphonate internucleotide linkage modifications. In some embodiments, the single-stranded oligonucleotide comprises at least three blocks of two consecutive phosphorothioate or methylphosphonate internucleotide linkage modifications.

In some embodiments, the single-stranded oligonucleotide of formula (II) (or IIa) or formula (III) (or (IIIa) has at least two phosphorothioate internucleotide linkages among the first six nucleotides on a nucleotide sequence (e.g., Z¹¹ and/or Z¹²).

In some embodiments, a nucleotide sequence of the single-stranded oligonucleotide (e.g., Z¹¹ and/or Z²) comprises two blocks of one, two, or three phosphorothioate internucleotide linkages separated by 1, 2, 3, 4, 5, 6, 7, 8, 9, 10, 11, 12, 13, 14, 15, 16, 17, or 18 phosphate internucleotide linkages.

In one embodiment, a nucleotide sequence of the single-stranded oligonucleotide of formula (II) (or IIa) or formula (III) (or (IIIa) (e.g., Z¹¹ and/or Z¹²) comprises at least two consecutive phosphorothioate internucleotide linkage modifications within positions 18-23 of the nucleotide sequence, counting from the 5′-end of the nucleotide sequence. In one embodiment, a nucleotide sequence of the single-stranded oligonucleotide (e.g., Z¹¹ and/or Z¹²) comprises at least two consecutive phosphorothioate internucleotide linkage modifications within position 1-5 of the nucleotide sequence, counting from the 5′-end of the nucleotide sequence.

In some embodiments, each of Z¹¹ and Z¹² of the single-stranded oligonucleotide comprises at least two consecutive phosphorothioate internucleotide linkage modifications. In one embodiment, each of Z¹¹ and Z¹² of the single-stranded oligonucleotide comprises: at least two consecutive phosphorothioate internucleotide linkage modifications within positions 18-23 of the nucleotide sequence, and at least two consecutive phosphorothioate internucleotide linkage modifications within position 1-5 of the nucleotide sequence, counting from the 5′-end of the nucleotide sequence.

In some embodiments, Z¹¹ at the non-loop terminal has an overhang of 1-3 nucleotides in length. In one embodiment, Z¹¹ at the non-loop terminal has an overhang of 2 nucleotides in length (e.g., at the 3′-end of Z¹¹) and has a phosphorothioate internucleotide linkage between the two overhang nucleotides. In one embodiment, Z¹¹ at the non-loop terminal has an overhang of 2 nucleotides in length and has two phosphorothioate internucleotide linkages between the terminal 3 nucleotides (e.g., at the 3′-end of Z¹¹), in which 2 of the 3 nucleotides are the overhang nucleotides, and the third is the paired nucleotide next to the overhang nucleotide.

In some embodiments, the single-stranded oligonucleotide of formula (II) (or IIa) or formula (III) (or (IIIa) further comprises a phosphate or phosphate mimic at the 5′-end of a nucleotide sequence (e.g., Z¹¹ and/or Z¹²). In some embodiments, the single-stranded oligonucleotide comprises a phosphate mimic at the 5′-end of a nucleotide sequence (e.g., Z¹¹ and/or Z¹²). In one embodiment, at least one phosphate mimic is at the 5′ end of Z¹¹. In one embodiment, the phosphate mimic is a 5′-vinyl phosphonate (VP). In one embodiment, the phosphate mimic is a 5′-cyclopropyl phosphonate. In one embodiment, the phosphate mimic is a 5′-vinyl phosphate.

In some embodiments, the single-stranded oligonucleotide of formula (II) (or IIa) or formula (III) (or (IIIa) further comprises at least one terminal, chiral phosphorus atom.

In some embodiments, the 5′-end or 3′-end nucleotide in the single-stranded oligonucleotide of formula (II) (or IIa) or formula (III) (or (IIIa) comprise a 2′-5′-linked nucleotide modification; or the 5′-end or 3′-end nucleotide is conjugated to an abasic nucleotide, an inverted nucleotide, or an inverted abasic nucleotide (e.g., ribonucleotide), optionally via a phosphodiester, phosphorothioate, or phosphodithioate linkage.

In some embodiments, the 5′-end or 3′-end nucleotide in the single-stranded oligonucleotide of formula (II) (or IIa) or formula (III) (or (IIIa) is modified to comprise a linking moiety containing a mono-, di-, tri-, tetra-, penta- or polyprolinol, or mono-, di-, tri-, tetra-, penta- or polyhydroxyprolinol.

In some embodiments, at least one, two, three, four, or each of the five terminal phosphorous-containing linkages of the 5′-end or the 3′-end of the single-stranded oligonucleotide (e.g., Z¹¹ and/or Z¹²) are not phosphorothioate linkages. In one embodiment, the at least one, two, three, four, or each of the five terminal phosphorous-containing linkages of the 5′-end or the 3′-end of the single-stranded oligonucleotide (e.g., Z¹¹ and/or Z¹²) are each independently a natural phosphate group or a phosphodiester linkage, or a PN-linkage.

In some embodiments, at least one, two, three, four, or each of the five terminal phosphorous-containing linkages of the 5′-end or the 3′-end of the single-stranded oligonucleotide (e.g., Z¹¹ and/or Z¹²) are PN-linkages having the formula of —N(R)P(═X)(OH)O— or —OP(═X)(OH)N(R)—, —O—P(NR)(═X)O—, —N(SO₂R)P(═X)(OH)O— or —OP(═X)(OH)N(SO₂R)—, or —O—P(NSO₂R)(═X)O—, wherein X is O or S; R may be optionally substituted alkyl, aryl, heteroaryl, or heterocyclyl; or NR may be an optionally substituted cyclic guanidine moiety, an optionally substituted triazolyl group, or a Tmg group

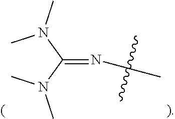 [SMILES: *N=C(N(C)C)N(C)C]

In some embodiments, at least one, two, three, four, or each of the five terminal phosphorous-containing linkages of the 5′-end or the 3′-end of the single-stranded oligonucleotide (e.g., Z¹¹ and/or Z¹²) are PN-linkages comprising an optionally substituted cyclic guanidine moiety, for instance, those having the structure of

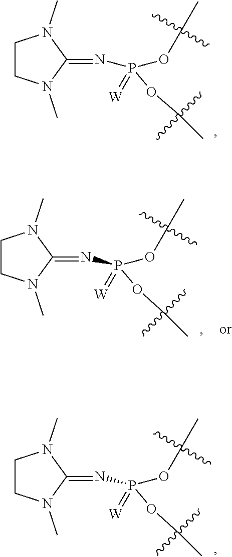

wherein W is O or S. In some embodiments, W is O. In some embodiments, W is S.

In some embodiments, at least one, two, three, four, or each of the five terminal phosphorous-containing linkages of the 5′-end or the 3′-end of the single-stranded oligonucleotide (e.g., Z¹¹ and/or Z¹²) are PN-linkages comprising a triazole moiety (e.g., an optionally substituted triazolyl group), such as those having the structure of

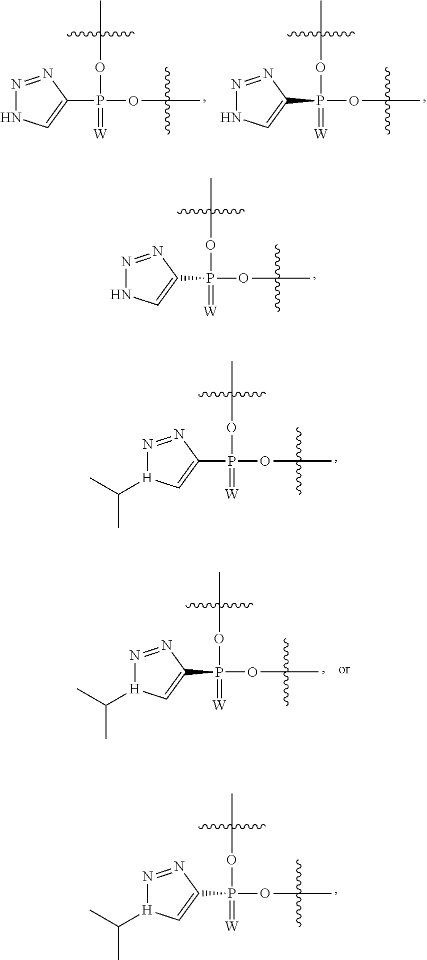

wherein W is O or S. In some embodiments, W is O. In some embodiments, W is S.

In some embodiments, at least one, two, three, four, or each of the five terminal phosphorous-containing linkages of the 5′-end or the 3′-end of the single-stranded oligonucleotide (e.g., Z¹¹ and/or Z¹²) are PN-linkages comprising an alkyne moiety (e.g., an optionally substituted alkynyl group), such as those having the structure of

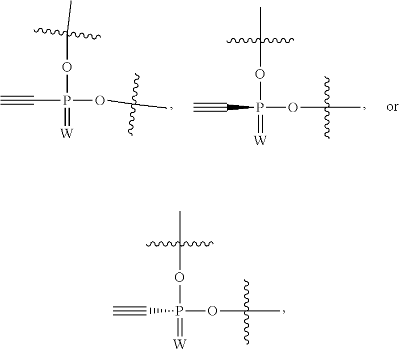

wherein W is O or S. In some embodiments, W is O. In some embodiments, W is S.

In some embodiments, at least one, two, three, four, or each of the five terminal phosphorous-containing linkages of the 5′-end or the 3′-end of the single-stranded oligonucleotide (e.g., Z¹¹ and/or Z¹²) are PN-linkages comprising a Tmg group

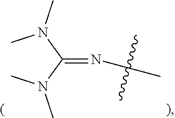 [SMILES: *N=C(N(C)C)N(C)C]

such as those having the structure of

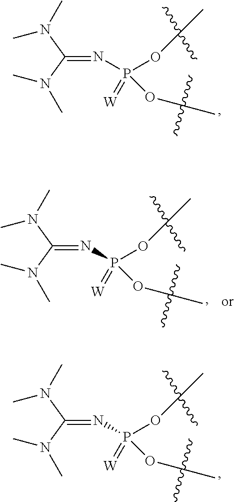

wherein W is O or S. In some embodiments, W is O. In some embodiments, W is S.

In all above embodiments, the PN-linkage may be stereochemically controlled.

In some embodiments, in the single-stranded oligonucleotide formula (II) (or IIa) or formula (III) (or (IIIa), the 3-5 terminal nucleotides of Z¹¹, connected to L, Q^{S}, or Z¹², contain modifications selected from the group consisting of 2′-deoxynucleotide (dN), a 2′-deoxy-2′-fluoronucleotide (fN), a ribonucleotide (rN), 2′-O-methylnucleotide (mN), and 2′-aranucleotide (aN).

In some embodiments, in the single-stranded oligonucleotide formula (II) (or IIa) or formula (III) (or (IIIa), the 3 terminal nucleotides of Z¹¹, connected to L, Q^{S}, or Z¹², have modifications selected from the group consisting of:
#-fN-fN-fN-**,
#-dN-dN-dN-**,
#-dN-dN-rN-**,
#-dN-rN-dN-**,
#-rN-dN-dN-**,
#-rN-rN-dN-**,
#-rN-dN-rN-**,
#-dN-rN-rN-**, and
#-rN-rN-rN-**,
wherein:
# is the bond to Z¹¹ and ** is the bond to L, Q^{S}, or Z²,
dN represents a 2′-deoxy nucleotide,
fN represents a 2′-deoxy-2′-fluoro nucleotide,
rN represents a ribonucleotide, and
mN represents a 2′-O-methyl nucleotide.

In some embodiments, in the single-stranded oligonucleotide formula (II) (or IIa) or formula (III) (or (IIIa), the 5 terminal nucleotides of Z¹¹, connected to L, Q^{S}, or Z¹², have modifications selected from the group consisting of:
#-dN-dN-fN-fN-fN-**,
#-dN-dN-rN-dN-dN-**,
#-dN-dN-rN-rN-rN-**,
#-dN-dN-dN-dN-dN-**,
#-mN-mN-fN-fN-fN-**,
#-mN-mN-dN-dN-dN-**,
#-mN-mN-rN-dN-dN-**,
#-mN-mN-rN-rN-rN-**, and
wherein:
# is the bond to Z¹¹ and ** is the bond to L, Q^{S}, or Z²,
dN represents a 2′-deoxy nucleotide,
fN represents a 2′-deoxy-2′-fluoro nucleotide,
rN represents a ribonucleotide, and
mN represents a 2′-O-methyl nucleotide.

In some embodiments, the first oligonucleotide Z¹¹ contains at least one motif of three consecutive 2′-O-methyl modifications at positions 11, 12, and 13 from the 5′-end of Z¹¹, and the nucleotide next to the motif is not 2′-O-methyl modified.

In some embodiments, the second oligonucleotide Z¹², optionally together with Q^{S}, contains at least one motif of three consecutive 2′-F modifications, and the nucleotide next to the motif is not 2′-F modified.

In some embodiments, the position of the motif of three consecutive modifications (three consecutive 2′-O-methyl modifications or three consecutive 2′-F modifications) is characterized by one or the followings:
the motif is at Q^{S}, positions 1 and 2 of Z¹², optionally Z¹¹ is 19 nucleotides in length;
the motif is at positions 1, 2, and 3 of Z¹², optionally Z¹¹ is 20 nucleotides in length;
the motif is at positions 2, 3, and 4 of Z¹², optionally Z¹¹ is 21 nucleotides in length;
the motif is at positions 3, 4, and 5 of Z¹², optionally Z¹¹ is 22 nucleotides in length; or
the motif is at positions 4, 5, and 6 of Z¹², optionally Z¹¹ is 23 nucleotides in length.

In some embodiments, Z¹², optionally together with Q^{S}, contains a 2′-O-methyl or 2′-F modification at a position that is 2 positions before the motif (position n-2, if the motif starts at position n), provided that the position is not part of Z¹¹.

In some embodiments, L is a cleavable linking group. In some embodiments, the cleavable linking group is cleavable in a homogenate, tritosome, cytosol, or endosome of any types of cells. For instance, the cleavable linking group may be cleavable in liver homogenates, liver tritosomes, liver lysosomes, liver cytosol, liver endosome, brain homogenates, brain tritosomes, brain lysosomes, brain cytosol, or brain endosome. In certain embodiments, the cleavable linking group is a redox cleavable linker (such as a reductively cleavable linker; e.g., a disulfide group), an acid cleavable linker (e.g., a hydrazone group, an ester group, an acetal group, or a ketal group), an esterase cleavable linker (e.g., an ester group), a phosphatase cleavable linker (e.g., an ester group), a peptidase cleavable linker (e.g., an ester group), or endosomal cleavable linker (or a protease cleavable linker, e.g., a carbohydrate linker).

In some embodiments, the cleavable linking group (tether) is an endosomal cleavable linker or a protease cleavable linker, for instance, a carbohydrate linker, wherein the linker is cleaved at least 1.25 times faster in the cell (or under in vitro conditions selected to mimic intracellular conditions) as compared to blood or serum (or under in vitro conditions selected to mimic extracellular conditions).

In some embodiments, L is present in formula (II) (or IIa) or formula III (or IIIa), and contains a linking moiety represented by a formula: #—(N)ₙ—**. In this formula, # is the bond to Z¹¹ and ** is the bond to Q^{S} or Z¹²; n is 3 to 12; and each N is independently a linking monomer having a chain length of 3 or more atoms. For instance, each N may be independently a linking monomer having a chain length of 3 or more atoms. The “chain length” has been defined herein above. In some embodiments, n is 3 to 8, 4 to 8, 3 to 7, 4 to 7, 3 to 6, 4 to 6, or 3 to 5. In one embodiment, n is 3.

In some embodiments, one or more linking moieties (N) in L of formula (II) (or IIa) or formula III (or IIIa) may be an optionally modified nucleotide.

In some embodiments, one or more linking moieties (N) in L of formula (II) (or IIa) or formula III (or IIIa) may be independently selected from the group consisting of a 2′-deoxynucleotide (dN), a 2′-deoxy-2′-fluoro nucleotide (fN), a ribonucleotide (rN), 2′-O-methylnucleotide (mN), and 2′-ara nucleotide (aN) (e.g., 2′-ara-2′-deoxy, 2′-ara-2′-F, 2′-ara-2′-OMe, or 2′-ara ribonucleotide).

In certain embodiments, one or more linking moieties (N) in L of formula (II) (or IIa) or formula III (or IIIa) may contain a modified internucleotide linkage selected from the group consisting of a phosphodiester, phosphotriester (optionally comprising the linkage phosphorus atom in either Rp configuration or Sp configuration), hydrogen phosphonate, alkyl or aryl phosphonate, phosphoramidate (optionally comprising the linkage phosphorus atom in either Rp configuration or Sp configuration), phosphorothioate (optionally comprising the linkage phosphorus atom in either Rp configuration or Sp configuration), methylenemethylimino, a nitrogen-modified phosphorous-containing linkage (PN-linkage) (optionally comprising the linkage phosphorus atom in either Rp configuration or Sp configuration), thiodiester, thionocarbamate, N,N′-dimethylhydrazine, phosphoroselenate, borano phosphate, borano phosphate ester, amide, hydroxylamino, siloxane, dialkylsiloxane, carboxamide, carbonate, carboxymethyl, carbamate, carboxylate ester, thioether, ethylene oxide linker, sulfide, sulfonate, sulfonamide, sulfonate ester, thioformacetal, formacetal, oxime, methyleneimino, methylenecarbonylamino, methylenehydrazo, methylenedimethylhydrazo, methyleneoxymethylimino, ether, thioacetamido, and combinations thereof.

In certain embodiments, one or more linking moieties (N) in L of formula (II) (or IIa) or formula III (or IIIa) may contain a moiety selected from the group consisting of an aliphatic saturated or unsaturated alkyl chain; a phosphorous-containing linkage, including a phosphate, a phosphonate, a phosphoramidate (optionally comprising the linkage phosphorus atom in either Rp configuration or Sp configuration), phosphodiester, phosphotriester (optionally comprising the linkage phosphorus atom in either Rp configuration or Sp configuration), phosphorothioate (optionally comprising the linkage phosphorus atom in either Rp configuration or Sp configuration), and a nitrogen-modified phosphorous-containing linkage (PN-linkage) (optionally comprising the linkage phosphorus atom in either Rp configuration or Sp configuration); a (poly)ethylene glycol chain, including diethylene glycol, triethylene glycol, tetra, penta, hexa, hepta, octa, nona, or deca ethylene glycol; glycerol or glycerol ester; an aminoalkyl ether; and combinations thereof.

In some embodiments, one or more linking moieties (N) in L of formula (II) (or IIa) or formula III (or IIIa) may contain a moiety selected from the group consisting of DNA, RNA, disulfide, amide, functionalized monosaccharides or oligosaccharides of galactosamine, glucosamine, glucose, galactose, mannose, and combinations thereof.

In some embodiments, one or more linking moieties (N) in L of formula (II) (or IIa) or formula III (or IIIa) may be independently selected from the group consisting of:

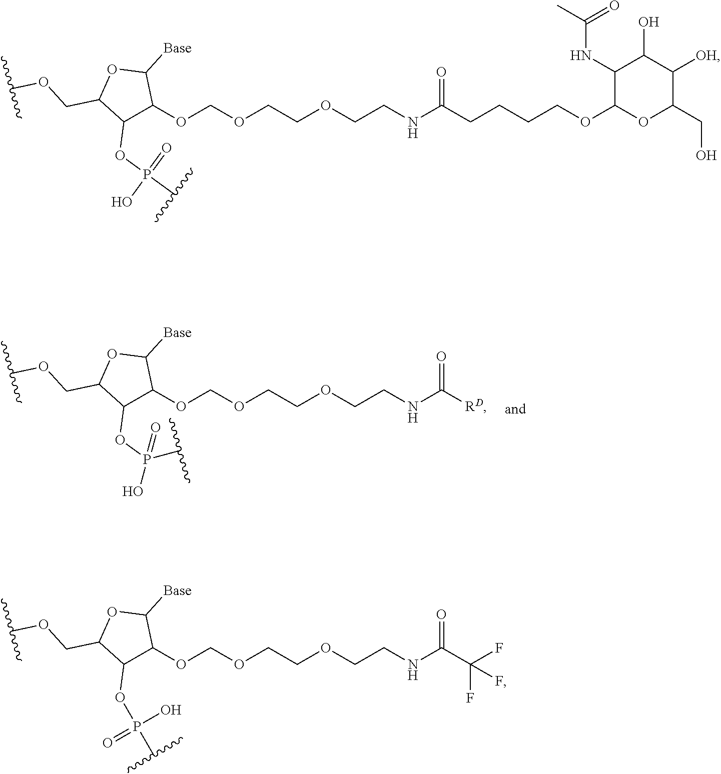

wherein:
Base is an optionally modified nucleobase, and
R^{D} is a C₄₋₃₀ alkyl, C₄₋₃₀ alkyenyl, or C₄₋₃₀ alkynyl.

In some embodiments, one or more linking moieties (N) in L of formula (II) (or IIa) or formula III (or IIIa) comprise a mono-, di-, tri-, tetra-, penta- or poly-prolinol, optionally conjugated with a ligand; a mono-, di-, tri-, tetra-, penta- or poly-hydroxyprolinol, optionally conjugated with a ligand; an optionally modified nucleotide; or combinations thereof.

In some embodiments, L of formula (II) (or IIa) or formula III (or IIIa) contains one or more of a mono-, di-, tri-, tetra-, penta- or poly-prolinol, optionally conjugated with a ligand; and one or more optionally modified nucleotides.

In some embodiments, L of formula (II) (or IIa) or formula III (or IIIa) contains one or more of a mono-, di-, tri-, tetra-, penta- or poly-hydroxyprolinol, optionally conjugated with a ligand; and one or more optionally modified nucleotides.

In some embodiments, one or more linking moieties (N) in L of formula (II) (or IIa) or formula III (or IIIa) may be independently selected from the group consisting of Y16, Y34, Q48, Q303, Q304, Q305, Q306, Q312, Q313, Q314, Q315, Q316, Q317, Q8, Q11, Q150, Q151, Q173, Q221, Q222, Q367, and Q368.

In some embodiments, each linking moiety (N) in L of formula (II) (or IIa) or formula III (or IIIa) is independently an optionally modified nucleotide, Y16, Y34, Q48, Q303, Q304, Q305, Q306, Q312, Q313, Q314, Q315, Q316, Q317, Q8, Q11, Q150, Q151, Q173, Q221, Q222, Q367, or Q368.

In some embodiments, L of formula (II) (or IIa) or formula III (or IIIa) contains 3-5 of 2′-deoxy nucleotides, a triplet of 2′-deoxy-2′-fluoro nucleotides, a triplet of ribonucleotides, a triplet of 2′-O-methyl nucleotides, or a triplet of Q304. In one embodiment, L contains a triplet of Q304.

In some embodiments, the position of L in formula (II) (or IIa) or formula III (or IIIa) is characterized by one of the followings:
all the linking monomer of L, together with LP, form a loop between W and Z¹²;
one or more of the linking monomers of L, together with LP, forms a loop between W and Z¹², and one or more of the linking monomers of L is not in the loop region;
one or more of the linking monomers of L, together with LP, forms a loop between W and Z¹², and one or more of the linking monomers of L is not in the loop and is connected to Q^{S}; and
one or more of the linking monomers of L, together with LP, forms a loop between W and Z¹², and one or more of the linking monomers of L is not in the loop and is connected to Z¹².

In the above embodiments, one or more internucleotide linkages between the nucleotides in L of formula (II) (or IIa) or formula III (or IIIa) may be modified internucleotide linkages independently selected from the group consisting of a phosphodiester, phosphotriester (optionally comprising the linkage phosphorus atom in either Rp configuration or Sp configuration), hydrogen phosphonate, alkyl or aryl phosphonate, phosphoramidate (optionally comprising the linkage phosphorus atom in either Rp configuration or Sp configuration), phosphorothioate (optionally comprising the linkage phosphorus atom in either Rp configuration or Sp configuration), and a nitrogen-modified phosphorous-containing linkage (PN-linkage) (optionally comprising the linkage phosphorus atom in either Rp configuration or Sp configuration).

In certain embodiments, L of formula (II) (or IIa) or formula III (or IIIa) may contain one or more linking moiety selected from the group consisting of a triazole linkage, an amide linkage, a sulfide or disulfide linkage, a phosphate linkage, an oxime linkage, a hydrazo linkage, a N,N′-dialkylenehydrazo linkage, a methyleneimino linkage, a methylenecarbonylamino linkage, a methylenemethylimino linkage, a methylenehydrazo linkage, a methylenedimethylhydrazo linkage, a methyleneoxymethylimino linkage, a hydroxylamino linkage, a formacetal linkage, an alkyl or aryl linkage, a PEG linkage, an ether linkage, a thioether linkage, a thiodiester linkage, a thionocarbamate linkage, a thioacetamido linkage, a sulfonate linkage, a sulfonamide linkage, a sulfonate ester linkage, a thioformacetal linkage, an urea linkage, a carbonate linkage, an amine linkage, a maleimide-thioether linkage, a phosphodiester linkage, a phosphotriester linkage, a hydrogen phosphonate linkage, an alkyl or aryl phosphonate linkage, a phosphoramidate linkage, a phosphorothioate linkage, a nitrogen-modified phosphorous-containing linkage (PN-linkage), a phosphoroselenate linkage, a borano phosphate linkage, a borano phosphate ester linkage, a sulfonamide linkage, a carbamate linkage, a carboxamide linkage, a carboxymethyl linkage, a carboxylate ester linkage, a siloxane linkage, a dialkylsiloxane linkage, an ethylene oxide linkage, and combinations thereof.

In certain embodiments, L of formula (II) (or IIa) or formula III (or IIIa) may contain one or more cyclic groups selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuranyl, and decalinyl.

In some embodiments, L of formula (II) (or IIa) or formula III (or IIIa) contains a nucleotide-based linker (tether). In some embodiments, L contains a non-nucleotide-based linker (tether).

In certain embodiments, the nucleotide-based or non-nucleotide-based linker (tether) contained in L of formula (II) (or IIa) or formula III (or IIIa) is a stable linker (tether) that is stable in a biological fluid. For instance, the nucleotide-based or non-nucleotide based stable linker (tether) is stable in plasma or artificial cerebrospinal fluid.

In certain embodiments, the cleavable linking group (tether) comprises a moiety of formula (CL-1) or (CL-2), as described above.

In certain embodiments, the cleavable linking group (tether) comprises a moiety selected from the following:
—(CH₂)₁₂— (C12 linker or Q50),
—(CH₂)₆—S—S—(CH₂)₆— (C6-S—S—C6 linker or Q51),
Q151,
Q173,
—CH₂CH₂O—(CH₂CH₂)ₙ—CH₂CH₂O—CH₂CH₂O—, wherein n is 0 or 1-20;
—(CH₂)₉(CH₂)ₙ—CH₂—, wherein n is 0 or 1-20;
mono-, di-, tri-, tetra-, penta- or polyprolinol, optionally conjugated with a ligand;
mono-, di-, tri-, tetra-, penta- or polyhydroxyprolinol, optionally conjugated with a ligand.

In certain embodiments, the cleavable linking group (tether) comprises a nucleic acid linker of 1 to 15 nucleotides in length. For instance, the nucleic acid linker may be 2 to 7, 5 to 7, 2 to 5, or 3, 4, or 5 optionally modified nucleotides in length.

In certain embodiments, the cleavable linking group (tether) comprises a nucleic acid linker comprising one or more nucleotides selected from the group consisting of 2′-O-methyl nucleotides, 2′-fluoro nucleotides, deoxyribonucleotides, and ribonucleotides. In one embodiment, all nucleic acid linker nucleotides are the same type of nucleotide. In one embodiment, the nucleic acid linker entirely comprises 2′-O-methyl nucleotides, entirely comprises 2′-fluoro nucleotides, or entirely comprises deoxyribonucleotides.

In certain embodiments, the cleavable linking group (tether) comprises a polynucleotide comprising a modified ribonucleotide sequence, optionally a polynucleotide comprising one or more modifications selected from the group consisting of a 2′-O-methyl ribonucleotide modification, a 2′-fluoro-ribonucleotide modification, a 2′-5′-linked nucleotide with different 3′-modification (3′-ribo, 3′-O-methyl, 3′-deoxy, 3′-fluoro), a glycol nucleic acid (GNA) modification, a locked nucleic acid (LNA) modification, a hexanol nucleic acid (HNA) modification, an abasic ribose modification, an abasic deoxyribose modification, and an abasic hydroxyprolinol modification.

In some embodiments, the linking group L in the single-stranded oligonucleotide of formula (II) (or IIa) or formula III (or IIIa) comprises a nucleotide-based cleavable linking group (tether) that is cleavable by DICER. In some embodiments, the single-stranded oligonucleotide comprises a substrate cleavable by DICER.

In certain embodiments, the single-stranded oligonucleotide of formula (II) (or IIa) or formula III (or IIIa) contains a cleavable linking group (nucleotide-based or non-nucleotide-based) capable of generating a metabolite of a 5′-monophosphate at at least one nucleotide sequence (e.g., Z¹¹ and/or Z¹²) of the single-stranded oligonucleotide.

In some embodiments, the single-stranded oligonucleotide of formula (II) (or IIa) or formula III (or IIIa) may further comprise one or more ligands (e.g., targeting ligands). In one embodiment, Z¹¹ comprises at least one ligand (e.g., a targeting ligand), at the 5′ or 3′ end of the sequence. In one embodiment, Z¹² comprises at least one ligand (e.g., a targeting ligand), at the 5′ or 3′ end of the sequence. In one embodiment, each of Z¹¹ and Z¹² comprises at least one ligand (e.g., a targeting ligand), at the 5′ or 3′ end of the sequence.

In some embodiments, at least one of the ligand is conjugated to an internal position of a nucleotide sequence (e.g., Z¹¹ and Z¹²), optionally via a linker or carrier. In some embodiments, at least one of the ligand is conjugated to the 3′-end or 5′-end of Z¹¹ or Z¹², optionally via a linker or carrier. In some embodiments, at least one of the ligands may be conjugated to the single-stranded oligonucleotide via a direct attachment to the ribosugar of the oligonucleotide. Alternatively, the ligand may be conjugated to the single-stranded oligonucleotide via one or more linkers (tethers), and/or a carrier.

In some embodiments, the internal position may refer to one of the positions 1-4 nucleotides upstream or downstream of the Q^{S}. In some embodiments, the internal position may refer to one of the positions 1-4 nucleotides upstream or downstream of nucleotides of Z¹² paired to positions 11, 12, and 13 from the 5′-end of Z¹¹.

For the purpose of counting internal positions for conjugation of ligands only, for Z¹², the terminal nucleotide (for Z¹²) that is connected to Q^{S} may be considered as internal positions.

In some embodiments, the internal position may be characterized by:
excluding the nucleotide of Q^{S} and/or Z¹² that is directly connected to the loop region; and/or
excluding position 2 or 14 from the 5′-end of Z¹¹; and/or
excludes positions 11, 12, and 13 from the 5′-end of Z¹¹; and/or
excluding the positions of Q^{S} and/or Z¹² paired to positions 11, 12, and 13 from the 5′-end of Z¹¹; and/or
excluding the two or three terminal positions from the 3′-end of Z¹² and the 5′-end of Z¹¹ for formula (II) or (IIa); and/or
excluding the two or three terminal positions from the 5′-end of Z¹² and the 3′-end of Z¹¹ for formula (III) or (IIIa).

In some embodiments, the ligand may be conjugated to the single-stranded oligonucleotide via a monovalent or branched bivalent or trivalent linker.

In some embodiments, the ligand may be conjugated to the single-stranded oligonucleotide via a carrier that replaces one or more nucleotide(s). The carrier can be a cyclic group or an acyclic group. In one embodiment, the cyclic group is selected from the group consisting of cyclohexyl, pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuranyl, and decalinyl. In one embodiment, the acyclic group is a moiety based on a serinol backbone or a diethanolamine backbone.

In certain embodiments, at least one of the ligands comprises a lipophilic moiety.

In one embodiment, the lipophilic moiety is lipid, cholesterol, retinoic acid, cholic acid, adamantane acetic acid, 1-pyrene butyric acid, docosanoic acid (DCA), dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, lithocholic acid, O3-(oleoyl)lithocholic acid, O3-(oleoyl)cholenic acid, dimethoxytrityl, or phenoxazine. In certain embodiments, the lipid is a fatty acid (an omega-3 fatty acid, for example), selected from the group consisting of eicosapentaenoic acid (EPA) and docosahexaenoic acid (DHA).

In some embodiments, the lipophilic moiety contains a saturated or unsaturated C₄-C₃₀ hydrocarbon chain (e.g., C₄-C₃₀ alkyl or alkenyl), and an optional functional group selected from the group consisting of hydroxyl, amine, carboxylic acid, sulfonate, phosphate, thiol, azide, and alkyne.

In some embodiments, the lipophilic moiety contains a saturated or unsaturated C₆-C₁₈ hydrocarbon chain (e.g., a linear C₆-C₁₈ alkyl or alkenyl), e.g., a saturated or unsaturated C₁₆ hydrocarbon chain (e.g., a linear C₁₆ alkyl or alkenyl). In some embodiments, the lipophilic moiety contains a saturated or unsaturated C₁₄-C₂₄ hydrocarbon chain (e.g., a linear C₁₄-C₂₄ alkyl or alkenyl), e.g., a saturated or unsaturated C₂₂ hydrocarbon chain (e.g., a linear C₂₂ alkyl or alkenyl). For example, one or more non-terminal positions of the single-stranded oligonucleotide may have a “2′-C₁₆” modification of formula (1), as described herein above, wherein B is a natural or modified nucleotide base (e.g., adenine, guanine, cytosine, thymine or uracil, or their modified derivatives), and the n-hexadecyl chain is the lipophilic moiety. In another example, one or more non-terminal positions of the single-stranded oligonucleotide may have “2′-C₂₂” modification of formula (2), as described herein above, wherein B is a natural or modified nucleotide base (e.g., adenine, guanine, cytosine, thymine or uracil, or their modified derivatives), and the n-docosanyl chain is the lipophilic moiety.

Similar modifications replacing the n-hexadecyl chain or the n-docosanyl chain with C₄-C₃₀ hydrocarbon chain is referred to as “2′-C₄-C₃₀ hydrocarbon chain” (or replacing with C₆-C₁₈ hydrocarbon chain or C₁₄-C₂₄ hydrocarbon chain is referred to as “2′-C₆-C₁₈ hydrocarbon chain” or “2′-C₁₄-C₂₄ hydrocarbon chain”).

In a related embodiment, one or more non-terminal nucleotide positions of at least one of Z¹¹ and Z¹² have the 2′-C₄-C₃₀ hydrocarbon chain structure, 2′-C₆-C₁₈ hydrocarbon chain structure, 2′-C₁₄-C₂₄ hydrocarbon chain structure, 2′-C₁₆ structure of formula (1), or 2′-C₂₂ structure of formula (2).

In some embodiments, the lipophilic moiety contains one or more phospholipids.

In some embodiments, the lipophilic moiety contains one or more lipids or lipophilic ligands disclosed in International PCT Application Publication Nos. WO 2019/232255A1 and WO 2021/108662A1, and U.S. Pat. No. 10,184,124; all of which are herein incorporated by reference in their entirety.

In some embodiments, the ligands include one or more of ligands of formulas (L-1), (L-2), (L-3), or (L-4), as described herein above.

In some embodiments, the ligands include those disclosed in International PCT Application Publication Nos. WO2017/053999, WO2019/118916, WO2022/031433, WO2022/056269, WO2022/056273, and WO2022/056277; all of which are herein incorporated by reference in their entirety.

In some embodiments, the lipophilic moiety comprises a saturated or unsaturated C₄-C₃₀ (e.g., C₄-C₁₈) hydrocarbon chain, and an optional functional group selected from the group consisting of hydroxyl, amine, carboylic acid, sulfonate, phostate, thiol, azide, and alkyne.

In some embodiments, the lipophilic moiety is conjugated to one or more of the internal positions on Z¹¹ or Z¹², optionally via a linker or carrier.

In some embodiments, at least one of Z¹¹ and Z¹² comprises one or more lipophilic moieties conjugated independently to one or more of the internal positions (i.e., non-terminal positions) excluding positions 9-12 on a nucleotide sequence; for instance, positions 4-8 and 13-18 on a nucleotide sequence; positions 5, 6, 7, 15, and 17 on a nucleotide sequence; or positions 4, 6, 7, and 8 on a nucleotide sequence, counting from the 5′-end of the nucleotide sequence as position 1.

In some embodiments, at least one of Z¹¹ and Z¹² comprises one or more lipophilic moieties conjugated independently to position 6 of the nucleotide sequence, counting from the 5′-end of the nucleotide sequence. In one embodiment, each of Z¹¹ and Z¹² comprises a lipophilic moiety conjugated to position 6 of the nucleotide sequence; optionally the lipophilic moiety comprises a saturated or unsaturated C₄-C₃₀ (e.g., C₄-C₁₈) hydrocarbon chain, or a saturated or unsaturated C₁₄-C₂₄ hydrocarbon chain; optionally the lipophilic moiety comprises a saturated or unsaturated C₁₆ hydrocarbon chain or a saturated or unsaturated C₂₂ hydrocarbon chain.

In some embodiments, at least one of Z¹¹ and Z¹² comprises one or more lipophilic moieties conjugated independently to one or more of non-terminal positions on a nucleotide sequence; for instance, positions 6-10 and 15-18 on a nucleotide sequence; and positions 15 and 17 on a nucleotide sequence, counting from the 5′-end of the nucleotide sequence as position 1.

In some embodiments, at least one lipophilic moiety is conjugated to an internal position the single-stranded oligonucleotide of formula (II) (or IIa) or formula (III) (or (IIIa)), wherein the internal position:
excludes position 2 or 14 from the 5′-end of Z¹¹; and/or
excludes positions 11, 12, and 13 from the 5′-end of Z¹¹; and/or
excludes the positions of Q^{S} and/or Z¹² paired to positions 11, 12, and 13 from the 5′-end of Z¹¹; and/or
optionally excludes the two or three terminal positions from the 3′-end of Z¹² and the 5′-end of Z¹¹ for formula (II) or (IIa); and/or
optionally excludes the two or three terminal positions from the 5′-end of Z¹² and the 3′-end of Z¹¹ for formula (III) or (IIIa).

In some embodiments, at least one of the ligands is a targeting ligand selected from the group consisting of an antibody, antigen, folate, receptor ligand, carbohydrate, aptamer, integrin receptor ligand, chemokine receptor ligand, transferrin, biotin, serotonin receptor ligand, PSMA, endothelin, GCPII, somatostatin, LDL and HDL ligand. In one embodiment, at least one of the ligands is an integrin receptor ligand.

The targeting ligand may be conjugated to an internal position of a nucleotide sequence (e.g., Z¹¹ and Z¹²), optionally via a linker or carrier. Alternatively, the targeting ligand may be conjugated to the 3′-end or 5′-end of Z¹¹ or Z¹², optionally via a linker or carrier.

In certain embodiments, at least one of the ligands is a carbohydrate-based ligand. The carbohydrate-based ligand may be D-galactose, multivalent galactose, N-acetyl-D-galactosamine (GalNAc), multivalent GalNAc, D-mannose, multivalent mannose, multivalent lactose, N-acetyl-glucosamine, glucose, multivalent glucose, multivalent fucose, glycosylated polyaminoacids, or lectins.

In some embodiments, the carbohydrate-based ligand is one or more GalNAc derivatives attached through a bivalent or trivalent branched linker, such as:

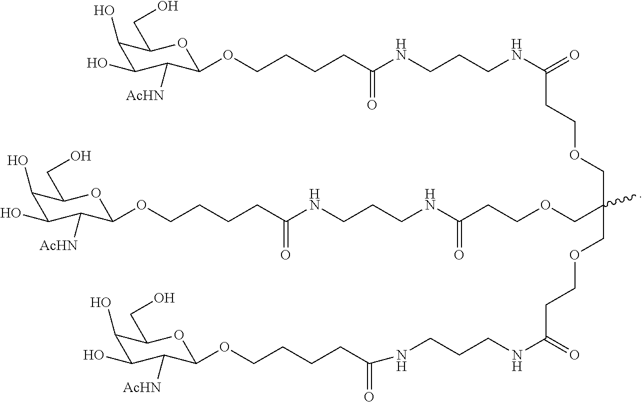 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the carbohydrate-based ligand is conjugated to the 3′-end of Z¹¹ or Z¹², or an internal position of Z¹¹ or Z¹². In one embodiment, the carbohydrate-based ligand is conjugated to the 3′-end of Z¹².

In some embodiments, one or more targeting ligands (e.g., carbohydrate-based ligands) are conjugated to an internal position of Z¹¹, excluding position 2 or 14.

In the above aspects of the invention relating to the single-stranded oligonucleotide, The phosphate mimic modification at the 5′-end of a nucleotide sequence, for the single-stranded oligonucleotide of formula (I), formula (II) (or IIa), or formula III (or IIIa), can be 5′-end phosphorothioate (5′-PS), 5′-end phosphorodithioate (5′-PS2), 5′ end vinylphosphonate (5′-VP), 5′-end methylphosphonate (MePhos), or 5′-deoxy-5′-C-malonyl. In one embodiment, the phosphate mimic is a 5′-vinylphosphonate (VP). The 5′-VP can be either 5′-E-VP isomer (i.e., trans-vinylphosphate), 5′-Z-VP isomer (i.e., cis-vinylphosphate), or mixtures thereof.

In one embodiment, the phosphate mimic is a 5′-vinyl phosphonate (VP). In one embodiment, the phosphate mimic is a 5′-cyclopropyl phosphonate. In one embodiment, the phosphate mimic is a 5′-vinyl phosphate.

In another embodiment, the single-stranded oligonucleotide further includes a phosphate or phosphate mimic at the 5′-end of the antisense strand (i.e., Z¹). Optionally, the phosphate mimic is a 5′-vinyl phosphonate (VP). When the phosphate mimic is a 5′-vinyl phosphonate (VP), the 5′-terminal nucleotide may have the following structure,

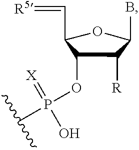

wherein:
X is O or S;
R is hydrogen, hydroxy, fluoro, or C₁₋₂₀alkoxy (e.g., methoxy or n-hexadecyloxy);
R^{5′} is ═C(H)—P(O)(OH)₂ and the double bond between the C5′ carbon and R^{5′} is in the E or Z orientation (e.g., E orientation); and
B is a nucleobase or a modified nucleobase, optionally where B is adenine, guanine, cytosine, thymine, or uracil.

In one embodiment, R^{5′} is =C(H)—P(O)(OH)₂ and the double bond between the C5′ carbon and R5′ is in the E orientation. In another embodiment, R is methoxy and R^{5′} is =C(H)—P(O)(OH)₂ and the double bond between the C5′ carbon and R5′ is in the E orientation. In another embodiment, X is S, R is methoxy, and R^{5′} is =C(H)—P(O)(OH)₂ and the double bond between the C5′ carbon and R^{5′} is in the E orientation.

In some embodiments, the —CH₂OH group at the 4′-position of the 5′-terminal nucleotide is replaced with a phosphate mimic of the formula —O—CH₂—P(O)(OR)₂, wherein each R is independently hydrogen or C₁₋₄ alkyl (e.g., one R group is hydrogen and one R group is methyl; or both R groups are hydrogen).

In one embodiment, the phosphate mimic is a 5′-cyclopropyl phosphonate (VP) (i.e., the CH₂OH group at the 4′-position of the 5′-terminal nucleotide is replaced with a group of the formula -Cy-P(O)(OR)₂, wherein Cy is a cyclopropyl ring and each R is independently hydrogen or C₁₋₄ alkyl (e.g., one R group is hydrogen or both R groups are hydrogen).

In some exemplary embodiments, the 5′-end phosphate mimic is

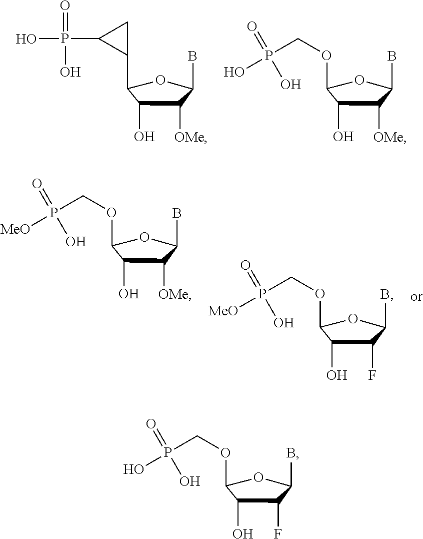

or a salt (e.g., sodium salt) thereof, wherein B is an optionally modified nucleobase (e.g., U).

In some embodiments, the 5′-end phosphate mimic is part of a modified 5′-terminal nucleotide. For example, the phosphate mimic may be part of a modified 5′-terminal nucleotide having the structure

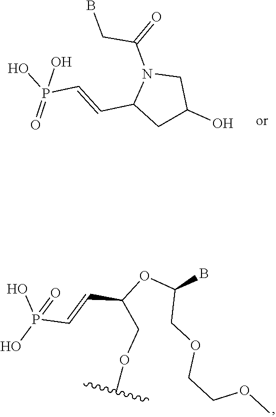

wherein B is an optionally modified nucleobase.

In some embodiments, the 5′-end phosphate mimic can also include a 5′-phosphate prodrug or 5′-phosphonate prodrug. In some embodiments, the 5′-phosphate prodrug or 5′-phosphonate prodrug has a structure of formulas disclosed in WO2022/147214, which is incorporated herein by reference. In some exemplary embodiments, the 5′-phosphate prodrug or 5′-phosphonate prodrug is:

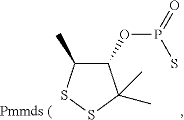

((4SR,5SR)-3,3,5-trimethyl-1,2-dithiolan-4-ol) phosphodiester);

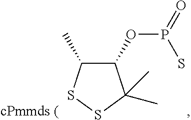

((4SR,5RS)-3,3,5-trimethyl-1,2-dithiolan-4-ol) phosphodiester (Cis Pmmds));

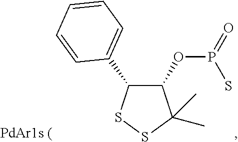

((4SR,5RS)-5-phenyl-3,3-dimethyl-1,2-dithiolan-4-ol) phosphodiester);

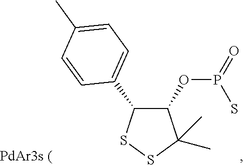

((4SR,5RS)-5-(4-methylphenyl)-3,3-dimethyl-1,2-dithiolan-4-ol) phosphodiester);

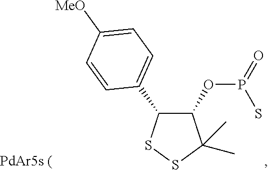

((4SR,5RS)-5-(4-methoxyphenyl)-3,3-dimethyl-1,2-dithiolan-4-ol) phosphodiester);

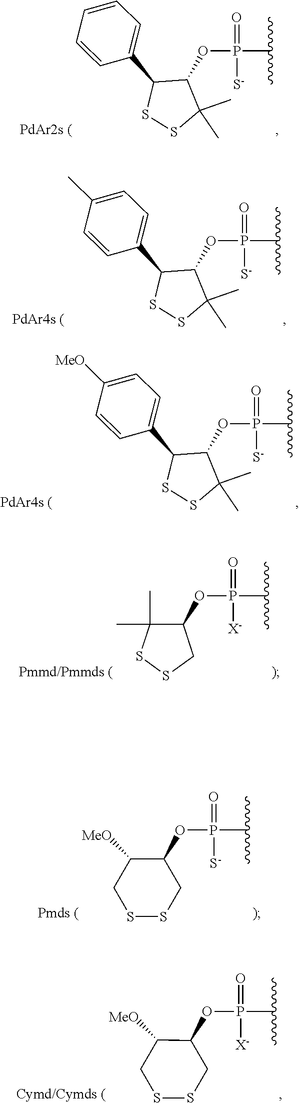

### X is O/S); or

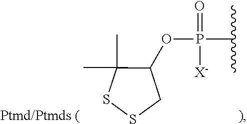

### X is O/S),

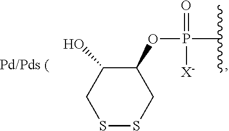

### X is O/S).

In some exemplary embodiments, the 5′-phosphate prodrug or 5′-phosphonate prodrug is:

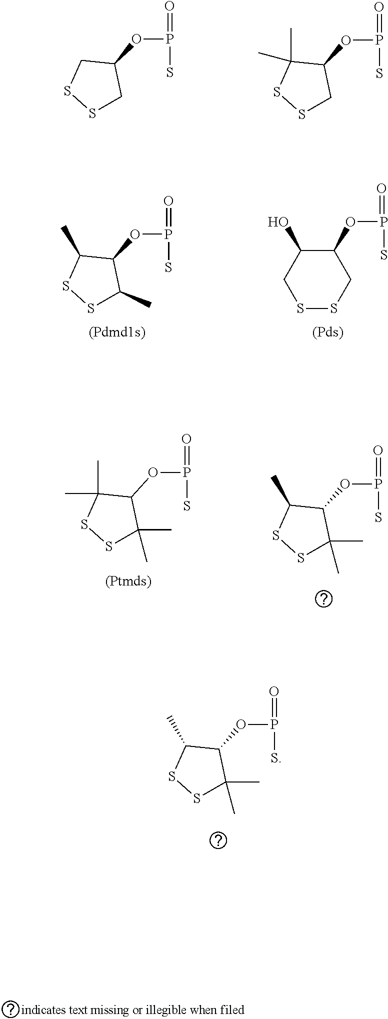

The siRNA containing one of the above list of 5′ modified phosphate prodrugs generally has an activity comparable to that of the siRNA containing 5′-VP. In some exemplary embodiments, the 5′-phosphate prodrug or 5′-phosphonate prodrug is.

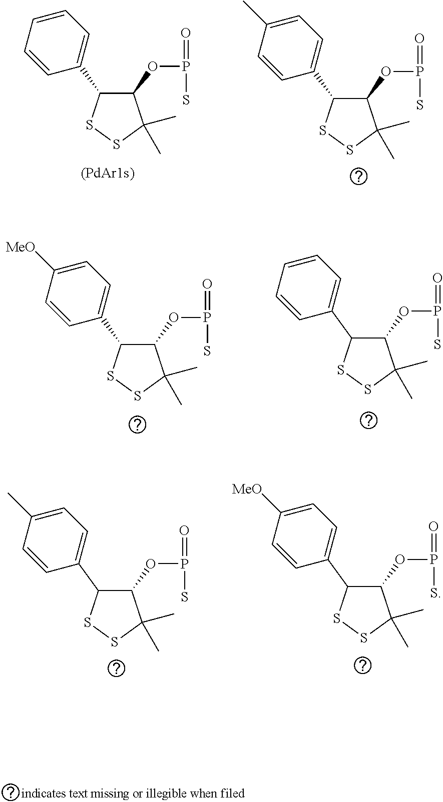

The siRNA containing one of the above list of 5′ modified phosphate prodrugs generally has an improved stability than that of the siRNA containing 5′-VP and has a better or comparable activity than that of the siRNA containing 5′-VP.

Another aspect of the invention relates to an oligonucleotide construct comprising two single-stranded oligonucleotides of formula (I) as described above, wherein the two single-stranded oligonucleotides are covalently bonded.

Another aspect of the invention relates to an oligonucleotide construct comprising two single-stranded oligonucleotides of formula (II) (or IIa) or (III) (or IIIa) as described above, wherein the two single-stranded oligonucleotides are covalently bonded.

In some embodiments, at least one of the single-stranded oligonucleotides forming the oligonucleotide construct is one from formula (I). In some embodiments, at least one of the single-stranded oligonucleotides forming the oligonucleotide construct is one from formula (II) (or IIa). In some embodiments, at least one of the single-stranded oligonucleotides forming the oligonucleotide construct is one from formula (III) (or IIIa).

In some embodiments, the covalent bonding of the two single-stranded oligonucleotides occurs at the linking group L for each single-stranded oligonucleotide.

In some embodiments, the two single-stranded oligonucleotides are covalently bonded via a tethering group selected from the group consisting of oxime, aminooxy, a triazole or fused triazole, phosphodiester, phosphotriester, hydrogen phosphonate, alkyl or aryl phosphonate, phosphoramidate, phosphorothioate, a nitrogen-modified phosphorous-containing linkage (PN-linkage), methylenemethylimino, thiodiester, thionocarbamate, N,N′-dimethylhydrazine, phosphoroselenate, borano phosphate, borano phosphate ester, amide, hydroxylamino, siloxane, dialkylsiloxane, carboxamide, carbonate, carboxymethyl, carbamate, carboxylate ester, thioether, ethylene oxide linker, sulfide, sulfonate, sulfonamide, sulfonate ester, thioformacetal, formacetal, methyleneimino, methylenecarbonylamino, methylenehydrazo, methylenedimethylhydrazo, methyleneoxymethylimino, ether, thioacetamido, and a combination thereof.

In some embodiments, the tethering group is oxime, aminooxy, or a triazole or fused triazole. Exemplary tethering groups and exemplary process for covalently bonding two single-stranded oligonucleotides to form an oligonucleotide construct are shown in Schemes 7.1-7.4 below.

The two single-stranded oligonucleotides may be the same or different.

In some embodiments, the two single-stranded oligonucleotides forming the oligonucleotide construct are the same. In one embodiment, the single-stranded oligonucleotide forming the oligonucleotide construct is one from formula (I). In one embodiment, the single-stranded oligonucleotide forming the oligonucleotide construct is one from formula (II) (or IIa). In one embodiment, the single-stranded oligonucleotide forming the oligonucleotide construct is one from formula (III) (or IIIa).

In some embodiments, the two single-stranded oligonucleotides forming the oligonucleotide construct are different.

In some embodiments, the two single-stranded oligonucleotides forming the oligonucleotide construct are two different single-stranded oligonucleotides from formula (I). In some embodiments, Z¹ and/or Z² of one single-stranded oligonucleotide contains different modifications than Z¹ and/or Z² of the other single-stranded oligonucleotide. In some embodiments, L of one single-stranded oligonucleotide is different than L of the other single-stranded oligonucleotide. In some embodiments, Q¹ and/or Q² of one single-stranded oligonucleotide is different than Q¹ and/or Q² of the other single-stranded oligonucleotide. In some embodiments, one single-stranded oligonucleotide contains a ligand that is different than the other single-stranded oligonucleotide. For instance, one single-stranded oligonucleotide contains a ligand, and the other single-stranded oligonucleotide does not contain a ligand or contains a different ligand. In some embodiments, one single-stranded oligonucleotide contains a ligand that is at a different location than the ligand on the other single-stranded oligonucleotide.

In some embodiments, the two single-stranded oligonucleotides forming the oligonucleotide construct are two different single-stranded oligonucleotides from formula (II) (or IIa) or (III)(or IIIa). In some embodiments, one single-stranded oligonucleotide is from formula (II) (or IIa), and the other single-stranded oligonucleotide is from formula (III)(or IIIa). In some embodiments, Z¹¹ and/or Z¹² of one single-stranded oligonucleotide contains different modifications than Z¹¹ and/or Z¹² of the other single-stranded oligonucleotide. In some embodiments, L of one single-stranded oligonucleotide is different than L of the other single-stranded oligonucleotide. For instance, one single-stranded oligonucleotide contains a L, and the other single-stranded oligonucleotide does not contain a L or contains a different L. In some embodiments, Q^{S} of one single-stranded oligonucleotide is different than Q^{S} of the other single-stranded oligonucleotide. For instance, one single-stranded oligonucleotide contains a Q^{S}, and the other single-stranded oligonucleotide does not contain a Q^{S} or contains a different Q^{S}. In some embodiments, one single-stranded oligonucleotide contains a ligand that is different than the other single-stranded oligonucleotide. For instance, one single-stranded oligonucleotide contains a ligand, and the other single-stranded oligonucleotide does not contain a ligand or contains a different ligand. In some embodiments, one single-stranded oligonucleotide contains a ligand that is at a different location than the ligand on the other single-stranded oligonucleotide.

In some embodiments, the two single-stranded oligonucleotides forming the oligonucleotide construct are two different single-stranded oligonucleotides, having one single-stranded oligonucleotide from formula (I) and another single-stranded oligonucleotide from formula (II) (or IIa) or (III)(or IIIa).

Another aspect of the invention relates to a pharmaceutical composition comprising the single-stranded oligonucleotide described above according to formula (I), and a pharmaceutically acceptable excipient.

Another aspect of the invention relates to a pharmaceutical composition comprising the single-stranded oligonucleotide described above according to formula (II) (or (IIa)) or (III) (or (IIIa)), and a pharmaceutically acceptable excipient.

Another aspect of the invention relates to a pharmaceutical composition comprising the oligonucleotide construct described above, comprising two single-stranded oligonucleotides according to formula (I), (II) (or (IIa)), or (III) (or (IIIa)), and a pharmaceutically acceptable excipient.

All the above embodiments relating to the single-stranded oligonucleotide, the nucleotide sequence(s), formula (I) and all variables defined in formula (I), formula (II)-(IIa) and all variables defined in formula (II)-(IIa), formula (III)-(IIIa) and all variables defined in formula (III)-(IIIa), the chemical modifications on the nucleotide sequences, the linking groups L within the oligonucleotide, the tethering groups colvalently bonding the two single-stranded oligonucleotides, and the ligand and ligand conjugation disclosed in the above aspect of the invention relating to the single-stranded oligonucleotide are suitable in this aspect of the invention relating to a pharmaceutical composition.

Another aspect of the invention relates to a method for inhibiting the expression of one or more target genes in a subject, comprising contacting the cell of the subject with, or administering to the subject, the single-stranded oligonucleotide described above according to formula (I), in an amount sufficient to inhibit the activity or expression of the one or more target genes in the cell of the subject.

Another aspect of the invention relates to a method for inhibiting the expression of one or more target genes in a subject, comprising contacting the cell of the subject with, or administering to the subject, the single-stranded oligonucleotide described above according to formula (II) (or (IIa)) or (III) (or (IIIa)), in an amount sufficient to inhibit the activity or expression of the one or more target genes in the cell of the subject.

Another aspect of the invention relates to a method for inhibiting the expression of one or more target genes in a subject, comprising contacting the cell of the subject with, or administering to the subject, the oligonucleotide construct described above, comprising two single-stranded oligonucleotides according to formula (I), (II) (or (IIa)), or (III) (or (IIIa)), in an amount sufficient to inhibit the activity or expression of the one or more target genes in the cell of the subject.

All the above embodiments relating to the single-stranded oligonucleotide, the nucleotide sequence(s), formula (I) and all variables defined in formula (I), formula (II)-(IIa) and all variables defined in formula (II)-(IIa), formula (III)-(IIIa) and all variables defined in formula (III)-(IIIa), the chemical modifications on the nucleotide sequences, the linking groups L within the oligonucleotide, the tethering groups colvalently bonding the two single-stranded oligonucleotides, and the ligand and ligand conjugation disclosed in the above aspect of the invention relating to the single-stranded oligonucleotide are suitable in this aspect of the invention relating to a method for inhibiting the expression of one or more target genes in a subject.

In some embodiments, the cell is within a subject. In one embodiment, the subject is a human. In one embodiment, the subject is a non-human mammal, e.g., a rhesus monkey, a cynomolgous monkey, a mouse, or a rat.

In all the above aspects of the invention, the single-stranded oligonucleotide is capable of inhibiting the activity or expression of the one or more target genes in a tissue of the subject by at least 15% each relative to an appropriate control (e.g., as compared to an untreated or placebo-treated subject, or as compared to a reference value, including, e.g., target mRNA or protein levels in the treated subject measured before the treatment with the single-stranded oligonucleotide or the double-stranded nucleic acid agent occurred), optionally by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% each relative to an appropriate control. In one embodiment, the appropriate control is an untreated subject. In one embodiment, the appropriate control is a reference value, e.g., a value obtained for the subject prior to administration of the single-stranded oligonucleotide to the subject.

### BRIEF DESCRIPTION OF THE DRAWINGS

FIG. 1A is a scheme showing the sequence design of the exemplary single-stranded oligonucleotides, and their conjugations to the GalNAc ligands. FIG. 1B is a scheme showing the sequence design of the exemplary single-stranded oligonucleotides, as shown in FIG. 1A, but in the form of a loopmer having a loop and an intra-strand duplexed region between the corresponding antisense and sense nucleotides.

FIG. 2 shows the loss of full-length for the exemplary single-stranded oligonucleotides and strands of the parent siRNA duplex listed in Tables 1 and 3, after incubation of the oligonucleotides in plasma.

FIG. 3 is a scheme showing the plasma metabolism summary for the exemplary single-stranded oligonucleotides listed in Table 1.

FIG. 4 is a scheme showing the liver homogenate metabolism summary for the exemplary single-stranded oligonucleotides and the parent siRNA duplex listed in Tables 1 and 3.

FIG. 5A-C illustrates the gene silencing effects of the exemplary single-stranded loop oligonucleotides compared against the parent siRNAs in mice. A single dose of siRNA or single-stranded loop oligonucleotides at 1 mg/kg (FIG. 5A), 0.4 mg/kg (FIG. 5B), and 0.2 mg/kg (FIG. 5C) were administered to mice on Day 0, and serum was collected on Days 0 (pre-dose), 7, 14, and 24. Circulating serum protein levels for TTR mRNA were determined relative to PBS-treated groups. Error bars are SD (n=3). GalNAc-siRNAs with double standard sense and antisense strands (On-1); GalNAc-single-stranded loop oligonucleotides (On-2 to On-11).

FIG. 6 shows the total ion chromatograms illustrating the major metabolites of parent siRNA or the exemplary single-stranded loop oligonucleotides (a. On-1 b. On-2 c. On-3 d. On-5 e. On-6 f. On-7 g. On-8 h. On-9 i. On-10 j. On-11) formed at 24 hours in rat plasma.

FIG. 7 shows the total ion chromatograms illustrating the major metabolites of parent siRNA or the exemplary single-stranded loop oligonucleotides (a. On-1 b. On-2 c. On-3 d. On-5 e. On-6 f. On-7 g. On-8 h. On-9 i. On-10 j. On-11) formed at 24 hours in rat liver homogenate.

FIGS. 8A and 8B show the intensity of parent loopmerRNA/formation of 22-23mer antisense metabolite correlates with % mTTR knockdown. FIG. 9A shows the loss of intensity of parent RNA from in vitro liver incubations correlated with % mTTR knockdown in vivo. An intensity of zero is used for loopmers where full length loopmerRNA was not identified. FIG. 9B shows the formation of 22/23 antisense for each single-stranded loop oligonucleotide from in vitro liver incubations correlated with % mTTR knockdown in vivo.

FIG. 9 shows the results of knockdown of TTR mRNA with and without VP, comparing On-2, On-3, On-9, and On-10.

FIG. 10 is a scheme showing the sequence design of exemplary single-stranded oligonucleotides in the form of a loopmer having a loop and an intra-strand duplexed region between the corresponding antisense and sense nucleotides, and their conjugations to the GalNAc ligands, as compared to the parent siRNA duplex. Various loop design and various chemistries for the single-stranded oligonucleotides in connection with their stability in liver homogenate and plasma are illustrated in the figure. The single-stranded oligonucleotides having a loop region containing a 3-nt loop (A-1700636) was stable in liver homogenate for 24 hours and in plasma for 8 hours. The single-stranded oligonucleotides having a loop region containing a 7-nt loop (A-492540) was semi-labile in liver homogenate for 24 hours and in plasma for 8 hours. The single-stranded oligonucleotides having a loop region containing a 7-nt loop (A-511271) was labile in liver homogenate for 24 hours and in plasma for 8 hours.

FIGS. 11A-11B show the results of the metabolism and in vivo knockdown of the exemplary single-stranded oligonucleotides listed in FIG. 10. FIG. 11A shows the inhibition of mTTR expression by the exemplary single-stranded oligonucleotides (listed in FIG. 10) in a mouse at a single dosage of 0.2 mg/kg. The label for “stable loop” corresponds to A-1700636 shown in FIG. 10; the label for “semi-labile loop” corresponds to A-492540 in FIG. 10; the label for “labile loop” corresponds to A-511271 in FIG. 10; the label for “canonical duplex” corresponds to the parent siRNA AD-64228. FIG. 11B shows the inhibition of mTTR expression by certain exemplary single-stranded oligonucleotides (listed in FIG. 10) in a mouse at a single dosage of 0.2 mg/kg, as compared to the same oligonucleotide but with a 5′-(E)-vinylphosphonate (VP) modification. The label “semi-labile loop+5′-VP” refers to the sequence of A-492540 (“semi-labile loop”) but with a 5′-(E)-VP modification. The label “canonical duplex+5′-VP” refers to the sequence of parent siRNA AD-64228 (“canonical duplex”) but with a 5′-(E)-VP modification.

FIG. 12 show the results of in vivo knockdown of the exemplary single-stranded oligonucleotides as compared to parent duplexes and controls (Table 7), in a mouse at a single dosage of 2.5 mg/ml. The samples in the graph from left to right are PBS, AD-579804, A-4102742, A-3903365, A-3903366, AD-1953663, A-3903367, A-3903368, AD-1983263, AD-1983265, respectively.

### DETAILED DESCRIPTION

The inventors have designed a novel strategy to prepare a single-stranded loop oligonucleotide using two chemically modified oligonucleotides capable of forming an intra-strand duplexed region and connecting the two oligonucleotides by a cleavable linking group, generating a single-stranded construct. The single-stranded loop oligonucleotide is designed to cleave at a suitable rate for the single-stranded construct to be cleaved into a double-stranded RNAi agent that is effective in vivo. The single-stranded loop oligonucleotide are synthesized as single strands and self-anneal due to sequence complementarity and are purified as single strands. Delivery ligands such as triantennary GalNAc can be readily incorporated during synthesis. The single-stranded loop oligonucleotide described herein has the stability in plasma with the ability to metabolize and release siRNAs efficiently in vivo. The single-stranded loop oligonucleotide discussed herein provides an improved design to simplify the manufacture and purification of RNAi agent by increasing the throughput and reducing the overall synthesis time, yet at the same time preserving or improving the efficacy of the RNAi agent when being cleaved in vivo.

### Single-Stranded Oligonucleotide Structure Design

One aspect of the invention relates to a single-stranded oligonucleotide capable of inhibiting the expression of a target gene, having a sequence represented by formula (I):

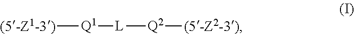

wherein:
Z¹ is a first oligonucleotide, comprising 15-100 optionally modified nucleotides that is substantially complementary to a target gene;
Z² is a second oligonucleotide, comprising 15-100 optionally modified nucleotides that is substantially complementary to Z¹;
Z¹ and Z² are capable of forming an intra-strand duplexed region comprising 3 or more consecutive base pairs;
L is a linking group;
Q¹ and Q² each independently represent 0 to 12 optionally modified nucleotides; and
at least one nucleotide in formula (I) is a modified nucleotide.

The single-stranded oligonucleotide is formed by connecting the two oligonucleotides by a linking group L. Some exemplary single-stranded oligonucleotide constructs are illustrated in Schemes 1 and 2.

As shown in Schemes 1 and 2, in some embodiments, Z¹ represents a first oligonucleotide that is substantially complementary to a target gene (e.g., an antisense strand); and Z² represents is a second oligonucleotide that is substantially complementary to Z¹ (e.g., a sense strand). In some embodiments, Z¹ and Z² can form an intra-strand duplexed region between the corresponding nucleotides of Z¹ and Z², and the single-stranded oligonucleotide contains a loop region formed by the linking group L (and possibly Q¹ and Q²). In some embodiments, Q¹ and Q² each independently can be absent. In some embodiments, Q¹ and Q² each independently can be present as an overhang to the first oligonucleotide Z¹ and the second oligonucleotide Z², respectively. As shown in Schemes 1 and 2, L is a linking group that can contain modified or unmodified nucleotides. In some embodiments, as shown in Scheme 2, L can contain non-nucleotide based linkers, such as Q304. In some embodiments, as shown in Schemes 1 and 2, the nucleotides of the entire single-strand oligonucleotide (including Z¹, Z², Q¹, Q², and L) can contain various chemical modifications such as DNA, RNA, 2′-F, or 2′-OMe. In some embodiments, as shown in Schemes 1 and 2, the single-strand oligonucleotide further comprises a ligand, e.g., 3 GalNAc derivatives attached through a trivalent branched linker,

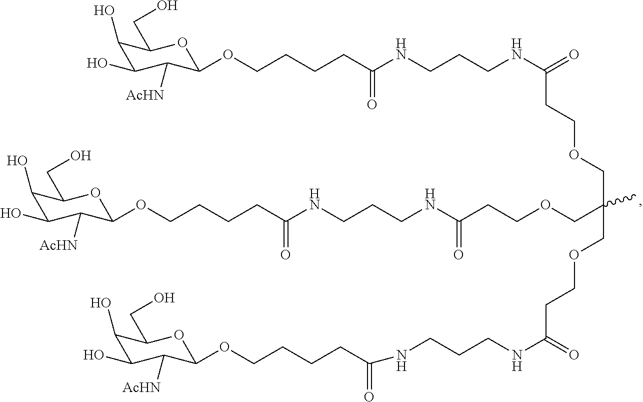 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

at the 3′ end of Z² (e.g., a sense strand). In some embodiments, as shown in Scheme 2, the single-strand oligonucleotide further comprises a phosphate or phosphate mimic (e.g., 5′ end vinylphosphonate (5′-VP)) at the 5′-end of Z¹ (e.g., an antisense strand).

Each of the first oligonucleotide Z¹ and second oligonucleotide Z² can be at least 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. Each of the first oligonucleotide Z¹ and second oligonucleotide Z² may have about 10 to about 50 nucleotides, about 10 to about 40 nucleotides, about 10 to about 35 nucleotides, about 10 to about 30 nucleotides, about 10 to about 25 nucleotides, about 10 to about 20 nucleotides, about 15 to about 50 nucleotides, about 15 to about 40 nucleotides, about 15 to about 35 nucleotides, about 15 to about 30 nucleotides, about 15 to about 25 nucleotides, about 15 to about 20 nucleotides, or about 18 to about 20 nucleotides in length. In one embodiment, each of the first oligonucleotide Z¹ and second oligonucleotide Z² is at least 15 nucleotides in length. In one embodiment, each of the first oligonucleotide Z¹ and second oligonucleotide Z² is at least 18 nucleotides in length.

Another aspect of the invention relates to a single-stranded oligonucleotide according to formula (II) or (III):

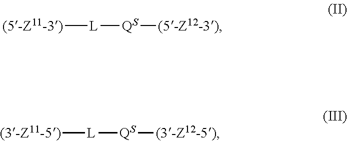

wherein:
Z¹¹ is a first oligonucleotide, comprising 15-100 optionally modified nucleotides that is substantially complementary to a target gene;
Z¹² is a second oligonucleotide, comprising 10-100 optionally modified nucleotides that is substantially complementary to Z¹¹;
Z¹¹ and Z¹² are capable of forming an intra-strand duplexed region comprising 7 or more consecutive base pairs;
Q^{S} represents 0 to 12 optionally modified nucleotides;
L is an optional linking group;
at least one nucleotide in formula (II) is a modified nucleotide; and
at least one nucleotide in formula (III) is a modified nucleotide, wherein
   at least one nucleotide at the 3′ end of Z¹¹, for formula (II), at least one nucleotide at the 5′ end of Z¹¹, for formula (III), in either case together with L and
Q^{S} form a loop region connecting Z¹¹ and Z¹².

The single-stranded oligonucleotide is formed by connecting the two oligonucleotides, optionally by a linking group L.

In some embodiments, the single-stranded oligonucleotide is represented by formula (IIa) or formula (IIIa):

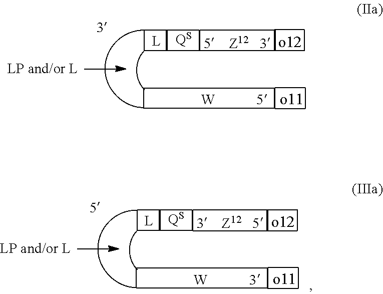

wherein:
Z¹¹ comprises W-LP,
W forms an intra-strand duplexed region at least 7 base pairs with Z¹²,
LP, optionally together with L, forms a loop between W and Z¹² at the 3′-end or 5′-end,
[symbol] represents an optional presence of L,
[symbol] represents an optional presence of Q^{S},
[symbol] represents an optional overhang at 5′-end or 3′-end of Z¹¹, and
[symbol] represents an optional overhang at 5′-end or 3′-end of Z¹².

Some exemplary single-stranded oligonucleotides are illustrated in Schemes 1B.1-1B.5 and Schemes 2B.1-2B.4.

Some exemplary single-stranded oligonucleotides may have the orientation (e.g., 5′-3′ orientation) and connections of Z¹¹ and Z¹², as defined in formula (II) or (IIa), as illustrated by Schemes 1B.1-113.5. The PS internucleotide linkages illustrated in each of Schemes 1B.1-113.5 are exemplary and may be present or absent. In certain embodiments, the illustrated PS internucleotide linkages at the 3′- and 5′-ends are present, while the internal PS internucleotide linkages are absent.

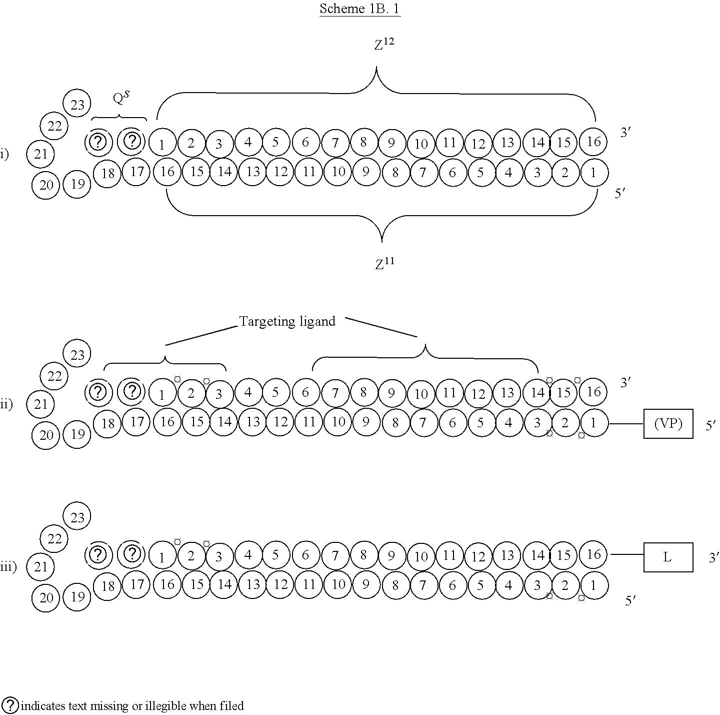

As shown in Scheme 1B.1, in some embodiments, Z¹¹ represents a first oligonucleotide that is substantially complementary to a target gene (e.g., an antisense strand); and Z¹² represents is a second oligonucleotide that is substantially complementary to Z¹¹ (e.g., a sense strand). In some embodiments, Z¹¹ and Z¹² can form an intra-strand duplexed region between the corresponding nucleotides of Z¹¹ and Z¹², and the single-stranded oligonucleotide contains a loop region LP (possibly including a linking group L; not marked).

In some embodiments, Q^{S} can be absent. In some embodiments, Q^{S} is represented by a and b, which may be spacers and can be any optionally modified nucleotide that form matched or mismatched base pairs with their opposite nucleotides in Z¹¹ (e.g., the two corresponding nucleotides at positions 17 and 18, in Scheme 1B.1). In one embodiment, both a and b form matched base pairs with their opposite nucleotides in Z¹¹. In one embodiment, both a and b form mismatched base pairs with their opposite nucleotides in Z¹¹. In one embodiment, one of a and b forms a matched base pair with its opposite nucleotide in Z¹¹; and another of a and b forms a mismatched base pair with its opposite nucleotide in Z¹¹. In one embodiment, b forms a mismatched base pair with its opposite nucleotide in Z¹¹ (e.g., b is mismatched to the nucleotide at position 17, as shown in Scheme 1B.1). In one embodiment, a forms a mismatched base pair with its opposite nucleotide in Z¹¹ (e.g., a is mismatched to the nucleotide at position 18, as shown in Scheme 1B.1).

In some embodiments, the single-stranded oligonucleotide contains one or two phosphorothioate internucleotide linkage modifications (e.g., two consecutive phosphorothioate internucleotide linkage modifications) within first 6 nucleotides or last 6 nucleotides of Z¹¹ or Z¹² (i.e., one or two phosphorothioate internucleotide linkage modifications between nucleotides at terminal 6 positions from either the 5′ end or the 3′ end for either Z¹¹ or Z¹²). In one embodiment, the single-stranded oligonucleotide contains two consecutive phosphorothioate internucleotide linkage modifications within first 3 nucleotides or last 3 nucleotides of Z¹¹ or Z¹² (i.e., the internucleotide linkages between terminal 3 positions from either the 5′ end or the 3′ end for either Z¹¹ or Z¹² are modified with wo consecutive phosphorothioate internucleotide linkage modifications, e.g., the phosphorothioate internucleotide linkage modifications shown as “stars” in iii) of Scheme 1B.1).

In some embodiments, the single-stranded oligonucleotide contains one or two phosphorothioate internucleotide linkage modifications (e.g., two consecutive phosphorothioate internucleotide linkage modifications) within last 8 nucleotides of Z¹¹. In one embodiment, the single-stranded oligonucleotide has Z¹¹ of 23 nucleotides in length, and contains two phosphorothioate internucleotide linkage modifications between nucleotides at positions 16-23 (e.g., two phosphorothioate internucleotide linkage modifications between nucleotides at positions 16-17, 17-18, 18-19, 19-20, 20-21, 21-22 of Z¹¹, as shown in Scheme 1B.1).

In some embodiments, the single-stranded oligonucleotide contains one or two phosphorothioate internucleotide linkage modifications (e.g., two consecutive phosphorothioate internucleotide linkage modifications) within first 3 nucleotides of Z¹² (e.g., one or two phosphorothioate internucleotide linkage modifications between nucleotides at positions 1-2 and/or 2-3 of Z¹², as shown in Scheme 1B.1). In one embodiment, the single-stranded oligonucleotide contains two consecutive phosphorothioate internucleotide linkage modifications between nucleotides at positions 1-2 and 2-3 of Z¹², two consecutive phosphorothioate internucleotide linkage modifications between last 3 nucleotides (e.g., at positions 14-15 and 15-16) of Z¹², and two consecutive phosphorothioate internucleotide linkage modifications between nucleotides at positions 1-2 and 2-3 of Z¹¹, as shown in ii) of Scheme 1B.1.

In some embodiments, as shown in ii) of Scheme 1B.1, the single-stranded oligonucleotide can comprise a 5′-phosphate or 5-phosphate mimic modification, as described herein, at the 5′-end of a nucleotide sequence (e.g., Z¹¹ and/or Z¹²) (e.g., a 5′-end vinylphosphonate (5′-VP), at the 5′-end of Z¹¹, as shown in ii) of Scheme 1B.1).

In some embodiments, the single-stranded oligonucleotide may further comprise one or more ligands (e.g., a lipophilic moiety for extrahepatic delivery, as described herein). In some embodiments, one or more lipophilic moieties are conjugated independently to one or more of the internal positions (i.e., non-terminal positions) of Z¹¹. In one embodiment, one or more lipophilic moieties are conjugated independently to one or more of positions 11, 12, 13 from 5′-end of Z¹¹, as shown in ii) of Scheme 1B.1. In one embodiment, one or more lipophilic moieties are conjugated independently to one or more of the internal positions of Z¹¹, excluding position 2 or 14. In one embodiment, one or more lipophilic moieties are conjugated independently to one or more positions of Z² and/or Q^{S}, excluding the positions of Q^{S} and/or Z² paired to positions 11, 12, and 13 from the 5′-end of Z¹¹, as shown in ii) of Scheme 1B.1.

In some embodiments, the single-stranded oligonucleotide may further comprise one or more targeting ligands (e.g., liver targeting carbohydrate-based ligand, as described herein). In some embodiments, one or more targeting ligands (e.g., carbohydrate-based ligands) are conjugated to the 3′-end of Z¹¹ or Z¹² (as shown in iii) of Scheme 1B.1), or an internal position of Z¹¹ or Z¹². In some embodiments, one or more targeting ligands (e.g., carbohydrate-based ligands) are conjugated to an internal position of Z¹¹, excluding position 2 or 14. In some embodiments, one or more targeting ligands (e.g., carbohydrate-based ligands) are conjugated to the 3′-end of Z², as shown in iii) of Scheme 1B.1.

In some embodiments, when the single-stranded oligonucleotide contains a terminal conjugation of a ligand to the 5′-end or 3′-end nucleotide, or contains a terminal conjugation of an abasic nucleotide, an inverted nucleotide, or an inverted abasic nucleotide to the 5′-end or 3′-end nucleotide, then at that terminus, the above internucleotide linkage modifications to the terminal nucleotide can be omitted (e.g., one or two phosphorothioate internucleotide linkage modifications between nucleotides at terminal 6 or 3 positions to the 3′-end of Z² can be omitted, due to the conjugation of a ligand to the 3′-end of Z¹², as shown in iii) of Scheme 1B.1).

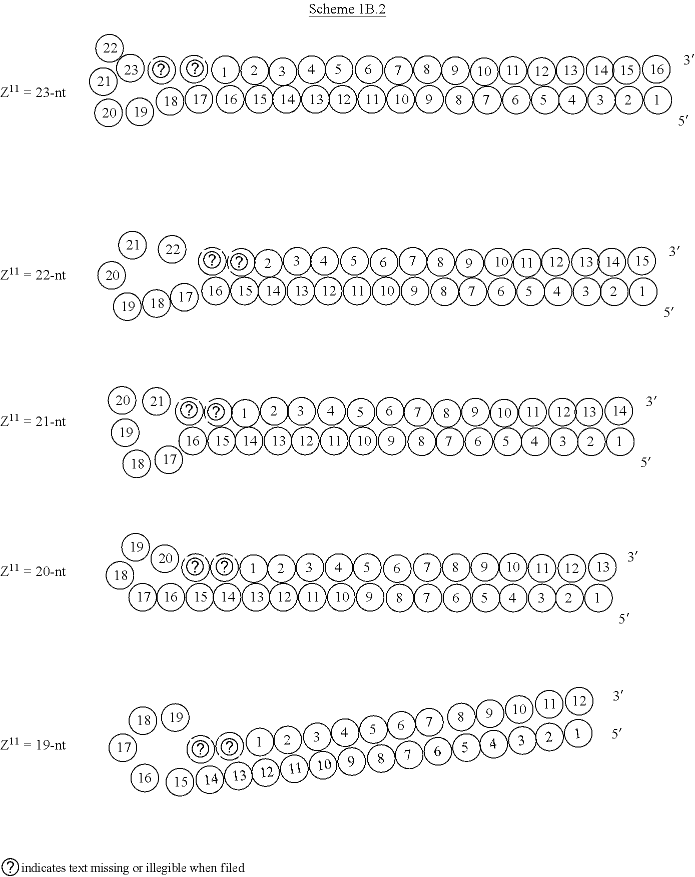

In some embodiments, Z¹¹ comprises 19-23 optionally modified nucleotides, Z¹² comprises 12-16 optionally modified nucleotides, and Q^{S} comprises 2 optionally modified nucleotides, as shown in Scheme 1B3.2.

In some embodiments, the duplexed region formed by Z¹¹ and Z¹² at the non-loop terminal (e.g., the 5′-end of Z¹¹) has a blunt end, as shown in Scheme 1B3.2.

In some embodiments, as shown in Scheme B.3, Z¹¹ comprises 19-23 optionally modified nucleotides, Z¹² comprises 12-16 optionally modified nucleotides, and Q^{S} comprises 2 optionally modified nucleotides.

In some embodiments, as shown in Scheme 1B.3, the 3-5 terminal nucleotides of Z¹¹, connected to L, Q^{S}, or Z¹², contain modifications selected from the group consisting of 2′-deoxynucleotide (dN), a 2′-deoxy-2′-fluoronucleotide (fN), a ribonucleotide (rN), 2′-O-methylnucleotide (mN), and 2′-aranucleotide (aN), to encourage cleavage. In some embodiments, the 5 terminal nucleotides of Z¹¹, connected to L, Q^{S}, or Z¹², have modifications selected from the group consisting of:
#-dN-dN-fN-fN-fN-**,
#-dN-dN-rN-dN-dN-**,
#-dN-dN-rN-rN-rN-**,
#-dN-dN-dN-dN-dN-**,
#-mN-mN-fN-fN-fN-**,
#-mN-mN-dN-dN-dN-**,
#-mN-mN-rN-dN-dN-**,
#-mN-mN-rN-rN-rN-**, and
wherein:
# is the bond to Z¹¹ and ** is the bond to L, Q^{S}, or Z²,
dN represents a 2′-deoxy nucleotide,
fN represents a 2′-deoxy-2′-fluoro nucleotide,
rN represents a ribonucleotide, and
mN represents a 2′-O-methyl nucleotide.

In some embodiments, as shown in Scheme 1B.3, the 3 terminal nucleotides of Z¹¹, connected to L, Q^{S}, or Z¹², have modifications independently selected from the group consisting of 2′-fluoro, 2′-deoxy, and 2′-OH, such as:
#-fN-fN-fN-**,
#-dN-dN-dN-**,
#-dN-dN-rN-**,
#-dN-rN-dN-**,
#-rN-dN-dN-**, and
#-rN-rN-dN-**
#-rN-dN-rN-**
#-dN-rN-rN-**
#-rN-rN-rN-**.

In one embodiment, the single-stranded oligonucleotide contains two consecutive phosphorothioate internucleotide linkage modifications between nucleotides at positions 1-2 and 2-3 of Z¹¹, and two consecutive phosphorothioate internucleotide linkage modifications between last 3 nucleotides of Z², as shown in Scheme 1B.3.

In one embodiment, the single-stranded oligonucleotide contains six terminal phosphorothioate internucleotide linkages modifications: two consecutive phosphorothioate internucleotide linkage modifications between nucleotides at positions 1-2 and 2-3 of Z¹¹, two consecutive phosphorothioate internucleotide linkage modifications between last 3 nucleotides of Z¹², and two consecutive phosphorothioate internucleotide linkage modifications between nucleotides at positions 1-2 and 2-3 of Z², as shown in Scheme 1B.3.

In some embodiments, as shown in Scheme 1B.3, the second oligonucleotide Z¹², optionally together with Q^{S}, contains at least one motif of three consecutive 2′-F modifications, and the nucleotide next to the motif is not 2′-F modified. In some embodiments, the position of the motif of three consecutive modifications is characterized by one or the followings:
the motif is at Q^{S}, positions 1 and 2 of Z¹², optionally Z¹¹ is 19 nucleotides in length;
the motif is at positions 1, 2, and 3 of Z¹², optionally Z¹¹ is 20 nucleotides in length;
the motif is at positions 2, 3, and 4 of Z¹², optionally Z¹¹ is 21 nucleotides in length;
the motif is at positions 3, 4, and 5 of Z¹², optionally Z¹¹ is 22 nucleotides in length; or
the motif is at positions 4, 5, and 6 of Z¹², optionally Z¹¹ is 23 nucleotides in length.

In some embodiments, as shown in Scheme 1B.3, the first oligonucleotide Z¹¹ contains a modification that is not 2′-O-methyl at positions 2 and 14. In one embodiment, as shown in Scheme 1B.3, the first oligonucleotide Z¹ contains a 2′-F modification at position 14.

In some embodiments, as shown in Scheme 1B.3, the first oligonucleotide Z¹¹ contains one or more 2′-deoxy (DNA) modifications at positions 2, 5, 7, and 12.

In some embodiments, as shown in Scheme 1B.3, all the remaining modifications on Z¹¹ and Z¹² are 2′-O-methyl modifications.

In some embodiments, as shown in Scheme 1B.4, Z¹ comprises 19-23 optionally modified nucleotides, Z² comprises 12-16 optionally modified nucleotides, and Q^{S} comprises 2 optionally modified nucleotides.

In some embodiments, as shown in Scheme 1B.4, the 3-5 terminal nucleotides of Z¹¹, connected to L, Q^{S}, or Z¹², contain modifications selected from the group consisting of 2′-deoxynucleotide (dN), a 2′-deoxy-2′-fluoronucleotide (fN), a ribonucleotide (rN), 2′-O-methylnucleotide (mN), and 2′-aranucleotide (aN), to encourage cleavage. In some embodiments, the 5 terminal nucleotides of Z¹¹, connected to L, Q^{S}, or Z¹², have modifications selected from the group consisting of:
#-dN-dN-fN-fN-fN-**,
#-dN-dN-rN-dN-dN-**,
#-dN-dN-rN-rN-rN-**,
#-dN-dN-dN-dN-dN-**,
#-mN-mN-fN-fN-fN-**,
#-mN-mN-dN-dN-dN-**,
#-mN-mN-rN-dN-dN-**,
#-mN-mN-rN-rN-rN-**, and
wherein:
# is the bond to Z¹¹ and ** is the bond to L, Q^{S}, or Z²,
dN represents a 2′-deoxy nucleotide,
fN represents a 2′-deoxy-2′-fluoro nucleotide,
rN represents a ribonucleotide, and
mN represents a 2′-O-methyl nucleotide.

In some embodiments, as shown in Scheme 1B.4, the 3 terminal nucleotides of Z¹¹, connected to L, Q^{S}, or Z¹², have modifications independently selected from the group consisting of 2′-fluoro, 2′-deoxy, and 2′-OH, such as:
#-fN-fN-fN-**,
#-dN-dN-dN-**,
#-dN-dN-rN-**,
#-dN-rN-dN-**,
#-rN-dN-dN-**,
#-rN-rN-dN-**
#-rN-dN-rN-**
#-dN-rN-rN-**, and
#-rN-rN-rN-**.

In one embodiment, the single-stranded oligonucleotide contains two consecutive phosphorothioate internucleotide linkage modifications between nucleotides at positions 1-2 and 2-3 of Z¹¹, and two consecutive phosphorothioate internucleotide linkage modifications between last 3 nucleotides of Z², as shown in Scheme 1B.4.

In one embodiment, the single-stranded oligonucleotide contains six terminal phosphorothioate internucleotide linkages modifications: two consecutive phosphorothioate internucleotide linkage modifications between nucleotides at positions 1-2 and 2-3 of Z¹¹, two consecutive phosphorothioate internucleotide linkage modifications between last 3 nucleotides of Z¹², and two consecutive phosphorothioate internucleotide linkage modifications between nucleotides at positions 1-2 and 2-3 of Z², as shown in Scheme 1B.4.

In some embodiments, as shown in Scheme 1B.4, the second oligonucleotide Z¹², optionally together with Q^{S}, contains at least one motif of three consecutive 2′-F modifications, and the nucleotide next to the motif is not 2′-F modified. In some embodiments, the position of the motif of three consecutive modifications is characterized by one or the followings:
the motif is at Q^{S}, positions 1 and 2 of Z¹², optionally Z¹¹ is 19 nucleotides in length;
the motif is at positions 1, 2, and 3 of Z¹², optionally Z¹¹ is 20 nucleotides in length;
the motif is at positions 2, 3, and 4 of Z¹², optionally Z¹¹ is 21 nucleotides in length;
the motif is at positions 3, 4, and 5 of Z¹², optionally Z¹¹ is 22 nucleotides in length; or
the motif is at positions 4, 5, and 6 of Z¹², optionally Z¹¹ is 23 nucleotides in length.

In some embodiments, as shown in Scheme 1B.4, Z¹², optionally together with Q^{S}, contains a 2′-O-methyl or 2′-F modification at a position that is 2 positions before the motif of three consecutive 2′-F modifications (position n-2, if the motif starts at position n), provided that the position is not part of Z¹¹. In one embodiment, as shown in Scheme 1B.4, Z¹², optionally together with Q^{S}, contains a 2′-F modification at a position that is 2 positions before the motif of three consecutive 2′-F modifications (position n-2, if the motif starts at position n), provided that the position is not part of Z¹¹.

In some embodiments, as shown in Scheme 1B.4, the first oligonucleotide Z¹¹ contains a modification that is not 2′-O-methyl at positions 2 and 14. In one embodiment, as shown in Scheme 1B.4, the first oligonucleotide Z¹¹ contains a 2′-F modification at position 14.

In some embodiments, as shown in Scheme 1B.4, the first oligonucleotide Z¹¹ contains one or more 2′-F modifications at positions 2, 6, 8, 9, 14, and 16.

In some embodiments, as shown in Scheme 1B.4, all the remaining modifications on Z¹¹ and Z¹² are 2′-O-methyl modifications.

In some embodiments, L is present in formula (II) (or IIa) or formula III (or IIIa), and contains a linking moiety represented by a formula: #—(N)ₙ—**. In this formula, # is the bond to Z¹¹ and ** is the bond to Q^{S} or Z¹¹; n is 3 to 12; and each N is independently a linking monomer having a chain length of 3 or more atoms. In some embodiments, n is 3 to 8, 4 to 8, 3 to 7, 4 to 7, 3 to 6, 4 to 6, or 3 to 5. In one embodiment, n is 3.

In some embodiments, as shown in Scheme 1B.5, all the linking monomer of L (e.g., Q304), together with LP, form a loop between W (Z¹¹) and Z². In some embodiments, as shown in Scheme 1B.5, one or more of the linking monomers of L (e.g., Q304), together with LP, forms a loop between W (Z¹¹) and Z², and one or more of the linking monomers of L (e.g., Q304) is not in the loop region. In some embodiments, as shown in Scheme 1B.5, one or more of the linking monomers of L (e.g., Q304), together with LP, forms a loop between W (Z¹¹) and Z², and one or more of the linking monomers of L (e.g., Q304) is not in the loop and is connected to Q^{S} (a). In some embodiments, as shown in Scheme 1B.5, one or more of the linking monomers of L (e.g., Q304), together with LP, forms a loop between W (Z¹¹) and Z², and one or more of the linking monomers of L (e.g., Q304) is not in the loop and is connected to Z¹².

In some embodiments, one or more linking moieties (N) in L may be an optionally modified nucleotide. In some embodiments, one or more linking moieties (N) in L may be independently selected from the group consisting of a 2′-deoxynucleotide (dN), a 2′-deoxy-2′-fluoro nucleotide (fN), a ribonucleotide (rN), 2′-O-methylnucleotide (mN), and 2′-ara nucleotide (aN) (e.g., 2′-ara-2′-deoxy, 2′-ara-2′-F, 2′-ara-2′-OMe, or 2′-ara ribonucleotide).

In some embodiments, one or more linking moieties (N) in L may be independently selected from the group consisting of Y16, Y34, Q48, Q303, Q304, Q305, Q306, Q312, Q313, Q314, Q315, Q316, Q317, Q8, Q11, Q150, Q151, Q173, Q221, Q222, Q367, and Q368.

In one embodiment, as shown in Scheme 1B.5, L contains a triplet of Q304.

In some embodiments, the single-stranded oligonucleotide contains two consecutive phosphorothioate internucleotide linkage modifications between nucleotides at positions 1-2 and 2-3 of Z¹¹, two consecutive phosphorothioate internucleotide linkage modifications between last 3 nucleotides of Z¹², and two consecutive phosphorothioate internucleotide linkage modifications between nucleotides at positions 1-2 and 2-3 of Z², as shown in Scheme 1B.4.

In some embodiments, the single-stranded oligonucleotide contains one or two phosphorothioate internucleotide linkage modifications (e.g., two consecutive phosphorothioate internucleotide linkage modifications) within last 5, 6, or 7 nucleotides of Z¹¹, as shown in Scheme 1B.5.

In one embodiment, the single-stranded oligonucleotide contains six terminal phosphorothioate internucleotide linkages modifications: two consecutive phosphorothioate internucleotide linkage modifications between nucleotides at positions 1-2 and 2-3 of Z¹¹, two consecutive phosphorothioate internucleotide linkage modifications between last 3 nucleotides of Z¹², and two consecutive phosphorothioate internucleotide linkage modifications between nucleotides at positions 1-2 and 2-3 of Z², as shown in Scheme B.5.

In one embodiment, the single-stranded oligonucleotide contains eight terminal phosphorothioate internucleotide linkages modifications: two consecutive phosphorothioate internucleotide linkage modifications between nucleotides at positions 1-2 and 2-3 of Z¹¹; two consecutive phosphorothioate internucleotide linkage modifications between last 3 nucleotides of Z¹²; two consecutive phosphorothioate internucleotide linkage modifications between nucleotides at positions 1-2 and 2-3 of Z²; and two consecutive phosphorothioate internucleotide linkage modifications) within last 5, 6, or 7 nucleotides of Z¹¹, as shown in Scheme 1B.5.

Some exemplary single-stranded oligonucleotides may have the orientation (e.g., 5′-3′ orientation) and connections of Z¹¹ and Z¹², as defined in formula (III) or (IIIa), as illustrated by Schemes 2B.1-2B.4.

In some embodiments, Z¹¹ comprises 19-23 optionally modified nucleotides, Z¹² comprises 16-19 optionally modified nucleotides, and Q^{S} may be absent or present comprising 2 optionally modified nucleotides.

In some embodiments, as shown in Scheme 2B.1, Z¹¹ comprises 23 optionally modified nucleotides, Z¹² comprises 16-19 optionally modified nucleotides. In some embodiments, as shown in Scheme 2B.2, Z¹¹ comprises 21 optionally modified nucleotides, Z¹² comprises 14-17 optionally modified nucleotides. In some embodiments, as shown in Scheme 2B.3, Z¹¹ comprises 23 optionally modified nucleotides, Z¹² comprises 18-21 optionally modified nucleotides. In some embodiments, as shown in Scheme 2B.4, Z¹¹ comprises 21 optionally modified nucleotides, Z¹² comprises 16-19 optionally modified nucleotides.

In some embodiments, the duplexed region formed by Z¹¹ and Z¹² at the non-loop terminal (e.g., the 3′-end of Z¹¹) has a blunt end, as shown in Schemes 2B.3 and 2B.4.

In some embodiments, Z¹¹ at the non-loop terminal has an overhang of 1-3 nucleotides in length. In one embodiment, Z¹¹ at the non-loop terminal has an overhang of 2 nucleotides in length (e.g., at the 3′-end of Z¹¹, as shown in Schemes 2B.1 and 2B.2). In one embodiment, Z¹¹ at the non-loop terminal has an overhang of 2 nucleotides in length and has a phosphorothioate internucleotide linkage between the two overhang nucleotides, as shown in Schemes 2B.1 and 2B.2.

In one embodiment, Z¹¹ at the non-loop terminal has an overhang of 2 nucleotides in length (e.g., at the 3′-end of Z¹¹) and has two phosphorothioate internucleotide linkages between the terminal 3 nucleotides (e.g., at the 3′-end of Z¹¹), in which 2 of the 3 nucleotides are the overhang nucleotides, and the third is the paired nucleotide next to the overhang nucleotide, as shown in Schemes 2B.1 and 2B.2.

In some embodiments, the single-stranded oligonucleotide contains one or two phosphorothioate internucleotide linkage modifications (e.g., two consecutive phosphorothioate internucleotide linkage modifications) within first 4 nucleotides of Z¹¹ or within first 3 nucleotides of Z², as shown in Schemes 2B.1 and 2B.2.

In some embodiments, the single-stranded oligonucleotide contains six terminal phosphorothioate internucleotide linkages modifications: two consecutive phosphorothioate internucleotide linkage modifications between nucleotides at positions 1-2 and 2-3 of Z¹²; two consecutive phosphorothioate internucleotide linkage modifications between nucleotides between first 4 nucleotides of Z¹¹; and two consecutive phosphorothioate internucleotide linkage modifications between last 3 nucleotides of Z¹¹, as shown in Schemes 2B.1-2B.4.

In some embodiments, the single-stranded oligonucleotide contains eight terminal phosphorothioate internucleotide linkages modifications: two consecutive phosphorothioate internucleotide linkage modifications between nucleotides at positions 1-2 and 2-3 of Z¹²; two consecutive phosphorothioate internucleotide linkage modifications between last 3 nucleotides of Z¹¹; two consecutive phosphorothioate internucleotide linkage modifications between nucleotides between first 4 nucleotides of Z¹¹; and two consecutive phosphorothioate internucleotide linkage modifications between last 3 nucleotides of Z¹¹, as shown in Schemes 2B.1-2B.4.

In some embodiments, when the single-stranded oligonucleotide contains a terminal conjugation of a ligand to the 5′-end or 3′-end nucleotide, or contains a terminal conjugation of an abasic nucleotide, an inverted nucleotide, or an inverted abasic nucleotide to the 5′-end or 3′-end nucleotide, then at that terminus, the above internucleotide linkage modifications to the terminal nucleotide can be omitted (e.g., one or two phosphorothioate internucleotide linkage modifications between nucleotides at terminal 6 or 3 positions to the 5′-end of Z² can be omitted, due to the conjugation of a ligand to the 5′-end of Z², as shown in Schemes 2B.1-2B.4).

In some embodiments, as shown in Schemes 2B.1-2B.4, the intra-strand duplexed region formed by Z¹¹ and Z¹² may contain all consecutive base pairs, or may contain up to 3 (e.g., 0, 1, 2, or 3) mismatch based pairs. In one embodiment, Z² may contain one nucleotide that forms mismatched base pair with the opposite nucleotide in Z¹¹ (e.g., the last nucleotide of Z², or the n−1^{th} nucleotide if the last nucleotide is the n^{th} nucleotide).

In some embodiments, as shown in Schemes 2B.1-2B.4, the 3-5 terminal nucleotides of Z¹¹, connected to L, Q^{S}, or Z², contain modifications selected from the group consisting of 2′-deoxynucleotide (dN), a 2′-deoxy-2′-fluoronucleotide (fN), a ribonucleotide (rN), 2′-O-methylnucleotide (mN), and 2′-aranucleotide (aN), to encourage cleavage. In some embodiments, the 5 terminal nucleotides of Z¹¹, connected to L, Q^{S}, or Z², have modifications selected from the group consisting of:
#-dN-dN-fN-fN-fN-**,
#-dN-dN-rN-dN-dN-**,
#-dN-dN-rN-rN-rN-**,
#-dN-dN-dN-dN-dN-**,
#-mN-mN-fN-fN-fN-**,
#-mN-mN-dN-dN-dN-**,
#-mN-mN-rN-dN-dN-**, and
#-mN-mN-rN-rN-rN-**.

In some embodiments, as shown in Schemes 2B.1-2B.4, the 3 terminal nucleotides of Z¹¹, connected to L, Q^{S}, or Z¹², have modifications independently selected from the group consisting of 2′-fluoro, 2′-deoxy, and 2′-OH, such as:
#-fN-fN-fN-**,
#-dN-dN-dN-**,
#-dN-dN-rN-**,
#-dN-rN-dN-**,
#-rN-dN-dN-**,
#-rN-rN-dN-**
#-rN-dN-rN-**
#-dN-rN-rN-**, and
#-rN-rN-rN-**.

In some embodiments, L is present in formula (II) (or IIa) or formula III (or IIIa), and contains a linking moiety represented by a formula: #—(N)ₙ—**. In this formula, # is the bond to Z¹¹ and ** is the bond to Q^{S} or Z¹²; n is 3 to 12; and each N is independently a linking monomer having a chain length of 3 or more atoms. In some embodiments, n is 3 to 8, 4 to 8, 3 to 7, 4 to 7, 3 to 6, 4 to 6, or 3 to 5. In one embodiment, n is 3.

In some embodiments, as shown in Schemes 2B.1-2B.4, all the linking monomer of L (e.g., Q304), together with LP, form a loop between W (Z¹¹) and Z¹². In some embodiments, as shown in Schemes 2B.1-2B.4, one or more of the linking monomers of L (e.g., Q304), together with LP, forms a loop between W (Z¹¹) and Z¹², and one or more of the linking monomers of L (e.g., Q304) is not in the loop region. In some embodiments, as shown in Schemes 2B.1-2B.4, one or more of the linking monomers of L (e.g., Q304), together with LP, forms a loop between W (Z¹¹) and Z¹², and one or more of the linking monomers of L (e.g., Q304) is not in the loop and is connected to Q^{S} (a). In some embodiments, as shown in Schemes 2B.1-2B.4, one or more of the linking monomers of L (e.g., Q304), together with LP, forms a loop between W (Z¹¹) and Z¹², and one or more of the linking monomers of L (e.g., Q304) is not in the loop and is connected to Z¹².

In some embodiments, one or more linking moieties (N) in L may be an optionally modified nucleotide. In some embodiments, one or more linking moieties (N) in L may be independently selected from the group consisting of a 2′-deoxynucleotide (dN), a 2′-deoxy-2′-fluoro nucleotide (fN), a ribonucleotide (rN), 2′-O-methylnucleotide (mN), and 2′-ara nucleotide (aN) (e.g., 2′-ara-2′-deoxy, 2′-ara-2′-F, 2′-ara-2′-OMe, or 2′-ara ribonucleotide).

In some embodiments, one or more linking moieties (N) in L may be independently selected from the group consisting of Y16, Y34, Q48, Q303, Q304, Q305, Q306, Q312, Q313, Q314, Q315, Q316, Q317, Q8, Q11, Q150, Q151, Q173, Q221, Q222, Q367, and Q368.

In one embodiment, as shown in Schemes 2B.1-2B.4, L contains a triplet of Q304.

The single-stranded oligonucleotide nucleotide sequence may be a substrate cleavable by DICER.

Another aspect of the invention relates to an oligonucleotide construct comprising two single-stranded oligonucleotides of formula (I) as described above, wherein the two single-stranded oligonucleotides are covalently bonded. In some embodiments, the two single-stranded oligonucleotides are covalently bonded via a tethering group. Exemplary tethering groups and exemplary process for covalently bonding two single-stranded oligonucleotides to form an oligonucleotide construct are shown in Schemes 7.1-7.4 below.

Certain embodiments of the invention relate to a linking group design for connecting the two oligonucleotides to form the single-stranded oligonucleotide nucleotide. Certain embodiments of the invention relate to a tethering group design, for connecting the two oligonucleotides to form the oligonucleotide construct (i.e., the gemini style).

### Linkers/Tethers

Linkers/Tethers may be contained in the linking group L in the single-stranded oligonucleotide to connect the two oligonucleotides to form the single-stranded oligonucleotide.

Linkers/Tethers may be contained in the tethering group in the oligonucleotide construct (i.e., the gemini style) to connect the two single-stranded oligonucleotides to form the oligonucleotide construct.

Linkers/tethers can also be used to connect the ligand to the single-stranded oligonucleotide, e.g., via a carrier.

The terms “linker,” “linkage,” “linking group,” “linking moiety,” and “tether” can be used interchangeably.

The linking group L may contain multiple linkers/tethers, each may be the same or different.

The linking group L in the single-stranded oligonucleotide may be a nucleotide-based or non-nucleotide-based linker. The linking group L may be a stable linker that is stable in a biological fluid (e.g., in plasma or artificial cerebrospinal fluid). Alternatively, the linking group L may be a cleavable linking group (e.g., a bio-cleavable linker).

Linkers/tethers may be connected to a ligand at a “tethering attachment point (TAP).” Linkers/Tethers may include any C₁-C₁₀₀ carbon-containing moiety, (e.g. C₁-C₇₅, C₁-C₅₀, C₁-C₂₀, C₁-C₁₀; C₁, C₂, C₃, C₄, C₅, C₆, C₇, C₈, C₉, or C₁₀), and may have at least one nitrogen atom. In certain embodiments, the nitrogen atom forms part of a terminal amino or amido (NHC(O)—) group on the linker/tether, which may serve as a connection point for the ligand. Non-limited examples of linkers/tethers (underlined) include TAP-(CH₂)ₙNH—; TAP-C(O)(CH₂)ₙNH—; TAP-NR″″CH₂)ₙNH—, TAP-C(O)—(CH₂)ₙ—C(O)—; TAP-C(O)—(CH₂)ₙ—C(O)O—; TAP-C(O)—O—; TAP-C(O)—(CH₂)ₙ—NH—C(O)—; TAP-C(O)—(CH₂)ₙ—; TAP-C(O)—NH—; TAP-C(O)—; TAP-(CH₂)ₙ—C(O)—; TAP-(CH₂)ₙ—C(O)O—; TAP-(CH₂)ₙ—; or TAP-(CH₂)ₙ—NH—C(O)—; in which n is 1-20 (e.g., 1, 2, 3, 4, 5, 6, 7, 8, 9, 10, 11, 12, 13, 14, 15, 16, 17, 18, 19, or 20) and R″″ is C₁-C₆ alkyl. Preferably, n is 5, 6, or 11. In other embodiments, the nitrogen may form part of a terminal oxyamino group, e.g., —ONH₂, or hydrazino group, —NHNH₂. The linker/tether may optionally be substituted, e.g., with hydroxy, alkoxy, perhaloalkyl, and/or optionally inserted with one or more additional heteroatoms, e.g., N, O, or S. Preferred tethered ligands may include, e.g., TAP-(CH₂)ₙNH(LIGAND); TAP-C(O)(CH₂)ₙNH(LIGAND); TAP-NR″″(CH₂)ₙNH(LIGAND); TAP-(CH₂)ₙONH(LIGAND); TAP-C(O)(CH₂)ₙONH(LIGAND); TAP-NR″″(CH₂)ₙONH(LIGAND); TAP-(CH₂)ₙNHNH₂(LIGAND), TAP-C(O)(CH₂)ₙNHNH₂(LIGAND); TAP-NR″″(CH₂)ₙNHNH₂(LIGAND); TAP-C(O)—(CH₂)ₙ—C(O)(LIGAND); TAP-C(O)—(CH₂)ₙ—C(O)O(LIGAND); TAP-C(O)—O(LIGAND); TAP-C(O)—(CH₂)ₙ—NH—C(O)(LIGAND); TAP-C(O)—(CH₂)ₙ(LIGAND); TAP-C(O)—NH(LIGAND); TAP-C(O)(LIGAND); TAP-(CH₂)ₙ—C(O) (LIGAND); TAP-(CH₂)ₙ—C(O)O(LIGAND); TAP-(CH₂)ₙ(LIGAND); or TAP-(CH₂)ₙ—NH—C(O)(LIGAND). In some embodiments, amino terminated linkers/tethers (e.g., NH₂, ONH₂, NH₂NH₂) can form an imino bond (i.e., C═N) with the ligand. In some embodiments, amino terminated linkers/tethers (e.g., NH₂, ONH₂, NH₂NH₂) can acylated, e.g., with C(O)CF₃.

In some embodiments, the linker/tether can terminate with a mercapto group (i.e., SH) or an olefin (e.g., CH═CH₂). For example, the tether can be TAP-(CH₂)ₙ—SH, TAP-C(O)(CH₂)ₙSH, TAP-(CH₂)ₙ—(CH═CH₂), or TAP-C(O)(CH₂)ₙ(CH═CH₂), in which n can be as described elsewhere. The tether may optionally be substituted, e.g., with hydroxy, alkoxy, perhaloalkyl, and/or optionally inserted with one or more additional heteroatoms, e.g., N, O, or S. The double bond can be cis or trans or E or Z.

In other embodiments, the linker/tether may include an electrophilic moiety, preferably at the terminal position of the linker/tether. Exemplary electrophilic moieties include, e.g., an aldehyde, alkyl halide, mesylate, tosylate, nosylate, or brosylate, or an activated carboxylic acid ester, e.g. an NHS ester, or a pentafluorophenyl ester. Preferred linkers/tethers (underlined) include TAP-(CH₂)ₙCHO; TAP-C(O)(CH₂)ₙCHO; or TAP-NR″″(CH₂)ₙCHO, in which n is 1-6 and R″″ is C₁-C₆ alkyl; or TAP-(CH₂)ₙC(O)ONHS; TAP-C(O)(CH₂)ₙC(O)ONHS; or TAP-NR″″(CH₂)ₙC(O)ONHS, in which n is 1-6 and R″″ is C₁-C₆ alkyl; TAP-(CH₂)ₙC(O)OC₆F₅; TAP-C(O)(CH₂)ₙC(O) OC₆F₅; or TAP-NR″″(CH₂)ₙC(O)OC₆F₅, in which n is 1-11 and R″″ is C₁-C₆ alkyl; or —(CH₂)ₙCH₂LG; TAP-C(O)(CH₂)ₙCH₂LG; or TAP-NR″″(CH₂)ₙCH₂LG, in which n can be as described elsewhere and R″″ is C₁-C₆ alkyl (LG can be a leaving group, e.g., halide, mesylate, tosylate, nosylate, brosylate). Tethering can be carried out by coupling a nucleophilic group of a ligand, e.g., a thiol or amino group with an electrophilic group on the tether.

In other embodiments, it can be desirable for the monomer to include a

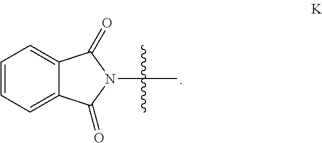 [SMILES: *N1C(=O)c2ccccc2C1=O]

phthalimido group (K) at the terminal position of the linker/tether.

In other embodiments, other protected amino groups can be at the terminal position of the linker/tether, e.g., alloc, monomethoxy trityl (MMT), trifluoroacetyl, Fmoc, or aryl sulfonyl (e.g., the aryl portion can be ortho-nitrophenyl or ortho, para-dinitrophenyl).

Any of the linkers/tethers described herein may further include one or more additional linking groups, e.g., —O—(CH₂)ₙ—, —(CH₂)ₙ—SS—, —(CH₂)ₙ—, or —(CH═CH)—.

### Cleavable Linkers/Tethers

In some embodiments, at least one of the linkers/tethers can be a redox cleavable linker, an acid cleavable linker, an esterase cleavable linker, a phosphatase cleavable linker, a peptidase cleavable linker, or endosomal cleavable linker.

In one embodiment, at least one of the linkers/tethers can be a reductively cleavable linker (e.g., a disulfide group).

In one embodiment, at least one of the linkers/tethers can be an acid cleavable linker (e.g., a hydrazone group, an ester group, an acetal group, or a ketal group).

In one embodiment, at least one of the linkers/tethers can be an esterase cleavable linker (e.g., an ester group).

In one embodiment, at least one of the linkers/tethers can be a phosphatase cleavable linker (e.g., a phosphate group).

In one embodiment, at least one of the linkers/tethers can be a peptidase cleavable linker (e.g., a peptide bond).

In one embodiment, at least one of the linkers/tethers can be an endosomal cleavable linker (or a protease cleavable linker, e.g., a carbohydrate linker). For instance, a carbohydrate linker is cleaved at least 1.25 times faster in the cell (or under in vitro conditions selected to mimic intracellular conditions) as compared to blood or serum (or under in vitro conditions selected to mimic extracellular conditions).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some tethers will have a linkage group that is cleaved at a preferred pH, thereby releasing the iRNA agent from a ligand (e.g., a targeting or cell-permeable ligand, such as cholesterol) inside the cell, or into the desired compartment of the cell.

A chemical junction (e.g., a linking group) that links a ligand to an iRNA agent can include a disulfide bond. When the iRNA agent/ligand complex is taken up into the cell by endocytosis, the acidic environment of the endosome will cause the disulfide bond to be cleaved, thereby releasing the iRNA agent from the ligand (Quintana et al., Pharm Res. 19:1310-1316, 2002; Patri et al., Curr. Opin. Curr. Biol. 6:466-471, 2002). The ligand can be a targeting ligand or a second therapeutic agent that may complement the therapeutic effects of the iRNA agent.

A tether can include a linking group that is cleavable by a particular enzyme. The type of linking group incorporated into a tether can depend on the cell to be targeted by the iRNA agent. For example, an iRNA agent that targets an mRNA in liver cells can be conjugated to a tether that includes an ester group. Liver cells are rich in esterases, and therefore the tether will be cleaved more efficiently in liver cells than in cell types that are not esterase-rich. Cleavage of the tether releases the iRNA agent from a ligand that is attached to the distal end of the tether, thereby potentially enhancing silencing activity of the iRNA agent. Other cell-types rich in esterases include cells of the lung, renal cortex, and testis.

Tethers that contain peptide bonds can be conjugated to iRNA agents target to cell types rich in peptidases, such as liver cells and synoviocytes. For example, an iRNA agent targeted to synoviocytes, such as for the treatment of an inflammatory disease (e.g., rheumatoid arthritis), can be conjugated to a tether containing a peptide bond.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue, e.g., tissue the iRNA agent would be exposed to when administered to a subject. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

The cleavable linker may be cleavable in various tissue and cell structures, e.g., in a homogenate, tritosome, cytosol, or endosome of any types of cells, such as in liver homogenates, liver tritosomes, liver lysosomes, liver cytosol, liver endosomes, brain homogenates, brain tritosomes, brain lysosomes, brain cytosol, or brain endosomes.

### Redox Cleavable Linking Groups

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### Phosphate-Based Cleavable Linking Groups

Phosphate-based linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)O—, —O—P(S)(SH)O—, —S—P(O)(OH)O—, —O—P(O)(OH)—S—, —S—P(O)(OH)—S—, —O—P(S)(OH)—S—, —S—P(S)(OH)O—, —O—P(O)(H)O—, —O—P(S)(H)O—, —S—P(O)(H)O—, —S—P(S)(H)O—, —S—P(O)(H)—S—, —O—P(S)(H)—S—. A preferred embodiment is O—P(O)(OH)—O—. These candidates can be evaluated using methods analogous to those described above.

### Acid Cleavable Linking Groups

Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, ketals, acetals, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### Ester-Based Linking Groups

Ester-based linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

### Peptide-Based Cleaving Groups

Peptide-based linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynylene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide cleavable linking groups have the general formula —NHCHR¹C(O)NHCHR²C(O)—, where R¹ and R² are the R groups of the two adjacent amino acids. These candidates can be evaluated using methods analogous to those described above.

### Biocleavable Linkers Tethers

The linkers can also include biocleavable linkers that are nucleotide and non-nucleotide linkers, or combinations thereof, that connect two parts of a molecule. For example, a biocleavable linker may be used as part of the linking group L to connect the two oligonucleotides of the single-stranded oligonucleotide. In some embodiments, mere electrostatic or stacking interaction between two individual nucleotide sequences can represent a linker.

The non-nucleotide linkers include tethers or linkers derived from monosaccharides, disaccharides, oligosaccharides, and derivatives thereof, aliphatic, alicyclic, heterocyclic, and combinations thereof.

In some embodiments, at least one of the linkers (tethers) is a bio-cleaveable linker selected from the group consisting of DNA, RNA, disulfide, amide, functionalized monosaccharides or oligosaccharides of galactosamine, glucosamine, glucose, galactose, and mannose, and combinations thereof.

In some embodiments, the cleavable linker (or the bio-cleavable linker) contains one or more carbohydrate (saccharide) moieties and/or a peptide linker. The cleavable linker (or the bio-cleavable linker) may be used to connect two nucleotide sequences or oligonucleotides, connect a nucleotide sequence or an oligonucleotide with a ligand, or connect a ligand and endosomal cleavable agent.

In some embodiments, the bio-cleavable carbohydrate linker has one or more of the following features:
i) the bio-cleavable carbohydrate linker may have 1 to 10 saccharide units,
ii) the saccharide moieties have at least one anomeric linkage capable of connecting two nucleotide sequences or oligonucleotides,
iii) when two or more saccharides are present, these nucleotide sequences or oligonucleotides can be linked via 1-3, 1-4, or 1-6 sugar linkages,
iv) when two or more saccharides are present, these nucleotide sequences or oligonucleotides may also be linked via alkyl chains.

Exemplary bio-cleavable linkers include:

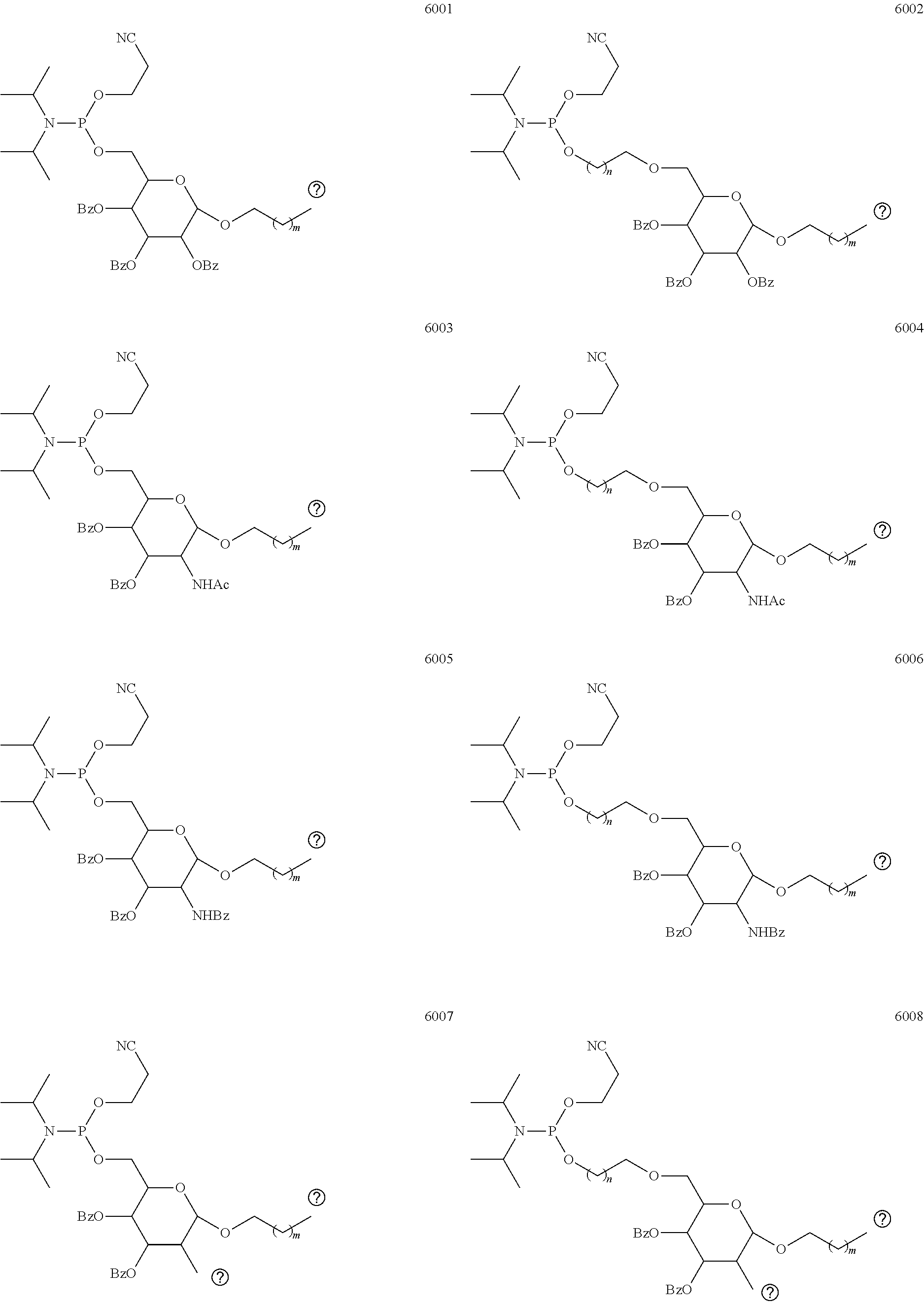

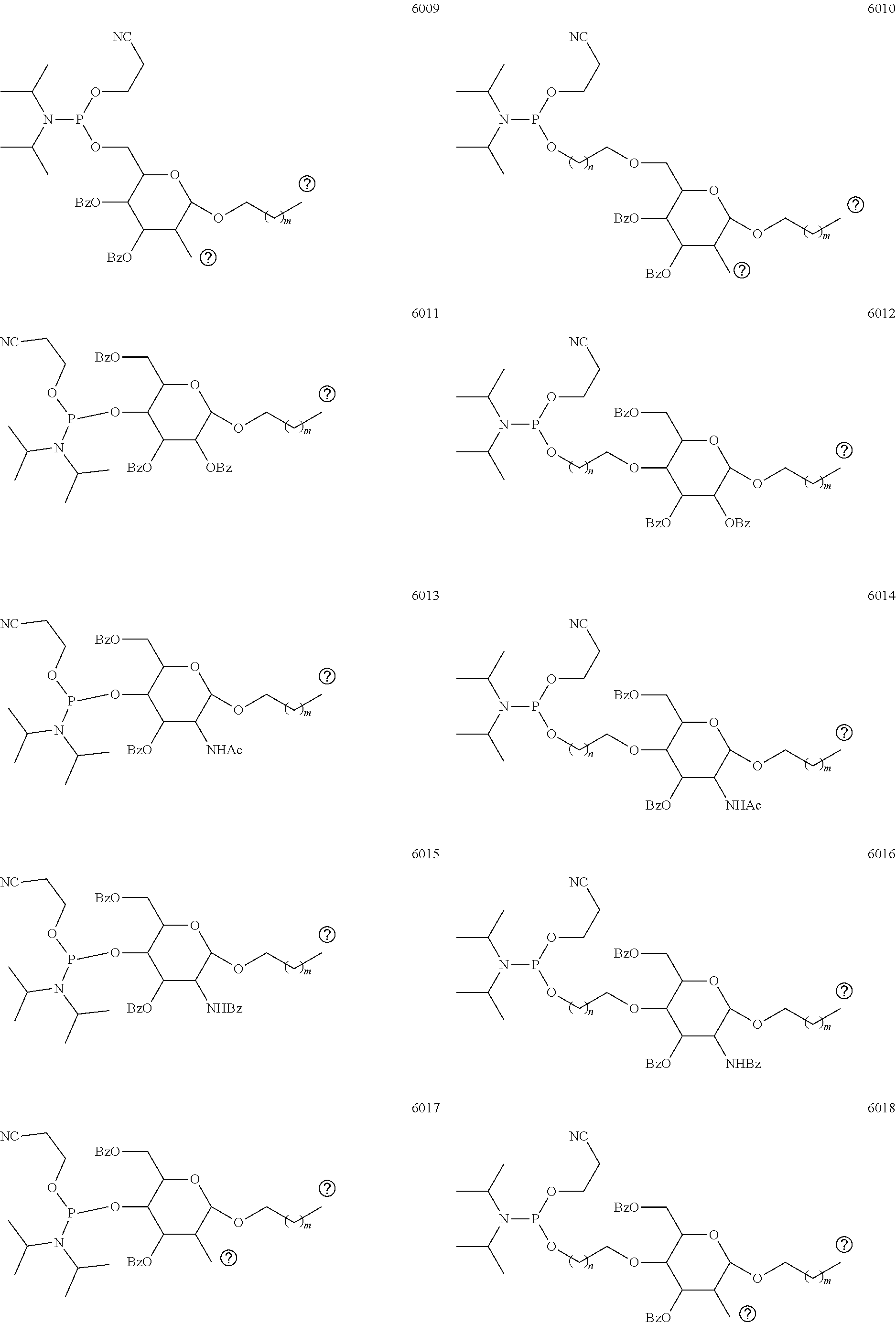

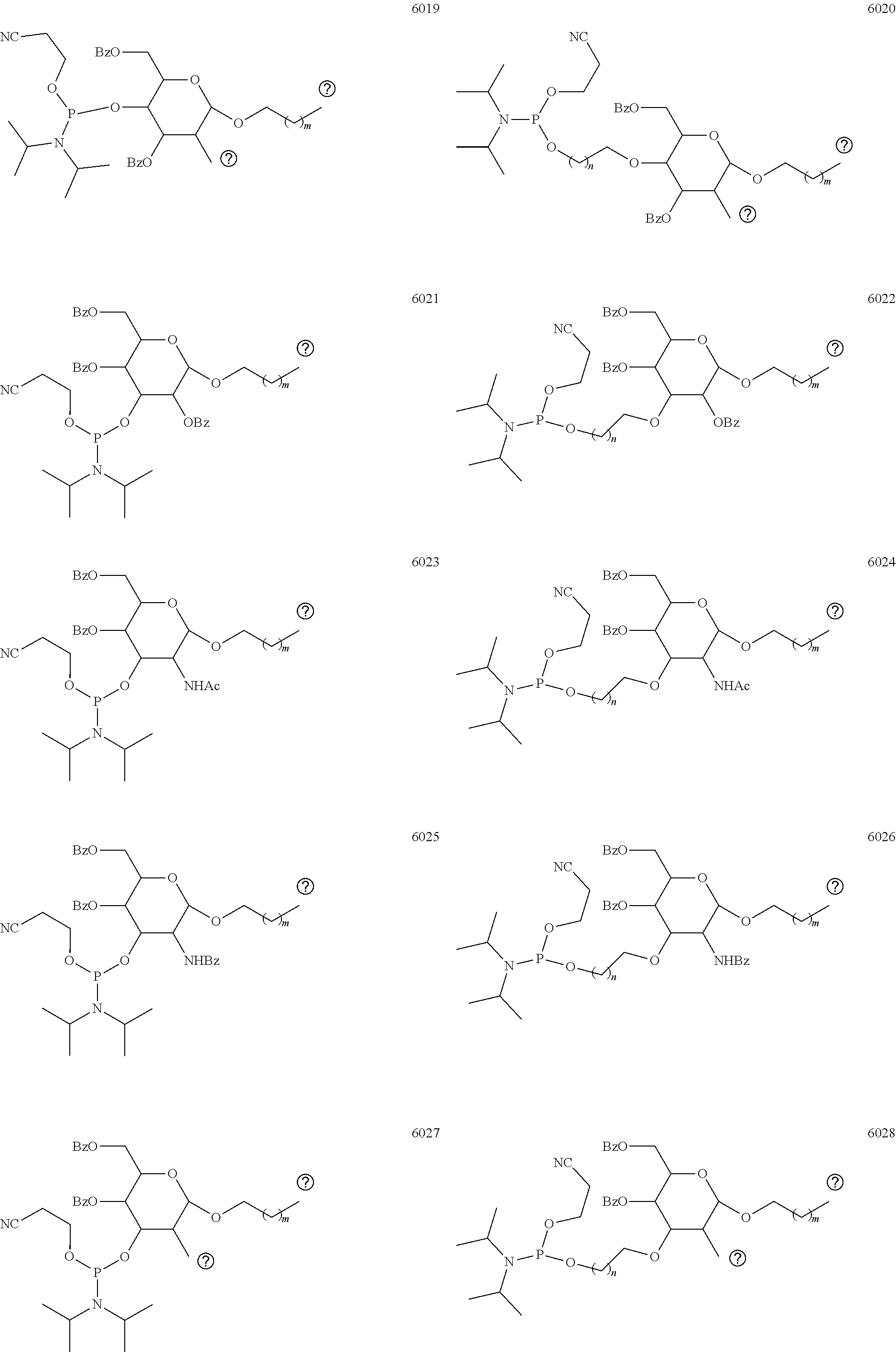

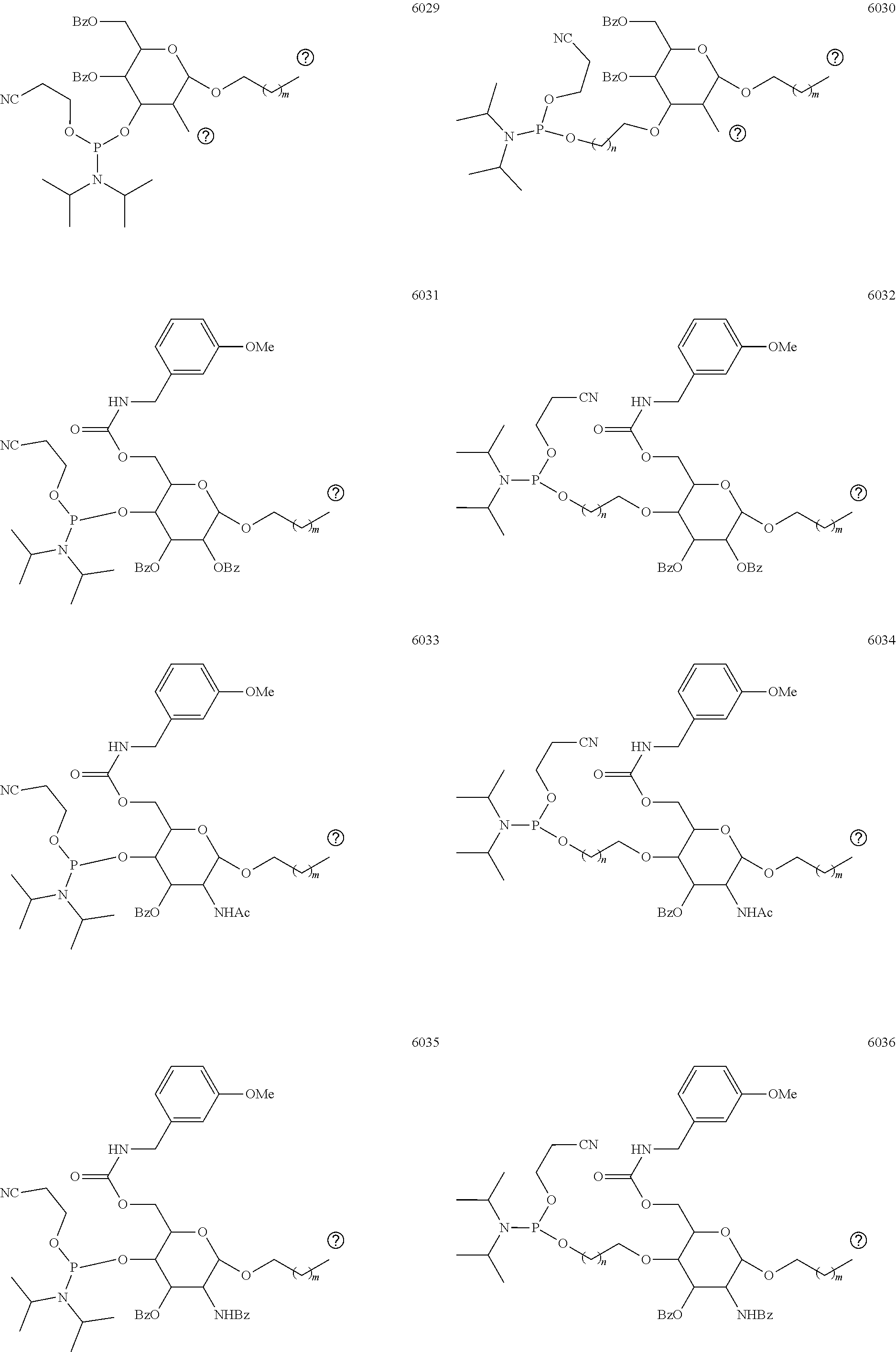

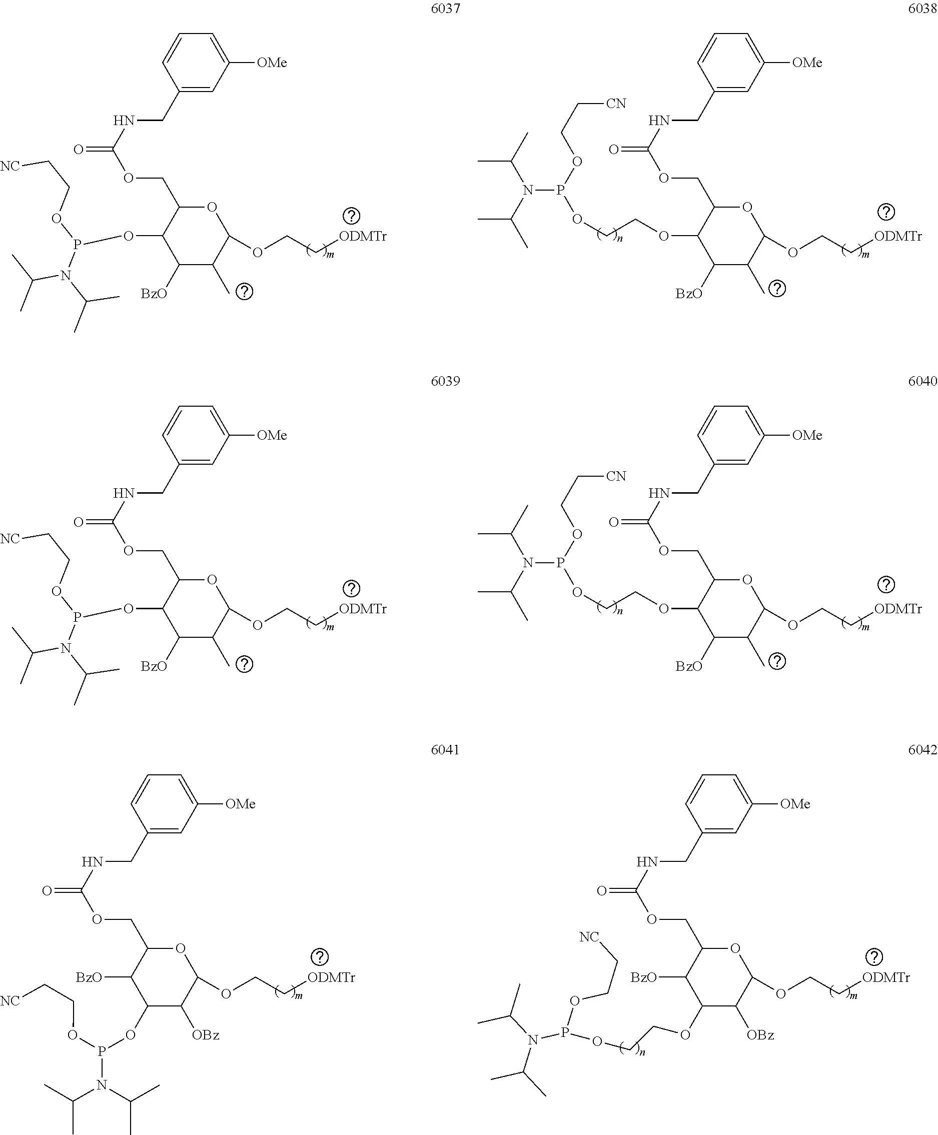

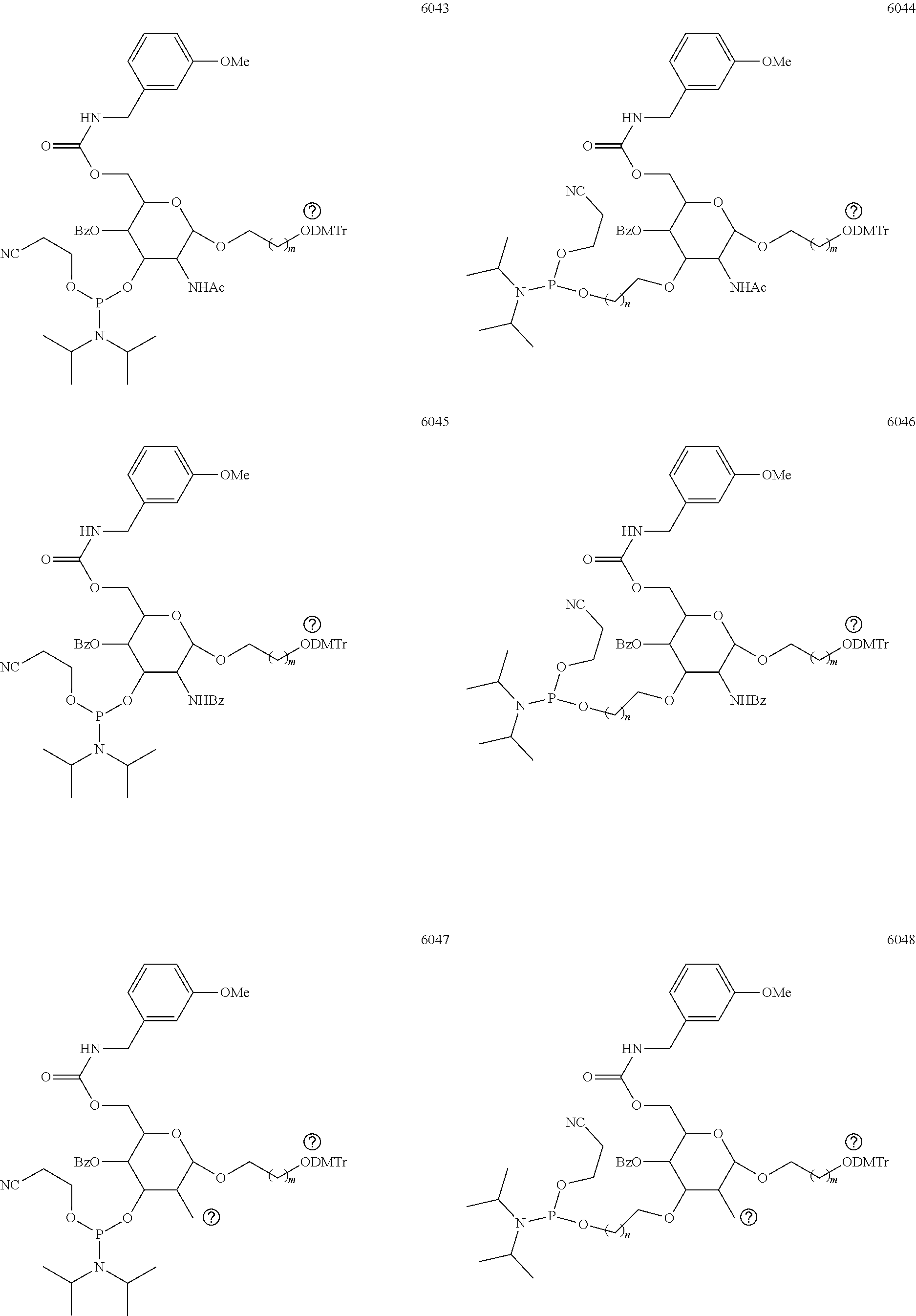

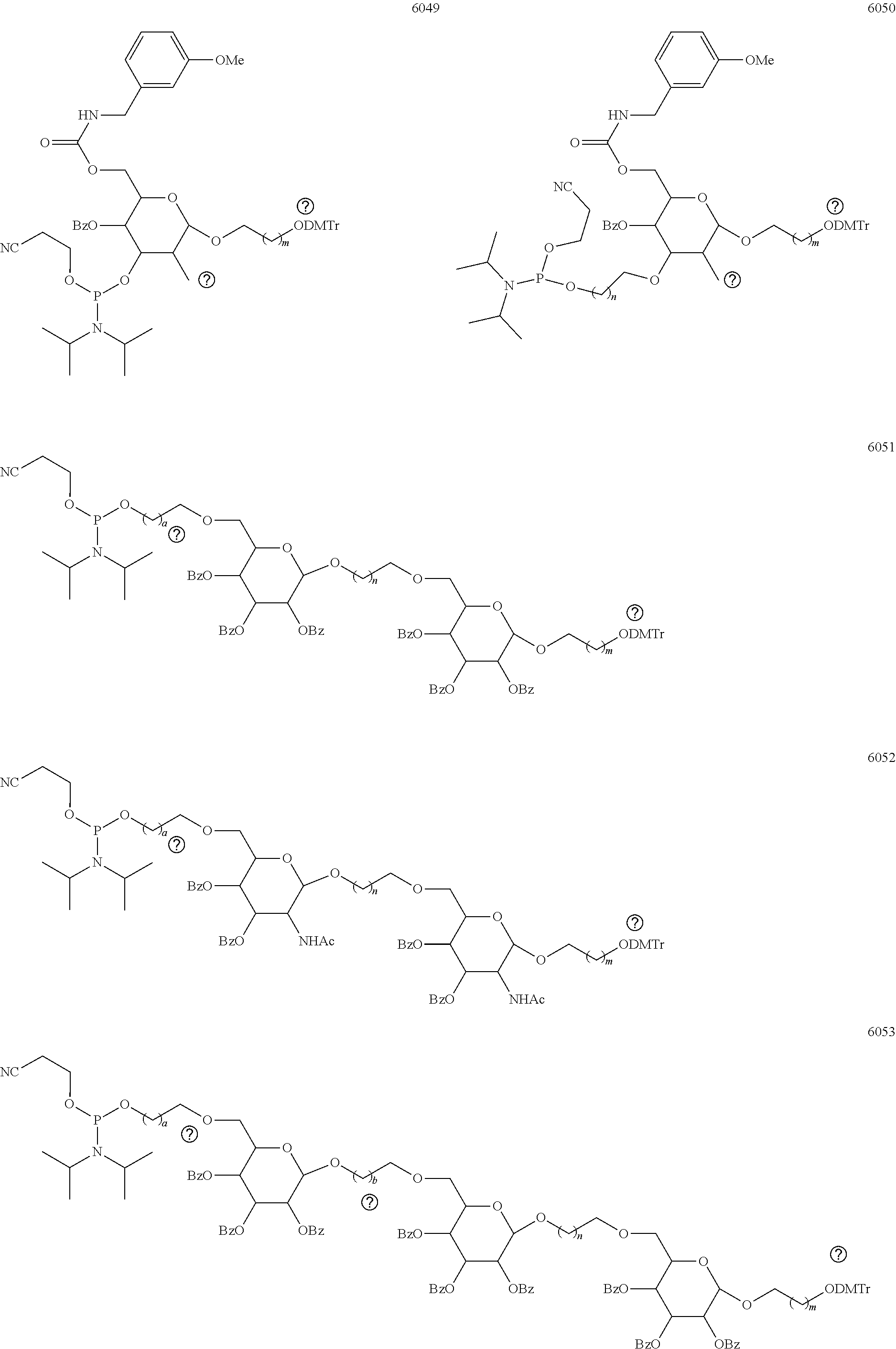

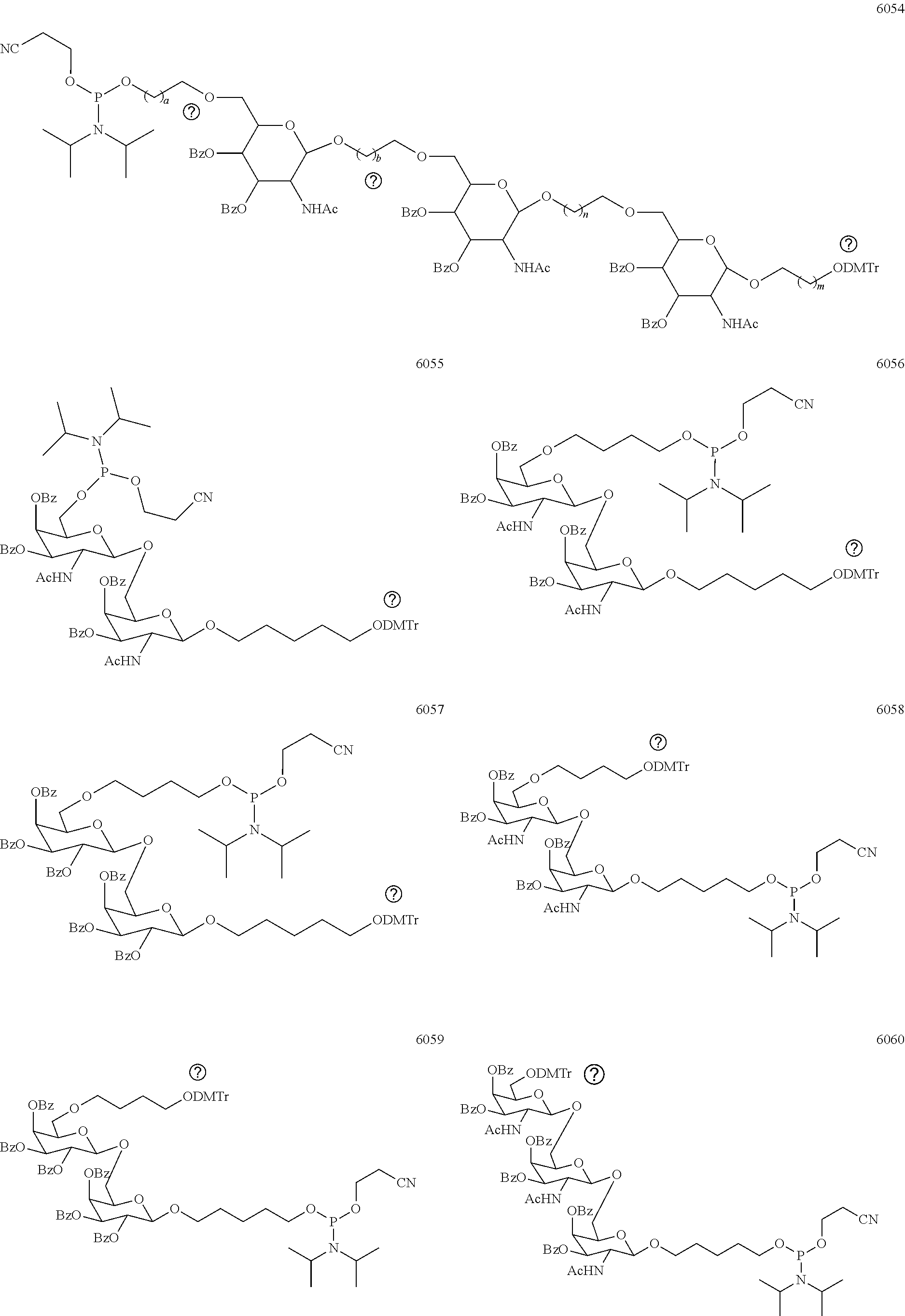

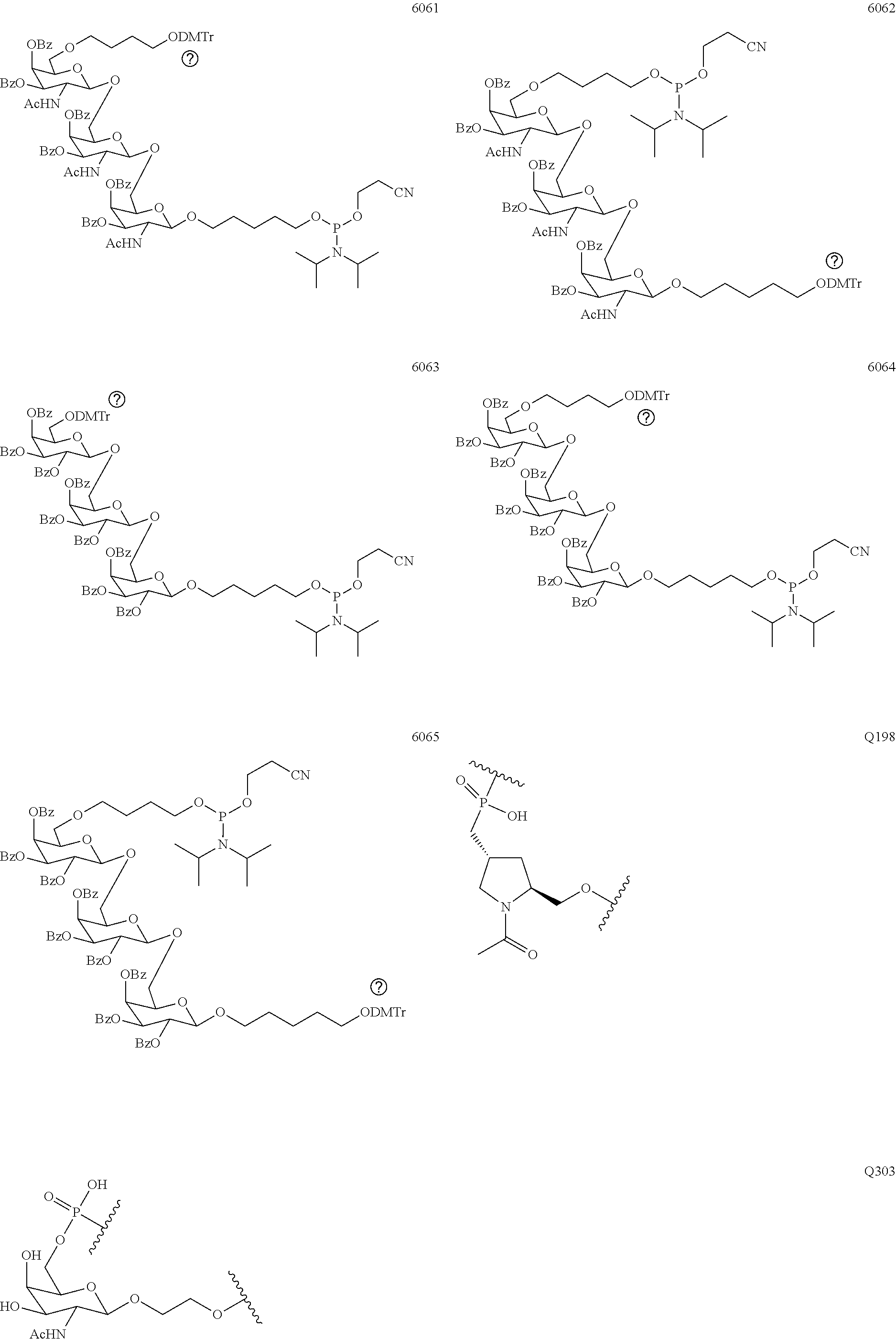

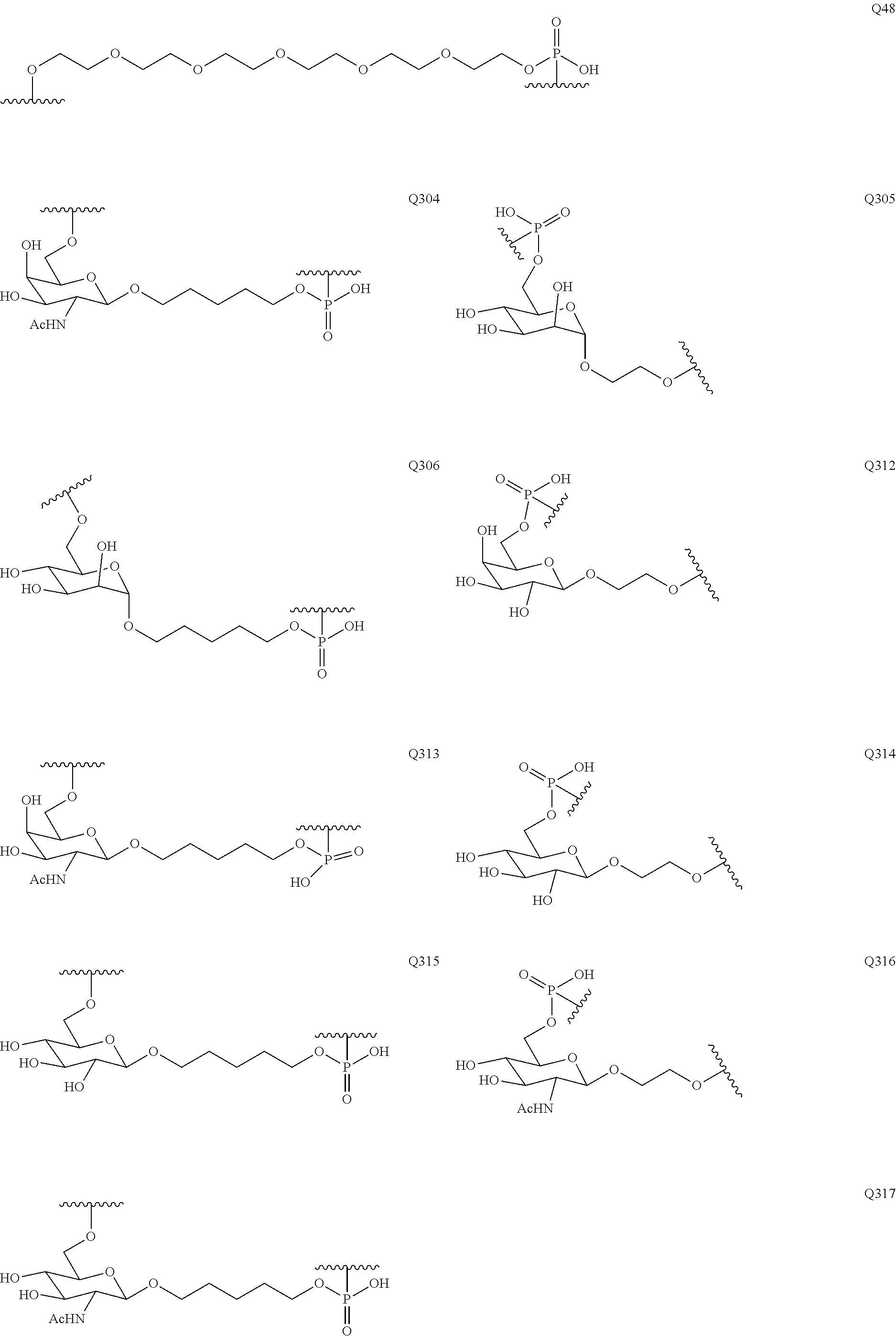

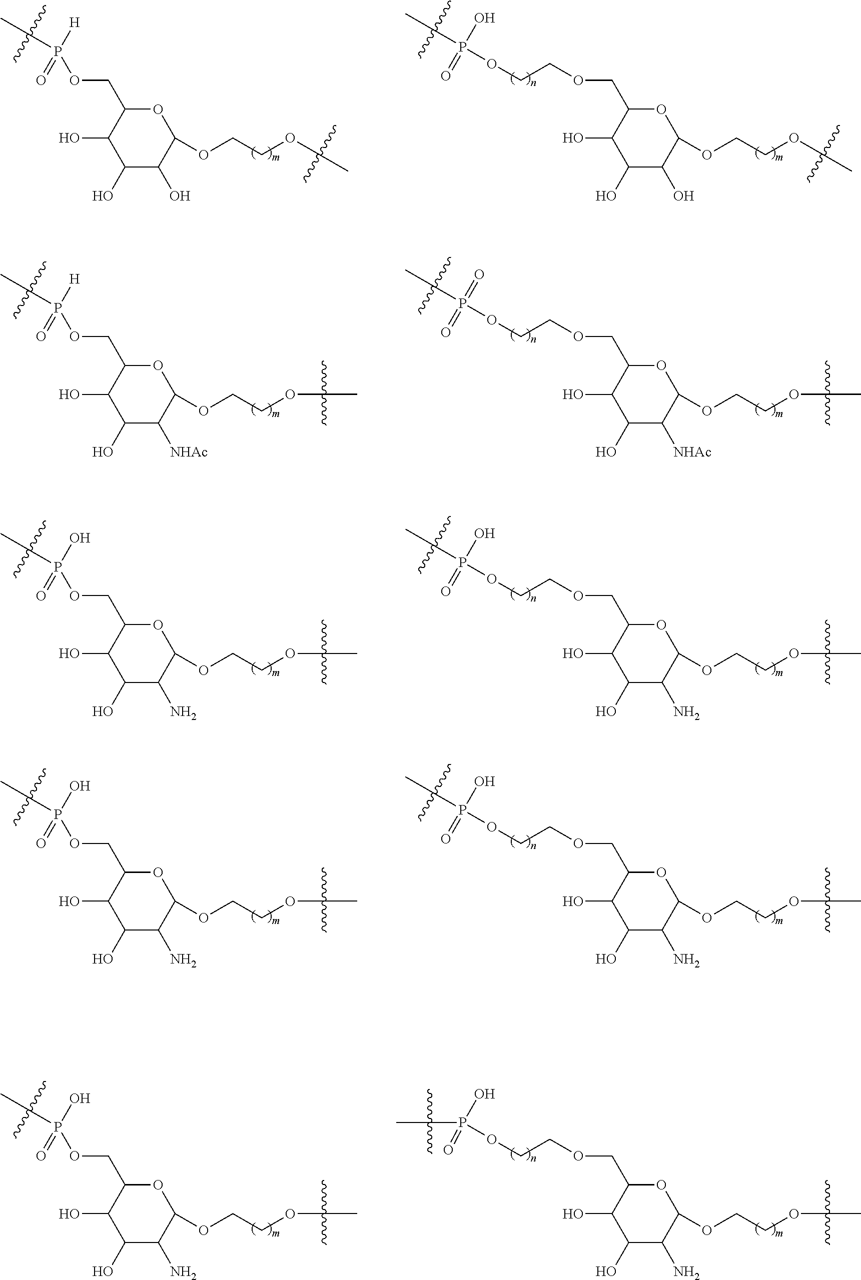

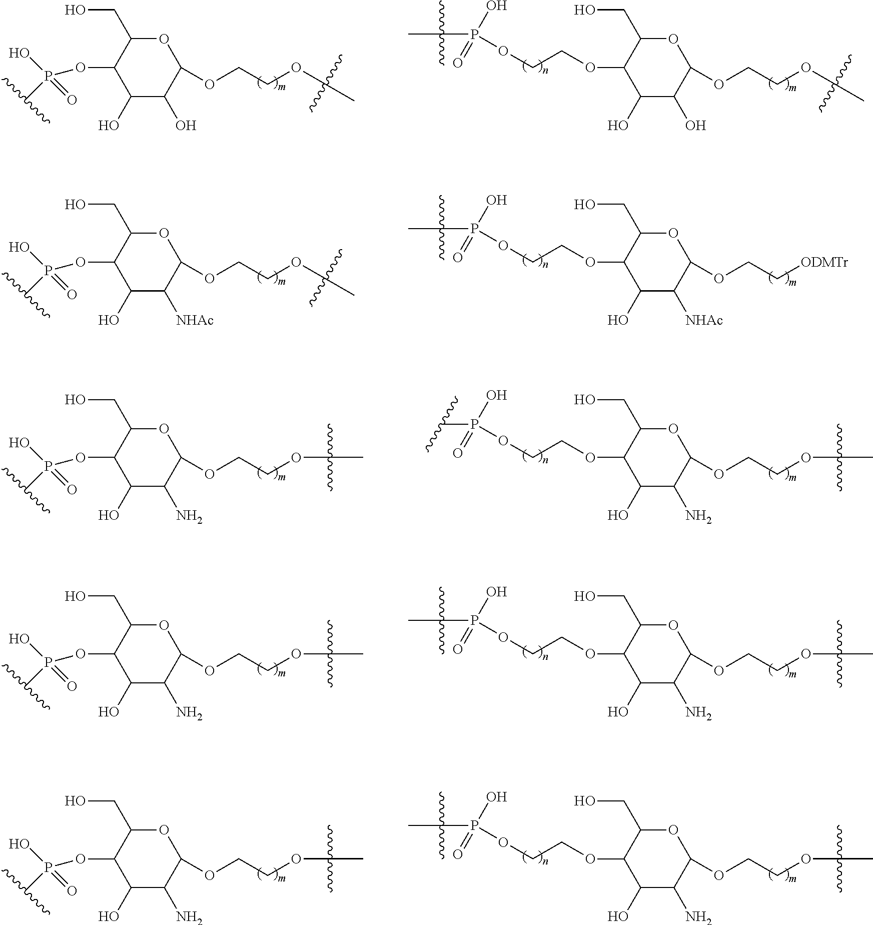

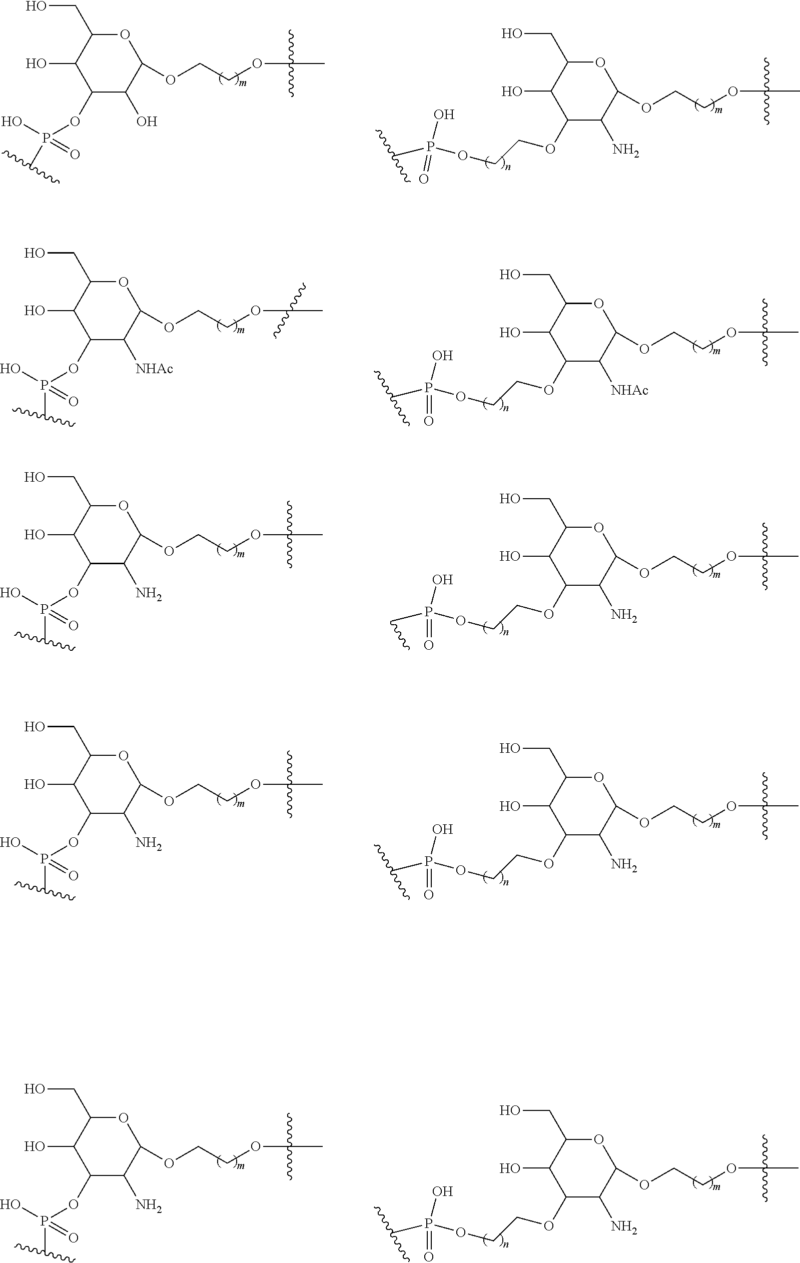

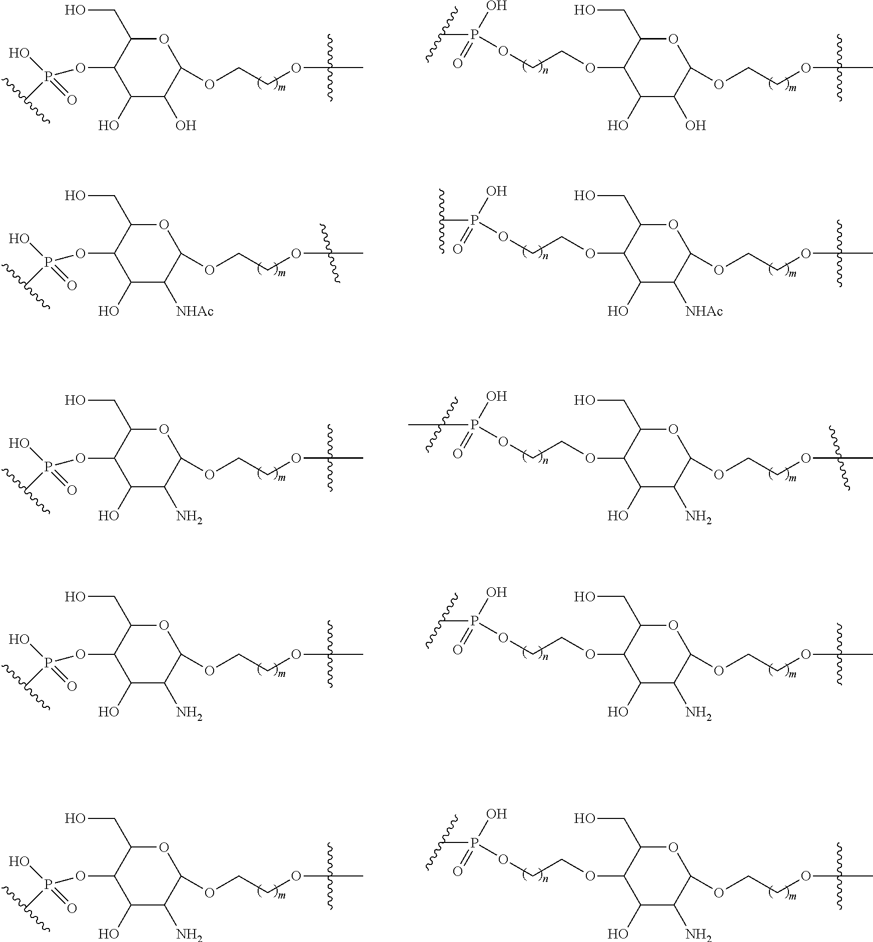

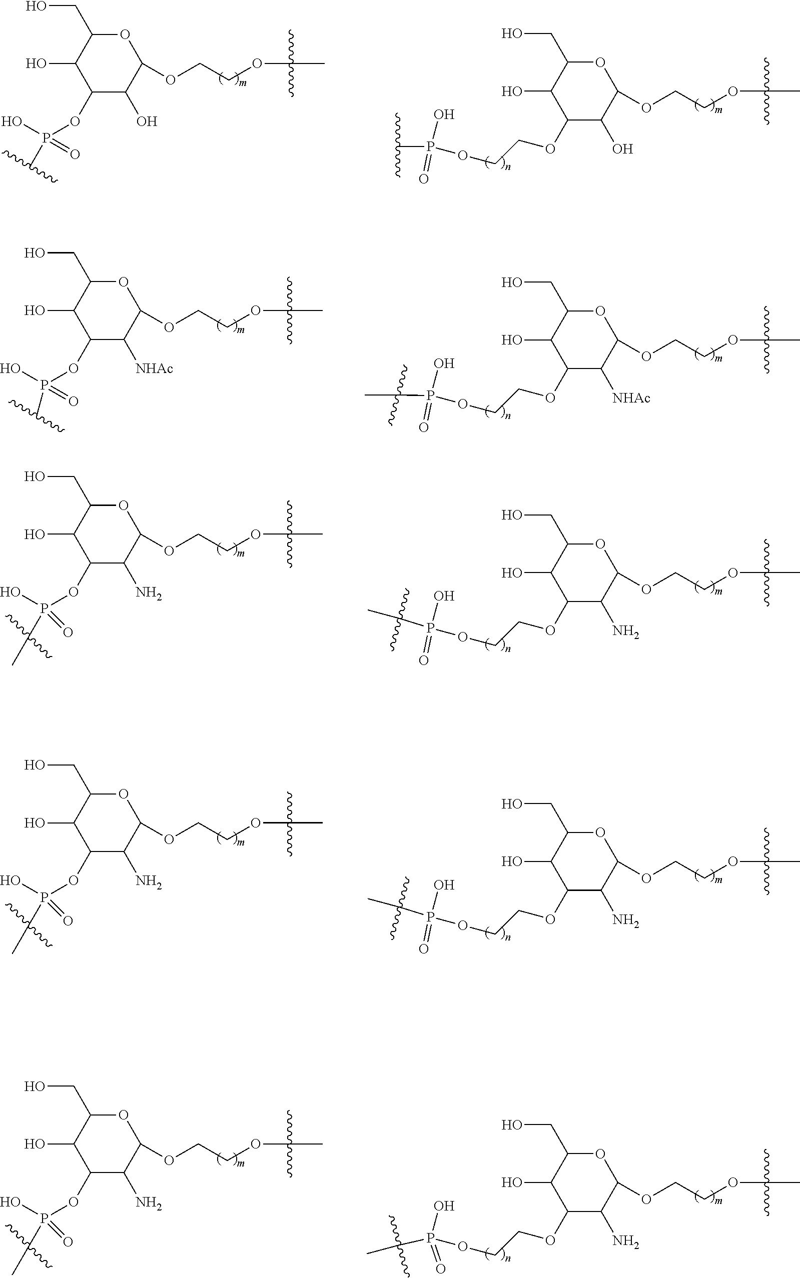

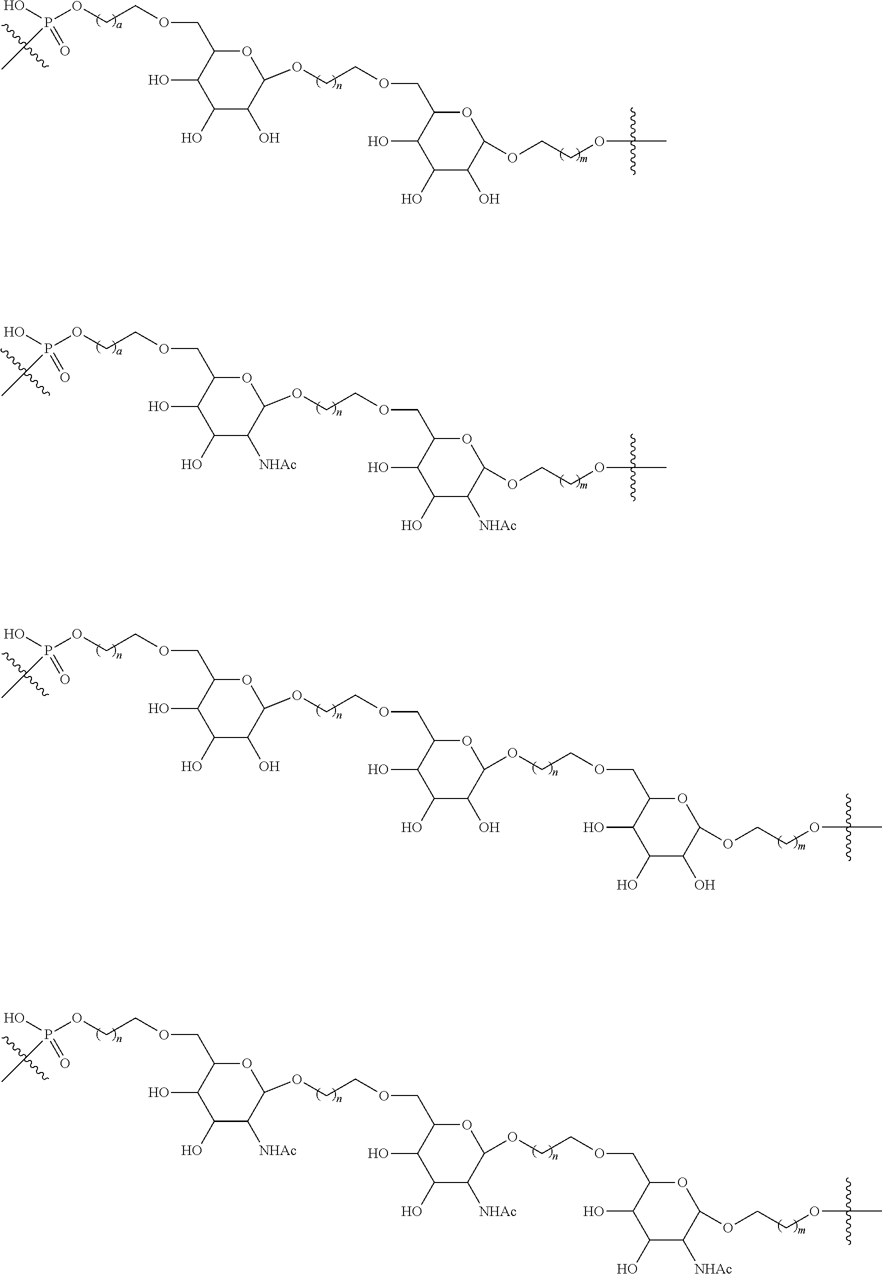

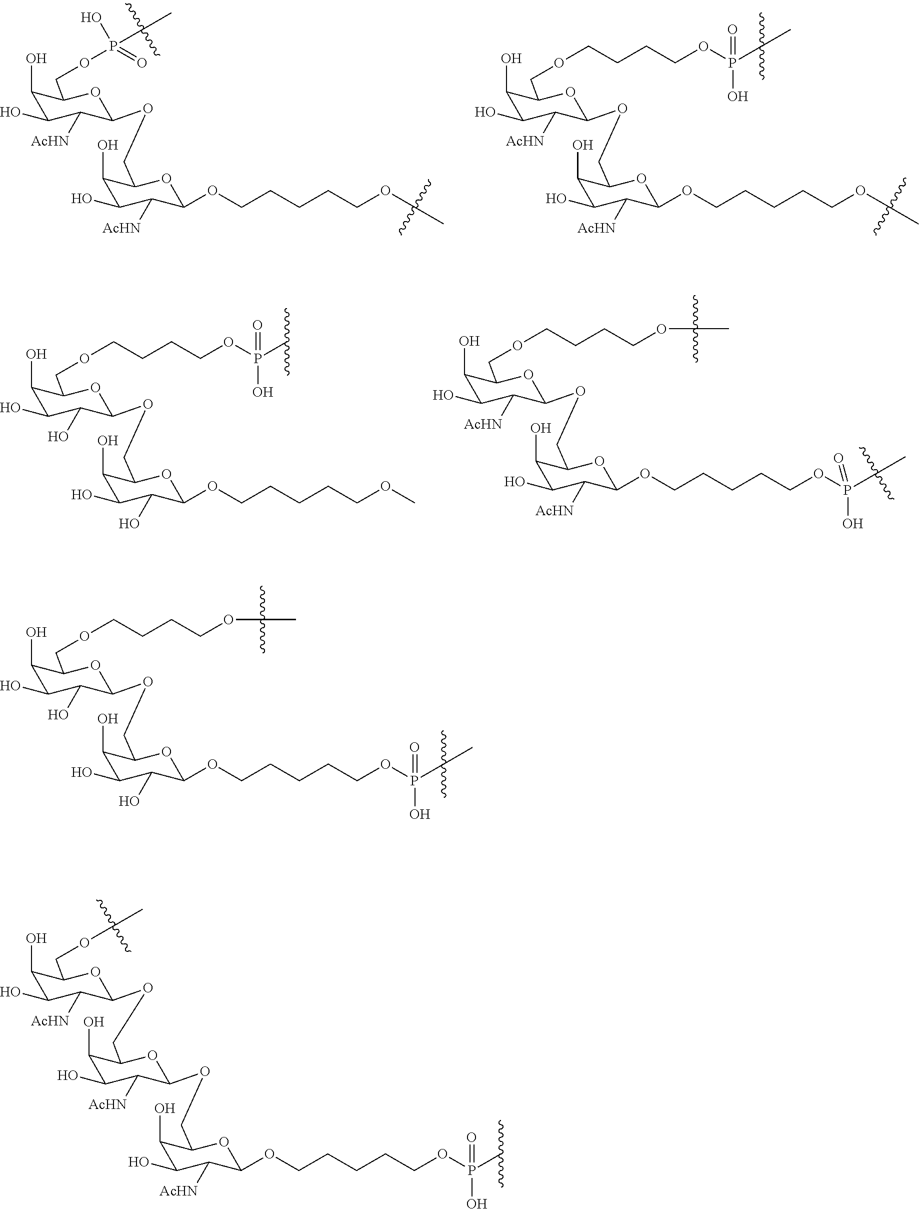

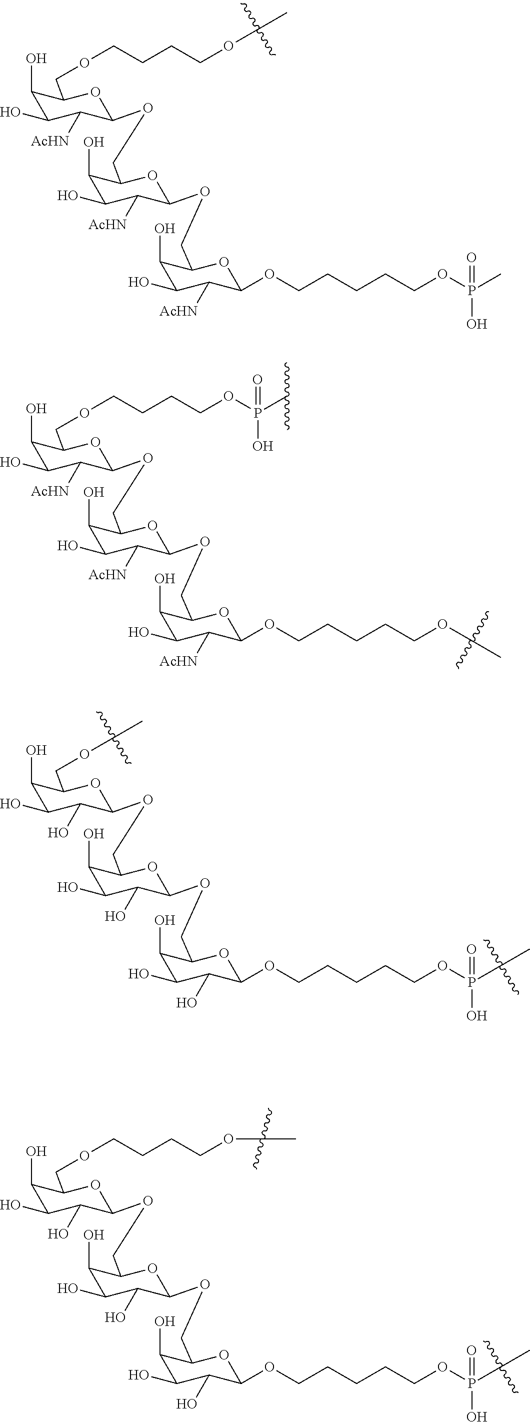

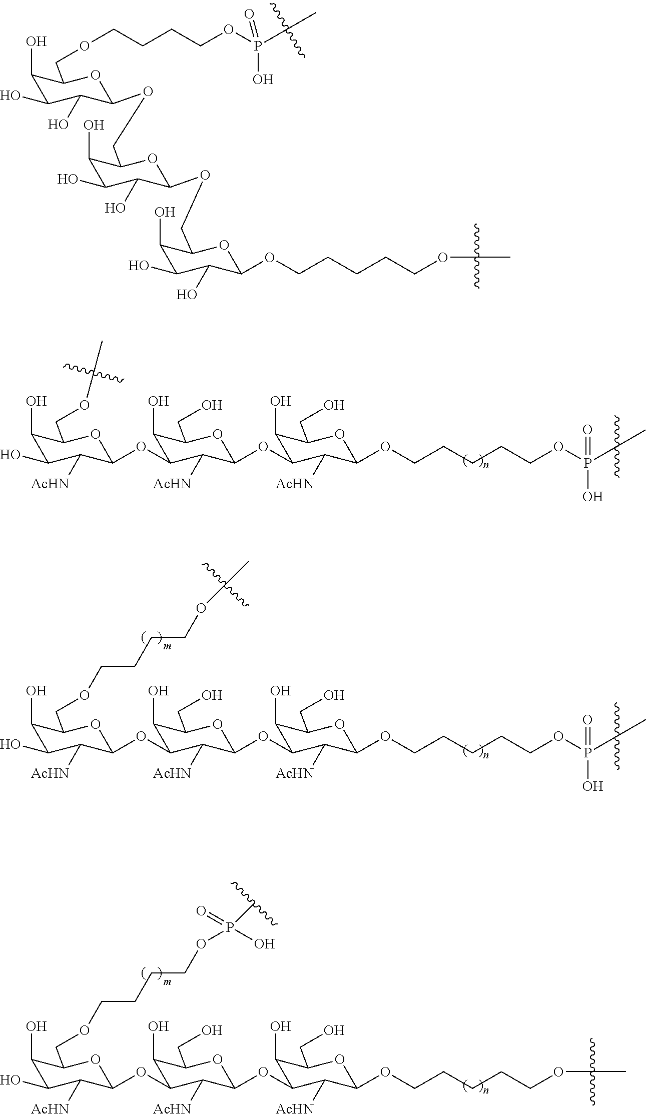

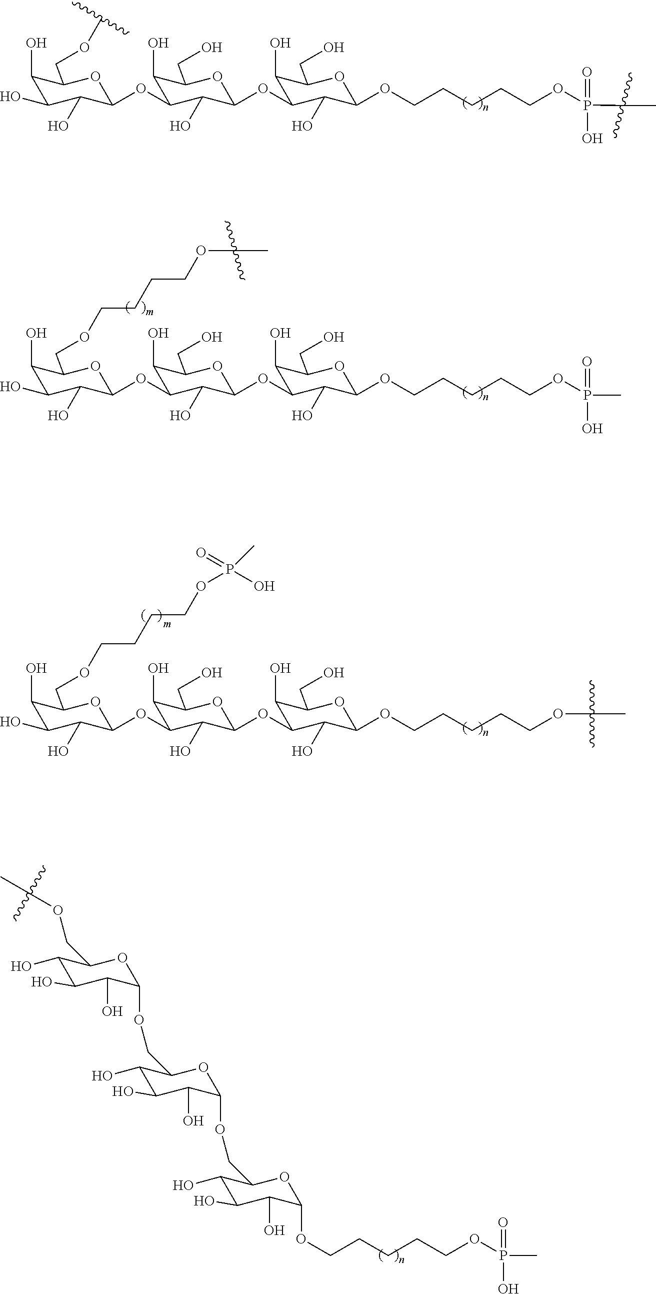

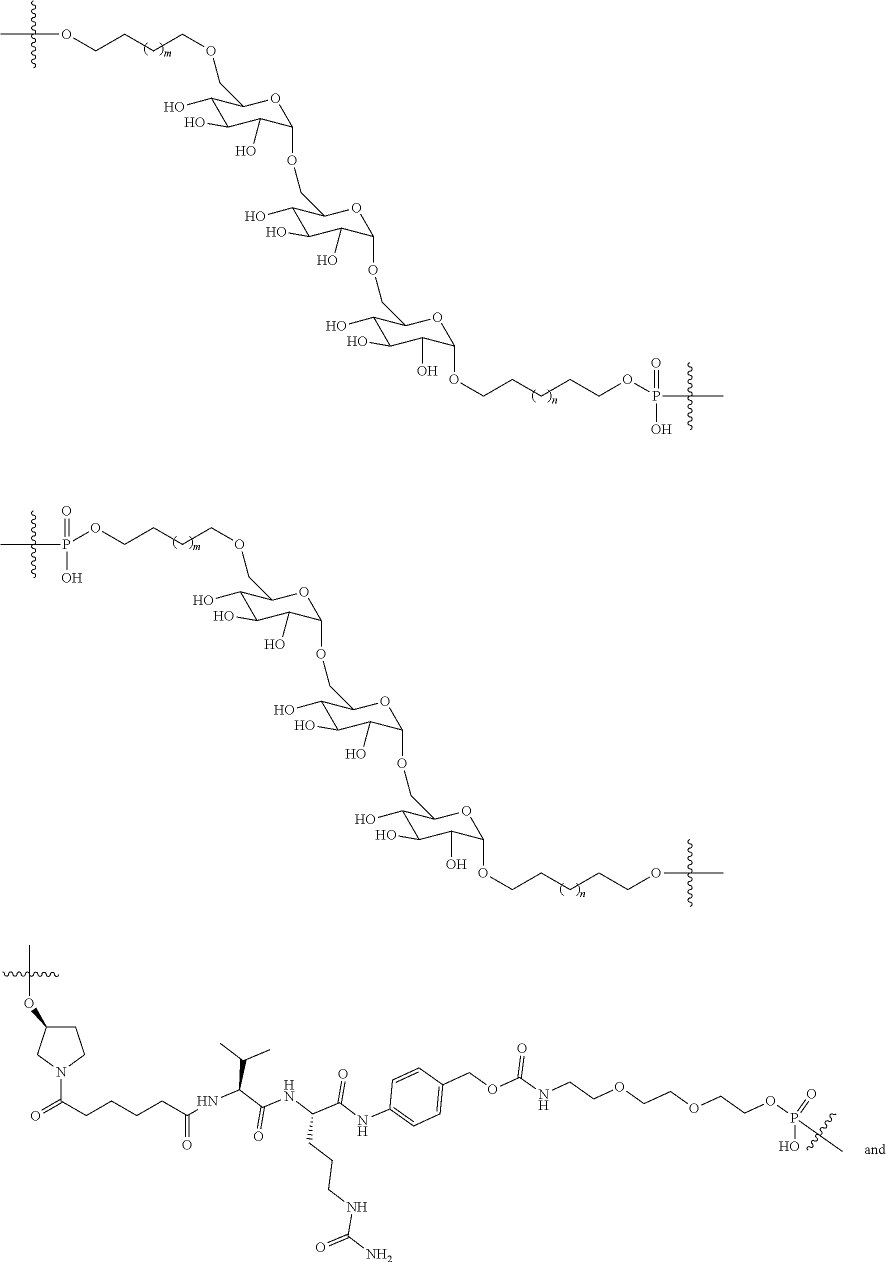

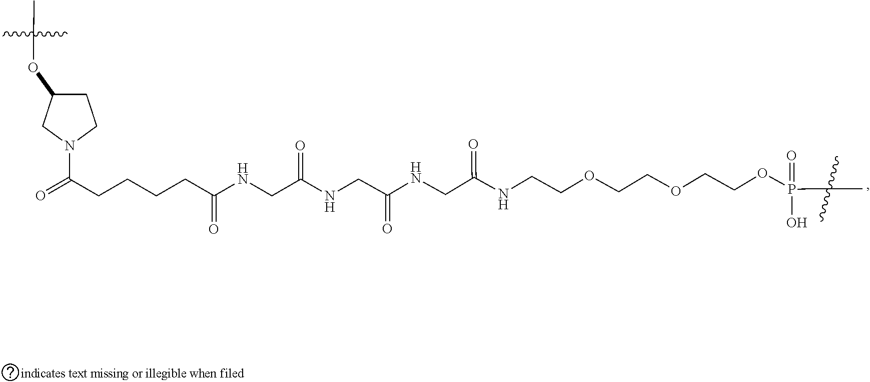

wherein n=1-12 and m=1-12.

In some embodiments, the cleavable linker (or the bio-cleavable linker) is an endosomal cleavable linker comprising one or more saccharide units independently selected from the following groups:

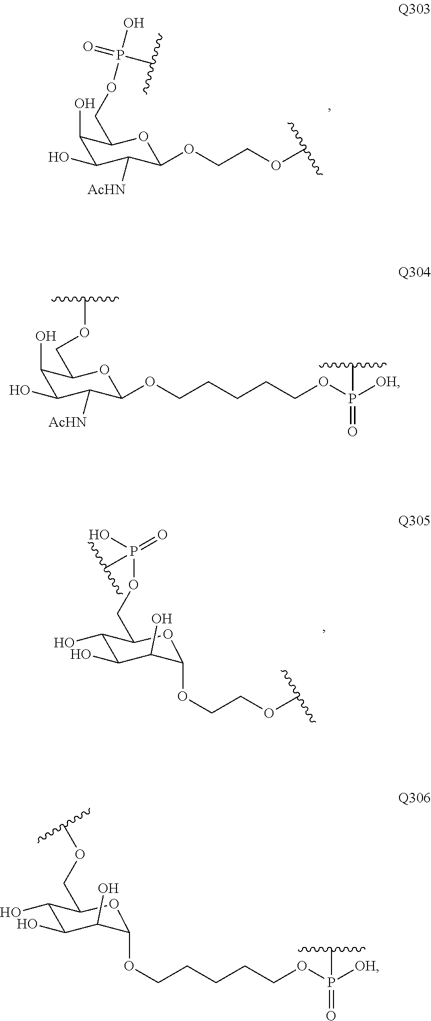 [SMILES: *OCCOC1OC(COP(*)(=O)O)C(O)C(O)C1NC(C)=O ; *OCC1OC(OCCCCCOP(*)(=O)O)C(NC(C)=O)C(O)C1O ; *OCCOC1OC(COP(*)(=O)O)C(O)C(O)C1O ; *OCC1OC(OCCCCCOP(*)(=O)O)C(O)C(O)C1O]

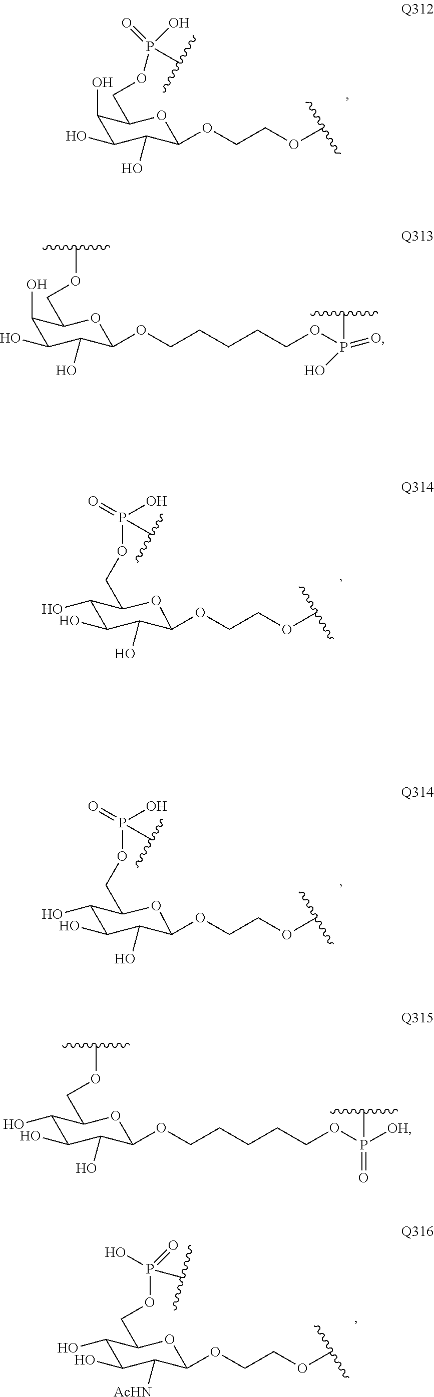 [SMILES: *OCCOC1OC(COP(*)(=O)O)C(O)C(O)C1O ; *OCC1OC(OCCCCCOP(*)(=O)O)C(O)C(O)C1O ; *OCCOC1OC(COP(*)(=O)O)C(O)C(O)C1O ; *OCCOC1OC(COP(*)(=O)O)C(O)C(O)C1O ; *OCC1OC(OCCCCCOP(*)(=O)O)C(O)C(O)C1O ; *OCCOC1OC(COP(*)(=O)O)C(O)C(O)C1NC(C)=O]

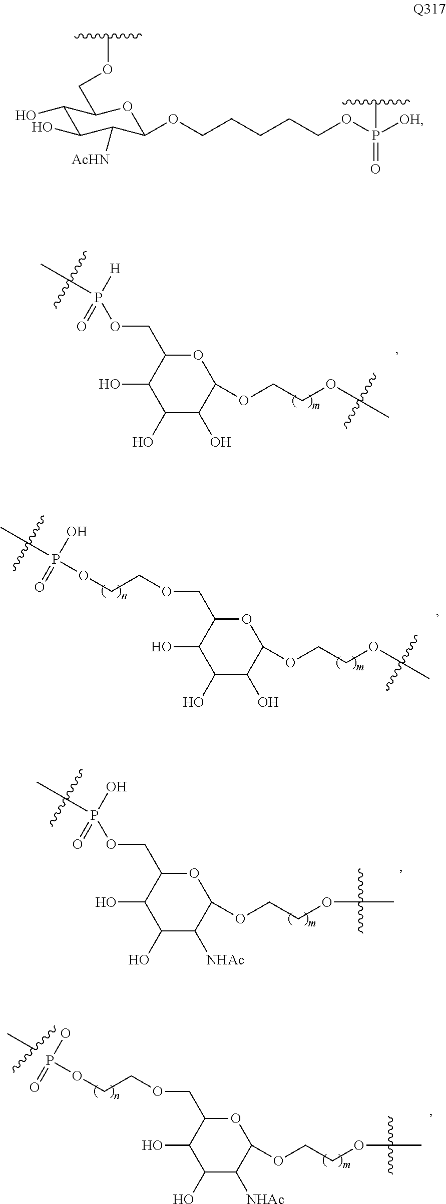

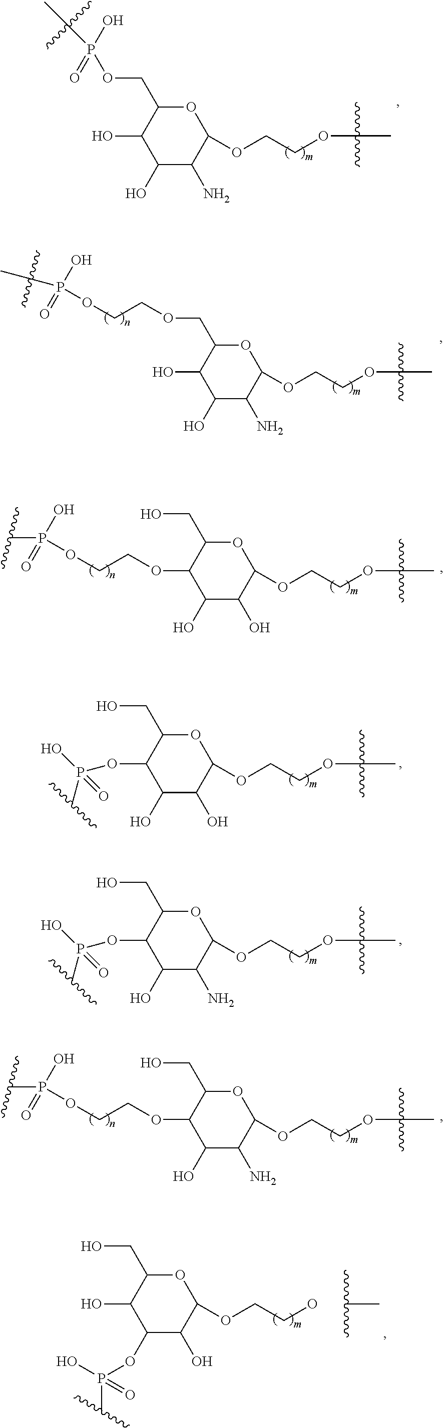

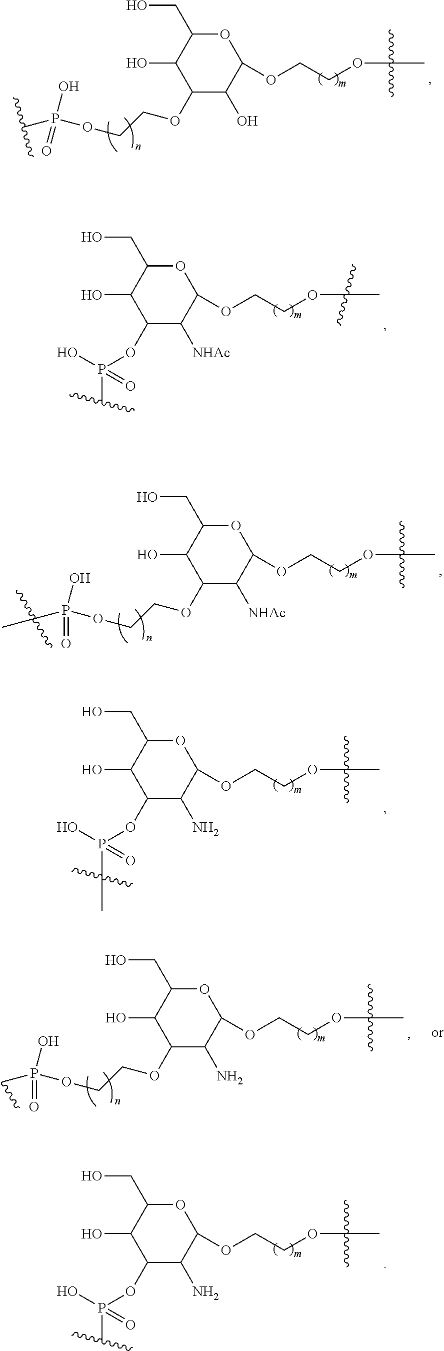

In some embodiments, the endosomal cleavable linker comprises two or more of the above saccharide units.

In some embodiments, the endosomal cleavable linker comprises 1-10 of the saccharide units.

In some embodiments, the endosomal cleavable linker comprises 2-10 of the saccharide units. In some embodiments, the saccharide units in the endosomal cleavable linker are selected from the group consisting of Q303, Q304, Q305, Q306, Q312, Q313, Q314, Q315, Q316 and Q317.

In some embodiments, the endosomal cleavable linker comprises 2, 3, or 4 of the saccharide units. In some embodiments, the saccharide units are selected from the group consisting of Q303, Q304, Q305, Q306, Q312, Q313, Q314, Q315, Q316 and Q317. For instance, the saccharide units may be Q304.

In one embodiment, the endosomal cleavable linker comprises -Q303Q303-, -Q303Q303Q303-, -Q303Q303Q303Q303-, -Q304Q304-, -Q304Q304Q304-, -Q304Q304Q304Q304-, -Q305Q305-, -Q305Q305Q305-, -Q306Q306-, -Q306Q306Q306-, -Q312Q312-, -Q312Q312Q312-, -Q313Q313-, -Q313Q313Q313-, -Q314Q314-, -Q314Q314Q314-, -Q315Q315-, -Q315Q315Q315-, -Q316Q316-, -Q316Q316Q316-, -Q317Q317-, or -Q317Q317Q317-.

In one embodiment, the endosomal cleavable linker further comprises

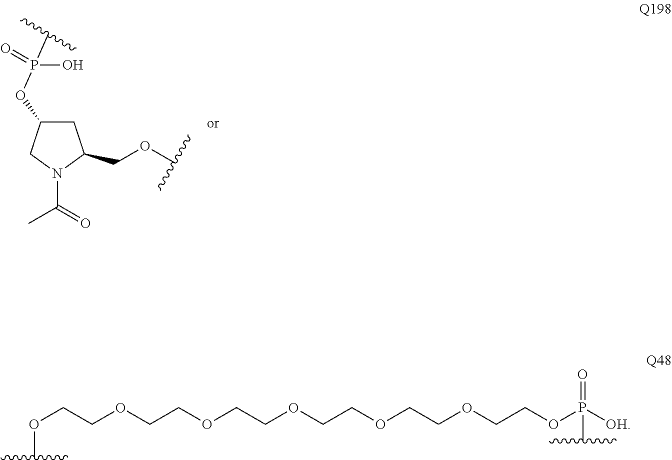 [SMILES: *OC[C@@H]1C[C@@H](OP(*)(=O)O)CN1C(C)=O ; *OCCOCCOCCOCCOCCOCCOP(*)(=O)O]

In one embodiment, the endosomal cleavable linker comprises: -Q198Q48Q303Q303Q48-, -Q198Q303Q48Q303-, -Q198Q48Q303Q303Q48-, -Q198Q303Q48Q303-, -Q198Q303Q303Q303Q303-, -Q198Q303Q303Q303-, -Q198Q303Q303-, -Q198Q304Q304Q304Q304-, -Q198Q304Q304Q304-, -Q198Q304Q304-, -Q198Q48Q303Q303Q48-, -Q198Q303Q48Q303-, -Q198Q303Q303-, -Q48Q303Q303Q48-, or -Q303Q48Q303-.

More discussion about the biocleavable linkers may be found in WO2018136620, the content of which is incorporated herein by reference in its entirety.

### Carriers

In certain embodiments, the linking group L connecting the two oligonucleotides of the single-stranded oligonucleotide contains one or more carriers. In certain embodiments, one or more ligands are conjugated to the single-stranded oligonucleotide via one or more carriers. In some embodiments, the carrier may replace one or more nucleotide(s).

The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalinyl. In one embodiment, the acyclic group is a moiety based on a serinol backbone or a diethanolamine backbone.

In some embodiments, the carrier replaces one or more nucleotide(s) in the internal position(s) of a nucleotide sequence of the single-stranded oligonucleotide (e.g., Z¹ and/or Z²).

A ribonucleotide subunit in which the ribose sugar of the subunit has been so replaced is referred to herein as a ribose replacement modification subunit (RRMS). The carrier can be a cyclic or acyclic moiety and include two “backbone attachment points” (e.g., hydroxyl groups) and a ligand. The ligand can be directly attached to the carrier or indirectly attached to the carrier by an intervening linker/tether, as described above.

The ligand-conjugated monomer subunit may be the 5′ or 3′ terminal subunit of a nucleotide sequence of the single-stranded oligonucleotide (e.g., Z¹ and/or Z²), i.e., one of the two “W” groups may be a hydroxyl group, and the other “W” group may be a chain of two or more unmodified or modified ribonucleotides. Alternatively, the ligand-conjugated monomer subunit may occupy an internal position, and both “W” groups may be one or more unmodified or modified ribonucleotides. More than one ligand-conjugated monomer subunit may be present in a single-stranded oligonucleotide.

### Sugar Replacement-Based Monomers, e.g., Ligand-Conjugated Monomers (Cyclic)

Cyclic sugar replacement-based monomers, e.g., sugar replacement-based ligand-conjugated monomers, are also referred to herein as RRMS monomer compounds. The carriers may have the general formula (LCM-2) provided below (In that structure preferred backbone attachment points can be chosen from R¹ or R²; R³ or R⁴; or R⁹ and R¹⁰ if Y is CR⁹R¹⁰ (two positions are chosen to give two backbone attachment points, e.g., R¹ and R⁴, or R⁴ and R⁹)). Preferred tethering attachment points include R⁷; R⁵ or R⁶ when X is CH₂. The carriers are described below as an entity, which can be incorporated into a strand. Thus, it is understood that the structures also encompass the situations wherein one (in the case of a terminal position) or two (in the case of an internal position) of the attachment points, e.g., R¹ or R²; R³ or R⁴; or R⁹ or R¹⁰ (when Y is CR⁹R¹⁰), is connected to the phosphate, or modified phosphate, e.g., sulfur containing, backbone. E.g., one of the above-named R groups can be —CH₂—, wherein one bond is connected to the carrier and one to a backbone atom, e.g., a linking oxygen or a central phosphorus atom.

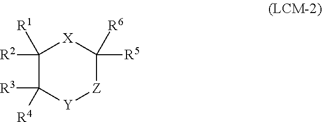

wherein:
X is N(CO)R⁷, NR⁷ or CH₂;
Y is NR⁸, O, S, CR⁹R¹⁰;
Z is CR¹¹R¹² or absent;
Each of R¹, R², R³, R⁴, R⁹, and R¹⁰ is, independently, H, OR^{a}, or (CH₂)ₙOR^{b}, provided that at least two of R¹, R², R³, R⁴, R⁹, and R¹⁰ are OR^{a} and/or (CH₂)ₙOR^{b};
Each of R⁵, R⁶, R¹¹, and R¹² is, independently, a ligand, H, C₁-C₆ alkyl optionally substituted with 1-3 R¹³, or C(O)NHR⁷; or R⁵ and R¹¹ together are C₃-C₈ cycloalkyl optionally substituted with R¹⁴;
R⁷ can be a ligand, e.g., R⁷ can be R^{d}, or R⁷ can be a ligand tethered indirectly to the carrier, e.g., through a tethering moiety, e.g., C₁-C₂₀ alkyl substituted with NR^{c}R^{d}; or
C₁-C₂₀ alkyl substituted with NHC(O)R^{d};
R⁸ is H or C₁-C₆ alkyl;
R¹³ is hydroxy, C₁-C₄ alkoxy, or halo;
R¹⁴ is NR^{c}R⁷;
R¹⁵ is C₁-C₆ alkyl optionally substituted with cyano, or C₂-C₆ alkenyl;
R¹⁶ is C₁-C₁₀ alkyl;
R¹⁷ is a liquid or solid phase support reagent;
L is —C(O)(CH₂)_{q}C(O)—, or —C(O)(CH₂)_{q}S—;
R^{a} is a protecting group, e.g., CAr₃; (e.g., a dimethoxytrityl group) or
Si(X^{5′})(X^{5″})(X^{5′″}) in which (X^{5′}), (X^{5″}), and (X^{5′″}) are as described elsewhere.
R^{b} is P(O)(O⁻)H, P(OR¹⁵)N(R¹⁶)₂ or L-R¹⁷;
R^{c} is H or C₁-C₆ alkyl;
R^{d} is H or a ligand;
Each Ar is, independently, C₆-C₁₀ aryl optionally substituted with C₁-C₄ alkoxy;
n is 1-4; and q is 0-4.

Exemplary carriers include those in which, e.g., X is N(CO)R⁷ or NR⁷, Y is CR⁹R¹⁰, and Z is absent; or X is N(CO)R⁷ or NR⁷, Y is CR⁹R¹⁰, and Z is CR¹¹R¹²; or X is N(CO)R⁷ or NR⁷, Y is NR⁸, and Z is CR¹¹R¹²; or X is N(CO)R⁷ or NR⁷, Y is O, and Z is CR¹¹R¹²; or X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

In certain embodiments, the carrier may be based on the pyrroline ring system or the 4-hydroxyproline ring system, e.g., X is N(CO)R⁷ or NR⁷, Y is CR⁹R¹⁰, and Z is absent (D).

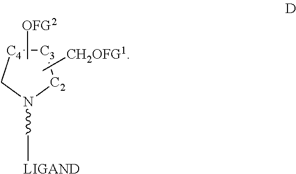

OFG¹ is preferably attached to a primary carbon, e.g., an exocyclic alkylene group, e.g., a methylene group, connected to one of the carbons in the five-membered ring (—CH₂OFG¹ in D). OFG² is preferably attached directly to one of the carbons in the five-membered ring (—OFG² in D). For the pyrroline-based carriers, —CH₂OFG¹ may be attached to C-2 and OFG² may be attached to C-3; or —CH₂OFG¹ may be attached to C-3 and OFG² may be attached to C-4. In certain embodiments, CH₂OFG¹ and OFG² may be geminally substituted to one of the above-referenced carbons. For the 3-hydroxyproline-based carriers, —CH₂OFG¹ may be attached to C-2 and OFG² may be attached to C-4. The pyrroline- and 4-hydroxyproline-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen. Preferred examples of carrier D include the following:

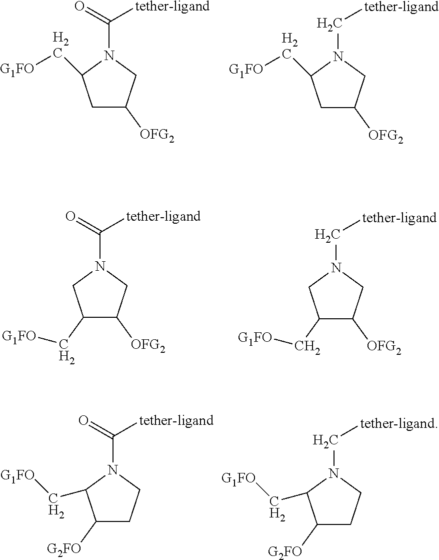

In certain embodiments, the carrier may be based on the piperidine ring system (E), e.g., X is N(CO)R⁷ or NR⁷, Y is CR⁹R¹⁰, and Z is CR¹¹R¹².

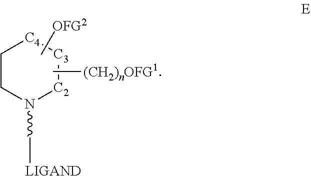

OFG¹ is preferably attached to a primary carbon, e.g., an exocyclic alkylene group, e.g., a methylene group (n=1) or ethylene group (n=2), connected to one of the carbons in the six-membered ring [—(CH₂)ₙOFG¹ in E]. OFG² is preferably attached directly to one of the carbons in the six-membered ring (—OFG² in E). —(CH₂)ₙOFG¹ and OFG² may be disposed in a geminal manner on the ring, i.e., both groups may be attached to the same carbon, e.g., at C-2, C-3, or C-4. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3. The piperidine-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

In certain embodiments, the carrier may be based on the piperazine ring system (F), e.g., X is N(CO)R⁷ or NR⁷, Y is NR⁸, and Z is CR¹¹R¹², or the morpholine ring system (G), e.g., X is N(CO)R⁷ or NR⁷, Y is O, and Z is CR¹¹R¹².

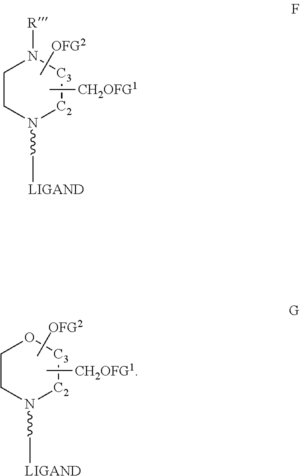

OFG¹ is preferably attached to a primary carbon, e.g., an exocyclic alkylene group, e.g., a methylene group, connected to one of the carbons in the six-membered ring (—CH₂OFG¹ in F or G). OFG² is preferably attached directly to one of the carbons in the six-membered rings (—OFG² in F or G). For both F and G, —CH₂OFG¹ may be attached to C-2 and OFG² may be attached to C-3; or vice versa. In certain embodiments, CH₂OFG¹ and OFG² may be geminally substituted to one of the above-referenced carbons. The piperazine- and morpholine-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen in both F and G.

In certain embodiments, the carrier may be based on the decalin ring system, e.g., X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

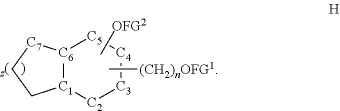

OFG¹ is preferably attached to a primary carbon, e.g., an exocyclic methylene group (n=1) or ethylene group (n=2) connected to one of C-2, C-3, C-4, or C-5 [—(CH₂)ₙOFG¹ in H]. OFG² is preferably attached directly to one of C-2, C-3, C-4, or C-5 (—OFG² in H). —(CH₂)ₙOFG¹ and OFG² may be disposed in a geminal manner on the ring, i.e., both groups may be attached to the same carbon, e.g., at C-2, C-3, C-4, or C-5. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3; —(CH₂)ₙOFG¹ may be attached to C-4 and OFG² may be attached to C-5; or —(CH₂)ₙOFG¹ may be attached to C-5 and OFG² may be attached to C-4. The decalin or indane-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). In a preferred embodiment, the substituents at C-1 and C-6 are trans with respect to one another. The tethering attachment point is preferably C-6 or C-7.

Other carriers may include those based on 3-hydroxyproline (J).

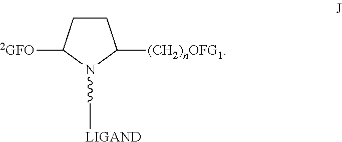

Thus, —(CH₂)ₙOFG¹ and OFG² may be cis or trans with respect to one another. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

Details about more representative cyclic, sugar replacement-based carriers can be found in U.S. Pat. Nos. 7,745,608 and 8,017,762, which are herein incorporated by reference in their entireties.

### Sugar Replacement-Based Monomers (Acyclic)

Acyclic sugar replacement-based monomers, e.g., sugar replacement-based ligand-conjugated monomers, are also referred to herein as ribose replacement monomer subunit (RRMS) monomer compounds. Preferred acyclic carriers can have formula LCM-3 or LCM-4:

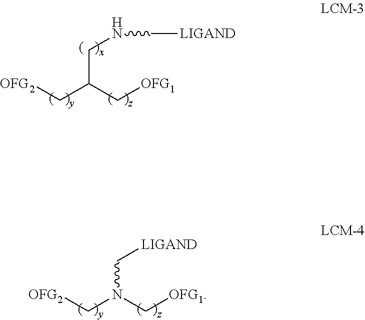

In some embodiments, each of x, y, and z can be, independently of one another, 0, 1, 2, or 3. In formula LCM-3, when y and z are different, then the tertiary carbon can have either the R or S configuration. In preferred embodiments, x is zero and y and z are each 1 in formula LCM-3 (e.g., based on serinol), and y and z are each 1 in formula LCM-3. Each of formula LCM-3 or LCM-4 below can optionally be substituted, e.g., with hydroxy, alkoxy, perhaloalkyl.

Details about more representative acyclic, sugar replacement-based carriers can be found in U.S. Pat. Nos. 7,745,608 and 8,017,762, which are herein incorporated by reference in their entireties.

In some embodiments, the single-stranded oligonucleotide comprises one or more ligands conjugated to the 5′ end of a nucleotide sequence (e.g., Z¹ and/or Z²).

In certain embodiments, the ligand is conjugated to the 5′-end of a nucleotide sequence (e.g., Z¹ and/or Z²) via a carrier and/or linker. In one embodiment, the ligand is conjugated to the 5′-end of a nucleotide sequence (e.g., Z¹ and/or Z²) via a carrier of a formula:

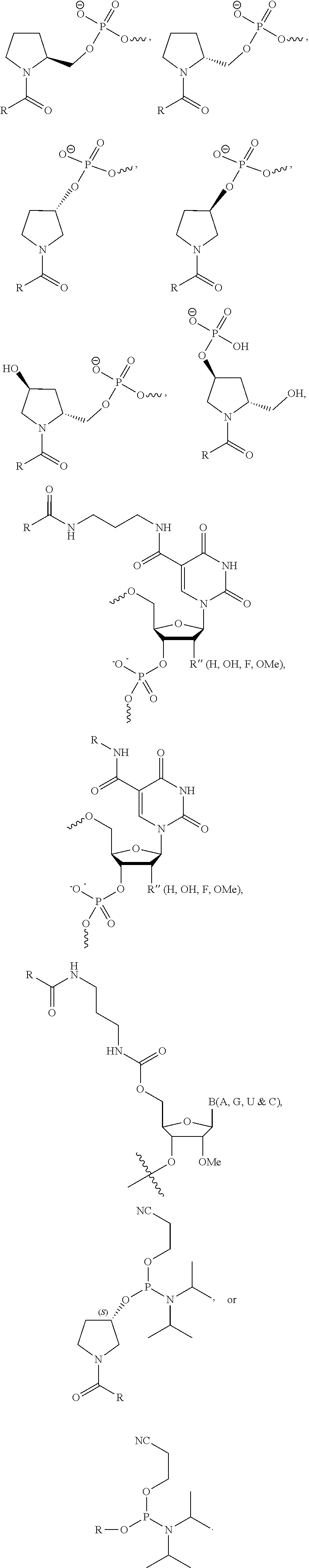

R is a ligand.

In some embodiments, the single-stranded oligonucleotide comprises one or more ligands conjugated to the 3′ end of a nucleotide sequence (e.g., Z¹ and/or Z²).

In certain embodiments, the ligand is conjugated to the 3′-end of a nucleotide sequence (e.g., Z¹ and/or Z²) via a carrier and/or linker. In one embodiment, the ligand is conjugated to the 3′-end of a nucleotide sequence (e.g., Z¹ and/or Z²) via a carrier of a formula:

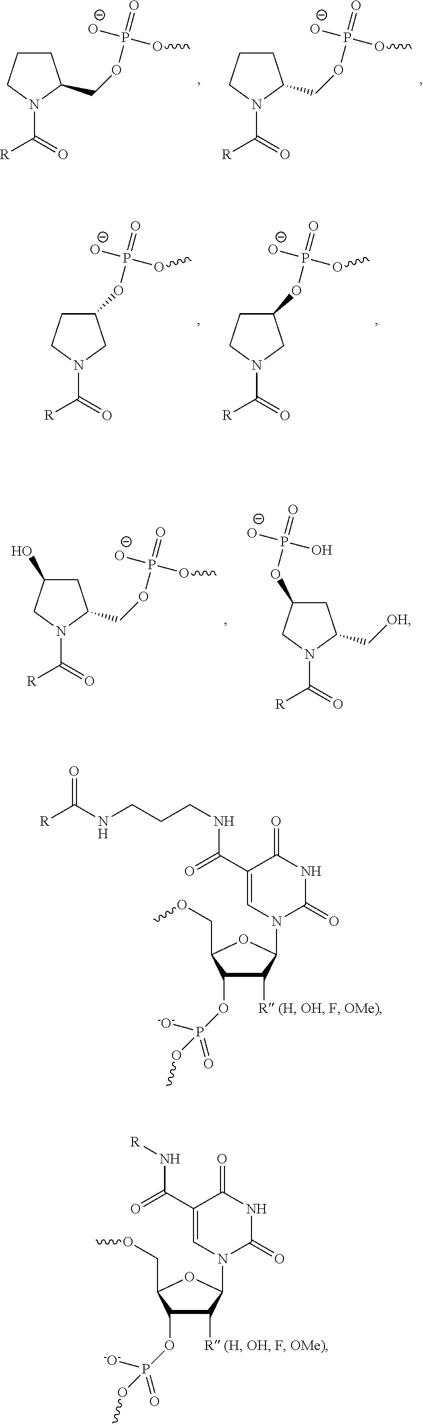

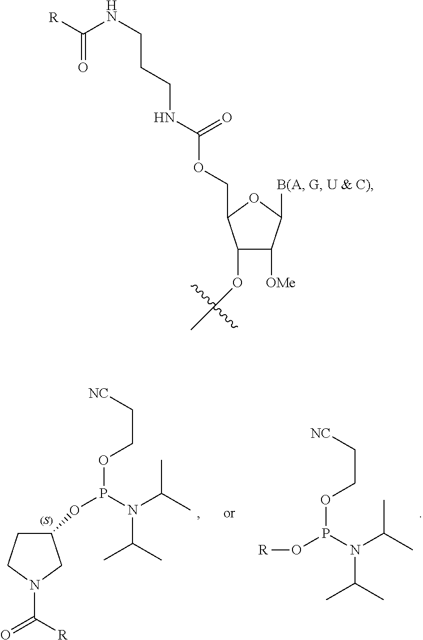

R is a ligand.

In some embodiments, the ligand is conjugated to a nucleotide sequence (e.g., Z¹ and/or Z²) via one or more linkers (tethers) and/or a carrier. In one embodiment, the ligand is conjugated to a nucleotide sequence (e.g., Z¹ and/or Z²) via one or more linkers (tethers).

In one embodiment, the ligand is conjugated to the 5′ end or 3′ end of a nucleotide sequence (e.g., Z¹ and/or Z²) via a cyclic carrier, optionally via one or more intervening linkers (tethers).

In some embodiments, the ligand is conjugated to one or more internal positions on at least one nucleotide sequence (e.g., Z¹ and/or Z²). Internal positions of a nucleotide sequence refer to the nucleotide on any position of the nucleotide sequence, except the terminal position from the 3′ end and 5′ end of the nucleotide sequence (e.g., excluding 2 positions: position 1 counting from the 3′ end and position 1 counting from the 5′ end).

In one embodiment, the ligand is conjugated to one or more internal positions on at least one nucleotide sequence (e.g., Z¹ and/or Z²), which include all positions except the terminal two positions from each end of the nucleotide sequence (e.g., excluding 4 positions: positions 1 and 2 counting from the 3′ end and positions 1 and 2 counting from the 5′ end). In one embodiment, the lipophilic moiety is conjugated to one or more internal positions on at least one nucleotide sequence (e.g., Z¹ and/or Z²), which include all positions except the terminal three positions from each end of the nucleotide sequence (e.g., excluding 6 positions: positions 1, 2, and 3 counting from the 3′ end and positions 1, 2, and 3 counting from the 5′ end).

In one embodiment, the ligand is conjugated to one or more internal positions on at least one nucleotide sequence (e.g., Z¹ and/or Z²), except the cleavage site region of a nucleotide sequence, for instance, the ligand is not conjugated to positions 9-12 counting from the 5′-end of a nucleotide sequence, for example, the ligand is not conjugated to positions 9-11 counting from the 5′-end of a nucleotide sequence (e.g., Z¹ and/or Z²). Alternatively, the internal positions exclude positions 11-13 counting from the 3′-end of a nucleotide sequence (e.g., Z¹ and/or Z²). In one embodiment, the internal positions exclude positions 12-14 counting from the 5′-end of a nucleotide sequence.

In one embodiment, the ligand is conjugated to one or more internal positions on at least one nucleotide sequence (e.g., Z¹ and/or Z²), which exclude positions 11-13 on a nucleotide sequence, counting from the 3′-end, and positions 12-14 on a nucleotide sequence (e.g., Z¹ and/or Z²), counting from the 5′-end.

In one embodiment, one or more ligands are conjugated to one or more of the following internal positions: positions 4-8 and 13-18 on a nucleotide sequence (e.g., Z¹ and/or Z²), and positions 6-10 and 15-18 on a nucleotide sequence (e.g., Z¹ and/or Z²), counting from the 5′ end.

In one embodiment, one or more ligands are conjugated to one or more of the following internal positions: positions 5, 6, 7, 15, and 17 on a nucleotide sequence (e.g., Z¹ and/or Z²), and positions 15 and 17 on a nucleotide sequence (e.g., Z¹ and/or Z²), counting from the 5′ end.

In some embodiments, the ligand is conjugated to a nucleobase, sugar moiety, or internucleosidic linkage of the single-stranded oligonucleotide.

### Ligands

In certain embodiments, the single-stranded oligonucleotide is further modified by covalent attachment of one or more conjugate groups. In general, conjugate groups modify one or more properties of the attached single-stranded oligonucleotide including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an oligomeric compound. A preferred list of conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

In some embodiments, the single-stranded oligonucleotide further comprises a targeting ligand that targets a receptor which mediates delivery to a specific CNS tissue. These targeting ligands can be conjugated in combination with the lipophilic moiety to enable specific local (e.g., intrathecal) and systemic delivery.

Exemplary targeting ligands that targets the receptor mediated delivery to a CNS tissue are peptide ligands such as Angiopep-2, lipoprotein receptor related protein (LRP) ligand, bEnd.3 cell binding ligand; transferrin receptor (TfR) ligand (which can utilize iron transport system in brain and cargo transport into the brain parenchyma); manose receptor ligand (which targets olfactory ensheathing cells, glial cells), glucose transporter protein, and LDL receptor ligand.

In some embodiments, the single-stranded oligonucleotide further comprises a targeting ligand that targets a receptor which mediates delivery to a specific ocular tissue. These targeting ligands can be conjugated in combination with the lipophilic moiety to enable specific local (e.g., intravitreal) and systemic delivery. Exemplary targeting ligands that targets the receptor mediated delivery to a ocular tissue are lipophilic ligands such as all-trans retinol (which targets the retinoic acid receptor); RGD peptide (which targets retinal pigment epithelial cells), such as H-Gly-Arg-Gly-Asp-Ser-Pro-Lys-Cys-OH (SEQ ID NO: 1) or Cyclo(-Arg-Gly-Asp-D-Phe-Cys) (SEQ ID NO: 2); LDL receptor ligands; and carbohydrate based ligands (which targets endothelial cells in posterior eye).

Preferred conjugate groups amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Generally, a wide variety of entities, e.g., ligands, can be coupled to the oligomeric compounds described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g., steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-1B, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a.helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and branched polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to, AALEALAEALEALAEALEALAEAAAAGGC (GALA) (SEQ ID NO: 3); AALAEALAEALAEALAEALAEALAAAAGGC (EALA) (SEQ ID NO: 4); ALEALAEALEALAEA (SEQ ID NO: 5); GLFEAIEGFIENGWEGMIWDYG (INF-7) (SEQ ID NO: 6); GLFGAIAGFIENGWEGMIDGWYG (Inf HA-2) (SEQ ID NO: 7); GLFEAIEGFIENGWEGMIDGWYGCGLFEAIEGFIENGWEGMID GWYGC (diINF-7) (SEQ ID NO: 8); GLFEAIEGFIENGWEGMIDGGCGLFEAIEGFIENGWEGMIDGGC (diINF-3) (SEQ ID NO: 9); GLFGALAEALAEALAEHLAEALAEALEALAAGGSC (GLF) (SEQ ID NO: 10); GLFEAIEGFIENGWEGLAEALAEALEALAAGGSC (GALA-INF3) (SEQ ID NO: 11); GLF EAI EGFI ENGW EGnI DG K GLF EAI EGFI ENGW EGnI DG (INF-5, n is norleucine) (SEQ ID NO: 12); LFEALLELLESLWELLLEA (JTS-1) (SEQ ID NO: 13); GLFKALLKLLKSLWKLLLKA (ppTG1) (SEQ ID NO: 14); GLFRALLRLLRSLWRLLLRA (ppTG20) (SEQ ID NO: 15); WEAKLAKALAKALAKHLAKALAKALKACEA (KALA) (SEQ ID NO: 16); GLFFEAIAEFIEGGWEGLIEGC (HA) (SEQ ID NO: 17); GIGAVLKVLTTGLPALISWIKRKRQQ (Melittin) (SEQ ID NO: 18); HsWYG (SEQ ID NO: 19); and CHK₆HC (SEQ ID NO: 20).

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also referred to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to, RQIKIWFQNRRMKWKK (penetratin) (SEQ ID NO: 21); GRKKRRQRRRPPQC (Tat fragment 48-60) (SEQ ID NO: 22); GALFLGWLGAAGSTMGAWSQPKKKRKV (signal sequence based peptide) (SEQ ID NO: 23); LLIILRRRIRKQAHAHSK (PVEC) (SEQ ID NO: 24); GWTLNSAGYLLKINLKALAALAKKIL (transportan) (SEQ ID NO: 25); KLALKLALKALKAALKLA (amphiphilic model peptide) (SEQ ID. NO: 26); RRRRRRRRR (Arg9) (SEQ ID NO: 27); KFFKFFKFFK (Bacterial cell wall permeating peptide) (SEQ ID NO: 28); LLGDFFRKSKEKIGKEFKRIVQRIKDFLRNLVPRTES (LL-37) (SEQ ID NO: 29); SWLSKTAKKLENSAKKRISEGIAIAIQGGPR (cecropin P1) (SEQ ID NO: 30); ACYCRIPACIAGERRYGTCIYQGRLWAFCC (α-defensin) (SEQ ID NO: 31); DHYNCVSSGGQCLYSACPIFTKIQGTCYRGKAKCCK (β-defensin) (SEQ ID NO: 32); RRRPRPPYLPRPRPPPFFPPRLPPRIPPGFPPRFPPRFPGKR-NH2 (PR-39) (SEQ ID NO: 33); ILPWKWPWWPWRR-NH2 (indolicidin) (SEQ ID NO: 34); AAVALLPAVLLALLAP (RFGF) (SEQ ID NO: 35); AALLPVLLAAP (RFGF analogue) (SEQ ID NO: 36); and RKCRIVVIRVCR (bactenecin) (SEQ ID NO: 37).

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc₂ and GalNAc₃ (GalNAc and multivalent GalNAc are collectively referred to herein as GalNAc conjugates); D-mannose, multivalent mannose, multivalent lactose, N-acetyl-glucosamine, Glucose, multivalent Glucose, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of the composition of the invention. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

The ligand or tethered ligand can be present on a monomer when said monomer is incorporated into a component of the single-stranded oligonucleotide. In some embodiments, the ligand can be incorporated via coupling to a “precursor” monomer after said “precursor” monomer has been incorporated into a component of the single-stranded oligonucleotide. For example, a monomer having, e.g., an amino-terminated tether (i.e., having no associated ligand), e.g., monomer-linker-NH₂ can be incorporated into a component of the single-stranded oligonucleotide. In a subsequent operation, i.e., after incorporation of the precursor monomer into a component of the single-stranded oligonucleotide, a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

In some embodiments, ligand can be conjugated to nucleobases, sugar moieties, or internucleosidic linkages of the single-stranded oligonucleotide. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligonucleotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

Representative U.S. patents that teach the preparation of conjugates of nucleic acids include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,672,662; 5,688,941; 5,714,166; 6,153,737; 6,172,208; 6,300,319; 6,335,434; 6,335,437; 6,395,437; 6,444,806; 6,486,308; 6,525,031; 6,528,631; 6,559,279; contents of which are herein incorporated in their entireties by reference.

In some embodiments, the single-stranded oligonucleotide further comprises one or more targeting ligands that target a liver tissue. In some embodiments, at least one of the targeting ligands is a carbohydrate-based ligand. In some embodiments, the carbohydrate-based ligand is an ASGPR ligand. In one embodiment, at least one of the targeting ligands is a GalNAc-based conjugate.

In some embodiments, the carbohydrate-based ligand is any one of the ligands listed in Table 2, Table 2A, Table 3, Table 3A, Table 4, or Table 4A of WO2015/006740, which is incorporated herein by reference in its entirety.

In some embodiments, the linkers including branched linkers such as a bivalent or trivalent branched linker for attaching these carbohydrate-based ligands include the linker(s) listed in Table 1 or Table 1A and the spacer(s) listed in Table 5 of WO2015/006740, which is incorporated herein by reference in its entirety.

In some embodiments, the GalNAc-based conjugate is a GalNAc analog containing a S or N atom, or a —CH₂— group in the glycosidic linkage to change a metabolically labile glycosidic linkage to a metabolically stable glycosidic linkage, e.g., having “0” in the glycosidic linkage being replaced by S or N atom, or a —CH₂— group, as shown in the scheme below.

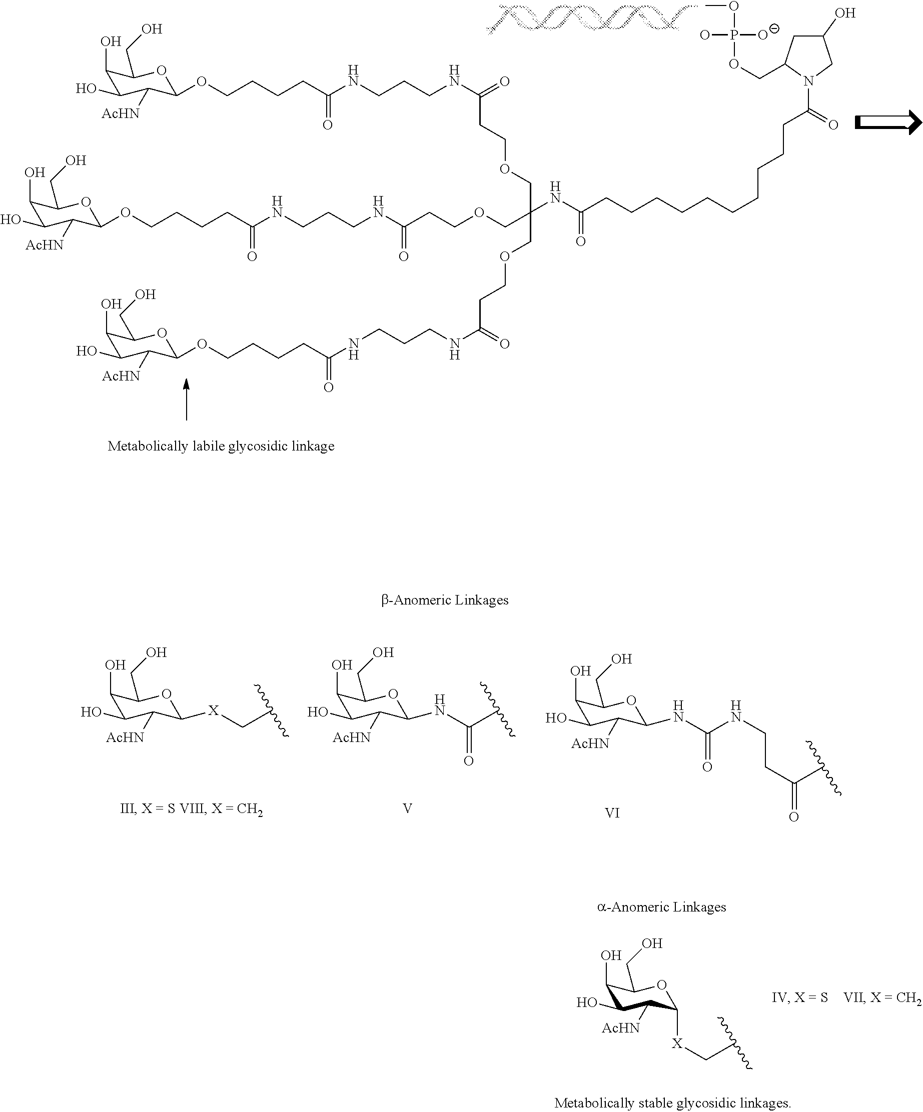

See the synthesis procedures of these GalNAc analog in Kandasamy et al., “Metabolically Stable Anomeric Linkages Containing GalNAc—siRNAConjugates: An Interplay among ASGPR, Glycosidase, and RISC Pathywas,” J. Med. Chem. 66:2506-23 (2023), which is incorporated by reference in its entirety.

In some embodiments, the GalNAc-based conjugate is a GalNAc analog having one of the following structures:

| | |
|---|---|
| 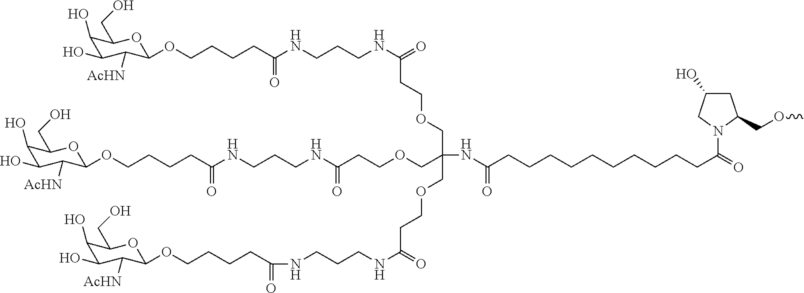 | L96 |
| 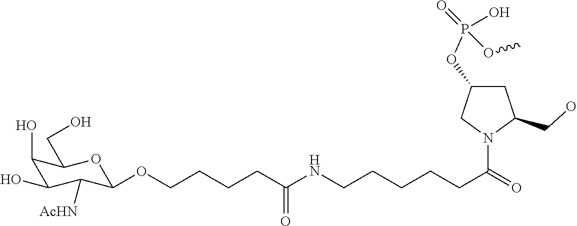 | Q150 |
| 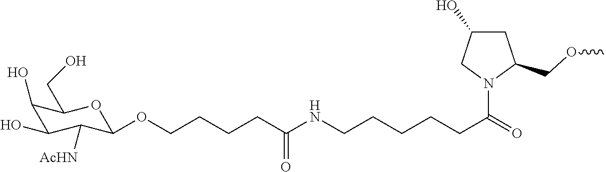 | L193 |
| 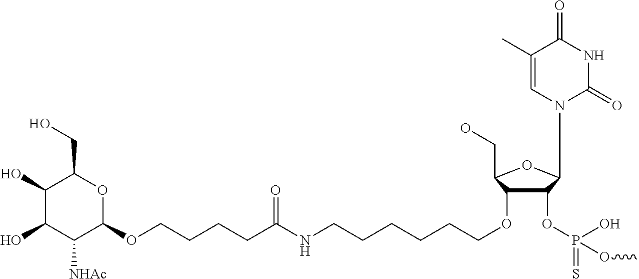 | (T3gs) |
| 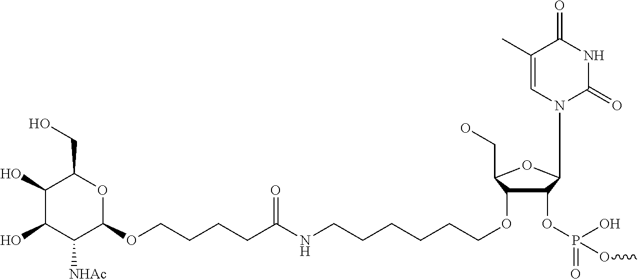 | (T3g) |
| 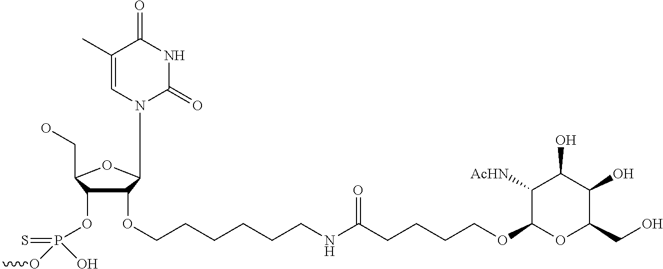 | (Tgs) |
| 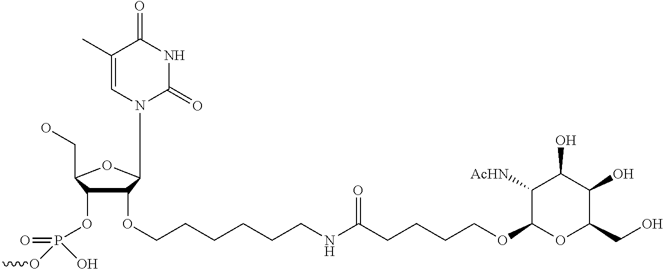 | (Tg) |
| 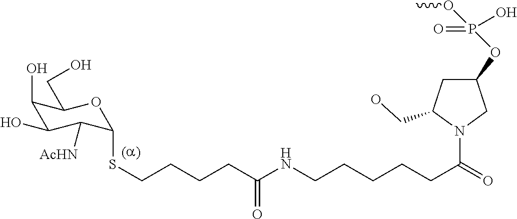 | Q155 |
| 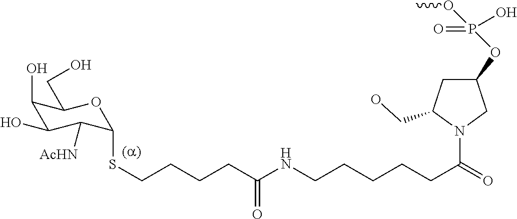 | L199 |
| 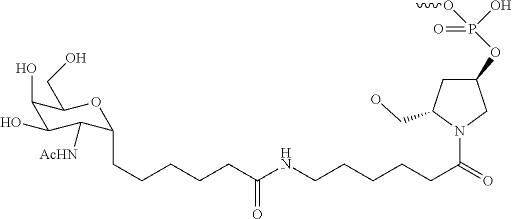 | Q160 |
| 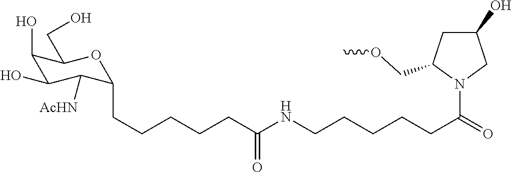 | L204 |
| 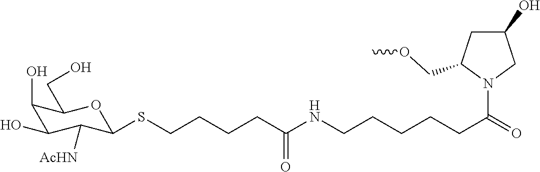 | L200 |
| 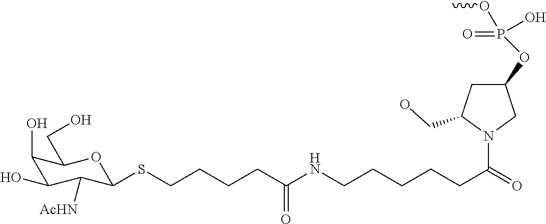 | Q156 |
| 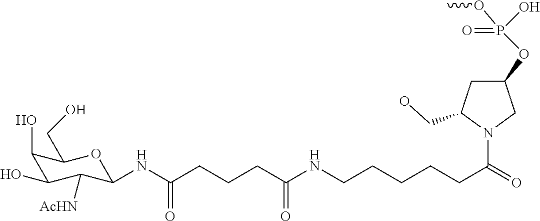 | Q154 |
| 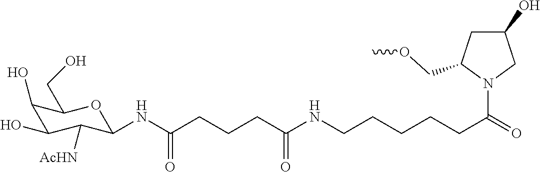 | L198 |
| 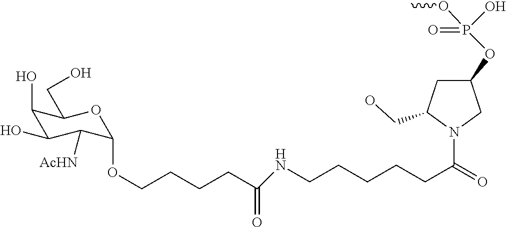 | Q161 |
| 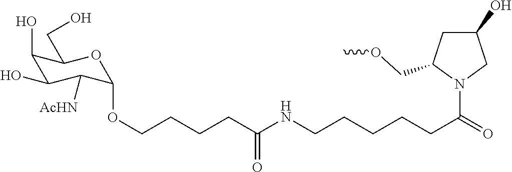 | L207 |
| 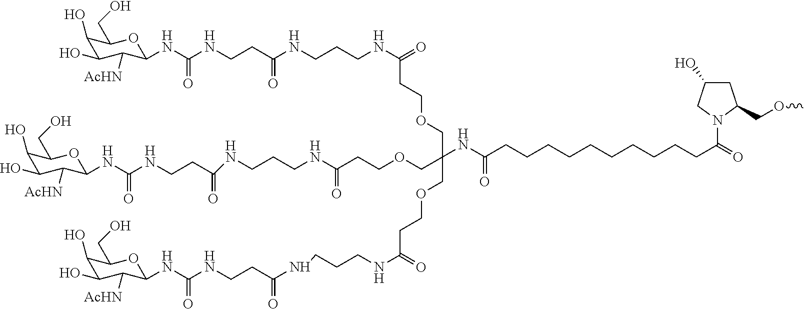 | L206 |
| 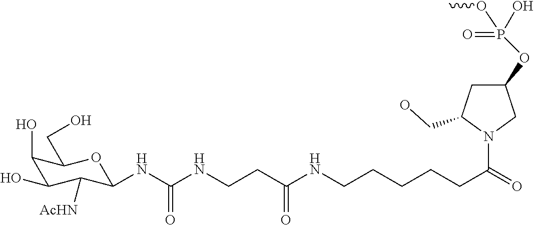 | Q159 |
| 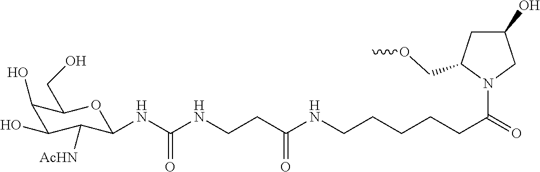 | L203 |
| 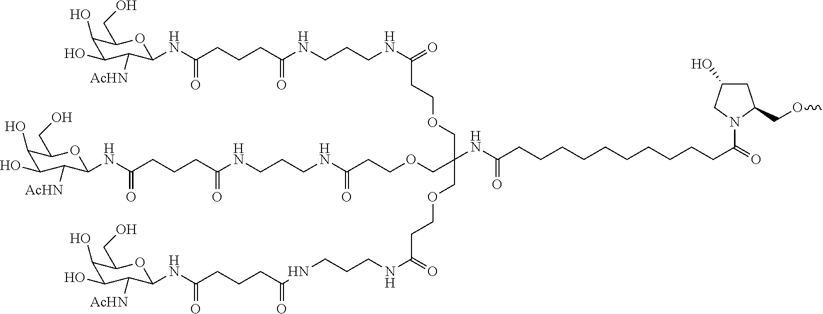 | L197 |
| 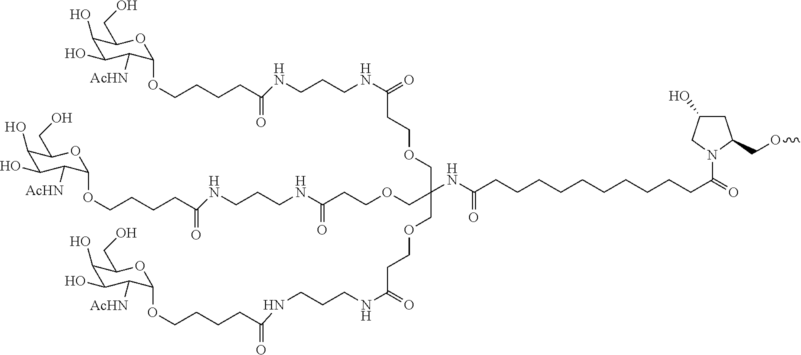 | L208 |
| 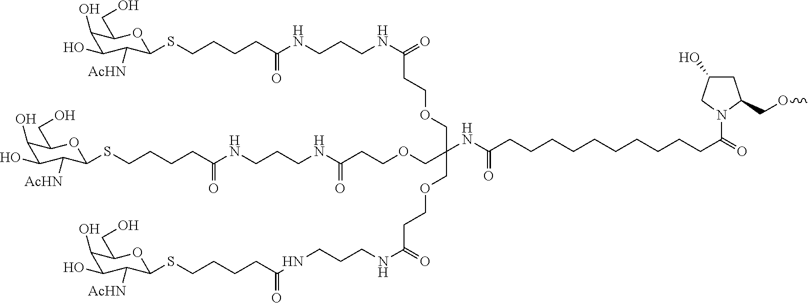 | L202 |
| 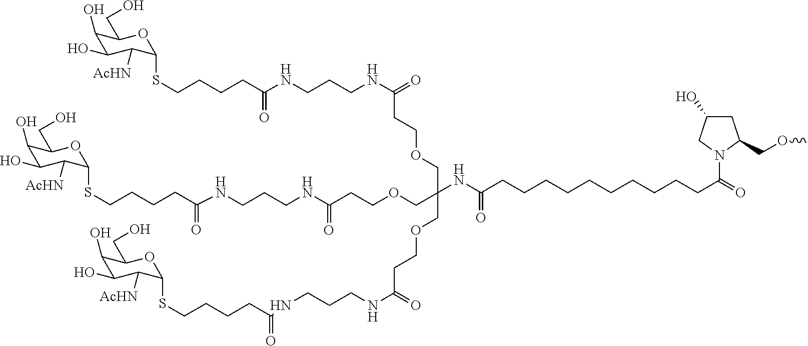 | L201 |
| 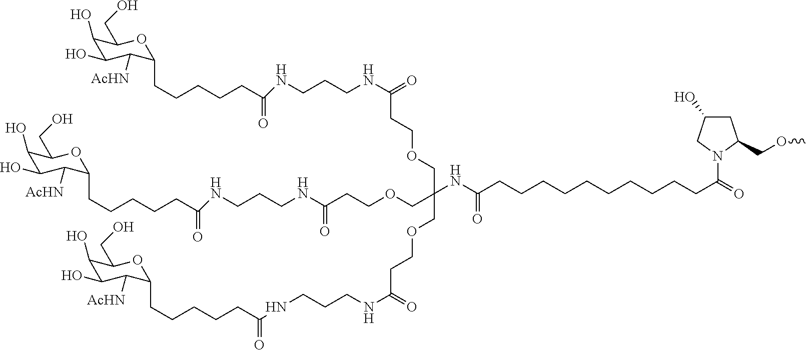 | L205 |
| 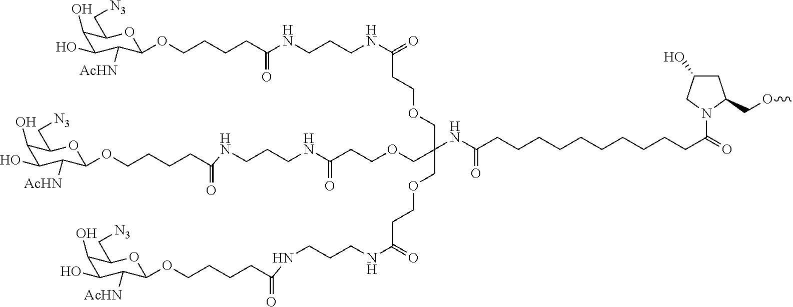 | L223 |
| 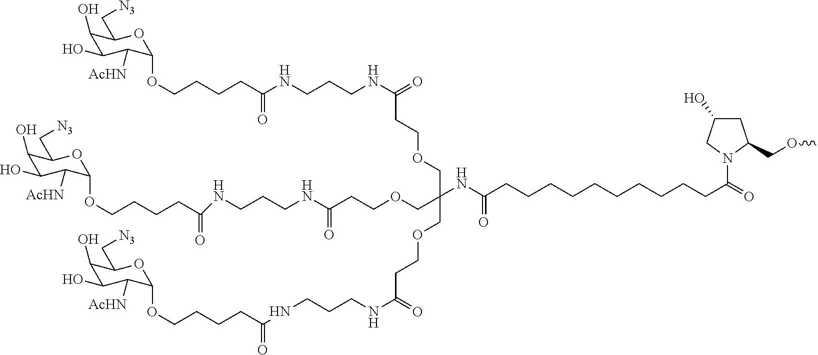 | L224 |
| 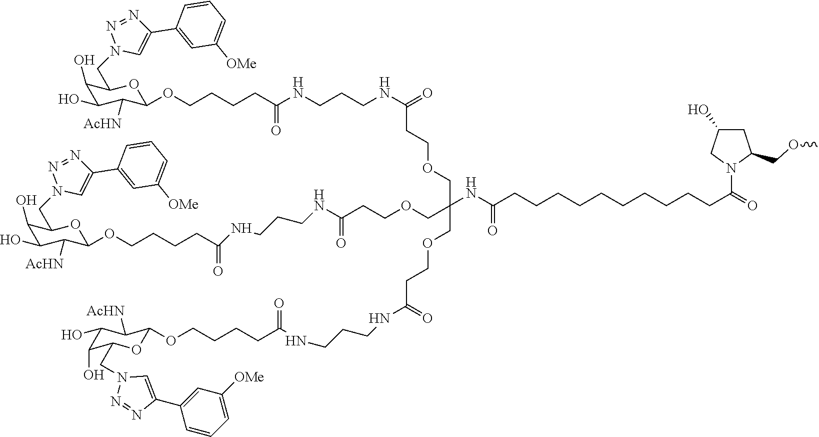 | L221 |
| 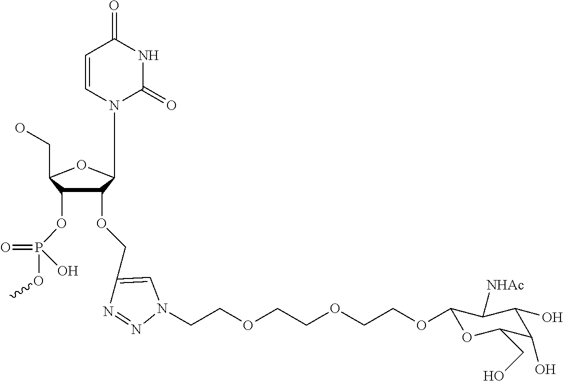 | (Uyg) |
| 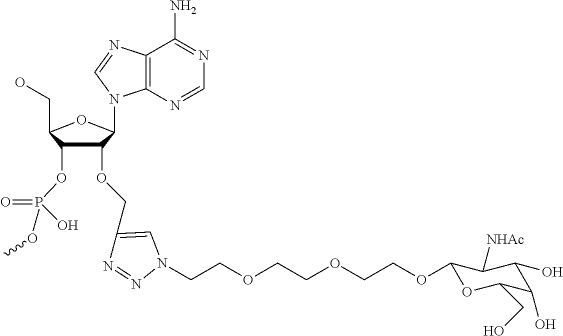 | (Ayg) |
| 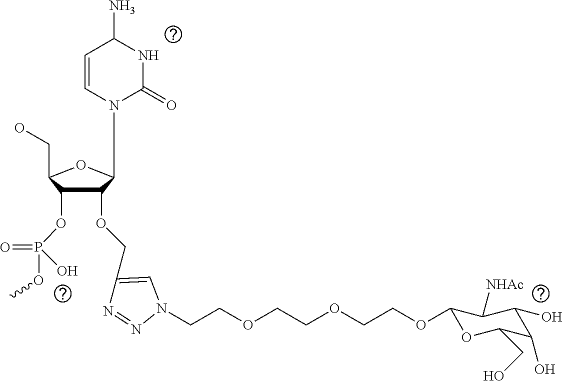 | (Cyg) |
| 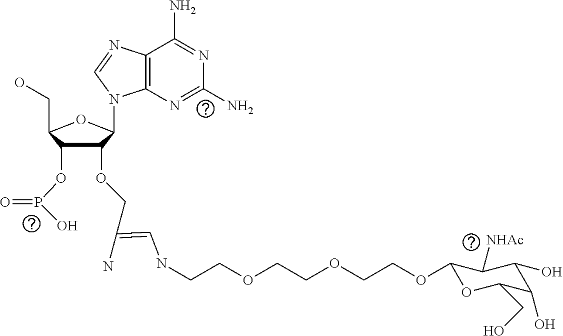 | (Gyg) |
| 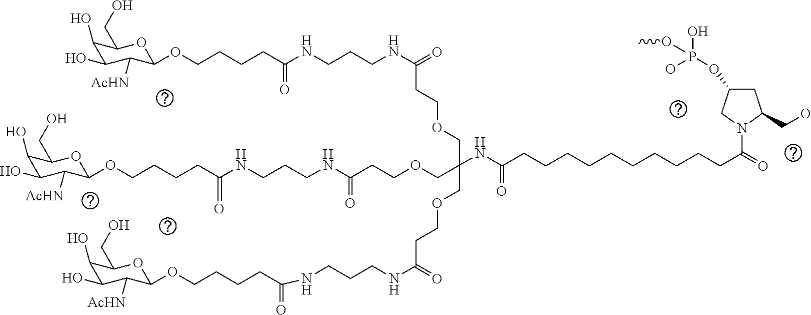 | Q151 |

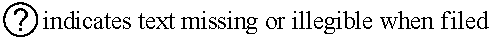

The GalNAc analogs listed in the above table may be prepared using the methods described in WO2015/006740, which is incorporated herein by reference in its entirety.

In some embodiments, the GalNAc-based conjugate is a GalNAc analog having one of the following structures.

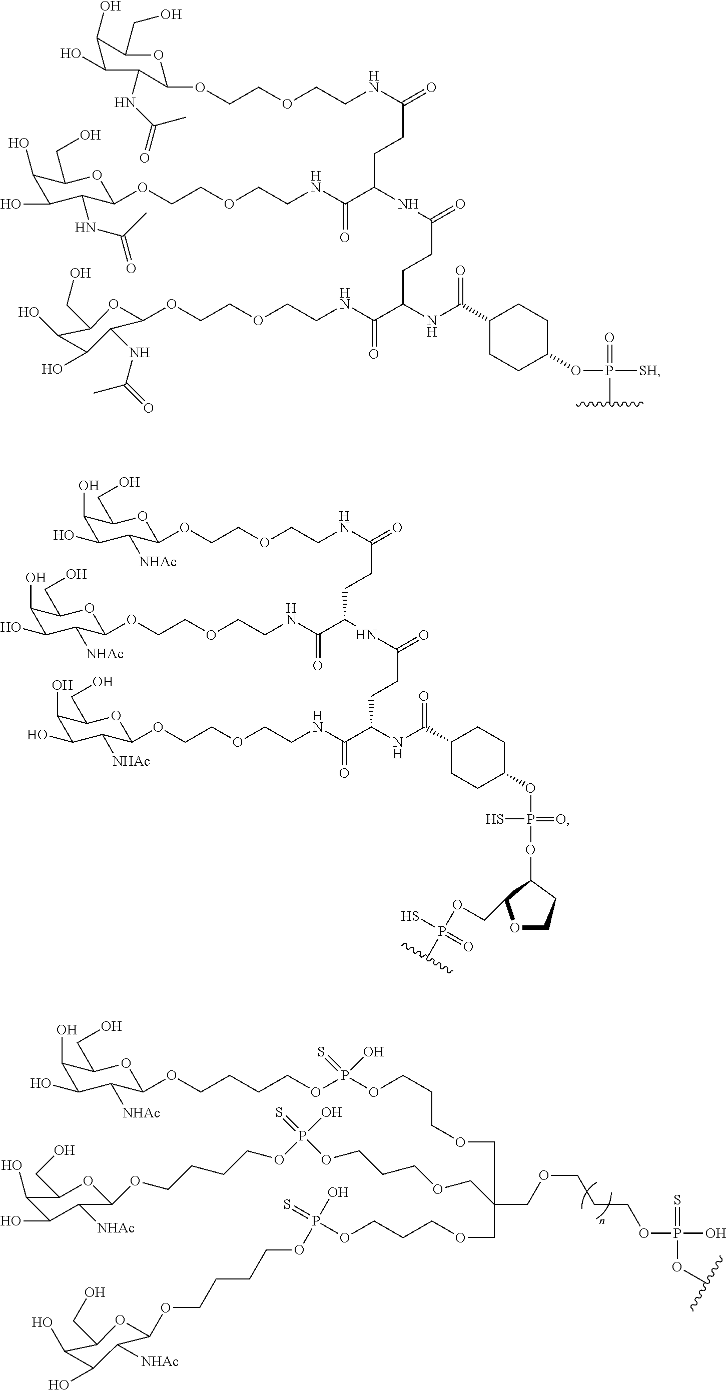

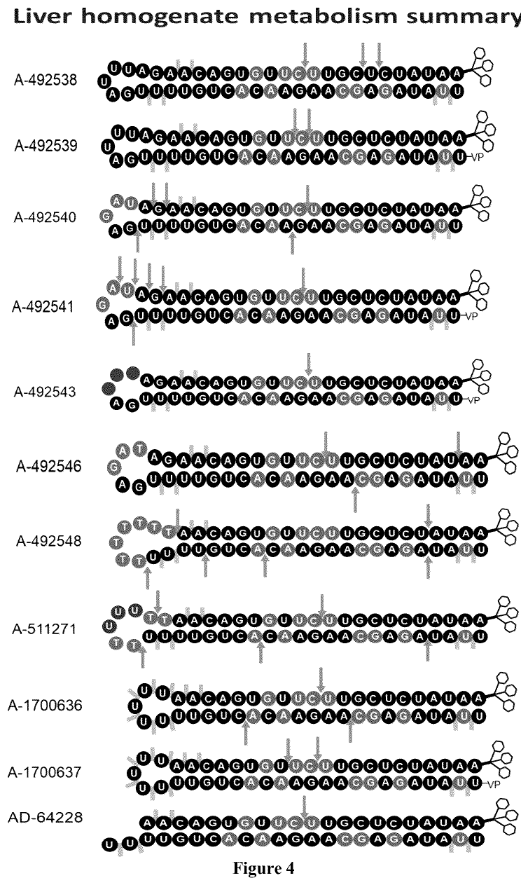

(e.g., 1 or 4). See FIGS. 4A and 4B of US2021/0123048A1, which is incorporated herein by reference in its entirety),

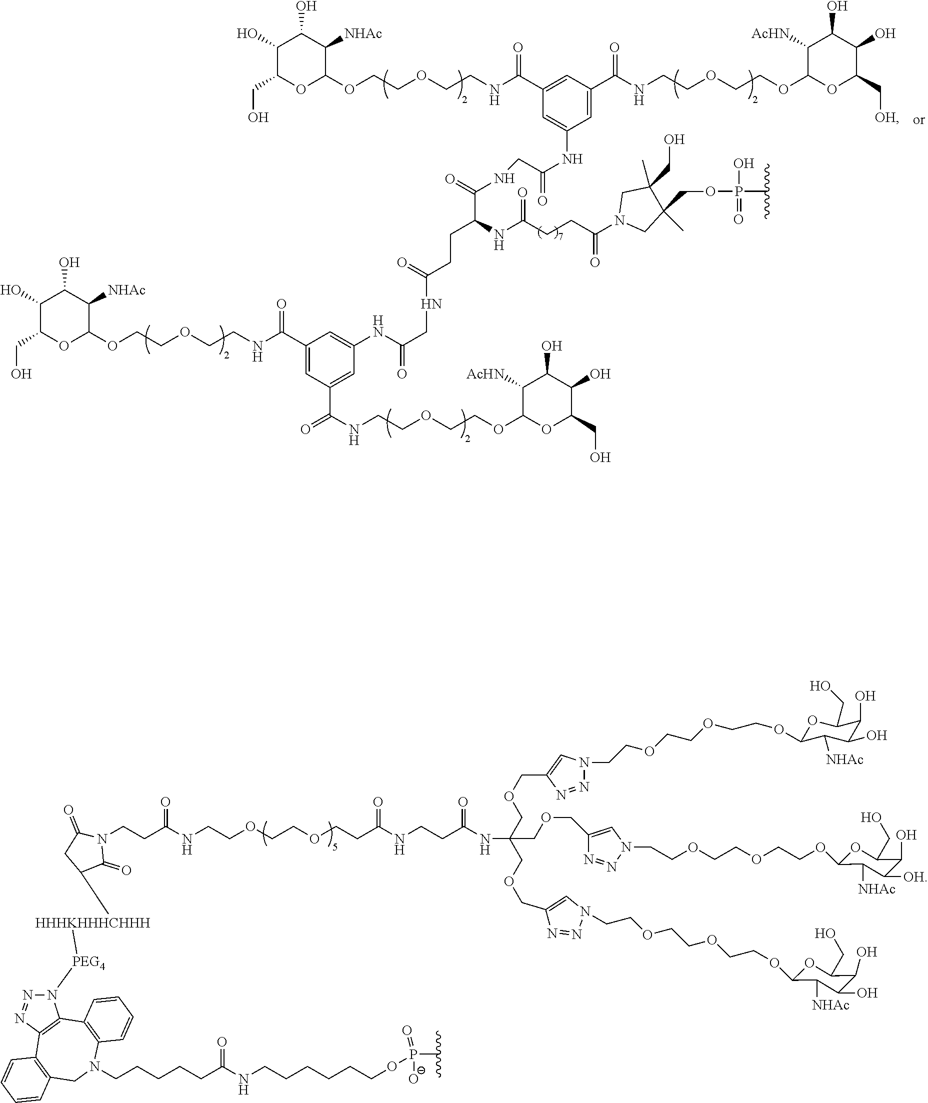

In certain embodiments, the single-stranded oligonucleotide further comprises a ligand having a structure shown below:

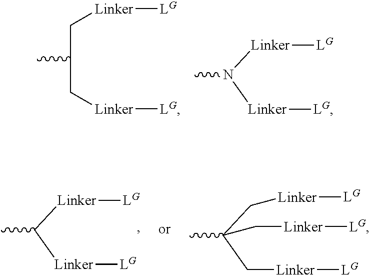

wherein:
L^{G} is independently for each occurrence a ligand, e.g., carbohydrate, e.g. monosaccharide, disaccharide, trisaccharide, tetrasaccharide, polysaccharide; and
Z′, Z″, Z′″ and Z″″ are each independently for each occurrence 0 or S.

In certain embodiments, the single-stranded oligonucleotide comprises a ligand of Formula (II), (III), (IV) or (V):

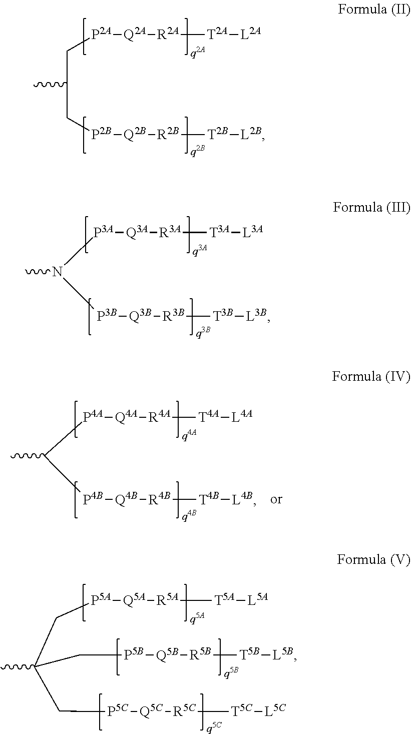

wherein:
q^{2A}, q^{2B}, q^{3A}, q^{3B}, q^{4A}, q^{4B}, q^{5A}, q^{5B} and q^{5C} represent independently for each occurrence 0-20 and wherein the repeating unit can be the same or different;
Q and Q′ are independently for each occurrence is absent, —(P⁷-Q⁷-R⁷)ₚ-T⁷- or -T⁷-Q⁷-T^{7′}-B-T^{8′}-Q⁸-T⁸;
P^{2A}, P^{2B}, P^{3A}, P^{3B}, P^{4A}, P^{4B}, P^{5A}, P^{5B}, P^{5C}, P⁷, T^{2A}, T^{2B}, T^{3A}, T^{3B}, T^{4A}, T^{4B}, T^{4A}, T^{5B}, T^{5C}, T⁷, T^{7′}, T⁸ and T^{8′} are each independently for each occurrence absent, CO, NH, O, S, OC(O), NHC(O), CH₂, CH₂NH or CH₂O;
B is —CH₂—N(B^{L})—CH₂—;
BL is -T^{B}_Q^{B}-T^{B′}-R^{x};
Q^{2A}, Q^{2B}, Q^{3A}, Q^{3B}, Q^{4A}, Q^{4B}, Q^{5A}, Q^{5B}, Q^{5C}, Q⁷, Q⁸ and Q^{B} are independently for each occurrence absent, alkylene, substituted alkylene and wherein one or more methylenes can be interrupted or terminated by one or more of O, S, S(O), SO₂, N(R^{N}), C(R′)═C(R′), C≡C or C(O);
T^{B} and T^{B′} are each independently for each occurrence absent, CO, NH, O, S, OC(O), OC(O)O, NHC(O), NHC(O)NH, NHC(O)O, CH₂, CH₂NH or CH₂O;
R^{x} is a lipophile (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, Friedelin, epifriedelanol derivatized lithocholic acid), or a cationic lipid;
R¹, R², R^{2A}, R^{2B}, R^{3A}, R^{3B}, R^{4A}, R^{4B}, R^{5A}, R^{5B}, R^{5C}, R⁷ are each independently for each occurrence absent, NH, O, S, CH₂, C(O)O, C(O)NH, NHCH(R^{a})C(O), —C(O)—CH(R^{a})—NH—, CO, CH═N—O,

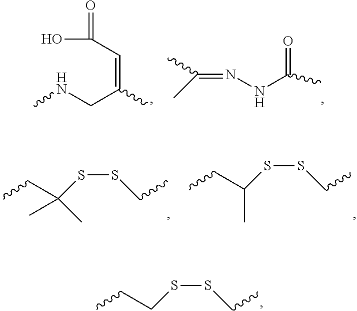

or heterocyclyl;
L¹, L^{2A}, L^{2B}, L^{3A}, L^{3B}, L^{4A}, L^{4B}, L^{5A}, L^{5B} and L^{5C} are each independently for each occurrence a carbohydrate, e.g., monosaccharide, disaccharide, trisaccharide, tetrasaccharide, oligosaccharide and polysaccharide;
R′ and R″ are each independently H, C₁-C₆ alkyl, OH, SH, or N(R^{N})₂;
R^{N} is independently for each occurrence H, methyl, ethyl, propyl, isopropyl, butyl or benzyl;
R^{a} is H or amino acid side chain;
Z′, Z″, Z′″ and Z″″ are each independently for each occurrence 0 or S;
p represents independently for each occurrence 0-20.

As discussed above, because the ligand can be conjugated to the single-stranded oligonucleotide via a linker or carrier, and because the linker or carrier can contain a branched linker, the single-stranded oligonucleotide can then contain multiple ligands via the same or different backbone attachment points to the carrier, or via the branched linker(s). For instance, the branchpoint of the branched linker may be a bivalent, trivalent, tetravalent, pentavalent, or hexavalent atom, or a group presenting such multiple valencies. In certain embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In other embodiment, the branchpoint is glycerol or glycerol derivative.

In certain embodiments, the ASGPR ligand conjugated to the single-stranded oligonucleotide is one or more GalNAc derivatives attached through a bivalent or trivalent branched linker.

In certain embodiments, the single-stranded oligonucleotide comprises a ligand of:

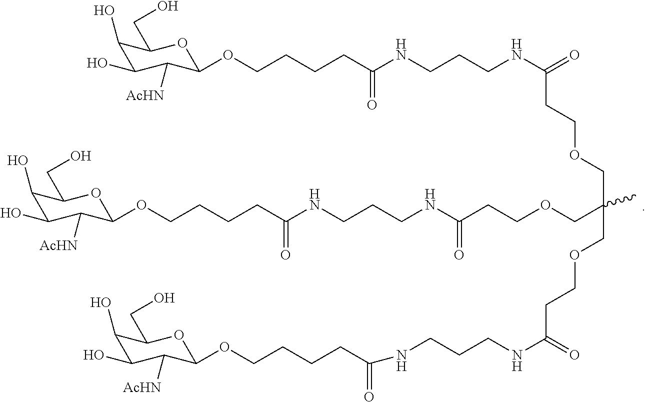 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In certain embodiments, the single-stranded oligonucleotide comprises a ligand of:

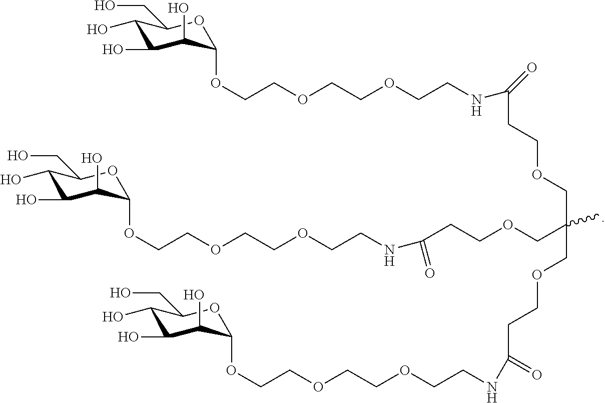

In certain embodiments, the single-stranded oligonucleotide comprises a ligand of:

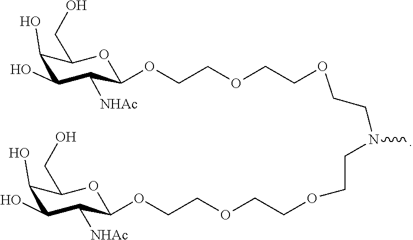

In certain embodiments, the single-stranded oligonucleotide comprises a ligand of:

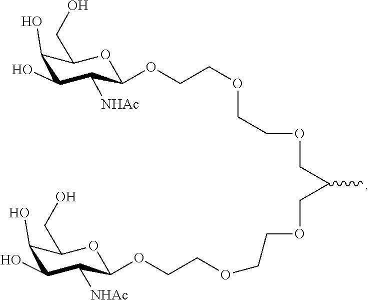

In certain embodiments, the single-stranded oligonucleotide comprises a ligand of:

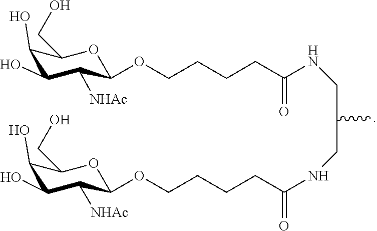

In certain embodiments, the single-stranded oligonucleotide comprises a ligand of:

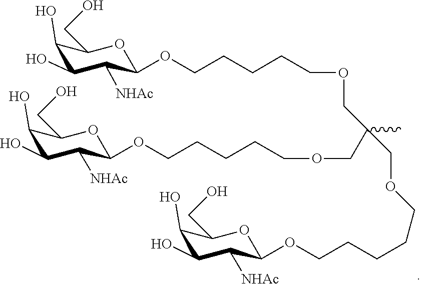

In certain embodiments, the single-stranded oligonucleotide comprises a ligand of:

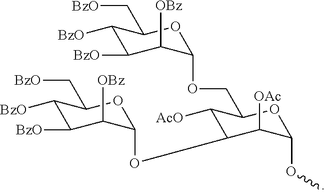

In certain embodiments, the single-stranded oligonucleotide comprises a ligand of:

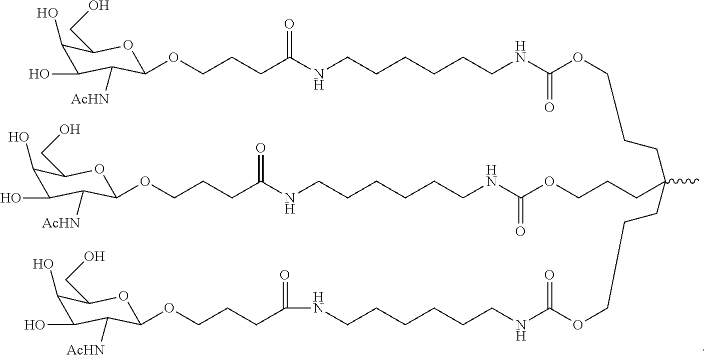 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

In certain embodiments, the single-stranded oligonucleotide comprises a ligand of:

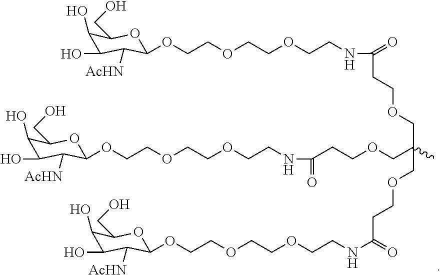 [SMILES: CC(=O)NC1C(OCCOCCOCCNC(=O)CCOCC(C)(COCCC(=O)NCCOCCOCCOC2OC(CO)C(O)C(O)C2NC(C)=O)COCCC(=O)NCCOCCOCCOC2OC(CO)C(O)C(O)C2NC(C)=O)OC(CO)C(O)C1O]

In certain embodiments, the single-stranded oligonucleotide comprises a ligand of:

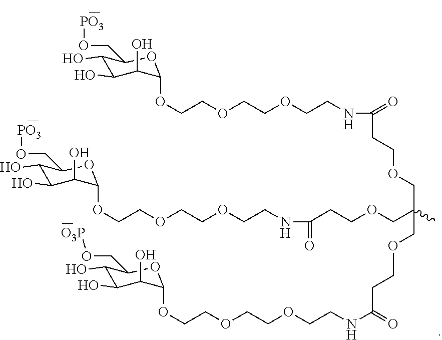

In certain embodiments, the single-stranded oligonucleotide comprises a ligand of:

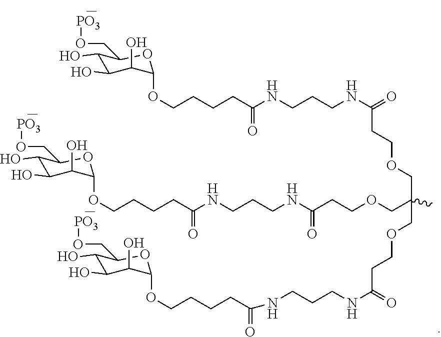

In certain embodiments, the single-stranded oligonucleotide comprises a monomer of:

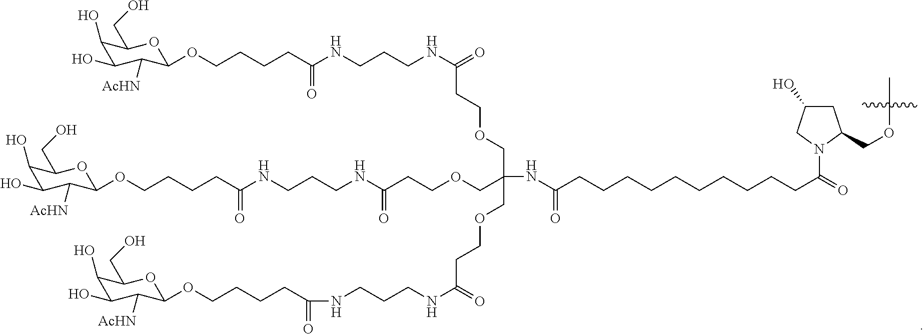 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In certain embodiments, the single-stranded oligonucleotide comprises a ligand of:

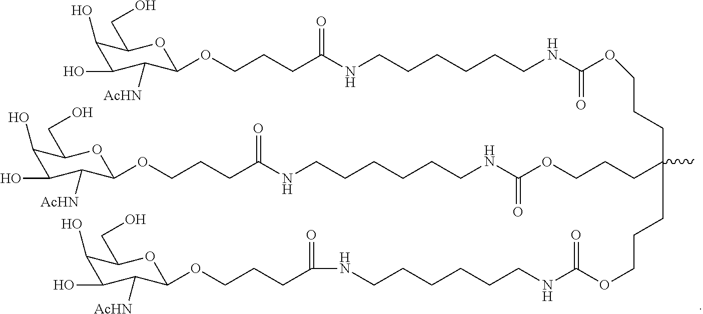 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

### Exemplary Ligand Monomers

In certain embodiments, the single-stranded oligonucleotide comprises a monomer of:

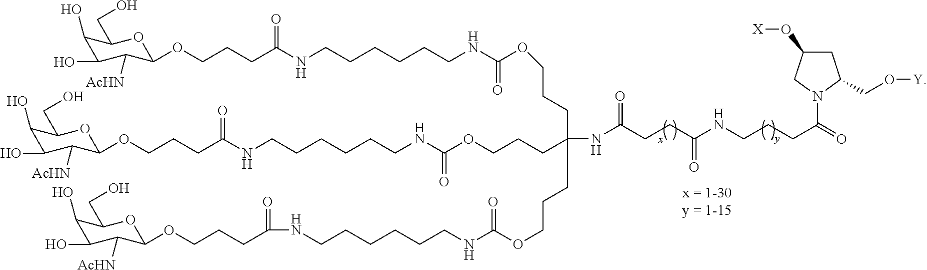

In certain embodiments, the single-stranded oligonucleotide comprises a monomer of:

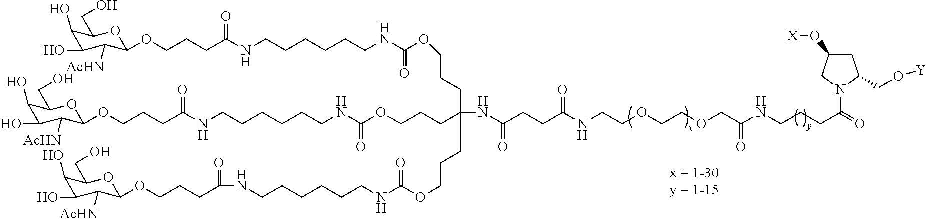

In certain embodiments, the single-stranded oligonucleotide comprises a monomer of:

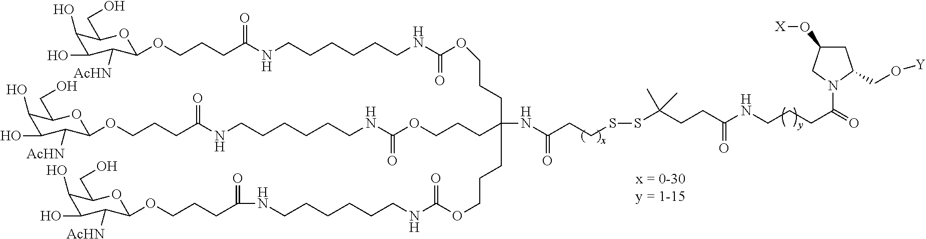

In certain embodiments, the single-stranded oligonucleotide comprises a monomer of:

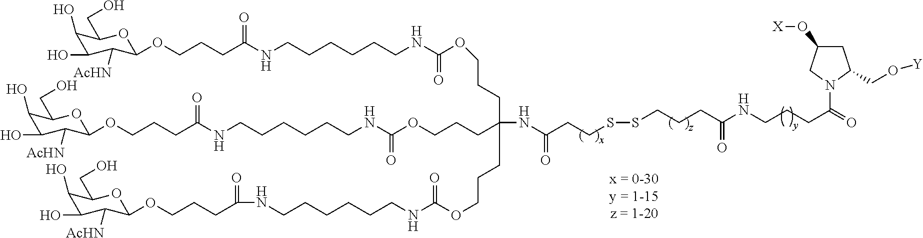

In certain embodiments, the single-stranded oligonucleotide comprises a monomer of:

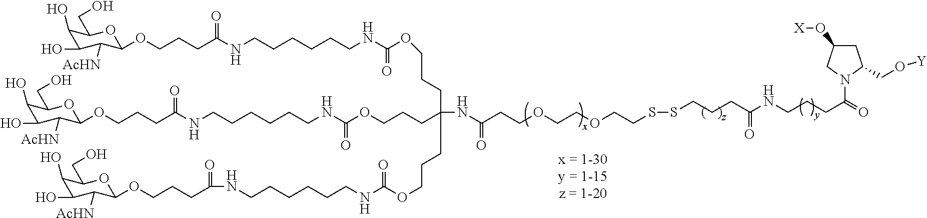

In certain embodiments, the single-stranded oligonucleotide comprises a monomer of:

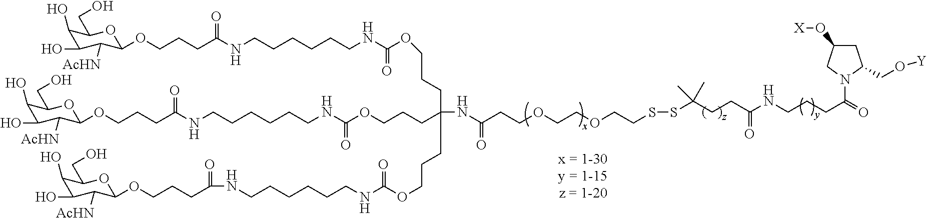

In certain embodiments, the single-stranded oligonucleotide comprises a ligand of:

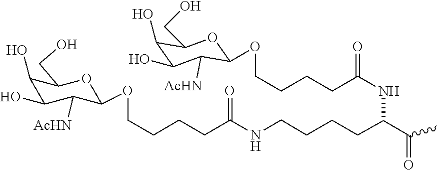

In certain embodiments, the single-stranded oligonucleotide comprises a ligand of:

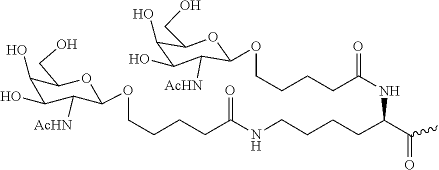

In certain embodiments, the single-stranded oligonucleotide comprises a ligand of:

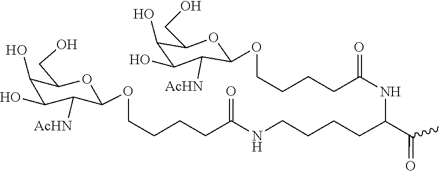

In certain embodiments, the single-stranded oligonucleotide comprises a ligand of:

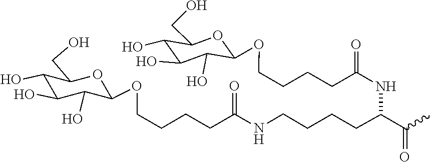

In certain embodiments, the single-stranded oligonucleotide comprises a ligand of:

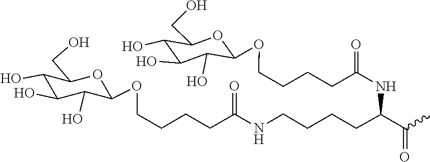

In certain embodiments, the single-stranded oligonucleotide comprises a ligand of:

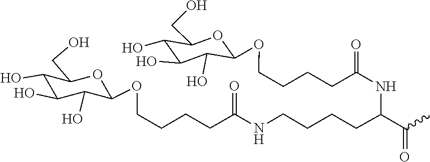

In certain embodiments, the single-stranded oligonucleotide comprises a ligand of:

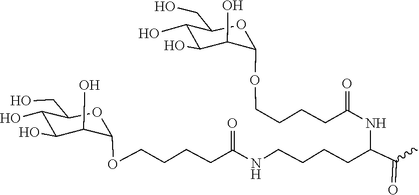

In certain embodiments, the single-stranded oligonucleotide comprises a ligand of:

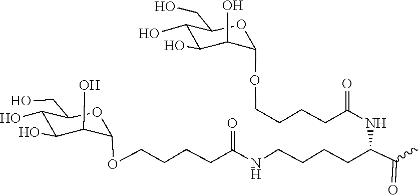

In certain embodiments, the single-stranded oligonucleotide comprises a ligand of:

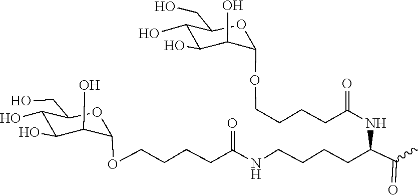

In certain embodiments, the single-stranded oligonucleotide comprises a monomer of:

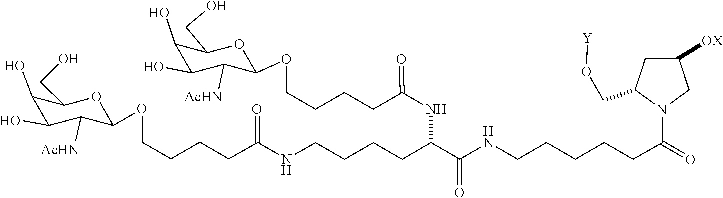

In certain embodiments, the single-stranded oligonucleotide comprises a monomer of:

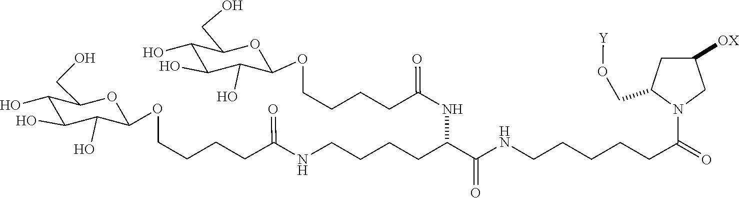

In certain embodiments, the single-stranded oligonucleotide comprises a monomer of:

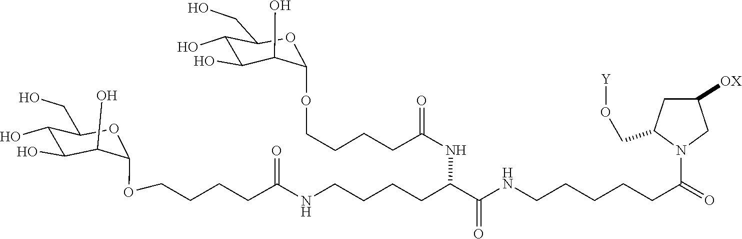

In certain embodiments, the single-stranded oligonucleotide comprises a monomer of:

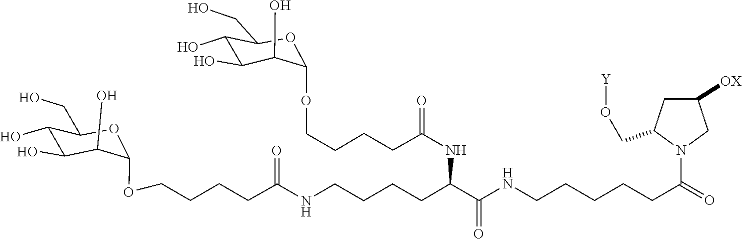

In certain embodiments, the single-stranded oligonucleotide of the invention comprises a monomer of:

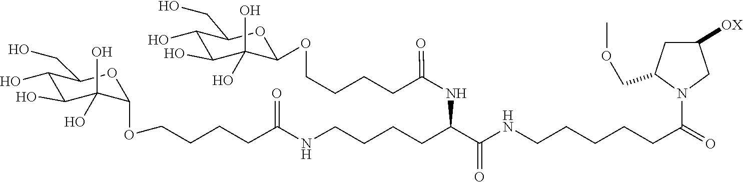

In certain embodiments, the single-stranded oligonucleotide comprises a monomer of:

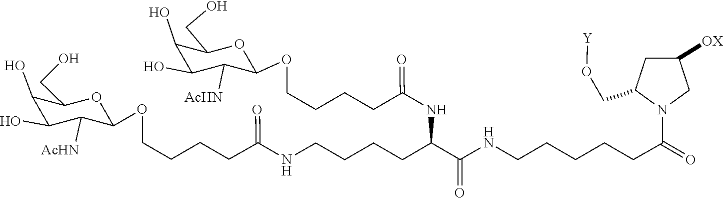

In some embodiments, both L^{2A} and L^{2B} are the same. In some embodiments, both L^{2A} and L^{2B} are different.

In some embodiments, both L^{3A} and L^{3B} are the same. In some embodiments, both L^{3A} and L^{3B} are different.

In some embodiments, both L^{4A} and L^{4B} are the same. In some embodiments, both L^{4A} and L^{4B} are different.

In some embodiments, all of L^{5A}, L^{5B} and L^{5C} are the same. In some embodiments, two of L^{5A}, L^{5B} and L^{5C} are the same. In some embodiments, L^{5A} and L^{5B} are the same. In some embodiments, L^{5A} and L^{5C} are the same. In some embodiments, L^{5B} and L^{5C} are the same.

In certain embodiments, the single-stranded oligonucleotide comprises a monomer of:

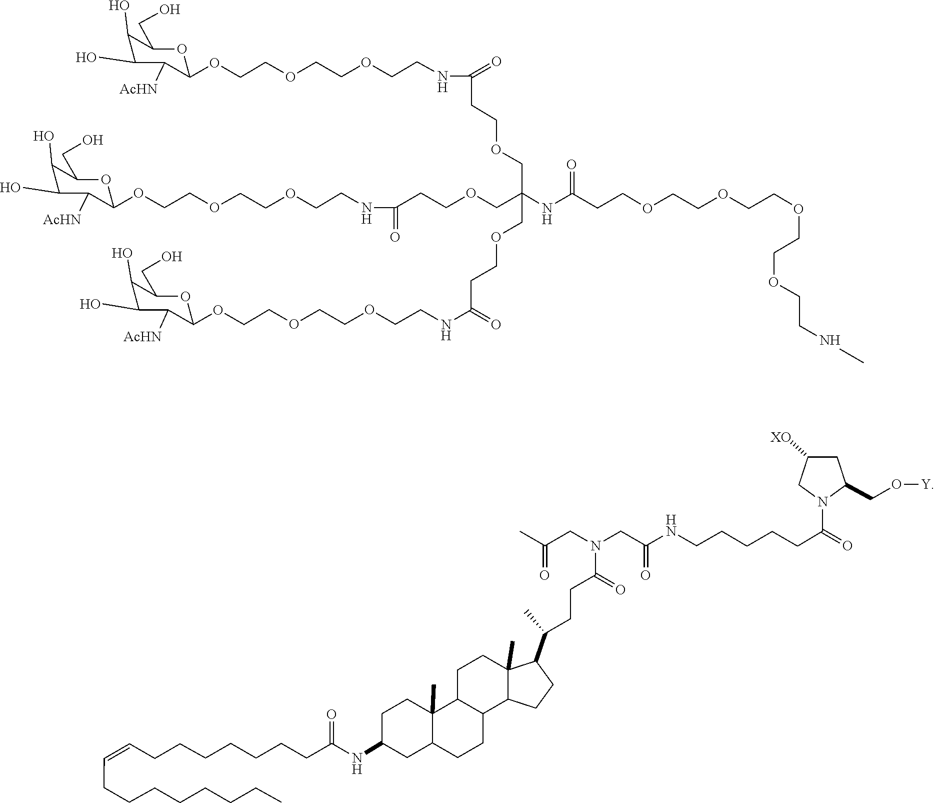

In certain embodiments, the single-stranded oligonucleotide comprises a monomer of:

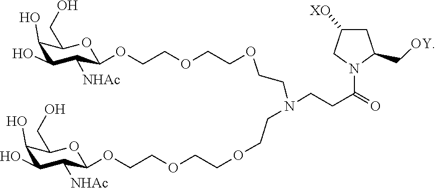

In certain embodiments, the single-stranded oligonucleotide comprises a monomer of:

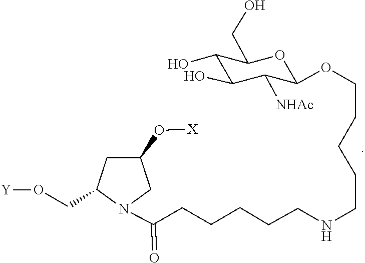

In certain embodiments, the single-stranded oligonucleotide comprises a monomer of:

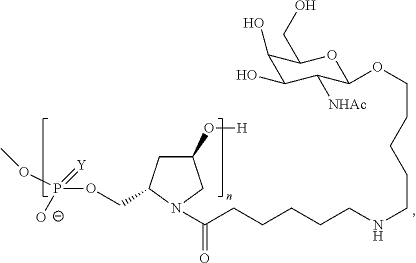

wherein Y is O or S, and n is 1-6.

In certain embodiments, the single-stranded oligonucleotide comprises a monomer of:

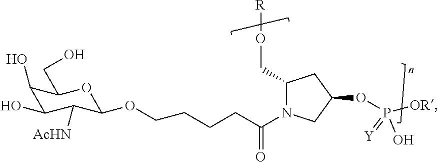

wherein Y is O or S, n is 1-6, R is hydrogen or nucleic acid, and R′ is nucleic acid.

In certain embodiments, the single-stranded oligonucleotide comprises a monomer of:

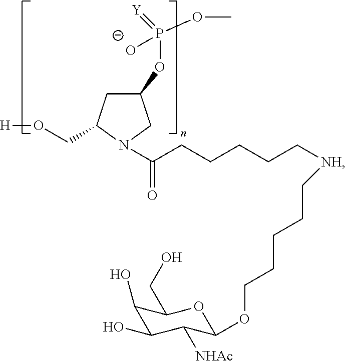

wherein Y is O or S, and n is 1-6.

In certain embodiments, the single-stranded oligonucleotide comprises a monomer of structure:

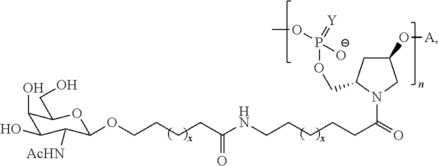

wherein Y is O or S, n is 2-6, x is 1-6, and A is H or a phosphate linkage.

In some embodiments, the single-stranded oligonucleotide comprises at least 1, 2, 3 or 4 monomer of:

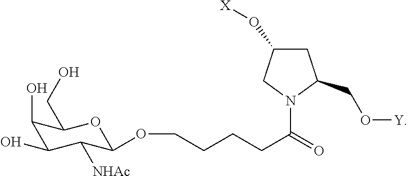

In some embodiments, the single-stranded oligonucleotide comprises a monomer

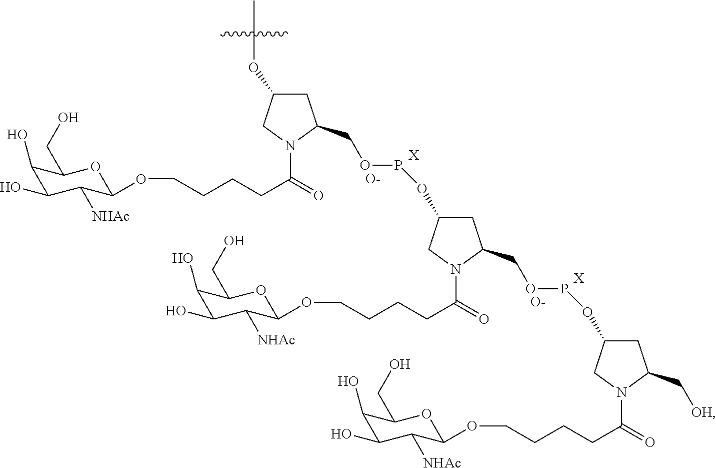

wherein X is O or S.

In some embodiments, the single-stranded oligonucleotide comprises a monomer of:

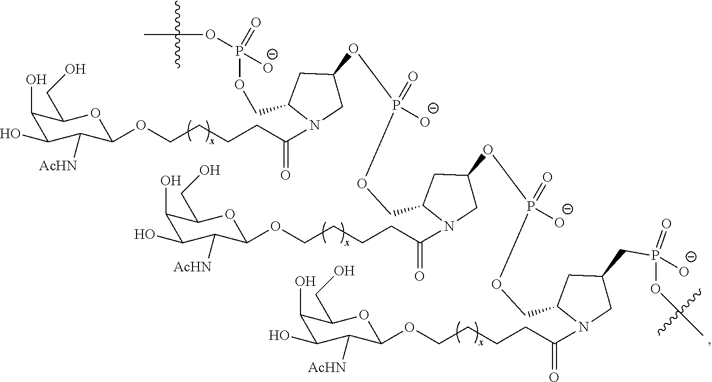

wherein x is 1-12.

In some embodiments, the single-stranded oligonucleotide comprises a monomer of:

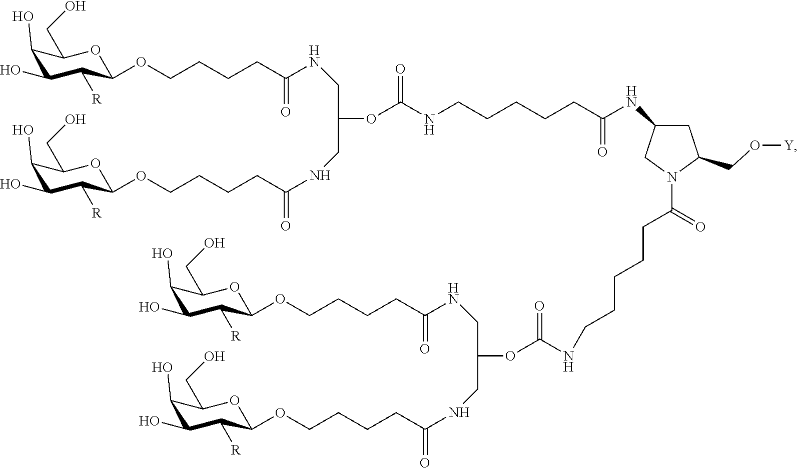

wherein R is OH or NHCOCH₃.

In certain embodiments, the single-stranded oligonucleotide comprises a monomer of:

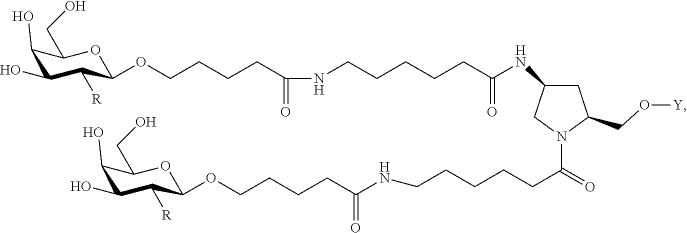

wherein R is OH or NHCOCH₃.

In certain embodiments, the single-stranded oligonucleotide comprises a monomer of:

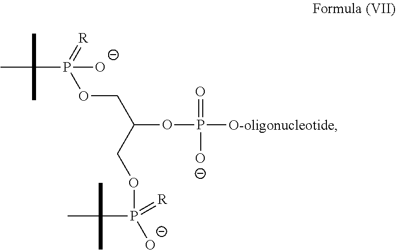

wherein R is O or S.

In certain embodiments, the single-stranded oligonucleotide comprises a monomer of:

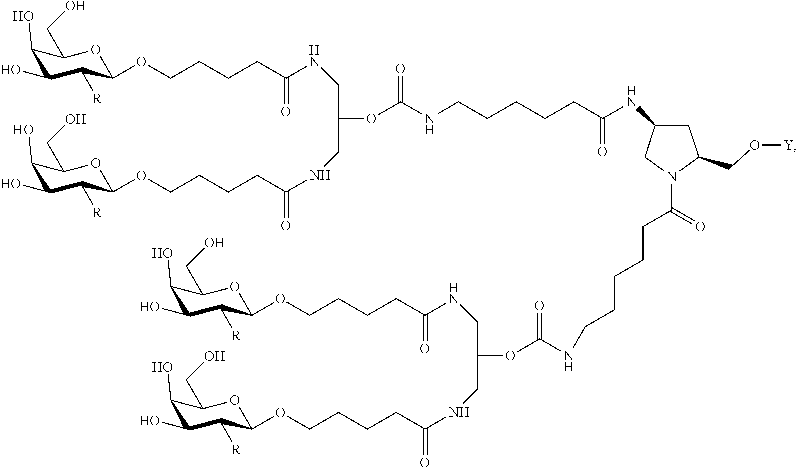

wherein R is OH or NHCOCH₃.

In certain embodiments, the single-stranded oligonucleotide comprises a monomer of:

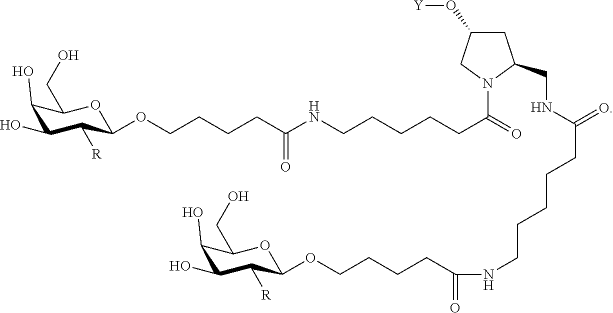

In some embodiments, the single-stranded oligonucleotide comprises a monomer of:

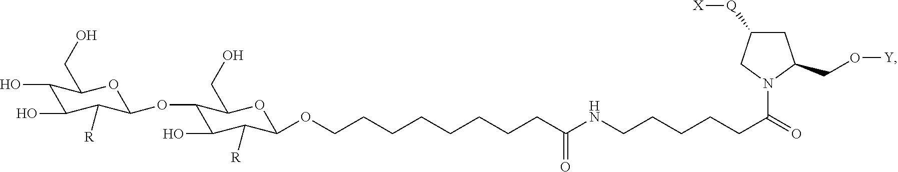

wherein R is OH or NHCOCH₃.

In some embodiments, the single-stranded oligonucleotide comprises a monomer of:

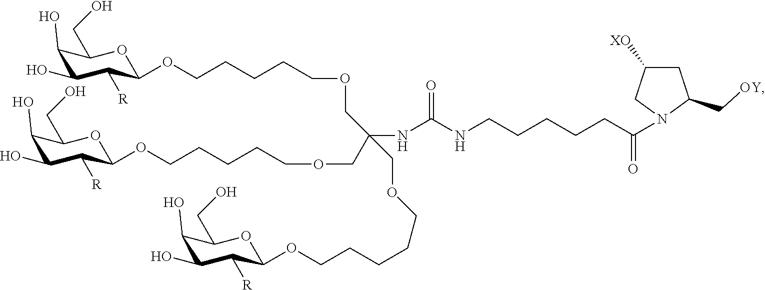

wherein R is OH or NHCOCH₃.

In some embodiments, the single-stranded oligonucleotide comprises a monomer of:

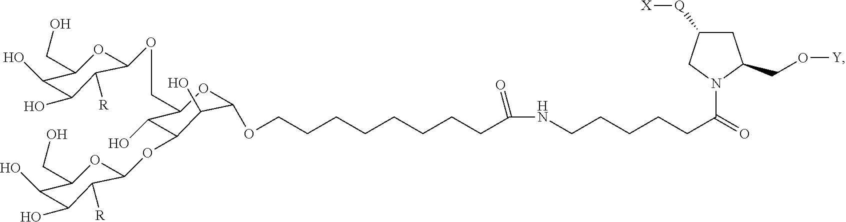

wherein R is OH or NHCOCH₃.

In certain embodiments, the single-stranded oligonucleotide comprises a monomer of:

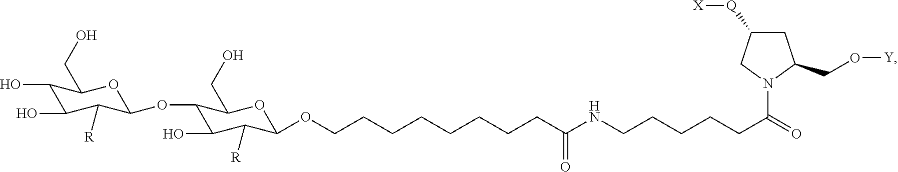

wherein R is OH or NHCOCH₃.

In certain embodiments, the single-stranded oligonucleotide comprises a monomer of:

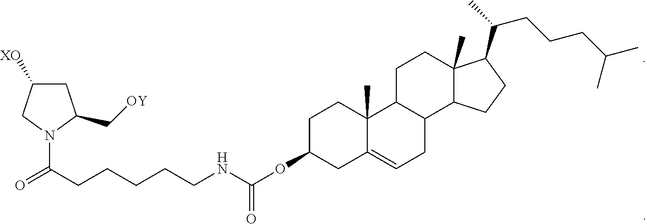

In the above described monomers, X and Y are each independently for each occurrence H, a protecting group, a phosphate group, a phosphodiester group, an activated phosphate group, an activated phosphite group, a phosphoramidite, a solid support, —P(Z′)(Z″)O-nucleoside, —P(Z′)(Z″)O-oligonucleotide, a lipid, a PEG, a steroid, a polymer, a nucleotide, a nucleoside, or an oligonucleotide; and Z′ and Z″ are each independently for each occurrence O or S.

In some embodiments, the single-stranded oligonucleotide is conjugated with a ligand of:

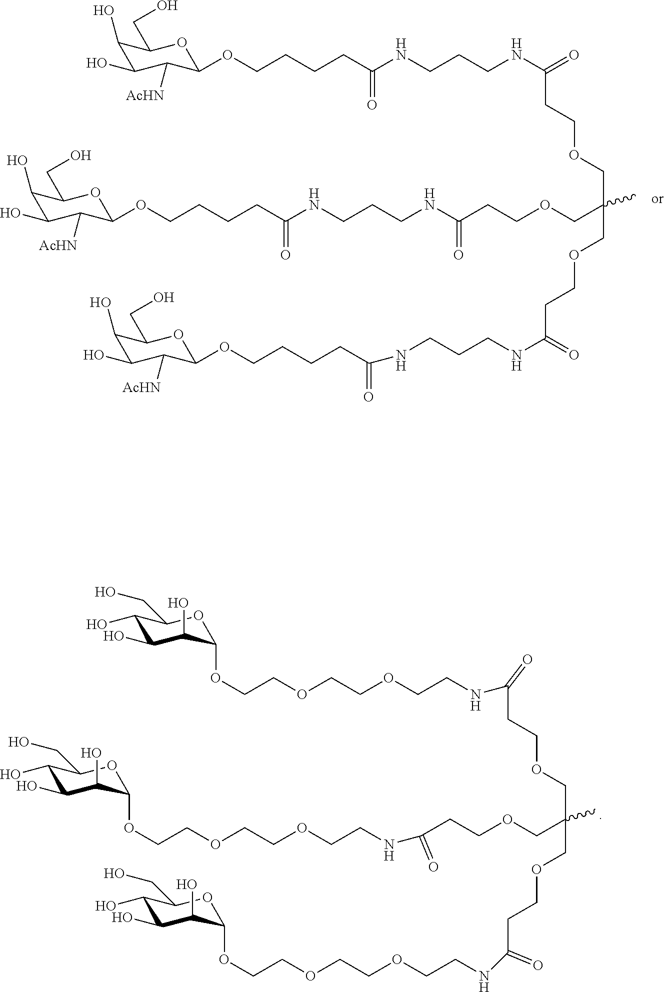

In certain embodiments, the single-stranded oligonucleotide comprises a ligand of:

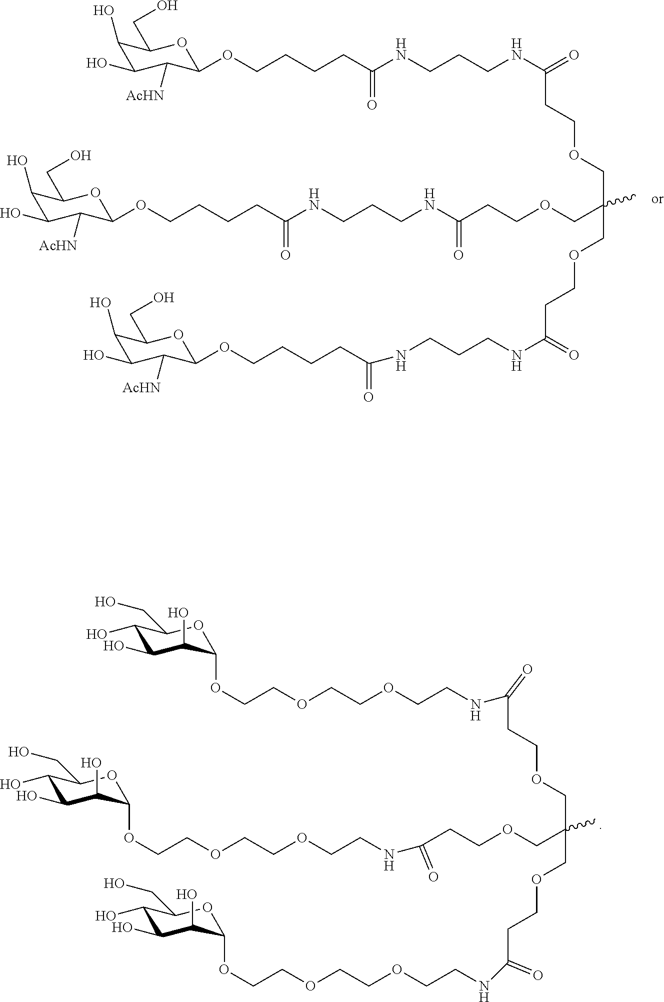

In certain embodiments, the single-stranded oligonucleotide comprises a monomer of:

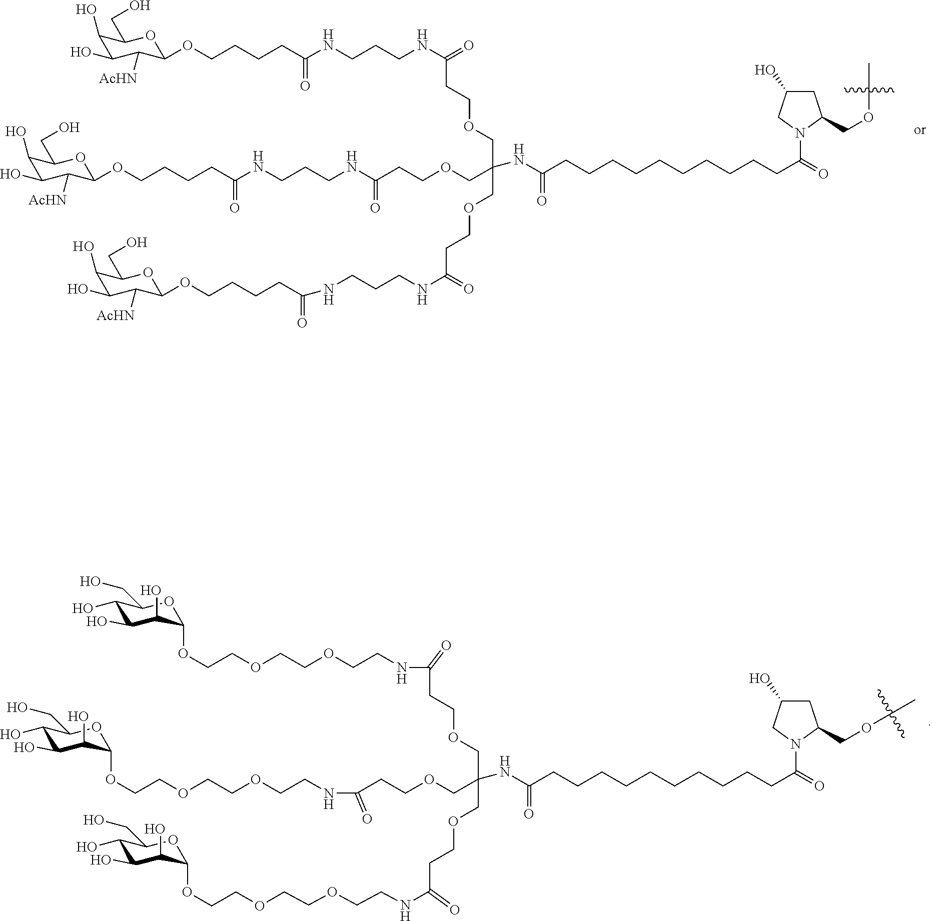

Synthesis of above described ligands and monomers is described, for example, in U.S. Pat. No. 8,106,022, content of which is incorporated herein by reference in its entirety.

In certain embodiments, at least one of the ligands conjugated to the single-stranded oligonucleotide is a lipophilic moiety.

The term “lipophile” or “lipophilic moiety” broadly refers to any compound or chemical moiety having an affinity for lipids. One way to characterize the lipophilicity of the lipophilic moiety is by the octanol-water partition coefficient, log K_{ow}, where K_{ow} is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko et al., J Chem. Inf Comput. Sci. 41:1407-21 (2001), which is incorporated herein by reference in its entirety). It provides a thermodynamic measure of the tendency of the substance to prefer a non-aqueous or oily milieu rather than water (i.e. its hydrophilic/lipophilic balance). In principle, a chemical substance is lipophilic in character when its log K_{ow} exceeds 0. Typically, the lipophilic moiety possesses a log K_{ow} exceeding 1, exceeding 1.5, exceeding 2, exceeding 3, exceeding 4, exceeding 5, or exceeding 10. For instance, the log K_{ow} of 6-amino hexanol, for instance, is predicted to be approximately 0.7. Using the same method, the log K_{ow} of cholesteryl N-(hexan-6-ol) carbamate is predicted to be 10.7.

The lipophilicity of a molecule can change with respect to the functional group it carries. For instance, adding a hydroxyl group or amine group to the end of a lipophilic moiety can increase or decrease the partition coefficient (e.g., log K_{ow}) value of the lipophilic moiety.

Alternatively, the hydrophobicity of the single-stranded oligonucleotide, conjugated to one or more lipophilic moieties, can be measured by its protein binding characteristics. For instance, the unbound fraction in the plasma protein binding assay of the single-stranded oligonucleotide can be determined to positively correlate to the relative hydrophobicity of the single-stranded oligonucleotide, which can positively correlate to the silencing activity of the single-stranded oligonucleotide.

In one embodiment, the plasma protein binding assay determined is an electrophoretic mobility shift assay (EMSA) using human serum albumin protein. The hydrophobicity of the single-stranded oligonucleotide, measured by fraction of unbound single-stranded oligonucleotide in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of single-stranded oligonucleotide.

Accordingly, conjugating the lipophilic moieties to the internal position(s) of the single-stranded oligonucleotide provides optimal hydrophobicity for the enhanced in vivo delivery of single-stranded oligonucleotide.

In certain embodiments, the lipophilic moiety is an aliphatic, cyclic such as alicyclic, or polycyclic such as polyalicyclic compound, such as a steroid (e.g., sterol) or a linear or branched aliphatic hydrocarbon. The lipophilic moiety may generally comprise a hydrocarbon chain, which may be cyclic or acyclic. The hydrocarbon chain may comprise various substituents and/or one or more heteroatoms, such as an oxygen or nitrogen atom. Such lipophilic aliphatic moieties include, without limitation, saturated or unsaturated C₄-C₃₀ hydrocarbon (e.g., C₆-C₁₈ hydrocarbon or C₁₄-C₂₄ hydrocarbon), saturated or unsaturated fatty acids, waxes (e.g., monohydric alcohol esters of fatty acids and fatty diamides), terpenes (e.g., C₁₀ terpenes, C₁₅ sesquiterpenes, C₂₀ diterpenes, C₃₀ triterpenes, and C₄₀ tetraterpenes), and other polyalicyclic hydrocarbons. For instance, the lipophilic moiety may contain a C₄—C₃₀ hydrocarbon chain (e.g., C₄-C₃₀ alkyl or alkenyl). In some embodiment the lipophilic moiety contains a saturated or unsaturated C₆-C₁₈ hydrocarbon chain (e.g., a linear C₆-C₁₈ alkyl or alkenyl) or a saturated or unsaturated C₁₄-C₂₄ hydrocarbon (e.g., a linear C₁₄-C₂₄ alkyl or alkenyl). In one embodiment, the lipophilic moiety contains a saturated or unsaturated C₁₆ hydrocarbon chain (e.g., a linear C₁₆ alkyl or alkenyl) or a saturated or unsaturated C₂₂ hydrocarbon chain (e.g., a linear C₂₂ alkyl or alkenyl).

The lipophilic moiety may be attached to the single-stranded oligonucleotide by any method known in the art, including via a functional grouping already present in the lipophilic moiety or introduced into the single-stranded oligonucleotide, such as a hydroxy group (e.g., —CO—CH₂—OH). The functional groups already present in the lipophilic moiety or introduced into the single-stranded oligonucleotide include, but are not limited to, hydroxyl, amine, carboxylic acid, sulfonate, phosphate, thiol, azide, and alkyne.

Conjugation of the single-stranded oligonucleotide and the lipophilic moiety may occur, for example, through formation of an ether or a carboxylic or carbamoyl ester linkage between the hydroxy and an alkyl group R—, an alkanoyl group RCO— or a substituted carbamoyl group RNHCO—. The alkyl group R may be cyclic (e.g., cyclohexyl) or acyclic (e.g., straight-chained or branched; and saturated or unsaturated). Alkyl group R may be a butyl, pentyl, hexyl, heptyl, octyl, nonyl, decyl, undecyl, dodecyl, tridecyl, tetradecyl, pentadecyl, hexadecyl, heptadecyl or octadecyl group, or the like.

In some embodiments, the lipophilic moiety is conjugated to the single-stranded oligonucleotide via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In another embodiment, the lipophilic moiety is a steroid, such as sterol. Steroids are polycyclic compounds containing a perhydro-1,2-cyclopentanophenanthrene ring system. Steroids include, without limitation, bile acids (e.g., cholic acid, deoxycholic acid and dehydrocholic acid), cortisone, digoxigenin, testosterone, cholesterol, and cationic steroids, such as cortisone. A “cholesterol derivative” refers to a compound derived from cholesterol, for example by substitution, addition or removal of substituents.

In another embodiment, the lipophilic moiety is an aromatic moiety. In this context, the term “aromatic” refers broadly to mono- and polyaromatic hydrocarbons. Aromatic groups include, without limitation, C₆-C₁₄ aryl moieties comprising one to three aromatic rings, which may be optionally substituted; “aralkyl” or “arylalkyl” groups comprising an aryl group covalently linked to an alkyl group, either of which may independently be optionally substituted or unsubstituted; and “heteroaryl” groups. As used herein, the term “heteroaryl” refers to groups having 5 to 14 ring atoms, preferably 5, 6, 9, or 10 ring atoms; having 6, 10, or 14π electrons shared in a cyclic array, and having, in addition to carbon atoms, between one and about three heteroatoms selected from the group consisting of nitrogen (N), oxygen (O), and sulfur (S).

As employed herein, a “substituted” alkyl, cycloalkyl, aryl, heteroaryl, or heterocyclic group is one having between one and about four, preferably between one and about three, more preferably one or two, non-hydrogen substituents. Suitable substituents include, without limitation, halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

In some embodiments, the lipophilic moiety is an aralkyl group, e.g., a 2-arylpropanoyl moiety. The structural features of the aralkyl group are selected so that the lipophilic moiety will bind to at least one protein in vivo. In certain embodiments, the structural features of the aralkyl group are selected so that the lipophilic moiety binds to serum, vascular, or cellular proteins. In certain embodiments, the structural features of the aralkyl group promote binding to albumin, an immunoglobulin, a lipoprotein, α-2-macroglubulin, or α-1-glycoprotein.

In certain embodiments, the ligand is naproxen or a structural derivative of naproxen. Procedures for the synthesis of naproxen can be found in U.S. Pat. Nos. 3,904,682 and 4,009,197, which are herein incorporated by reference in their entirety. Naproxen has the chemical name (S)-6-Methoxy-α-methyl-2-naphthaleneacetic acid and the structure is

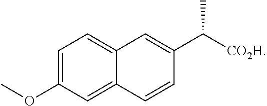 [SMILES: COc1ccc2cc([C@H](C)C(=O)O)ccc2c1]

In certain embodiments, the ligand is ibuprofen or a structural derivative of ibuprofen. Procedures for the synthesis of ibuprofen can be found in U.S. Pat. No. 3,228,831, which are herein incorporated by reference in their entirety. The structure of ibuprofen is

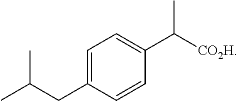

Additional exemplary aralkyl groups are illustrated in U.S. Pat. No. 7,626,014, which is incorporated herein by reference in its entirety.

In another embodiment, suitable lipophilic moieties include lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, ibuprofen, naproxen, dimethoxytrityl, or phenoxazine.

In some embodiments, the lipophilic moiety is a C₆-C₃₀ acid (e.g., hexanoic acid, heptanoic acid, octanoic acid, nonanoic acid, decanoic acid, undecanoic acid, dodcanoic acid, tridecanoic acid, tetradecanoic acid, pentadecanoic acid, hexadecanoic acid, heptadecanoic acid, octadecanoic acid, oleic acid, linoleic acid, arachidonic acid, cis-4,7,10,13,16,19-docosahexanoic acid, vitamin A, vitamin E, cholesterol etc.) or a C₆-C₃₀ alcohol (e.g., hexanol, heptanol, octanol, nonanol, decanol, undecanol, dodcanol, tridecanol, tetradecanol, pentadecanol, hexadecanol, heptadecanol, octadecanol, oleyl alcohol, linoleyl alcohol, arachidonic alcohol, cis-4,7,10,13,16,19-docosahexanol, retinol, vitamin E, cholesterol etc.). In one example, the lipophilic moiety is docosahexaenoic acid.

In certain embodiments, more than one lipophilic moieties can be incorporated into the single-stranded oligonucleotide, particularly when the lipophilic moiety has a low lipophilicity or hydrophobicity. In one embodiment, two or more lipophilic moieties are incorporated into the same strand of the single-stranded oligonucleotide. In one embodiment, each strand of the single-stranded oligonucleotide has one or more lipophilic moieties incorporated. In one embodiment, two or more lipophilic moieties are incorporated into the same position (i.e., the same nucleobase, same sugar moiety, or same internucleosidic linkage) of the single-stranded oligonucleotide. This can be achieved by, e.g., conjugating the two or more lipophilic moieties via a carrier, and/or conjugating the two or more lipophilic moieties via a branched linker, and/or conjugating the two or more lipophilic moieties via one or more linkers, with one or more linkers linking the lipophilic moieties consecutively.

The lipophilic moiety may be conjugated to the single-stranded oligonucleotide via a direct attachment to the ribosugar of the single-stranded oligonucleotide. Alternatively, the lipophilic moiety may be conjugated to the single-stranded oligonucleotide via a linker or a carrier.

In certain embodiments, the lipophilic moiety may be conjugated to the single-stranded oligonucleotide via one or more linkers (tethers).

In one embodiment, the lipophilic moiety is conjugated to the single-stranded oligonucleotide via a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

### Definitions

Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 18th edition, 1990; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2d Edition, Cold Spring Harbor Laboratory Press, 1989, which are hereby incorporated by reference for any purpose. Where permitted, all patents, applications, published applications and other publications and other data referred to throughout in the disclosure herein are incorporated by reference in their entirety.

Unless otherwise indicated, the following terms have the following meanings:

As used herein, the term “target nucleic acid” refers to any nucleic acid molecule the expression or activity of which is capable of being modulated by an siRNA compound. Target nucleic acids include, but are not limited to, RNA (including, but not limited to pre-mRNA and mRNA or portions thereof) transcribed from DNA encoding a target protein, and also cDNA derived from such RNA, and miRNA. For example, the target nucleic acid can be a cellular gene (or mRNA transcribed from the gene) whose expression is associated with a particular disorder or disease state. In some embodiments, a target nucleic acid can be a nucleic acid molecule from an infectious agent.

As used herein, the term “iRNA” refers to an agent that mediates the targeted cleavage of an RNA transcript. These agents associate with a cytoplasmic multi-protein complex known as RNAi-induced silencing complex (RISC). Agents that are effective in inducing RNA interference are also referred to as siRNA, RNAi agent, or iRNA agent, herein. Thus, these terms can be used interchangeably herein. As used herein, the term iRNA includes microRNAs and pre-microRNAs. Moreover, the “compound” or “compounds” of the invention as used herein, also refers to the iRNA agent, and can be used interchangeably with the iRNA agent.

The iRNA agent should include a region of sufficient homology to the target gene, and be of sufficient length in terms of nucleotides, such that the iRNA agent, or a fragment thereof, can mediate downregulation of the target gene. (For ease of exposition the term nucleotide or ribonucleotide is sometimes used herein in reference to one or more monomeric subunits of an iRNA agent. It will be understood herein that the usage of the term “ribonucleotide” or “nucleotide”, herein can, in the case of a modified RNA or nucleotide surrogate, also refer to a modified nucleotide, or surrogate replacement moiety at one or more positions.) Thus, the iRNA agent is or includes a region which is at least partially, and in some embodiments fully, complementary to the target RNA. It is not necessary that there be perfect complementarity between the iRNA agent and the target, but the correspondence must be sufficient to enable the iRNA agent, or a cleavage product thereof, to direct sequence specific silencing, e.g., by RNAi cleavage of the target RNA, e.g., mRNA. Complementarity, or degree of homology with the target strand, is most critical in the antisense strand. While perfect complementarity, particularly in the antisense strand, is often desired some embodiments can include, particularly in the antisense strand, one or more, or for example, 6, 5, 4, 3, 2, or fewer mismatches (with respect to the target RNA). The sense strand need only be sufficiently complementary with the antisense strand to maintain the overall double stranded character of the molecule.

iRNA agents include: molecules that are long enough to trigger the interferon response (which can be cleaved by Dicer (Bernstein et al. 2001. Nature, 409:363-366) and enter a RISC (RNAi-induced silencing complex)); and, molecules which are sufficiently short that they do not trigger the interferon response (which molecules can also be cleaved by Dicer and/or enter a RISC), e.g., molecules which are of a size which allows entry into a RISC, e.g., molecules which resemble Dicer-cleavage products. Molecules that are short enough that they do not trigger an interferon response are termed siRNA agents or shorter iRNA agents herein. “siRNA agent or shorter iRNA agent” as used herein, refers to an iRNA agent, e.g., a double stranded RNA agent or single strand agent, that is sufficiently short that it does not induce a deleterious interferon response in a human cell, e.g., it has a duplexed region of less than 60, 50, 40, or 30 nucleotide pairs. The siRNA agent, or a cleavage product thereof, can down regulate a target gene, e.g., by inducing RNAi with respect to a target RNA, wherein the target may comprise an endogenous or pathogen target RNA.

A “single-stranded oligonucleotide” or “single strand iRNA agent” as used herein, is an oligonucleotide or iRNA agent which is made up of a single molecule. It may include a duplexed region, formed by intra-strand pairing, e.g., it may be, or include, a hairpin, dumbbell, or pan-handle structure. Single-stranded oligonucleotide or iRNA agent may be antisense with regard to the target molecule. A single-stranded oligonucleotide or iRNA agent may be sufficiently long that it can enter the RISC and participate in RISC mediated cleavage of a target mRNA. A single-stranded oligonucleotide or iRNA agent is at least 14, and in other embodiments at least 15, 20, 25, 29, 35, 40, or 50 nucleotides in length. In certain embodiments, it is less than 200, 100, or 60 nucleotides in length. In certain embodiments, the single-stranded oligonucleotide contains two oligonucleotides, connected by a linking group.

A loop refers to a region of an oligonucleotide or iRNA strand that is unpaired with the opposing nucleotide in the duplex when a section of the oligonucleotide or the iRNA strand forms base pairs with another strand or with another section of the same strand.

Hairpin oligonucleotides or iRNA agents will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region, in some embodiments at the 3′, and in certain embodiments on the antisense side of the hairpin. In some embodiments, the overhangs are 2-3 nucleotides in length.

A “double-stranded oligonucleotide,” “double-stranded nucleic acid agent,” or “double stranded (ds) iRNA agent” as used herein, refers to an oligonucleotide, nucleic acid agent, or iRNA agent which includes more than one, and in some cases two, strands in which interchain hybridization can form a region of duplex structure.

As used herein, the terms “activity,” “siRNA activity,” or “RNAi activity” refer to gene silencing by an oligonucleotide, nucleic acid agent, or iRNA agent.

As used herein, “gene silencing” by an oligonucleotide, nucleic acid agent, or iRNA agent refers to a decrease in the mRNA level in a cell for a target gene by at least about 5%, at least about 10%, at least about 20%, at least about 30%, at least about 40%, at least about 50%, at least about 60%, at least about 70%, at least about 80%, at least about 90%, at least about 95%, at least about 99% up to and including 100%, and any integer in between of the mRNA level found in the cell without the presence of the miRNA or RNA interference molecule. In one preferred embodiment, the mRNA levels are decreased by at least about 70%, at least about 80%, at least about 90%, at least about 95%, at least about 99%, up to and including 100% and any integer in between 5% and 100%.”

As used herein the term “modulate gene expression” means that expression of the gene, or level of RNA molecule or equivalent RNA molecules encoding one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

As used herein, gene expression modulation happens when the expression of the gene, or level of RNA molecule or equivalent RNA molecules encoding one or more proteins or protein subunits is at least 5%, 10%, 20%, 30%, 40%, 50%, 60%, 70%, 80%, 90%, 100%, 2-fold, 3-fold, 4-fold, 5-fold or more different from that observed in the absence of the siRNA. The % and/or fold difference can be calculated relative to the control or the non-control, for example,

$$\% difference = \frac{[ expression with siRNA - expression without siRNA ]}{expression without siRNA} \\ or \\ \% difference = \frac{[ expression with siRNA - expression without siRNA ]}{expression without siRNA}$$

As used herein, the term “inhibit”, “down-regulate”, or “reduce” in relation to gene expression, means that the expression of the gene, or level of RNA molecules or equivalent RNA molecules encoding one or more proteins or protein subunits, or activity of one or more proteins or protein subunits, is reduced below that observed in the absence of modulator. The gene expression is down-regulated when expression of the gene, or level of RNA molecules or equivalent RNA molecules encoding one or more proteins or protein subunits, or activity of one or more proteins or protein subunits, is reduced at least 10% lower relative to a corresponding non-modulated control, and preferably at least 10%, 20%, 30%, 40%, 50%, 60%, 70%, 80%, 90%, 95%, 98%, 99% or most preferably, 100% (i.e., no gene expression).

As used herein, the term “increase” or “up-regulate” in relation to gene expression means that the expression of the gene, or level of RNA molecules or equivalent RNA molecules encoding one or more proteins or protein subunits, or activity of one or more proteins or protein subunits, is increased above that observed in the absence of modulator. The gene expression is up-regulated when expression of the gene, or level of RNA molecules or equivalent RNA molecules encoding one or more proteins or protein subunits, or activity of one or more proteins or protein subunits, is increased at least 10% relative to a corresponding non-modulated control, and preferably at least 10%, 20%, 30%, 40%, 50%, 60%, 70%, 80%, 90%, 95%, 98%, 100%, 1.1-fold, 1.25-fold, 1.5-fold, 1.75-fold, 2-fold, 3-fold, 4-fold, 5-fold, 10-fold, 50-fold, 100-fold or more.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced” or “reduce” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

A double-stranded nucleic acid agent comprises two oligonucleotide strands that are sufficiently complementary to hybridize to form a duplex structure. Generally, the duplex structure is between 8 and 30, between 15 and 30, between 18 and 25, between 19 and 24, or between 19 and 21 base pairs in length. In some embodiments, longer double-stranded nucleic acid agent of between 25 and 30 base pairs in length are preferred. In some embodiments, shorter double-stranded nucleic acid agent of between 10 and 15 base pairs in length are preferred. In another embodiment, the double-stranded nucleic acid agent is at least 21 nucleotides long.

The phrase “antisense strand” or “antisense oligonucleotide” as used herein, refers to an oligomeric compound that is substantially or 100% complementary to a target sequence of interest. The phrase “antisense strand” includes the antisense region of both oligomeric compounds that are formed from two separate strands, as well as unimolecular oligomeric compounds that are capable of forming hairpin or dumbbell type structures. The terms “antisense strand” and “guide strand” are used interchangeably herein.

The phrase “sense strand” refers to an oligomeric compound that has the same nucleoside sequence, in whole or in part, as a target sequence such as a messenger RNA or a sequence of DNA. The terms “sense strand” and “passenger strand” are used interchangeably herein.

By “specifically hybridizable” and “complementary” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

In some embodiments, the double-stranded region of a double-stranded nucleic acid agent is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the first oligonucleotide of a double-stranded nucleic acid agent is equal to or at least 14, 15, 16, 17, 18, 19, 20, 21, 22, 23, 23, 24, 25, 26, 27, 28, 29, or 30 nucleotides in length.

In some embodiments, the second oligonucleotide of a double-stranded nucleic acid agent is equal to or at least 10, 11, 12, 13, 14, 15, 16, 17, 18, 19, 20, 21, 22, 23, 23, 24, 25, 26, 27, 28, 29, or 30 nucleotides in length.

In one embodiment, the first and second oligonucleotides of the double-stranded nucleic acid agent are each 15 to 30 nucleotides in length.

In one embodiment, the first and second oligonucleotides of the double-stranded nucleic acid agent are each 19 to 25 nucleotides in length.

In one embodiment, the first and second oligonucleotides of the double-stranded nucleic acid agent are each 21 to 23 nucleotides in length.

In some embodiments, one oligonucleotide has at least one stretch of 1-5 single-stranded nucleotides in the double-stranded region. By “stretch of single-stranded nucleotides in the double-stranded region” is meant that there is present at least one nucleotide base pair at both ends of the single-stranded stretch. In some embodiments, both strands have at least one stretch of 1-5 (e.g., 1, 2, 3, 4, or 5) single-stranded nucleotides in the double stranded region. When both strands have a stretch of 1-5 (e.g., 1, 2, 3, 4, or 5) single-stranded nucleotides in the double stranded region, such single-stranded nucleotides can be opposite to each other (e.g., a stretch of mismatches) or they can be located such that the second oligonucleotide has no single-stranded nucleotides opposite to the single-stranded nucleotide of the first oligonucleotide and vice versa (e.g., a single-stranded loop). In some embodiments, the single-stranded nucleotides are present within 8 nucleotides from either end, for example 8, 7, 6, 5, 4, 3, or 2 nucleotides from either the 5′ or 3′ end of the region of complementarity between the two oligonucleotides.

In one embodiment, the double-stranded nucleic acid agent comprises a single-stranded overhang on at least one of the termini. In one embodiment, the single-stranded overhang is 1, 2, or 3 nucleotides in length.

In one embodiment, the second oligonucleotide of the double-stranded nucleic acid agent is 21-nucleotides in length, and the first oligonucleotide is 23-nucleotides in length, wherein the first and second oligonucleotides form a double-stranded region of 21 consecutive base pairs having a 2-nucleotide long single-stranded overhangs at the 3′-end.

In some embodiments, each oligonucleotide of the double-stranded nucleic acid agent has a ZXY structure, such as is described in PCT Publication No. 2004080406, which is hereby incorporated by reference in its entirety.

In certain embodiment, the two nucleotide sequences can be linked together to form a long strand. The two nucleotide sequences can be linked together by an oligonucleotide linker including, but not limited to, (N)ₙ; wherein N is independently a modified or unmodified nucleotide and n is 3-23. In some embodiments, n is 3-10, e.g., 3, 4, 5, 6, 7, 8, 9, or 10. In some embodiments, the oligonucleotide linker is selected from the group consisting of GNRA, (G)₄, (U)₄, and (dT)₄, wherein N is a modified or unmodified nucleotide and R is a modified or unmodified purine nucleotide. Some of the nucleotides in the linker can be involved in base-pair interactions with other nucleotides in the linker. The two nucleotide sequences can also be linked together by a non-nucleotide based linker, e.g. a linker described herein. It will be appreciated by one of skill in the art that any oligonucleotide chemical modifications or variations describe herein can be used in the oligonucleotide linker.

In certain embodiments, two strands specifically hybridize when there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

As used herein, “stringent hybridization conditions” or “stringent conditions” refers to conditions under which an antisense compound will hybridize to its target sequence, but to a minimal number of other sequences. Stringent conditions are sequence-dependent and will be different in different circumstances, and “stringent conditions” under which antisense compounds hybridize to a target sequence are determined by the nature and composition of the antisense compounds and the assays in which they are being investigated.

It is understood in the art that incorporation of nucleotide affinity modifications may allow for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tm). Tm or ΔTm can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

The single-stranded oligonucleotide can comprise a phosphorus-containing group at the 5′-end of a nucleotide sequence. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos), or 5′-deoxy-5′-C-malonyl

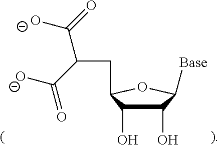

When the 5′-end phosphorus-containing group is 5′-end vinylphosphonate (5′-VP), the 5′-VP can be either 5′-E-VP isomer (i.e., trans-vinylphosphate,

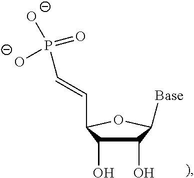

5′-Z-VP isomer (i.e., cis-vinylphosphate,

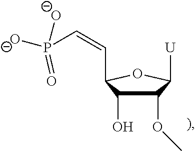

or mixtures thereof.

In one embodiment, the single-stranded oligonucleotide comprises a phosphorus-containing group at the 5′-end of a nucleotide sequence (e.g., Z¹ and/or Z²).

In one embodiment, the single-stranded oligonucleotide comprises a 5′-P in at least one nucleotide sequence (e.g., Z¹ and/or Z²).

In one embodiment, the single-stranded oligonucleotide comprises a 5′-PS in at least one nucleotide sequence (e.g., Z¹ and/or Z²).

In one embodiment, the single-stranded oligonucleotide comprises a 5′-VP in at least one nucleotide sequence (e.g., Z¹ and/or Z²). In one embodiment, the single-stranded oligonucleotide comprises a 5′-E-VP in at least one nucleotide sequence (e.g., Z¹ and/or Z²). In one embodiment, the single-stranded oligonucleotide comprises a 5′-Z-VP in at least one nucleotide sequence (e.g., Z¹ and/or Z²).

In one embodiment, the single-stranded oligonucleotide comprises a 5′-PS₂ in at least one nucleotide sequence (e.g., Z¹ and/or Z²).

In one embodiment, the single-stranded oligonucleotide comprises a 5′-deoxy-5′-C-malonyl in at least one nucleotide sequence (e.g., Z¹ and/or Z²).

In one embodiment, 100%, 95%, 90%, 85%, 80%, 75%, 70%, 65%, 60%, 55%, 50%, 45%, 40%, 35% or 30% of the single-stranded oligonucleotide is modified. For example, when 50% of the single-stranded oligonucleotide is modified, 50% of all nucleotides present in the single-stranded oligonucleotide contain a modification as described herein.

In one embodiment, each nucleotide of Z¹ and Z² of the single-stranded oligonucleotide is independently modified with acyclic nucleotides, LNA, HNA, CeNA, 2′-methoxyethyl, 2′-O-methyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F.

In one embodiment, the nucleotide sequence (e.g., Z¹ and/or Z²) of the single-stranded oligonucleotide contains at least two different modifications.

In one embodiment, the single-stranded oligonucleotide does not contain any 2′-F modification.

In one embodiment, the single-stranded oligonucleotide comprises one or more blocks of phosphorothioate or methylphosphonate internucleotide linkages. In one example, the single-stranded oligonucleotide comprises one block of two phosphorothioate or methylphosphonate internucleotide linkages. For example, the two blocks of phosphorothioate or methylphosphonate internucleotide linkages are separated by 16-18 phosphate internucleotide linkages.

In one embodiment, the nucleotide at position 1 of the 5′-end of a nucleotide sequence (e.g., Z¹ and/or Z²) is selected from the group consisting of A, dA, dU, U, and dT. In one embodiment, at least one of the first, second, and third base pair from the 5′-end of the nucleotide sequence (e.g., Z¹ and/or Z²) is an AU base pair.

In one embodiment, the single-stranded oligonucleotide is 100% complementary to a target RNA to hybridize thereto and inhibits its expression through RNA interference. In another embodiment, the single-stranded oligonucleotide is at least 95%, at least 90%, at least 85%, at least 80%, at least 75%, at least 70%, at least 65%, at least 60%, at least 55%, or at least 50% complementary to a target RNA.

In some embodiments, provided herein is a single-stranded oligonucleotide capable of inhibiting the expression of a target gene. The single-stranded oligonucleotide contains at least one thermally destabilizing nucleotide.

The thermally destabilizing nucleotide can occur, for example, between positions 14-17 of the 5′-end of a nucleotide sequence (e.g., Z¹ and/or Z²) of 21 nucleotides in length. The nucleotide sequence can contain at least two modified nucleic acids that are smaller than a sterically demanding 2′-OMe modification. Preferably, the two modified nucleic acids that are smaller than a sterically demanding 2′-OMe are separated by 11 nucleotides in length. For example, the two modified nucleic acids are at positions 2 and 14 of the 5′end.

In some embodiments, the single-stranded oligonucleotide contains a sequence that can be represented by formula (II):

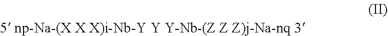

wherein:
i and j are each independently 0 or 1;
p and q are each independently 0-6;
each Na independently represents an oligonucleotide sequence comprising 0-25 modified nucleotides, each sequence comprising at least two differently modified nucleotides;
each Nb independently represents an oligonucleotide sequence comprising 1, 2, 3, 4, 5, or 6 modified nucleotides;
each np and nq independently represent an overhang nucleotide;
wherein Nb and Y do not have the same modification;
wherein XXX, YYY and ZZZ each independently represent one motif of three identical modifications on three consecutive nucleotides.

In some embodiments, the single-stranded oligonucleotide contains one, two, three, four, five, six, seven, eight, nine, ten, eleven or twelve 2′-F modification(s). In one example, the single-stranded oligonucleotide contains nine or ten 2′-F modifications.

The single-stranded oligonucleotide may further comprise at least one phosphorothioate or methylphosphonate internucleotide linkage. The phosphorothioate or methylphosphonate internucleotide linkage modification may occur on any nucleotide of the single-stranded oligonucleotide. For instance, the internucleotide linkage modification may occur on every nucleotide on at least one nucleotide sequence; each internucleotide linkage modification may occur in an alternating pattern on at least one nucleotide sequence.

In some embodiments, the compound of the invention disclosed herein is a miRNA mimic. In one design, miRNA mimics are double stranded molecules (e.g., with a duplex region of between about 16 and about 31 nucleotides in length) and contain one or more sequences that have identity with the mature strand of a given miRNA. Double-stranded miRNA mimics have designs similar to as described above for double-stranded iRNAs. In some embodiments, a miRNA mimic comprises a duplex region of between 16 and 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can comprise 2′ F modification of all of the Cs and Us, phosphorylation of the 5′ end of the oligonucleotide, and stabilized internucleotide linkages associated with a 2 nucleotide 3′ overhang.

In some embodiments, the compound of the invention disclosed herein is an antimir. In some embodiments, compound of the invention comprises at least two antimirs covalently linked to each other via a nucleotide-based or non-nucleotide-based linker, for example a linker described in the disclosure, or non-covalently linked to each other. The terms “antimir” “microRNA inhibitor” or “miR inhibitor” are synonymous and refer to oligonucleotides or modified oligonucleotides that interfere with the activity of specific miRNAs. Inhibitors can adopt a variety of configurations including single stranded, double stranded (RNA/RNA or RNA/DNA duplexes), and hairpin designs, in general, microRNA inhibitors comprise one or more sequences or portions of sequences that are complementary or partially complementary with the mature strand (or strands) of the miRNA to be targeted, in addition, the miRNA inhibitor can also comprise additional sequences located 5′ and 3′ to the sequence that is the reverse complement of the mature miRNA. The additional sequences can be the reverse complements of the sequences that are adjacent to the mature miRNA in the pri-miRNA from which the mature miRNA is derived, or the additional sequences can be arbitrary sequences (having a mixture of A, G, C, U, or dT). In some embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. MicroRNA inhibitors, when double stranded, can include mismatches between nucleotides on opposite strands. Furthermore, microRNA inhibitors can be linked to conjugate moieties in order to facilitate uptake of the inhibitor into a cell.

MicroRNA inhibitors, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the methods disclosed herein.

In some embodiments, compound of the invention disclosed herein is an antagomir. In some embodiments, the compound of the invention comprises at least two antagomirs covalently linked to each other via a nucleotide-based or non-nucleotide-based linker, for example a linker described in the disclosure, or non-covalently linked to each other. Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate intersugar linkage and, for example, a cholesterol-moiety at 3′-end. In a preferred embodiment, antagomir comprises a 2′-O-methyl modification at all nucleotides, a cholesterol moiety at 3′-end, two phosphorothioate intersugar linkages at the first two positions at the 5′-end and four phosphorothioate linkages at the 3′-end of the molecule. Antagomirs can be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al, Nature, 2005, 438: 685-689, which is expressly incorporated by reference herein in its entirety.

Recent studies have found that dsRNA can also activate gene expression, a mechanism that has been termed “small RNA-induced gene activation” or RNAa (activating RNA). See for example Li, L. C. et al. Proc Natl Acad Sci USA. (2006), 103(46):17337-42 and Li L. C. (2008). “Small RNA-Mediated Gene Activation”. RNA and the Regulation of Gene Expression: A Hidden Layer of Complexity. Caister Academic Press. ISBN 978-1-904455-25-7. It has been shown that dsRNAs targeting gene promoters induce potent transcriptional activation of associated genes. Endogenous miRNA that cause RNAa has also been found in humans. Check E. Nature (2007). 448 (7156): 855-858.

Another surprising observation is that gene activation by RNAa is long-lasting. Induction of gene expression has been seen to last for over ten days. The prolonged effect of RNAa could be attributed to epigenetic changes at dsRNA target sites. In some embodiments, the RNA activator can increase the expression of a gene. In some embodiments, increased gene expression inhibits viability, growth development, and/or reproduction.

Accordingly, in some embodiments, compound of the invention disclosed herein is activating RNA. In some embodiments, the compound of the invention comprises at least two activating RNAs covalently linked to each other via a nucleotide-based or non-nucleotide-based linker, for example a linker described in the disclosure, or non-covalently linked to each other.

Accordingly, in some embodiments, compound of the invention disclosed herein is a triplex forming oligonucleotide (TFO). In some embodiments, the compound of the invention comprises at least two TFOs covalently linked to each other via a nucleotide-based or non-nucleotide-based linker, for example a linker described in the disclosure, or non-covalently linked to each other. Recent studies have shown that triplex forming oligonucleotides can be designed which can recognize and bind to polypurine/polypyrimidine regions in double-stranded helical DNA in a sequence-specific manner. These recognition rules are outline by Maher III, L. J., et al., Science (1989) vol. 245, pp 725-730; Moser, H. E., et al., Science (1987) vol. 238, pp 645-630; Beal, P. A., et al., Science (1992) vol. 251, pp 1360-1363; Conney, M., et al., Science (1988) vol. 241, pp 456-459 and Hogan, M. E., et al., EP Publication 375408. Modification of the oligonucleotides, such as the introduction of intercalators and intersugar linkage substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 1 12:487-94). In general, the triplex-forming oligonucleotide has the sequence correspondence:

oligo
3′-A G G T
duplex
5′-A G C T
duplex
3′-T C G A

However, it has been shown that the A-AT and G-GC triplets have the greatest triple helical stability (Reither and Jeltsch, BMC Biochem, 2002, Septl2, Epub). The same authors have demonstrated that TFOs designed according to the A-AT and G-GC rule do not form non-specific triplexes, indicating that the triplex formation is indeed sequence specific.

Thus for any given sequence a triplex forming sequence can be devised. Triplex-forming oligonucleotides preferably are at least 15, more preferably 25, still more preferably 30 or more nucleotides in length, up to 50 or 100 nucleotides.

Formation of the triple helical structure with the target DNA induces steric and functional changes, blocking transcription initiation and elongation, allowing the introduction of desired sequence changes in the endogenous DNA and resulting in the specific down-regulation of gene expression. Examples of such suppression of gene expression in cells treated with TFOs include knockout of episomal supFGl and endogenous HPRT genes in mammalian cells (Vasquez et al., Nucl Acids Res. 1999; 27: 1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-I gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both down-regulation and up-regulation of expression of endogenous genes (Seidman and Glazer, J Clin Invest 2003; 112:487-94). Detailed description of the design, synthesis and administration of effective TFOs can be found in U.S. Pat. App. Nos. 2003 017068 and 2003 0096980 to Froehler et al, and 2002 0128218 and 2002 0123476 to Emanuele et al, and U.S. Pat. No. 5,721,138 to Lawn, contents of which are herein incorporated in their entireties.

### Nucleic Acid Modifications

In some embodiments, the single-stranded oligonucleotide comprises at least one nucleic acid modification described herein. For example, at least one modification selected from the group consisting of modified internucleoside linkage, modified nucleobase, modified sugar, and any combinations thereof. Without limitations, such a modification can be present anywhere in the single-stranded oligonucleotide. For example, the modification can be present in one of the RNA molecules.

### Nucleic Acid Modifications (Nucleobases)

The naturally occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleoside backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The unmodified or natural nucleobases can be modified or replaced to provide iRNAs having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the oligomer modifications described herein. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

An oligomeric compound described herein can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Exemplary modified nucleobases include, but are not limited to, other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyl)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine,2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil,4-(thio)pseudouracil,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl,para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho--(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any nucleobase that can base pair with all of the four naturally occurring nucleobases without substantially affecting the melting behavior, recognition by intracellular enzymes or activity of the iRNA duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic include more complicated structures, such as for example a tricyclic phenoxazine nucleobase mimetic. Methods for preparation of the above noted modified nucleobases are well known to those skilled in the art.

### Nucleic Acid Modifications (Sugar)

The single-stranded oligonucleotide provided herein can comprise one or more (e.g., 1, 2, 3, 4, 5, 6, 7, 8, 9, 10, 11, 12, 13, 14, 15 or more) monomer, including a nucleoside or nucleotide, having a modified sugar moiety. For example, the furanosyl sugar ring of a nucleoside can be modified in a number of ways including, but not limited to, addition of a substituent group, bridging of two non-geminal ring atoms to form a locked nucleic acid or bicyclic nucleic acid. In certain embodiments, oligomeric compounds comprise one or more (e.g., 1, 2, 3, 4, 5, 6, 7, 8, 9, 10, 11, 12, 13, 14, 15 or more) monomers that are LNA.

In some embodiments of a locked nucleic acid, the 2′ position of furanosyl is connected to the 4′ position by a linker selected independently from —[C(R1)(R2)]ₙ—, —[C(R1)(R2)]ₙ—O—, —[C(R1)(R2)]ₙ—N(R1)-, —[C(R1)(R2)]ₙ—N(R1)-O—, [C(R1R2)]ₙ—O—N(R1)—, —C(R1)=C(R2)-O—, —C(R1)=N—, —C(R1)=N—O—, —C(═NR1)-, —C(═NR1)-O—, —C(═O)—, —C(═O)O—, —C(═S)—, —C(═S)O—, —C(═S)S—, —O—, —Si(R1)2-, —S(═O)ₓ— and —N(R1)-;
wherein:
x is 0, 1, or 2;
n is 1, 2, 3, or 4;
each R1 and R2 is, independently, H, a protecting group, hydroxyl, C1-C12 alkyl, substituted C₁-C₁₂ alkyl, C2-C12 alkenyl, substituted C₂-C₁₂ alkenyl, C2-C12 alkynyl, substituted C₂-C₁₂ alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and
each J1 and J2 is, independently, H, C1-C12 alkyl, substituted C₁-C₁₂ alkyl, C2-C12 alkenyl, substituted C₂-C₁₂ alkenyl, C2-C12 alkynyl, substituted C₂-C₁₂ alkynyl, C5-C20 aryl, substituted C5-C20 aryl, acyl (C(═O)—H), substituted acyl, a heterocycle radical, a substituted heterocycle radical, C1-C12 aminoalkyl, substituted C₁-C₁₂ aminoalkyl or a protecting group.

In some embodiments, each of the linkers of the LNA compounds is, independently, [C(R1)(R2)]ₙ—, [C(R1)(R2)]ₙ—O—, C(R1R2)-N(R1)-O or C(R1R2)-O—N(R1)-. In another embodiment, each of said linkers is, independently, 4′-CH₂-2′, 4′-(CH₂)2-2′, 4′-(CH₂)3-2′, 4′-CH₂—O-2′, 4′-(CH₂)2-O-2′, 4′-CH₂—O—N(R1)-2′ and 4′-CH₂—N(R1)-O-2′-wherein each R1 is, independently, H, a protecting group or C1-C12 alkyl.

Certain LNA's have been prepared and disclosed in the patent literature as well as in scientific literature (Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039; Examples of issued US patents and published applications that disclose LNA s include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are LNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) LNA is used for the bicyclic moiety; in the case of an ethylene group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O-2′) LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., Proc. Natl. Acad. Sci. U.S.A., 2000, 97, 5633-5638).

An isomer of methyleneoxy (4′-CH₂—O-2′) LNA that has also been discussed is alpha-L-methyleneoxy (4′-CH₂—O-2′) LNA which has been shown to have superior stability against a 3′-exonuclease. The alpha-L-methyleneoxy (4′-CH₂—O-2′) LNA's were incorporated into antisense gapmers and chimeras that showed potent antisense activity (Frieden et al., Nucleic Acids Research, 2003, 21, 6365-6372).

The synthesis and preparation of the methyleneoxy (4′-CH₂—O-2′) LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) LNA, phosphorothioate-methyleneoxy (4′-CH₂—O-2′) LNA and 2′-thio-LNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-LNA, a novel comformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Modified sugar moieties are well known and can be used to alter, typically increase, the affinity of the antisense compound for its target and/or increase nuclease resistance. A representative list of preferred modified sugars includes but is not limited to bicyclic modified sugars, including methyleneoxy (4′-CH₂—O-2′) LNA and ethyleneoxy (4′-(CH₂)2-O-2′ bridge) ENA; substituted sugars, especially 2′-substituted sugars having a 2′-F, 2′-OCH₃ or a 2′-O(CH₂)2-OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)CH₂CH₂OR, n=1-50; “locked” nucleic acids (LNA) in which the furanose portion of the nucleoside includes a bridge connecting two carbon atoms on the furanose ring, thereby forming a bicyclic ring system; O-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

“Deoxy” modifications include hydrogen (i.e. deoxyribose sugars, which are of particular relevance to the single-strand overhangs); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

Other suitable 2′-modifications, e.g., modified MOE, are described in U.S. Patent Application Publication No. 20130130378, contents of which are herein incorporated by reference.

A modification at the 2′ position can be present in the arabinose configuration The term “arabinose configuration” refers to the placement of a substituent on the C2′ of ribose in the same configuration as the 2′-OH is in the arabinose.

The sugar can comprise two different modifications at the same carbon in the sugar, e.g., gem modification. The sugar group can also contain one or more carbons that possess the opposite stereochemical configuration than that of the corresponding carbon in ribose. Thus, an oligomeric compound can include one or more monomers containing e.g., arabinose, as the sugar. The monomer can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

The single-stranded oligonucleotide disclosed herein can also include abasic sugars, i.e., a sugar which lack a nucleobase at C-1′ or has other chemical groups in place of a nucleobase at C1′. See for example U.S. Pat. No. 5,998,203, content of which is herein incorporated in its entirety. These abasic sugars can also be further containing modifications at one or more of the constituent sugar atoms. The single-stranded oligonucleotide can also contain one or more sugars that are the L isomer, e.g. L-nucleosides. Modification to the sugar group can also include replacement of the 4′-O with a sulfur, optionally substituted nitrogen or CH₂ group. In some embodiments, linkage between C1′ and nucleobase is in a configuration.

Sugar modifications can also include acyclic nucleotides, wherein a C—C bonds between ribose carbons (e.g., C1′-C2′, C2′-C3′, C3′-C4′, C4′-O4′, C1′-O4′) is absent and/or at least one of ribose carbons or oxygen (e.g., C1′, C2′, C3′, C4′ or 04′) are independently or in combination absent from the nucleotide. In some embodiments, acyclic nucleotide is

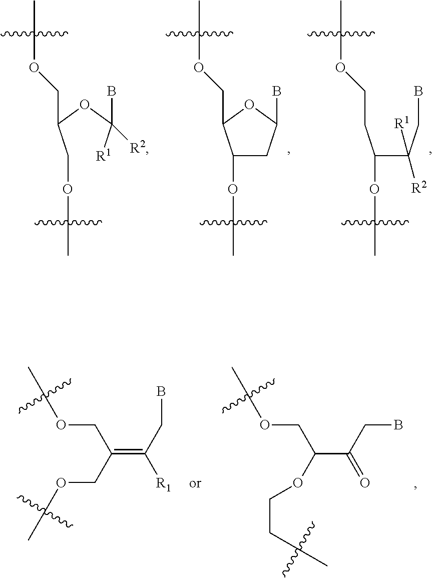

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

In some embodiments, sugar modifications are selected from the group consisting of 2′-H, 2′-O-Me (2′-O-methyl), 2′-O-MOE (2′-O-methoxyethyl), 2′-F, 2′-O-[2-(methylamino)-2-oxoethyl](2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE) and gem 2′-OMe/2′F with 2′-O-Me in the arabinose configuration.

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of 0, S, S(O), SO₂, N(R′), C(O), N(R′)C(O)O, OC(O)N(R′), CH(Z′), phosphorous containing linkage, optionally substituted aryl, optionally substituted heteroaryl, optionally substituted heterocyclic or optionally substituted cycloalkyl, where R′ is hydrogen, acyl or optionally substituted aliphatic, Z′ is selected from the group consisting of OR₁₁, COR₁₁, CO₂R₁₁,

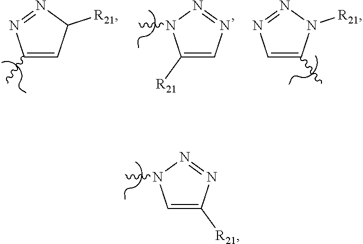

NR₂₁R₃₁, CONR₂₁R₃₁, CON(H)NR₂₁R₃₁, ONR₂₁R₃₁, CON(H)N=CR₄₁R₅₁, N(R₂₁)C(═NR₃₁)NR₂₁R₃₁, N(R₂₁)C(O)NR₂₁R₃₁, N(R₂₁)C(S)NR₂₁R₃₁, OC(O)NR₂₁R₃₁, SC(O)NR₂₁R₃₁, N(R₂₁)C(S)OR₁₁, N(R₂₁)C(O)OR₁₁, N(R₂₁)C(O)SR₁₁, N(R₂₁)N=CR₄₁R₅₁, ON═CR₄₁R₅₁, SO₂R₁₁, SOR₁₁, SR₁₁, and substituted or unsubstituted heterocyclic; R₂₁ and R₃₁ for each occurrence are independently hydrogen, acyl, unsubstituted or substituted aliphatic, aryl, heteroaryl, heterocyclic, OR₁₁, COR₁₁, CO₂R₁₁, or NR₁₁R₁₁′; or R₂₁ and R₃₁, taken together with the atoms to which they are attached, form a heterocyclic ring; R₄₁ and R₅₁ for each occurrence are independently hydrogen, acyl, unsubstituted or substituted aliphatic, aryl, heteroaryl, heterocyclic, OR₁₁, COR₁₁, or CO₂R₁₁, or NR₁₁R₁₁′; and R₁₁ and R₁₁′ are independently hydrogen, aliphatic, substituted aliphatic, aryl, heteroaryl, or heterocyclic. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alkali metal or transition metal with an overall charge of +1; and Y is O, S, or NR′, where R′ is hydrogen, optionally substituted aliphatic. Preferably this modification is at the 5 terminal of the iRNA.

In certain embodiments, LNA's include bicyclic nucleoside having the formula:

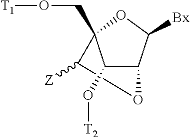

wherein:
Bx is a heterocyclic base moiety;
T₁ is H or a hydroxyl protecting group;
T₂ is H, a hydroxyl protecting group or a reactive phosphorus group;
Z is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In certain embodiments, the compounds of the invention comprise at least one monomer of the formula:

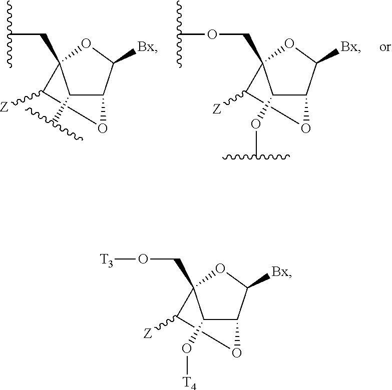

wherein
Bx is a heterocyclic base moiety;
T₃ is H, a hydroxyl protecting group, a linked conjugate group or an internucleoside linking group attached to a nucleoside, a nucleotide, an oligonucleoside, an oligonucleotide, a monomeric subunit or an oligomeric compound;
T₄ is H, a hydroxyl protecting group, a linked conjugate group or an internucleoside linking group attached to a nucleoside, a nucleotide, an oligonucleoside, an oligonucleotide, a monomeric subunit or an oligomeric compound;
wherein at least one of T₃ and T₄ is an internucleoside linking group attached to a nucleoside, a nucleotide, an oligonucleoside, an oligonucleotide, a monomeric subunit or an oligomeric compound; and
Z is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In certain such embodiments, LNAs include, but are not limited to, (A) α-L-Methyleneoxy (4′-CH₂—O-2′) LNA, (B) β-D-Methyleneoxy (4′-CH₂—O-2′) LNA, (C) Ethyleneoxy (4′-(CH₂)2-O-2′) LNA, (D) Aminooxy (4′-CH₂—O—N(R)-2′) LNA and (E) Oxyamino (4′-CH₂—N(R)—O-2′) LNA, as depicted below:

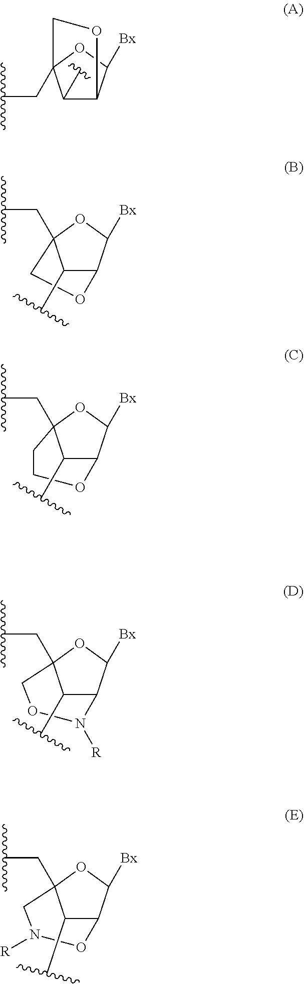

In certain embodiments, the single-stranded oligonucleotide comprises at least two regions of at least two contiguous monomers of the above formula. In certain embodiments, the single-stranded oligonucleotide comprises a gapped motif. In certain embodiments, the single-stranded oligonucleotide comprises at least one region of from about 8 to about 14 contiguous β-D-2′-deoxyribofuranosyl nucleosides. In certain embodiments, the single-stranded oligonucleotide comprises at least one region of from about 9 to about 12 contiguous β-D-2′-deoxyribofuranosyl nucleosides.

In certain embodiments, the single-stranded oligonucleotide comprises at least one (e.g., 1, 2, 3, 4, 5, 6, 7, 8, 9, 10, 11, 12, 13, 14, 15 or more) (S)-cEt monomer of the formula:

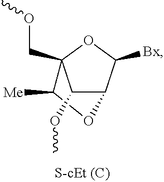

wherein Bx is heterocyclic base moiety.

In certain embodiments, monomers include sugar mimetics. In certain such embodiments, a mimetic is used in place of the sugar or sugar-internucleoside linkage combination, and the nucleobase is maintained for hybridization to a selected target. Representative examples of a sugar mimetics include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances, a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

### Nucleic Acid Modifications (Intersugar Linkage)

Described herein are linking groups that link monomers (including, but not limited to, modified and unmodified nucleosides and nucleotides) together, thereby forming an oligomeric compound, e.g., an oligonucleotide. Such linking groups are also referred to as intersugar linkage. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O CH₂—), thiodiester (—O—C(O)—S—), thionocarbamate (—O—C(O)(NH)—S—); siloxane (—O—Si(H)₂—O—); and N,N′-dimethylhydrazine (CH₂—N(CH₃)—N(CH₃)—). Modified linkages, compared to natural phosphodiester linkages, can be used to alter, typically increase, nuclease resistance of the oligonucleotides. In certain embodiments, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The phosphate group in the linking group can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words, a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the sugar of the monomer), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methylenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′-CH₂—NH—NH—C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phosphotriesters, aminoalkylphosphotrioesters, alkyl-phosphonaters (e.g., methyl-phosphonate), selenophosphates, phosphoramidates (e.g., N-alkylphosphoramidate), and boranophosphonates.

In some embodiments, the single-stranded oligonucleotide comprises at least one (e.g., 1, 2, 3, 4, 5, 6, 7, 8, 9, 10, 11, 12, 13, 14, 15 or more and up to including all) modified or nonphosphodiester linkages. In some embodiments, the single-stranded oligonucleotide comprises at least one (e.g., 1, 2, 3, 4, 5, 6, 7, 8, 9, 10, 11, 12, 13, 14, 15 or more and up to including all) phosphorothioate linkages.

The single-stranded oligonucleotide can also be constructed wherein the phosphate linker and the sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). It can be desirable, in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backbone-extended pyrrolidine PNA (bepPNA) nucleoside surrogates. A preferred surrogate is a PNA surrogate.

The single-stranded oligonucleotide described herein can contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids et al. Included in the single-stranded oligonucleotide provided herein are all such possible isomers, as well as their racemic and optically pure forms.

### Nucleic Acid Modifications (Terminal Modifications)

Ends of a sense or antisense nucleotide sequence of the single-stranded oligonucleotide can be modified. Such modifications can be at one end or both ends of the nucleotide sequence. For example, the 3′ and/or 5′ ends can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

Terminal modifications useful for modulating activity include modification of the 5′ end of a sequence with phosphate or phosphate analogs. In certain embodiments, the 5′end of sequence is phosphorylated or includes a phosphoryl analog. Exemplary 5′-phosphate modifications include those which are compatible with RISC mediated gene silencing. Modifications at the 5′-terminal end can also be useful in stimulating or inhibiting the immune system of a subject. In some embodiments, the 5′-end of the oligomeric compound comprises the modification

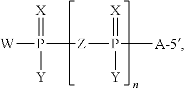

wherein W, X and Y are each independently selected from the group consisting of O, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments, n is 1 or 2. It is understood that A is replacing the oxygen linked to 5′ carbon of sugar. When n is 0, W and Y together with the P to which they are attached can form an optionally substituted 5-8 membered heterocyclic, wherein W an Y are each independently O, S, NR′ or alkylene. Preferably the heterocyclic is substituted with an aryl or heteroaryl. In some embodiments, one or both hydrogen on C5′ of the 5′-terminal nucleotides are replaced with a halogen, e.g., F.

Exemplary 5′-modifications include, but are not limited to, 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc. . . . ), 5′-alkyletherphosphonates (R(OH)(O)P—O-5′, R=alkylether, e.g., methoxymethyl (CH₂OMe), ethoxymethyl, etc. . . . ). Other exemplary 5′-modifications include where Z is optionally substituted alkyl at least once, e.g., ((HO)₂(X)P—O[—(CH₂)ₐ—O—P(X)(OH)—O]_{b}— 5′, ((HO)₂(X)P—O[—(CH₂)ₐ—P(X)(OH)—O]_{b}— 5′, ((HO)2(X)P—[—(CH₂)ₐ—O—P(X)(OH)—O]_{b}—5′; dialkyl terminal phosphates and phosphate mimics: HO[—(CH₂)ₐ—O—P(X)(OH)—O]_{b}—5′, H₂N[—(CH₂)ₐ—O—P(X)(OH)—O]_{b}—5′, H[—(CH₂)ₐ—O—P(X)(OH)—O]_{b}—5′, Me₂N[—(CH₂)ₐ—O—P(X)(OH)—O]_{b}—5′, HO[—(CH₂)ₐ—P(X)(OH)—O]_{b}—5′, H₂N[—(CH₂)ₐ—P(X)(OH)—O]_{b}—5′, H[—(CH₂)ₐ—P(X)(OH)—O]_{b}—5′, Me₂N[—(CH₂)ₐ—P(X)(OH)—O]_{b}—5′, wherein a and b are each independently 1-10. Other embodiments, include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

### Thermally Destabilizing Modifications

The compounds of the invention, such as double-stranded nucleic acid agent or single-stranded oligonucleotides, can be optimized for RNA interference by increasing the propensity of the iRNA duplex to disassociate or melt (decreasing the free energy of duplex association) by introducing a thermally destabilizing modification in the sense strand at a site opposite to the seed region of the antisense strand (i.e., at positions 2-8 of the 5′-end of the antisense strand). This modification can increase the propensity of the duplex to disassociate or melt in the seed region of the antisense strand.

The thermally destabilizing modifications can include abasic modification; mismatch with the opposing nucleotide in the opposing strand; and sugar modification such as 2′-deoxy modification or acyclic nucleotide, e.g., unlocked nucleic acids (UNA) or glycerol nucleic acid (GNA).

Exemplified abasic modifications are:

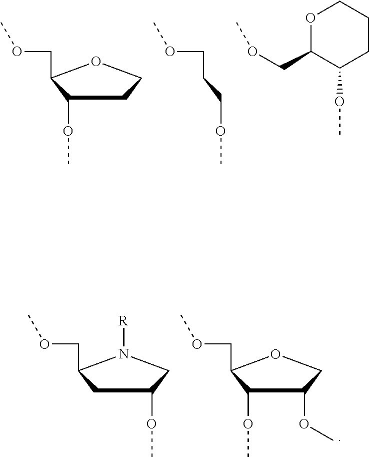

Exemplified sugar modifications are:

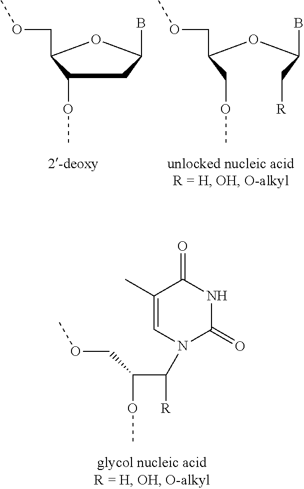

Optionally, the thermally destabilizing modification of the duplex is one or more

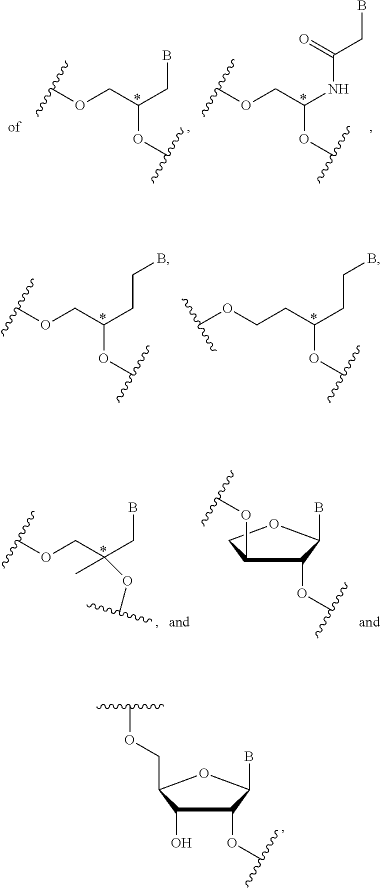

wherein B is an optionally modified nucleobase, and * represent (R)-, (S)- or racemic stereochemistry.

In certain embodiments, the thermally destabilizing modification of the duplex is one or more of

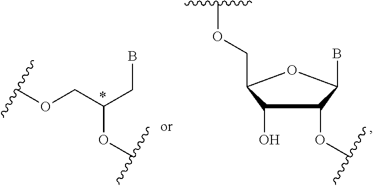

wherein B is an optionally modified nucleobase, and * represent (R)-, (S)- or racemic stereochemistry (e.g., S-stereochemistry).

The term “acyclic nucleotide” refers to any nucleotide having an acyclic ribose sugar, for example, where any of bonds between the ribose carbons (e.g., C1′-C2′, C2′-C3′, C3′-C4′, C4′-04′, or C1′-04′) is absent and/or at least one of ribose carbons or oxygen (e.g., C1′, C2′, C3′, C4′ or 04′) are independently or in combination absent from the nucleotide. In some embodiments, acyclic nucleotide is

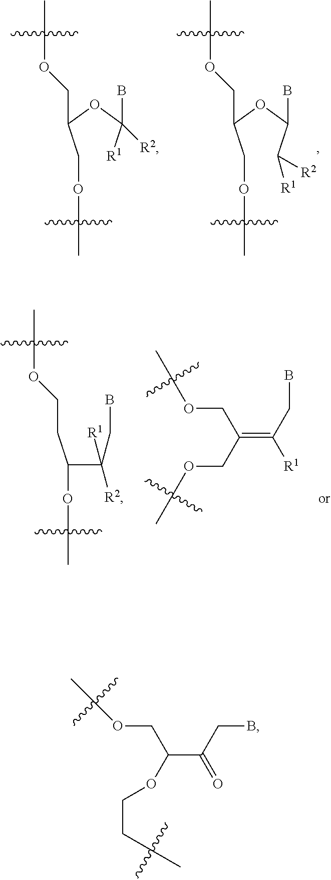

wherein B is a modified or unmodified nucleobase, R¹ and R² independently are H, halogen, OR₃, or alkyl; and R₃ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

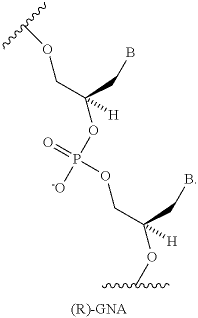

The thermally destabilizing modification can be mismatches (i.e., noncomplementary base pairs) between the thermally destabilizing nucleotide and the opposing nucleotide in the opposite strand within the duplex of a double-stranded nucleic acid agent. Exemplary mismatch basepairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the compounds of the invention contain at least one nucleobase in the mismatch pairing that is a 2′-deoxy nucleobase; e.g., the 2′-deoxy nucleobase is in the sense strand.

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

Nucleobase modifications with impaired or completely abolished capability to form hydrogen bonds with bases in the opposite strand have been evaluated for destabilization of the central region of the dsRNA duplex as described in WO 2010/0011895, which is herein incorporated by reference in its entirety. Exemplary nucleobase modifications are:

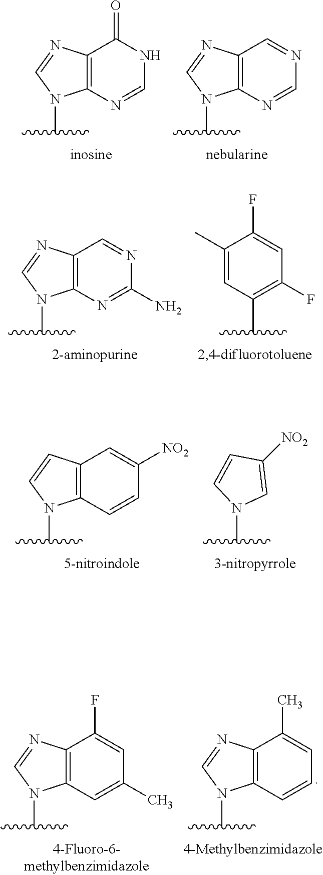

Exemplary phosphate modifications known to decrease the thermal stability of dsRNA duplexes compared to natural phosphodiester linkages are:

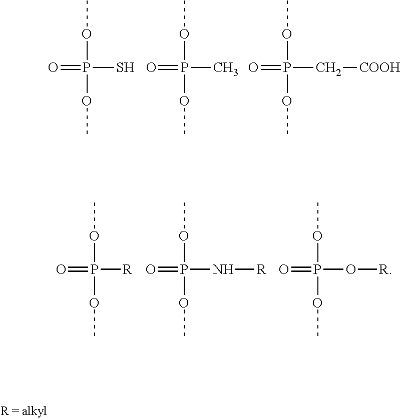

In some embodiments, compounds of the invention can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, compounds of the invention can comprise L sugars (e.g., L ribose, L-arabinose with 2′-H, 2′-OH and 2′-OMe). For example, these L sugar modifications can be used to promote nuclease resistance or to inhibit binding of the sense to the antisense strand, or can be used at the 5′ end of the sense strand to avoid sense strand activation by RISC.

In some embodiments, at least one nucleotide sequence of the single-stranded oligonucleotide disclosed herein is 5′ phosphorylated or includes a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, 5′-alkenylphosphonates (i.e. vinyl, substituted vinyl), (OH)₂(O)P-5′-CH₂—), 5′-alkyletherphosphonates (R=alkylether=methoxymethyl (MeOCH2-), ethoxymethyl, etc., e.g. RP(OH)(O)—O-5′-).

### Target Genes

Without limitations, target genes for single-stranded oligonucleotides include, but are not limited to genes promoting unwanted cell proliferation, growth factor gene, growth factor receptor gene, genes expressing kinases, an adaptor protein gene, a gene encoding a G protein super family molecule, a gene encoding a transcription factor, a gene which mediates angiogenesis, a viral gene, a gene required for viral replication, a cellular gene which mediates viral function, a gene of a bacterial pathogen, a gene of an amoebic pathogen, a gene of a parasitic pathogen, a gene of a fungal pathogen, a gene which mediates an unwanted immune response, a gene which mediates the processing of pain, a gene which mediates a neurological disease, an allene gene found in cells characterized by loss of heterozygosity, or one allege gene of a polymorphic gene.

Specific exemplary target genes for the single-stranded oligonucleotides include, but are not limited to, PCSK-9, ApoC3, AT3, AGT, ALAS1, TMPR, HAO1, AGT, C5, CCR-5, PDGF beta gene; Erb-B gene, Src gene; CRK gene; GRB2 gene; RAS gene; MEKK gene; JNK gene; RAF gene; Erk1/2 gene; PCNA(p21) gene; MYB gene; c-MYC gene; JUN gene; FOS gene; BCL-2 gene; Cyclin D gene; VEGF gene; EGFR gene; Cyclin A gene; Cyclin E gene; WNT-1 gene; beta-catenin gene; c-MET gene; PKC gene; NFKB gene; STAT3 gene; survivin gene; Her2/Neu gene; topoisomerase I gene; topoisomerase II alpha gene; p73 gene; p21(WAF1/CIP1) gene, p27(KIP1) gene; PPM1D gene; caveolin I gene; MIB I gene; MTAI gene; M68 gene; tumor suppressor genes; p53 gene; DN-p63 gene; pRb tumor suppressor gene; APC1 tumor suppressor gene; BRCA1 tumor suppressor gene; PTEN tumor suppressor gene; MLL fusion genes, e.g., MLL-AF9, BCR/ABL fusion gene; TEL/AML1 fusion gene; EWS/FLI1 fusion gene; TLS/FUS1 fusion gene; PAX3/FKHR fusion gene; AML1/ETO fusion gene; alpha v-integrin gene; Flt-1 receptor gene; tubulin gene; Human Papilloma Virus gene, a gene required for Human Papilloma Virus replication, Human Immunodeficiency Virus gene, a gene required for Human Immunodeficiency Virus replication, Hepatitis A Virus gene, a gene required for Hepatitis A Virus replication, Hepatitis B Virus gene, a gene required for Hepatitis B Virus replication, Hepatitis C Virus gene, a gene required for Hepatitis C Virus replication, Hepatitis D Virus gene, a gene required for Hepatitis D Virus replication, Hepatitis E Virus gene, a gene required for Hepatitis E Virus replication, Hepatitis F Virus gene, a gene required for Hepatitis F Virus replication, Hepatitis G Virus gene, a gene required for Hepatitis G Virus replication, Hepatitis H Virus gene, a gene required for Hepatitis H Virus replication, Respiratory Syncytial Virus gene, a gene that is required for Respiratory Syncytial Virus replication, Herpes Simplex Virus gene, a gene that is required for Herpes Simplex Virus replication, herpes Cytomegalovirus gene, a gene that is required for herpes Cytomegalovirus replication, herpes Epstein Barr Virus gene, a gene that is required for herpes Epstein Barr Virus replication, Kaposi's Sarcoma-associated Herpes Virus gene, a gene that is required for Kaposi's Sarcoma-associated Herpes Virus replication, JC Virus gene, human gene that is required for JC Virus replication, myxovirus gene, a gene that is required for myxovirus gene replication, rhinovirus gene, a gene that is required for rhinovirus replication, coronavirus gene, a gene that is required for coronavirus replication, West Nile Virus gene, a gene that is required for West Nile Virus replication, St. Louis Encephalitis gene, a gene that is required for St. Louis Encephalitis replication, Tick-borne encephalitis virus gene, a gene that is required for Tick-borne encephalitis virus replication, Murray Valley encephalitis virus gene, a gene that is required for Murray Valley encephalitis virus replication, dengue virus gene, a gene that is required for dengue virus gene replication, Simian Virus 40 gene, a gene that is required for Simian Virus 40 replication, Human T Cell Lymphotropic Virus gene, a gene that is required for Human T Cell Lymphotropic Virus replication, Moloney-Murine Leukemia Virus gene, a gene that is required for Moloney-Murine Leukemia Virus replication, encephalomyocarditis virus gene, a gene that is required for encephalomyocarditis virus replication, measles virus gene, a gene that is required for measles virus replication, Vericella zoster virus gene, a gene that is required for Vericella zoster virus replication, adenovirus gene, a gene that is required for adenovirus replication, yellow fever virus gene, a gene that is required for yellow fever virus replication, poliovirus gene, a gene that is required for poliovirus replication, poxvirus gene, a gene that is required for poxvirus replication, plasmodium gene, a gene that is required for plasmodium gene replication, Mycobacterium ulcerans gene, a gene that is required for Mycobacterium ulcerans replication, Mycobacterium tuberculosis gene, a gene that is required for Mycobacterium tuberculosis replication, Mycobacterium leprae gene, a gene that is required for Mycobacterium leprae replication, Staphylococcus aureus gene, a gene that is required for Staphylococcus aureus replication, Streptococcus pneumoniae gene, a gene that is required for Streptococcus pneumoniae replication, Streptococcus pyogenes gene, a gene that is required for Streptococcus pyogenes replication, Chlamydia pneumoniae gene, a gene that is required for Chlamydia pneumoniae replication, Mycoplasma pneumoniae gene, a gene that is required for Mycoplasma pneumoniae replication, an integrin gene, a selectin gene, complement system gene, chemokine gene, chemokine receptor gene, GCSF gene, Gro1 gene, Gro2 gene, Gro3 gene, PF4 gene, MIG gene, Pro-Platelet Basic Protein gene, MIP-1I gene, MIP-1J gene, RANTES gene, MCP-1 gene, MCP-2 gene, MCP-3 gene, CMBKR1 gene, CMBKR2 gene, CMBKR3 gene, CMBKR5v, AIF-1 gene, I-309 gene, a gene to a component of an ion channel, a gene to a neurotransmitter receptor, a gene to a neurotransmitter ligand, amyloid-family gene, presenilin gene, HD gene, DRPLA gene, SCA1 gene, SCA2 gene, MJD1 gene, CACNL1A4 gene, SCA7 gene, SCA8 gene, allele gene found in loss of heterozygosity (LOH) cells, one allele gene of a polymorphic gene and combinations thereof.

The loss of heterozygosity (LOH) can result in hemizygosity for sequence, e.g., genes, in the area of LOH. This can result in a significant genetic difference between normal and disease-state cells, e.g., cancer cells, and provides a useful difference between normal and disease-state cells, e.g., cancer cells. This difference can arise because a gene or other sequence is heterozygous in duploid cells but is hemizygous in cells having LOH. The regions of LOH will often include a gene, the loss of which promotes unwanted proliferation, e.g., a tumor suppressor gene, and other sequences including, e.g., other genes, in some cases a gene which is essential for normal function, e.g., growth. Methods of the invention rely, in part, on the specific modulation of one allele of an essential gene with a composition of the invention.

In certain embodiments, the invention provides a single-stranded oligonucleotide that modulates a micro-RNA.

In some embodiments, the invention provides a single-stranded oligonucleotide for extrahepatic delivery, and target a CNS gene or ocular gene.

In some embodiments, provided herein is a single-stranded oligonucleotide that targets APP for Early Onset Familial Alzheimer Disease, ATXN2 for Spinocerebellar Ataxia 2 and ALS, and C9orf72 for Amyotrophic Lateral Sclerosis and Frontotemporal Dementia.

In some embodiments, provided herein is a sciRNA agent that targets TARDBP for ALS, MAPT (Tau) for Frontotemporal Dementia, and HTT for Huntington Disease.

In some embodiments, provided herein is a single-stranded oligonucleotide that targets SNCA for Parkinson Disease, FUS for ALS, ATXN3 for Spinocerebellar Ataxia 3, ATXN1 for SCA1, genes for SCA7 and SCA8, ATN1 for DRPLA, MeCP2 for XLMR, PRNP for Prion Diseases, recessive CNS disorders: Lafora Disease, DMPK for DM1 (CNS and Skeletal Muscle), and TTR for hATTR (CNS, ocular and systemic).

Spinocerebellar ataxia is an inherited brain-function disorder. Dominantly inherited forms of spinocerebellar ataxias, such as SCA1-8, are devastating disorders with no disease-modifying therapy. Exemplary targets include SCA2, SCA3, and SCA1.

Additional examples of CNS gene and ocular gene can be found in WO 2019/217459, which is incorporated herein by reference in its entirety.

### Evaluation of Candidate Oligonucleotide

One can evaluate a candidate oligonucleotide or iRNA agent, e.g., a modified RNA, for a selected property by exposing the agent or modified molecule and a control molecule to the appropriate conditions and evaluating for the presence of the selected property. For example, resistance to a degradant can be evaluated as follows. A candidate modified RNA (and a control molecule, usually the unmodified form) can be exposed to degradative conditions, e.g., exposed to a milieu, which includes a degradative agent, e.g., a nuclease. E.g., one can use a biological sample, e.g., one that is similar to a milieu, which might be encountered, in therapeutic use, e.g., blood or a cellular fraction, e.g., a cell-free homogenate or disrupted cells. The candidate and control could then be evaluated for resistance to degradation by any of a number of approaches. For example, the candidate and control could be labeled prior to exposure, with, e.g., a radioactive or enzymatic label, or a fluorescent label, such as Cy3 or Cy5. Control and modified RNA's can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to silence gene expression. For example, a cell, e.g., a mammalian cell, such as a mouse or human cell, can be co-transfected with a plasmid expressing a fluorescent protein, e.g., GFP, and a candidate RNA agent homologous to the transcript encoding the fluorescent protein (see, e.g., WO 00/44914). For example, a modified dsiRNA homologous to the GFP mRNA can be assayed for the ability to inhibit GFP expression by monitoring for a decrease in cell fluorescence, as compared to a control cell, in which the transfection did not include the candidate dsiRNA, e.g., controls with no agent added and/or controls with a non-modified RNA added. Efficacy of the candidate agent on gene expression can be assessed by comparing cell fluorescence in the presence of the modified and unmodified dsiRNA compounds.

In an alternative functional assay, a candidate dsiRNA compound homologous to an endogenous mouse gene, for example, a maternally expressed gene, such as c-mos, can be injected into an immature mouse oocyte to assess the ability of the agent to inhibit gene expression in vivo (see, e.g., WO 01/36646). A phenotype of the oocyte, e.g., the ability to maintain arrest in metaphase II, can be monitored as an indicator that the agent is inhibiting expression. For example, cleavage of c-mos mRNA by a dsiRNA compound would cause the oocyte to exit metaphase arrest and initiate parthenogenetic development (Colledge et al. Nature 370: 65-68, 1994; Hashimoto et al. Nature, 370:68-71, 1994). The effect of the modified agent on target RNA levels can be verified by Northern blot to assay for a decrease in the level of target mRNA, or by Western blot to assay for a decrease in the level of target protein, as compared to a negative control. Controls can include cells in which with no agent is added and/or cells in which a non-modified RNA is added.

### Physiological Effects

The single-stranded oligonucleotide compounds described herein can be designed such that determining therapeutic toxicity is made easier by the complementarity of the siRNA with both a human and a non-human animal sequence. By these methods, an siRNA can consist of a sequence that is fully complementary to a nucleic acid sequence from a human and a nucleic acid sequence from at least one non-human animal, e.g., a non-human mammal, such as a rodent, ruminant or primate. For example, the non-human mammal can be a mouse, rat, dog, pig, goat, sheep, cow, monkey, Pan paniscus, Pan troglodytes, Macaca mulatto, or Cynomolgus monkey. The sequence of the siRNA compound could be complementary to sequences within homologous genes, e.g., oncogenes or tumor suppressor genes, of the non-human mammal and the human. By determining the toxicity of the siRNA compound in the non-human mammal, one can extrapolate the toxicity of the siRNA compound in a human. For a more strenuous toxicity test, the siRNA can be complementary to a human and more than one, e.g., two or three or more, non-human animals.

The methods described herein can be used to correlate any physiological effect of a single-stranded oligonucleotide on a human, e.g., any unwanted effect, such as a toxic effect, or any positive, or desired effect.

### Increasing Cellular Uptake of Oligonucleotides

Described herein are various single-stranded oligonucleotide compositions that contain covalently attached conjugates that increase cellular uptake and/or intracellular targeting of the single-stranded oligonucleotides.

Additionally provided are methods of the invention that include administering a single-stranded oligonucleotide compound and a drug that affects the uptake of the single-stranded oligonucleotide into the cell. The drug can be administered before, after, or at the same time that the single-stranded oligonucleotide compound is administered. The drug can be covalently or non-covalently linked to the single-stranded oligonucleotide compound. The drug can be, for example, a lipopolysaccharide, an activator of p38 MAP kinase, or an activator of NF-κB. The drug can have a transient effect on the cell. The drug can increase the uptake of the single-stranded oligonucleotide compound into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The drug can also increase the uptake of the single-stranded oligonucleotide compound into a given cell or tissue by activating an inflammatory response, for example. Exemplary drugs that would have such an effect include tumor necrosis factor alpha (TNFalpha), interleukin-1 beta, a CpG motif, gamma interferon or more generally an agent that activates a toll-like receptor.

### Pharmaceutical Compositions

In one aspect, the invention features a pharmaceutical composition that includes a single-stranded oligonucleotide described herein according to the above embodiments. The single-stranded oligonucleotide contains an antisense strand that can target a target gene. The target gene can be a transcript of an endogenous human gene. In one embodiment, the pharmaceutical composition can be an emulsion, microemulsion, cream, jelly, or liposome.

In one example, the pharmaceutical composition includes a single-stranded oligonucleotide mixed with a topical delivery agent. The topical delivery agent can be a plurality of microscopic vesicles. The microscopic vesicles can be liposomes. In some embodiments the liposomes are cationic liposomes.

In another aspect, the pharmaceutical composition includes a single-stranded oligonucleotide compound admixed with a topical penetration enhancer. In one embodiment, the topical penetration enhancer is a fatty acid. The fatty acid can be arachidonic acid, oleic acid, lauric acid, caprylic acid, capric acid, myristic acid, palmitic acid, stearic acid, linoleic acid, linolenic acid, dicaprate, tricaprate, monolein, dilaurin, glyceryl 1-monocaprate, 1-dodecylazacycloheptan-2-one, an acylcarnitine, an acylcholine, or a C₁₋₁₀ alkyl ester, monoglyceride, diglyceride or pharmaceutically acceptable salt thereof.

In another embodiment, the topical penetration enhancer is a bile salt. The bile salt can be cholic acid, dehydrocholic acid, deoxycholic acid, glucholic acid, glycholic acid, glycodeoxycholic acid, taurocholic acid, taurodeoxycholic acid, chenodeoxycholic acid, ursodeoxycholic acid, sodium tauro-24,25-dihydro-fusidate, sodium glycodihydrofusidate, polyoxyethylene-9-lauryl ether or a pharmaceutically acceptable salt thereof.

In another embodiment, the penetration enhancer is a chelating agent. The chelating agent can be EDTA, citric acid, a salicyclate, a N-acyl derivative of collagen, laureth-9, an N-amino acyl derivative of a beta-diketone or a mixture thereof.

In another embodiment, the penetration enhancer is a surfactant, e.g., an ionic or nonionic surfactant. The surfactant can be sodium lauryl sulfate, polyoxyethylene-9-lauryl ether, polyoxyethylene-20-cetyl ether, a perfluorochemical emulsion or mixture thereof.

In another embodiment, the penetration enhancer can be selected from a group consisting of unsaturated cyclic ureas, 1-alkyl-alkones, 1-alkenylazacyclo-alakanones, steroidal anti-inflammatory agents and mixtures thereof. In yet another embodiment the penetration enhancer can be a glycol, a pyrrol, an azone, or a terpene.

In one aspect, the invention features a pharmaceutical composition including an sciRNA compound in a form suitable for oral delivery. In one embodiment, oral delivery can be used to deliver a single-stranded oligonucleotide compound composition to a cell or a region of the gastro-intestinal tract, e.g., small intestine, colon (e.g., to treat a colon cancer), and so forth. The oral delivery form can be tablets, capsules or gel capsules. In one embodiment, the single-stranded oligonucleotide compound of the pharmaceutical composition modulates expression of a cellular adhesion protein, modulates a rate of cellular proliferation, or has biological activity against eukaryotic pathogens or retroviruses. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methylcellulose phthalate or cellulose acetate phthalate.

In another embodiment, the oral dosage form of the pharmaceutical composition includes a penetration enhancer. The penetration enhancer can be a bile salt or a fatty acid. The bile salt can be ursodeoxycholic acid, chenodeoxycholic acid, and salts thereof. The fatty acid can be capric acid, lauric acid, and salts thereof.

In another embodiment, the oral dosage form of the pharmaceutical composition includes an excipient. In one example the excipient is polyethyleneglycol. In another example the excipient is precirol.

In another embodiment, the oral dosage form of the pharmaceutical composition includes a plasticizer. The plasticizer can be diethyl phthalate, triacetin dibutyl sebacate, dibutyl phthalate or triethyl citrate.

In one aspect, the invention features a pharmaceutical composition including a single-stranded oligonucleotide compound and a delivery vehicle.

In one embodiment, the delivery vehicle can deliver a single-stranded oligonucleotide compound to a cell by a topical route of administration. The delivery vehicle can be microscopic vesicles. In one example the microscopic vesicles are liposomes. In some embodiments the liposomes are cationic liposomes. In another example the microscopic vesicles are micelles.

In one aspect, the invention features a pharmaceutical composition including a single-stranded oligonucleotide in an injectable dosage form. In one embodiment, the injectable dosage form of the pharmaceutical composition includes sterile aqueous solutions or dispersions and sterile powders. In some embodiments the sterile solution can include a diluent such as water; saline solution; fixed oils, polyethylene glycols, glycerin, or propylene glycol.

In one aspect, the invention features a pharmaceutical composition including a single-stranded oligonucleotide in oral dosage form. In one embodiment, the oral dosage form is selected from the group consisting of tablets, capsules and gel capsules. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methyl cellulose phthalate or cellulose acetate phthalate. In one embodiment, the oral dosage form of the pharmaceutical composition includes a penetration enhancer, e.g., a penetration enhancer described herein.

In one aspect, the invention features a pharmaceutical composition including a single-stranded oligonucleotide compound in a rectal dosage form. In one embodiment, the rectal dosage form is an enema. In another embodiment, the rectal dosage form is a suppository.

In one aspect, the invention features a pharmaceutical composition including a single-stranded oligonucleotide compound in a vaginal dosage form. In one embodiment, the vaginal dosage form is a suppository. In another embodiment, the vaginal dosage form is a foam, cream, or gel.

In one aspect, the invention features a pharmaceutical composition including a single-stranded oligonucleotide described herein according to the above embodiments in a pulmonary or nasal dosage form. In one embodiment, the single-stranded oligonucleotide compound is incorporated into a particle, e.g., a macroparticle, e.g., a microsphere. The particle can be produced by spray drying, lyophilization, evaporation, fluid bed drying, vacuum drying, or a combination thereof. The microsphere can be formulated as a suspension, a powder, or an implantable solid.

### Treatment Methods and Routes of Delivery

Another aspect of the invention relates to a method of reducing the expression of a target gene in a cell, comprising contacting said cell with the single-stranded oligonucleotide described herein according to the above embodiments. In one embodiment, the cell is a heptic cell. In one embodiment, the cell is an extraheptic cell.

Another aspect of the invention relates to a method of reducing the expression of a target gene in a subject, comprising administering to the subject the single-stranded oligonucleotide described herein according to the above embodiments.

The single-stranded oligonucleotide can be delivered to a subject by a variety of routes, depending on the type of genes targeted and the type of disorders to be treated. In some embodiments, the single-stranded oligonucleotide is administered hepatically. In some embodiments, the single-stranded oligonucleotide is administered extrahepatically, such as an ocular administration (e.g., intravitreal administration) or an intrathecal administration.

In one embodiment, the single-stranded oligonucleotide is administered intrathecally. By intrathecal administration of the single-stranded oligonucleotide, the method can reduce the expression of a target gene in a brain or spine tissue, for instance, cortex, cerebellum, cervical spine, lumbar spine, and thoracic spine.

For ease of exposition the formulations, compositions and methods in this section are discussed largely with regard to modified single-stranded oligonucleotide compounds. It may be understood, however, that these formulations, compositions and methods can be practiced with other compounds, e.g., unmodified single-stranded oligonucleotide compounds, and such practice is within the invention. A composition that includes a iRNA can be delivered to a subject by a variety of routes. Exemplary routes include: intravenous, topical, rectal, anal, vaginal, nasal, pulmonary, ocular.

The single-stranded oligonucleotide can be incorporated into pharmaceutical compositions suitable for administration. Such compositions typically include one or more species of single-stranded oligonucleotide and a pharmaceutically acceptable carrier. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the iRNA in aerosol form. The vascular endothelial cells could be targeted by coating a balloon catheter with the iRNA and mechanically introducing the DNA.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Compositions for intrathecal or intraventricular administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes may be controlled to render the preparation isotonic.

For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers.

In one embodiment, the administration of the single-stranded oligonucleotide composition is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

Intrathecal Administration. In one embodiment, the single-stranded oligonucleotide is delivered by intrathecal injection (i.e. injection into the spinal fluid which bathes the brain and spinal cord tissue). Intrathecal injection of single-stranded oligonucleotides into the spinal fluid can be performed as a bolus injection or via minipumps which can be implanted beneath the skin, providing a regular and constant delivery of single-stranded oligonucleotide into the spinal fluid. The circulation of the spinal fluid from the choroid plexus, where it is produced, down around the spinal cord and dorsal root ganglia and subsequently up past the cerebellum and over the cortex to the arachnoid granulations, where the fluid can exit the CNS, that, depending upon size, stability, and solubility of the compounds injected, molecules delivered intrathecally could hit targets throughout the entire CNS.

In some embodiments, the intrathecal administration is via a pump. The pump may be a surgically implanted osmotic pump. In one embodiment, the osmotic pump is implanted into the subarachnoid space of the spinal canal to facilitate intrathecal administration.

In some embodiments, the intrathecal administration is via an intrathecal delivery system for a pharmaceutical including a reservoir containing a volume of the pharmaceutical agent, and a pump configured to deliver a portion of the pharmaceutical agent contained in the reservoir. More details about this intrathecal delivery system may be found in PCT/US2015/013253, filed on Jan. 28, 2015, which is incorporated by reference in its entirety.

The amount of intrathecally injected single-stranded oligonucleotide may vary from one target gene to another target gene and the appropriate amount that has to be applied may have to be determined individually for each target gene. Typically, this amount ranges between 10 kg to 2 mg, preferably 50 kg to 1500 kg, more preferably 100 kg to 1000 kg.

Rectal Administration. The invention also provides methods, compositions, and kits, for rectal administration or delivery of single-stranded oligonucleotides described herein.

Accordingly, a single-stranded oligonucleotide compound can be administered rectally, e.g., introduced through the rectum into the lower or upper colon. This approach is particularly useful in the treatment of, inflammatory disorders, disorders characterized by unwanted cell proliferation, e.g., polyps, or colon cancer.

The medication can be delivered to a site in the colon by introducing a dispensing device, e.g., a flexible, camera-guided device similar to that used for inspection of the colon or removal of polyps, which includes means for delivery of the medication.

The rectal administration of the single-stranded oligonucleotide is by means of an enema. The single-stranded oligonucleotide of the enema can be dissolved in a saline or buffered solution. The rectal administration can also by means of a suppository, which can include other ingredients, e.g., an excipient, e.g., cocoa butter or hydropropylmethylcellulose.

Ocular Delivery. The single-stranded oligonucleotides described herein can be administered to an ocular tissue. For example, the medications can be applied to the surface of the eye or nearby tissue, e.g., the inside of the eyelid. They can be applied topically, e.g., by spraying, in drops, as an eyewash, or an ointment. Administration can be provided by the subject or by another person, e.g., a health care provider. The medication can be provided in measured doses or in a dispenser which delivers a metered dose. The medication can also be administered to the interior of the eye, and can be introduced by a needle or other delivery device which can introduce it to a selected area or structure. Ocular treatment is particularly desirable for treating inflammation of the eye or nearby tissue.

In certain embodiments, the single-stranded oligonucleotides may be delivered directly to the eye by ocular tissue injection such as periocular, conjunctival, subtenon, intracameral, intravitreal, intraocular, anterior or posterior juxtascleral, subretinal, subconjunctival, retrobulbar, or intracanalicular injections; by direct application to the eye using a catheter or other placement device such as a retinal pellet, intraocular insert, suppository or an implant comprising a porous, non-porous, or gelatinous material; by topical ocular drops or ointments; or by a slow release device in the cul-de-sac or implanted adjacent to the sclera (transscleral) or in the sclera (intrascleral) or within the eye. Intracameral injection may be through the cornea into the anterior chamber to allow the agent to reach the trabecular meshwork. Intracanalicular injection may be into the venous collector channels draining Schlemm's canal or into Schlemm's canal.

In one embodiment, the single-stranded oligonucleotides may be administered into the eye, for example the vitreous chamber of the eye, by intravitreal injection, such as with pre-filled syringes in ready-to-inject form for use by medical personnel.

For ophthalmic delivery, the single-stranded oligonucleotides may be combined with ophthalmologically acceptable preservatives, co-solvents, surfactants, viscosity enhancers, penetration enhancers, buffers, sodium chloride, or water to form an aqueous, sterile ophthalmic suspension or solution. Solution formulations may be prepared by dissolving the conjugate in a physiologically acceptable isotonic aqueous buffer. Further, the solution may include an acceptable surfactant to assist in dissolving the single-stranded oligonucleotides. Viscosity building agents, such as hydroxymethyl cellulose, hydroxyethyl cellulose, methylcellulose, polyvinylpyrrolidone, or the like may be added to the pharmaceutical compositions to improve the retention of the single-stranded oligonucleotides.

To prepare a sterile ophthalmic ointment formulation, the single-stranded oligonucleotides is combined with a preservative in an appropriate vehicle, such as mineral oil, liquid lanolin, or white petrolatum. Sterile ophthalmic gel formulations may be prepared by suspending the single-stranded oligonucleotides in a hydrophilic base prepared from the combination of, for example, CARBOPOL®-940 (BF Goodrich, Charlotte, N.C.), or the like, according to methods known in the art.

Topical Delivery. Any of the single-stranded oligonucleotide compounds described herein can be administered directly to the skin. For example, the medication can be applied topically or delivered in a layer of the skin, e.g., by the use of a microneedle or a battery of microneedles which penetrate into the skin, but, for example, not into the underlying muscle tissue. Administration of the single-stranded oligonucleotide composition can be topical. Topical applications can, for example, deliver the composition to the dermis or epidermis of a subject. Topical administration can be in the form of transdermal patches, ointments, lotions, creams, gels, drops, suppositories, sprays, liquids or powders. A composition for topical administration can be formulated as a liposome, micelle, emulsion, or other lipophilic molecular assembly. The transdermal administration can be applied with at least one penetration enhancer, such as iontophoresis, phonophoresis, and sonophoresis.

In some embodiments, a single-stranded oligonucleotide is delivered to a subject via topical administration. “Topical administration” refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver oligonucleotides to the epidermis or dermis of a subject, or to specific strata thereof, or to an underlying tissue.

The term “skin,” as used herein, refers to the epidermis and/or dermis of an animal. Mammalian skin consists of two major, distinct layers. The outer layer of the skin is called the epidermis. The epidermis is comprised of the Stratum corneum, the Stratum granulosum, the Stratum spinosum, and the Stratum basale, with the Stratum corneum being at the surface of the skin and the stratum basale being the deepest portion of the epidermis. The epidermis is between 50 μm and 0.2 mm thick, depending on its location on the body.

Beneath the epidermis is the dermis, which is significantly thicker than the epidermis. The dermis is primarily composed of collagen in the form of fibrous bundles. The collagenous bundles provide support for, inter alia, blood vessels, lymph capillaries, glands, nerve endings and immunologically active cells.

One of the major functions of the skin as an organ is to regulate the entry of substances into the body. The principal permeability barrier of the skin is provided by the Stratum corneum, which is formed from many layers of cells in various states of differentiation. The spaces between cells in the Stratum corneum is filled with different lipids arranged in lattice-like formations that provide seals to further enhance the skins permeability barrier.

The permeability barrier provided by the skin is such that it is largely impermeable to molecules having molecular weight greater than about 750 Da. For larger molecules to cross the skin's permeability barrier, mechanisms other than normal osmosis must be used.

Several factors determine the permeability of the skin to administered agents. These factors include the characteristics of the treated skin, the characteristics of the delivery agent, interactions between both the drug and delivery agent and the drug and skin, the dosage of the drug applied, the form of treatment, and the post treatment regimen. To selectively target the epidermis and dermis, it is sometimes possible to formulate a composition that comprises one or more penetration enhancers that will enable penetration of the drug to a preselected Stratum.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy.

In addition, iontophoresis (transfer of ionic solutes through biological membranes under the influence of an electric field) (Lee et al., Critical Reviews in Therapeutic Drug Carrier Systems, 1991, p. 163), phonophoresis or sonophoresis (use of ultrasound to enhance the absorption of various therapeutic agents across biological membranes, notably the skin and the cornea) (Lee et al., Critical Reviews in Therapeutic Drug Carrier Systems, 1991, p. 166), and optimization of vehicle characteristics relative to dose position and retention at the site of administration (Lee et al., Critical Reviews in Therapeutic Drug Carrier Systems, 1991, p. 168) may be useful methods for enhancing the transport of topically applied compositions across skin and mucosal sites.

The compositions and methods provided may also be used to examine the function of various proteins and genes in vitro in cultured or preserved dermal tissues and in animals. The invention can be thus applied to examine the function of any gene. The methods of the invention can also be used therapeutically or prophylactically. For example, for the treatment of animals that are known or suspected to suffer from diseases such as psoriasis, lichen planus, toxic epidermal necrolysis, ertythema multiforme, basal cell carcinoma, squamous cell carcinoma, malignant melanoma, Paget's disease, Kaposi's sarcoma, pulmonary fibrosis, Lyme disease and viral, fungal and bacterial infections of the skin.

Pulmonary Delivery. Any of the single-stranded oligonucleotides described herein can be administered to the pulmonary system. Pulmonary administration can be achieved by inhalation or by the introduction of a delivery device into the pulmonary system, e.g., by introducing a delivery device which can dispense the medication. Certain embodiments may use a method of pulmonary delivery by inhalation. The medication can be provided in a dispenser which delivers the medication, e.g., wet or dry, in a form sufficiently small such that it can be inhaled. The device can deliver a metered dose of medication. The subject, or another person, can administer the medication. Pulmonary delivery is effective not only for disorders which directly affect pulmonary tissue, but also for disorders which affect other tissue. sciRNA compounds can be formulated as a liquid or nonliquid, e.g., a powder, crystal, or aerosol for pulmonary delivery.

A composition that includes a single-stranded oligonucleotide can be administered to a subject by pulmonary delivery. Pulmonary delivery compositions can be delivered by inhalation by the patient of a dispersion so that the composition, for example, iRNA, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are may be used. One of the benefits of using an atomizer or inhaler is that the potential for contamination is minimized because the devices are self contained. Dry powder dispersion devices, for example, deliver drugs that may be readily formulated as dry powders. A iRNA composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” For example, the average particle size is less than about 10 μm in diameter with a relatively uniform spheroidal shape distribution. In some embodiments, the diameter is less than about 7.5 μm and in some embodiments less than about 5.0 μm. Usually the particle size distribution is between about 0.1 μm and about 5 μm in diameter, sometimes about 0.3 μm to about 5 μm.

The term “dry” means that the composition has a moisture content below about 10% by weight (% w) water, usually below about 5% w and in some cases less it than about 3% w. A dry composition can be such that the particles are readily dispersible in an inhalation device to form an aerosol.

The term “therapeutically effective amount” is the amount present in the composition that is needed to provide the desired level of drug in the subject to be treated to give the anticipated physiological response.

The term “physiologically effective amount” is that amount delivered to a subject to give the desired palliative or curative effect.

The term “pharmaceutically acceptable carrier” means that the carrier can be taken into the lungs with no significant adverse toxicological effects on the lungs.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. A group of carbohydrates may include lactose, threhalose, raffinose maltodextrins, and mannitol. Suitable polypeptides include aspartame. Amino acids include alanine and glycine, with glycine being used in some embodiments.

Additives, which are minor components of the composition of this invention, may be included for conformational stability during spray drying and for improving dispersibility of the powder. These additives include hydrophobic amino acids such as tryptophan, tyrosine, leucine, phenylalanine, and the like.

Suitable pH adjusters or buffers include organic salts prepared from organic acids and bases, such as sodium citrate, sodium ascorbate, and the like; sodium citrate may be used in some embodiments.

Pulmonary administration of a micellar iRNA formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

Oral or Nasal Delivery. Any of the single-stranded oligonucleotides described herein can be administered orally, e.g., in the form of tablets, capsules, gel capsules, lozenges, troches or liquid syrups. Further, the composition can be applied topically to a surface of the oral cavity.

Any of the single-stranded oligonucleotides described herein can be administered nasally. Nasal administration can be achieved by introduction of a delivery device into the nose, e.g., by introducing a delivery device which can dispense the medication. Methods of nasal delivery include spray, aerosol, liquid, e.g., by drops, or by topical administration to a surface of the nasal cavity. The medication can be provided in a dispenser with delivery of the medication, e.g., wet or dry, in a form sufficiently small such that it can be inhaled. The device can deliver a metered dose of medication. The subject, or another person, can administer the medication.

Nasal delivery is effective not only for disorders which directly affect nasal tissue, but also for disorders which affect other tissue single-stranded oligonucleotides can be formulated as a liquid or nonliquid, e.g., a powder, crystal, or for nasal delivery. As used herein, the term “crystalline” describes a solid having the structure or characteristics of a crystal, i.e., particles of three-dimensional structure in which the plane faces intersect at definite angles and in which there is a regular internal structure. The compositions of the invention may have different crystalline forms. Crystalline forms can be prepared by a variety of methods, including, for example, spray drying.

Both the oral and nasal membranes offer advantages over other routes of administration. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many drugs. Further, the sublingual mucosa is convenient, acceptable and easily accessible.

The ability of molecules to permeate through the oral mucosa appears to be related to molecular size, lipid solubility and peptide protein ionization. Small molecules, less than 1000 daltons appear to cross mucosa rapidly. As molecular size increases, the permeability decreases rapidly. Lipid soluble compounds are more permeable than non-lipid soluble molecules. Maximum absorption occurs when molecules are un-ionized or neutral in electrical charges. Therefore charged molecules present the biggest challenges to absorption through the oral mucosae.

A pharmaceutical composition of the single-stranded oligonucleotide may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity. For example, the medication can be sprayed into the buccal cavity or applied directly, e.g., in a liquid, solid, or gel form to a surface in the buccal cavity. This administration is particularly desirable for the treatment of inflammations of the buccal cavity, e.g., the gums or tongue, e.g., in one embodiment, the buccal administration is by spraying into the cavity, e.g., without inhalation, from a dispenser, e.g., a metered dose spray dispenser that dispenses the pharmaceutical composition and a propellant.

### Kits

In certain other aspects, the invention provides kits that include a suitable container containing a pharmaceutical formulation of a single-stranded oligonucleotide described herein according to the above embodiments. In certain embodiments the individual components of the pharmaceutical formulation may be provided in one container. Alternatively, it may be desirable to provide the components of the pharmaceutical formulation separately in two or more containers, e.g., one container for a single-stranded oligonucleotide preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The invention is further illustrated by the following examples, which should not be construed as further limiting. The contents of all references, pending patent applications and published patents, cited throughout this application are hereby expressly incorporated by reference.

### Examples

The invention now being generally described, it will be more readily understood by reference to the following examples which are included merely for purposes of illustration of certain aspects and embodiments of the present invention, and are not intended to limit the invention.

TABLE I
Abbreviations of nucleotide monomers used in nucleic acid sequence representation. It will be understood that these monomers,
when present in an oligonucleotide, are mutually linked by 5′-3′-phosphodiester bonds; and it is understood that when the nucleotide contains a
2′-fluoro modification, then the fluoro replaces the hydroxy at that position in the parent nucleotide (i.e., it is a 2′-deoxy-2′-fluoronucleotide).
The abbreviations are understood to omit the 3′-phosphate (i.e. they are 3′-OH) when placed at the 3′-terminal position of an oligonucleotide

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide, modified or unmodified |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol |
| | (Hyp-(GalNAc-alkyl)3) |
| | 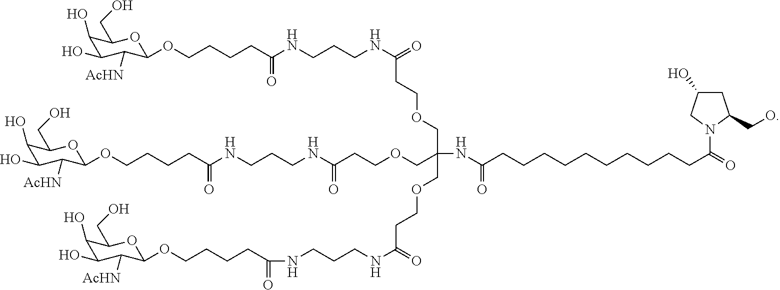 |
| | (2S,4R)-1-[29-[[2-(acetylamino)-2-deoxy-β-D-galactopyranosyl]oxy]- |
| | 14,14-bis[[3-[[3-[[5-[[2-(acetylamino)-2-deoxy-β-D- |
| | galactopyranosyl]oxy]-1-oxopentyl]amino]propyl]amino]-3- |
| | oxopropoxy]methyl]-1,12,19,25-tetraoxo-16-oxa-13,20,24- |
| | triazanonacos-1-yl]-4-hydroxy-2-hydroxymethylpyrrolidine |
| aL96 | 3′-terminal L-96 modified nucleotide (L96 modified a, u, c, g, or t, |
| uL96 | respectively at a 3′-terminal. E.g., uL96: |
| cL96 | 2′-O-methyluridine-3′-phosphate ((2S,4R)-1-[29-[[2-(acetylamino)-2- |
| gL96 | deoxy-β-D-galactopyranosyl]oxy]-14,14-bis[[3-[[3-[[5-[[2- |
| tL96 | (acetylamino)-2-deoxy-β-D-galactopyranosyl]oxy]-1- |
| | oxopentyl]amino]propyl]amino]-3-oxopropoxy]methyl]-1,12,19,25- |
| | tetraoxo-16-oxa-13,20,24-triazanonacos-1-yl]-4-hydroxy-2- |
| | pyrrolidinyl)methyl ester |
| | 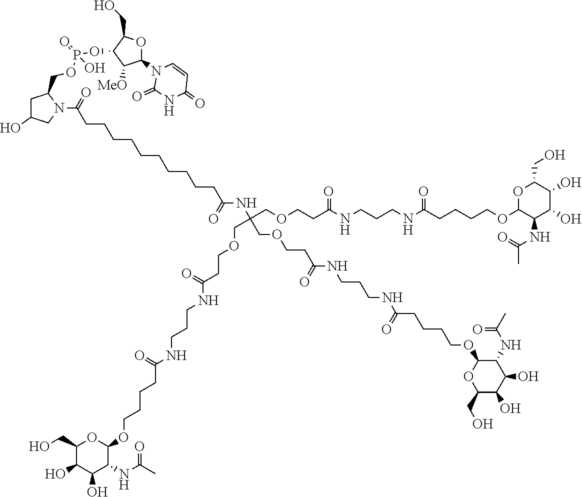 [SMILES: CO[C@@H]1[C@H](OP(=O)(O)OC[C@@H]2CC(O)CN2C(=O)CCCCCCCCCCC(=O)NC(COCCC(=O)NCCCNC(=O)CCCCOC2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)COCCC(=O)NCCCNC(=O)CCCCO[C@@H]2O[C@H](CO)[C@H](O)[C@H](O)[C@H]2NC(C)=O)[C@@H](CO)O[C@H]1n1ccc(=O)[nH]c1=O] |
| Y34 | 2-hydroxymethyl-tetrahydrofurane-4-methoxy-3-phosphate (abasic 2′- |
| | OMe furanose) |
| | 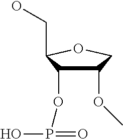 |
| Y44 | inverted abasic DNA (2-hydroxymethyl-tetrahydrofurane-5-phosphate) |
| | 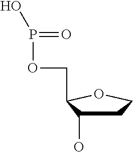 |
| L10 | N-(cholesterylcarboxamidocaproyl)-4-hydroxyprolinol (Hyp-C6-Chol) |
| | 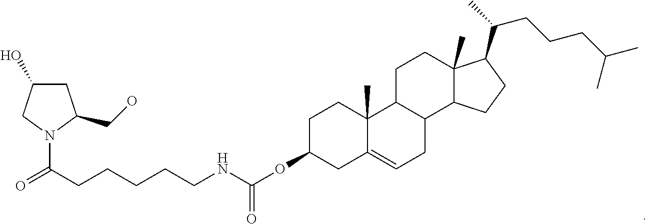 [SMILES: CC(C)CCC[C@@H](C)[C@H]1CCC2C3CC=C4C[C@@H](OC(=O)NCCCCCC(=O)N5C[C@H](O)C[C@H]5CO)CC[C@]4(C)C3CC[C@@]21C] |
| (Agn) | Adenosine-glycol nucleic acid (GNA) S-Isomer |
| (Cgn) | Cytidine-glycol nucleic acid (GNA) S-Isomer |
| (Ggn) | Guanosine-glycol nucleic acid (GNA) S-Isomer |
| (Tgn) | Thymidine-glycol nucleic acid (GNA) S-Isomer |
| P | Phosphate |
| VP | Vinyl-phosphonate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3′-phosphorothioate |
| (C2p) | cytidine-2′-phosphate |
| (G2p) | guanosine-2′-phosphate |
| (U2p) | uridine-2′-phosphate |
| (A2p) | adenosine-2′-phosphate |
| (Ahd) | 2′-O-hexadecyl-adenosine-3′-phosphate |
| (Ahds) | 2′-O-hexadecyl-adenosine-3′-phosphorothioate |
| (Chd) | 2′-O-hexadecyl-cytidine-3′-phosphate |
| (Chds) | 2′-O-hexadecyl-cytidine-3′-phosphorothioate |
| (Ghd) | 2′-O-hexadecyl-guanosine-3′-phosphate |
| (Ghds) | 2′-O-hexadecyl-guanosine-3′-phosphorothioate |
| (Uhd) | 2′-O-hexadecyl-uridine-3′-phosphate |
| (Uhds) | 2′-O-hexadecyl-uridine-3′-phosphorothioate |
| s | Phosphorothioate |
| Y211 | 2′-O-methyluridine-3′-(n-hexadecyl)phosphoramidate |
| | 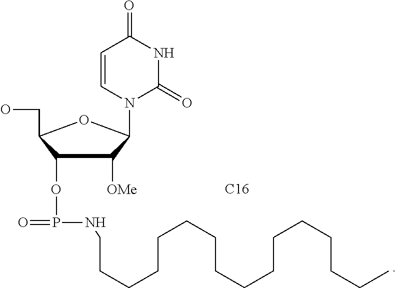 |

### Example 1: Single-Stranded Loop Oligonucleotides

Exemplary single-stranded loop oligonucleotides were synthesized in this example.

### Synthesis of Single-Stranded Loop Oligonucleotides

Oligonucleotides were synthesized on a MerMade-12 DNA/RNA synthesizer. Sterling solvents/reagents from Glen Research, 500-A controlled pore glass (CPG) solid supports from Prime Synthesis, 2′-deoxy 3′-phosphoramidites from Thermo, and 2′-OMe and 2′-F nucleoside 3′-phosphoramidites from Hongene were all used as received. The 2′-OMe-uridine-5′-bis-POM-(E) vinyl phosphonate (VP) 3′-phosphoramidite was synthesized according to previously published procedures (Parmar et al., J. Med. Chem., 61, 734-744 (2018), which is incorporated herein by reference in its entirety), dissolved to 0.15 M in 85% acetonitrile 15% dimethylformamide (DMF), and coupled using standard conditions on the synthesizer. GalNAc CPG support was prepared and used as previously described (Nair et al., J. Am. Chem. Soc., 136, 16958-16961 (2014), which is incorporated herein by reference in its entirety). Low-water content acetonitrile was purchased from EMD Chemicals. A solution of 0.6 M 5-(S-ethylthio)-1H-tetrazole in acetonitrile was used as the activator. The phosphoramidite solutions were 0.15 M in anhydrous acetonitrile with 15% DMF as a co-solvent for 2′-OMe uridine and cytidine. The oxidizing reagent was 0.02 M I₂ in THF/pyridine/water. N,N-Dimethyl-N′-(3-thioxo-3H-1,2,4-dithiazol-5-yl)methanimidamide (DDTT), 0.09 M in pyridine, was used as the sulfurizing reagent. The detritylation reagent was 3% dichloroacetic acid (DCA) in dichloromethane (DCM).

After completion of the solid-phase synthesis, the CPG solid support was washed with 5% (v/v) piperidine in anhydrous acetonitrile three times with 5-min holds after each flow. The support was then washed with anhydrous acetonitrile and dried with argon. The oligonucleotides were then incubated with 28-30% (w/v) NH₄OH, at 35° C. for 20 hours. For VP-containing oligonucleotides, the CPG solid support was incubated with 28-30% (w/v) NH₄OH containing 5% (v/v) of diethylamine at 35° C. for 20 hours (O'Shea et al., Tetrahedron, 74, 6182-6186 (2018), which is incorporated herein by reference in its entirety). The solvent was collected by filtration, and the support was rinsed with water prior to analysis. Oligonucleotide solutions of approximately 1 OD₂₆₀ units/mL were used for analysis of the crudes, and 30-50 μL of solution were injected. LC/ESI-MS was performed on an Agilent 6130 single quadrupole LC/MS system using an XBridge C8 column (2.1×50 mm, 2.5 μm) at 60° C. Buffer A consisted of 200 mM 1,1,1,3,3,3-hexafluoro-2-propanol and 16.3 mM triethylamine in water, and buffer B was 100% methanol. A gradient from 000 to 40% of buffer B over 10 minutes followed by washing and recalibration at a flow rate of 0.70 mL/minute. The column temperature was 75° C. All oligonucleotides were purified and desalted, and further annealed to form GalNAc-oligonucleotides as previously described (Nair et al., J. Am. Chem. Soc., 136, 16958-16961 (2014), which is incorporated herein by reference in its entirety).

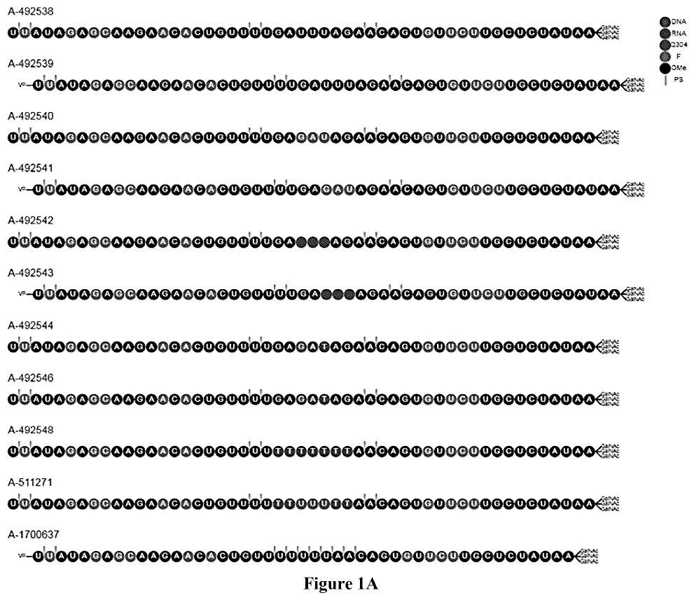

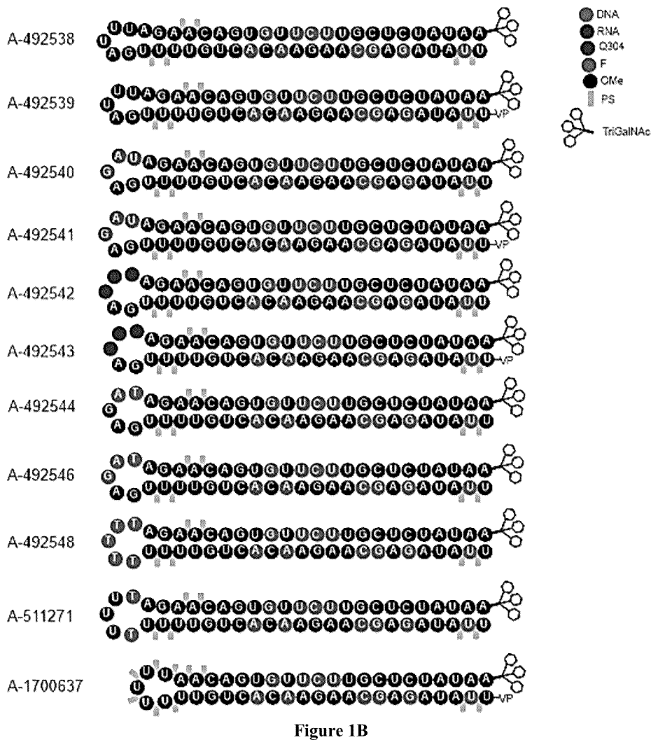

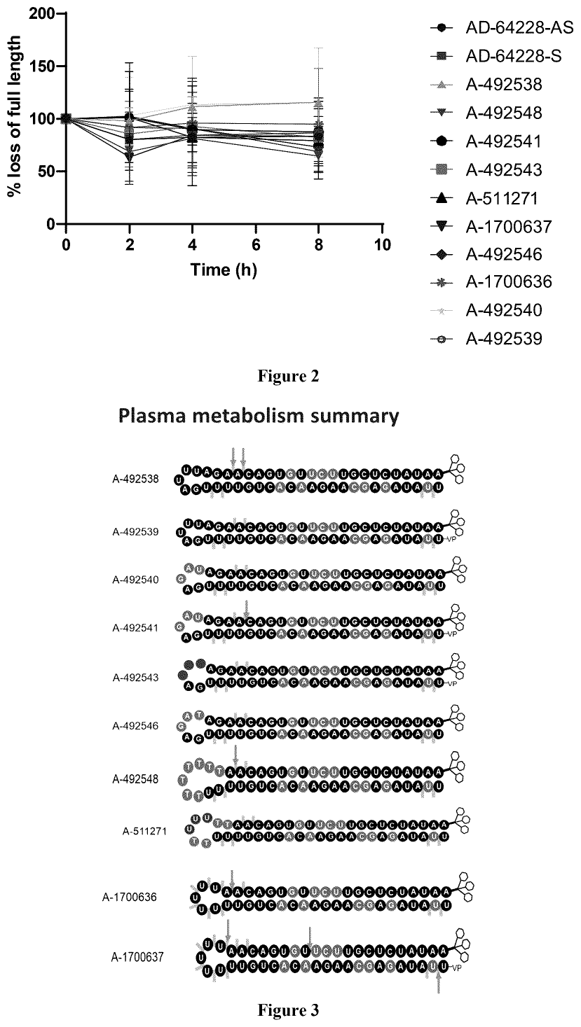

The sequence designs are shown in FIGS. 1-2 as well as in Table 1 below.

TABLE 1
Exemplary single-stranded oligonucleotides

| | | SEQ ID |
|---|---|---|
| Oligo # | OligoSeq (5′-3′)* | NO: |
| A-492538 | usUfsauaGfaGfCfaagaAfcAfcuguususugauuuagasascag | 38 |
| | uGfuUfCfUfugcucuauaaL96 | |
| A-492539 | VPusUfsauaGfaGfCfaagaAfcAfcuguususugauuuagasas | 39 |
| | caguGfuUfCfUfugcucuauaaL96 | |
| A-492540 | usUfsauaGfaGfCfaagaAfcAfcuguususugaGfAfUfagasa | 40 |
| | scaguGfuUfCfUfugcucuauaaL96 | |
| A-492541 | VPusUfsauaGfaGfCfaagaAfcAfcuguususugaGfAfUfag | 41 |
| | asascaguGfuUfCfUfugcucuauaaL96 | |
| A-492542 | usUfsauaGfaGfCfaagaAfcAfcuguususugaQ304Q304Q | 42, 131 |
| | 304agasascaguGfuUfCfUfugcucuauaaL96 | |
| A-492543 | VPusUfsauaGfaGfCfaagaAfcAfcuguususugaQ304Q30 | 43, 132 |
| | 4Q304agasascaguGfuUfCfUfugcucuauaaL96 | |
| A-492544 | usUfsauaGfaGfCfaagaAfcAfcuguususugaGdAdTagasa | 44 |
| | scaguGfuUfCfUfugcucuauaaL96 | |
| A-492546 | usUfsauaGfaGfCfaagaAfcAfcuguususugadGdAdTagas | 45 |
| | ascaguGfuUfCfUfugcucuauaaL96 | |
| A-492548 | usUfsauaGfaGfCfaagaAfcAfcuguususudTdTdTdTdTd | 46 |
| | TdTasascaguGfuUfCfUfugcucuauaaL96 | |
| A-511271 | usUfsauaGfaGfCfaagaAfcAfcuguususudTdTUUUdTd | 47 |
| | TasascaguGfuUfCfUfugcucuauaaL96 | |
| A-1700636 | usUfsauaGfaGfCfaagaAfcAfcuguususususususasascag | 48 |
| | uGfuUfCfUfugcucuauaaL96 | |
| A-1700637 | VPusUfsauaGfaGfCfaagaAfcAfcuguususususususasasc | 49 |
| | aguGfuUfCfUfugcucuauaaL96 | |

Chemistry modifications are indicated as follows:
S-PS linkage; lower case nucleotide—2′-OMe; upper case nucleotide—a ribonucleotide (RNA); “d” followed by upper case nucleotide—2′-deoxy (DNA); upper case nucleotide followed by “f”—2′-F; VP—5′-(E)-vinylphosphonate;

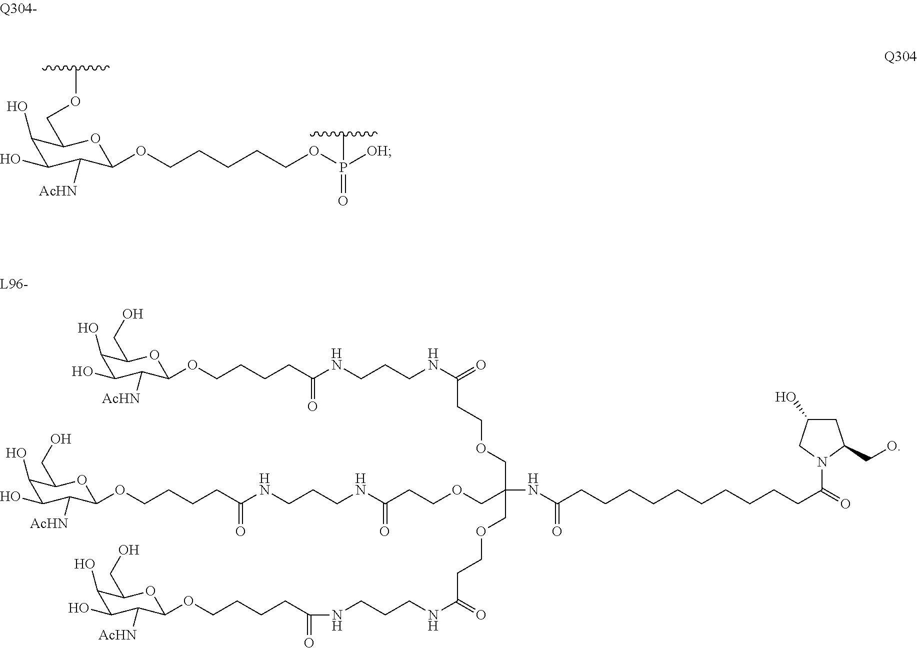

The base sequences for these single-stranded oligonucleotides in Table 1 (i.e., the sequence without modifications to the nucleotides) are show in Table 2.

TABLE 2
Base sequences for the exemplary single-stranded
oligonucleotides of Table 1

| A# | transSeq (5′-3′) | SEQ ID NO: |
|---|---|---|
| A-492538 | UUAUAGAGCAAGAACACUGUUUUGAUUUAGA | 50 |
| | ACAGUGUUCUUGCUCUAUAA | |
| A-492539 | UUAUAGAGCAAGAACACUGUUUUGAUUUAGA | 51 |
| | ACAGUGUUCUUGCUCUAUAA | |
| A-492540 | UUAUAGAGCAAGAACACUGUUUUGAGAUAGA | 52 |
| | ACAGUGUUCUUGCUCUAUAA | |
| A-492541 | UUAUAGAGCAAGAACACUGUUUUGAGAUAGA | 53 |
| | ACAGUGUUCUUGCUCUAUAA | |
| A-492542 | UUAUAGAGCAAGAACACUGUUUUGAXXXAGA | 54, 55 |
| | ACAGUGUUCUUGCUCUAUAA | |
| A-492543 | UUAUAGAGCAAGAACACUGUUUUGAXXXAGA | 56, 57 |
| | ACAGUGUUCUUGCUCUAUAA | |
| A-492544 | UUAUAGAGCAAGAACACUGUUUUGAGATAGA | 58 |
| | ACAGUGUUCUUGCUCUAUAA | |
| A-492546 | UUAUAGAGCAAGAACACUGUUUUGAGATAGA | 59 |
| | ACAGUGUUCUUGCUCUAUAA | |
| A-492548 | UUAUAGAGCAAGAACACUGUUUUTTTTTTTAA | 60 |
| | CAGUGUUCUUGCUCUAUAA | |
| A-511271 | UUAUAGAGCAAGAACACUGUUUUTTUUUTTA | 61 |
| | ACAGUGUUCUUGCUCUAUAA | |
| A-1700636 | UUAUAGAGCAAGAACACUGUUUUUUUAACAG | 62 |
| | UGUUCUUGCUCUAUAA | |
| A-1700637 | UUAUAGAGCAAGAACACUGUUUUUUUAACAG | |
| | UGUUCUUGCUCUAUAA | 63 |

The sequences of the parent siRNA duplex are shown in Table 3 below.

TABLE 3
The sequences of the wild-type dsRNA duplex

| Duplex # | Strand | oligoSeq (5′-3′) |
|---|---|---|
| AD-64228 | Sense | asascaguGfuUfCfUfugcucu |
| | | auaaL96 (SEQ ID NO: 64) |
| | Antisense | usUfsauaGfaGfCfaagaAfcA |
| | | fcuguususu (SEQ ID NO: 65) |

### In Vitro Stability/Metabolism of the Oligonucleotide in Plasma and Liver Homogenates

The in vitro metabolism of all above exemplary single-stranded oligonucleotides were assessed in mouse plasma and mouse liver homogenates, as compared to the parent siRNA duplex.

In vitro metabolism was performed in rat plasma (BioIVT, Cat #CUSTOMBIOFBLD) and liver homogenate (BioIVT, custom order) as described by Jahns et al., Nucleic Acids Research 49, 10250-10264 (2021), which is incorporated herein by reference in its entirety. The final reaction mixture consisted of 1 mM MgCl₂, 1 mM MnCl₂, and 2 mM CaCl₂. The single-stranded oligonucleotides were incubated at 20 μg/mL with either plasma or the liver homogenate. The reaction mixture was incubated by gently shaking at 37° C. for 24 hours. The reaction was stopped by adding 450 μL of Clarity OTX lysis-loading buffer (Phenomenex, Cat #ALO-8579) containing internal standard (oligonucleotide U₂₁ at 1 μg/mL final concentration) and frozen at −80° C. until analysis.

Clarity OTX 96-well solid-phase extraction plates were used to enrich the oligonucleotide from the reaction mixture as described by Liu et al., Bioanalysis, 11, 1967-1980 (2018), which is incorporated herein by reference in its entirety. The samples were loaded onto SPE columns preconditioned with methanol followed by 50 mM ammonium acetate with 2 mM sodium azide in HPLC-grade water. 50 mM ammonium acetate in 50/50 (v/v) water and acetonitrile (pH 5.5) was used to wash the columns. Then, oligonucleotides were eluted using elution buffer containing 10 mM EDTA, 100 mM ammonium bicarbonate in 40/10/50 (v/v/v) acetonitrile/tetrahydrofuran/water (pH 8.8). The eluant was dried under nitrogen and resuspended in 120 μL of LC-MS grade water for LC-MS analysis.

30 μL of sample was injected on to Waters X-Bridge BEH C8 XP Column (Cat #176002554, 130 Å, 2.5 μm, 2.1 mm×30 mm, 80° C.) and separated using a gradient of 16 mM triethylamine (Sigma, Cat #471283), 200 mM 1,1,1,3,3,3-hexafluoro-2-propanol (Fisher, Cat #67-56-1) in LC-MS grade water (Fisher, Cat #7732-18-5); mobile phase B was 100% methanol (Fisher, Cat #67-56-1). The gradient started with 1% mobile phase B and progressed to 35% B over 4.3 minutes, then the column was equilibrated with 1% mobile phase B for 1 minute. The data were acquired using full scan mode on high-resolution mass spectrometry (Thermo Scientific Q Exactive). The data were acquired with a scan range of 500-3000 m/z at a resolution setting of 70,000, the spray voltage was 2.8 kV. The auxiliary gas temperature and the capillary temperature were set to 300° C.

All data were processed using ProMass HR Deconvolution software (Novatia, LLC) to identify metabolites as described by Liu et al., Bioanalysis, 11, 1967-1980 (2018), which is incorporated herein by reference in its entirety.

As shown in FIG. 2, no degradation of either the single-stranded oligonucleotides or the parent siRNA strands was detected in plasma for up to 8 hours.

The plasma metabolism summary for the above exemplary single-stranded oligonucleotides and the parent siRNA duplex are shown in FIG. 3.

In metabolite analysis by LC-MS, no significant differences in degradation was observed in plasma and all the single-stranded oligonucleotides remained intact at 24 hours.

The single-stranded oligonucleotides having a loop region containing a triplet of 2′-F (A-492540); a triplet of 2′-deoxy (DNA) (A-492546); a poly dT (A-492548); and a triplet of RNA (A-511271) were observed to have metabolized (cleaved at the linking group L, i.e., the loop region) efficiently to yield a 23mer (e.g., an antisense strand as shown in parent AD-64228) by 24 hours in liver homogenate.

The single-stranded oligonucleotides having a more stable loop region containing 2′-OMe (A-492538, A-492539); Q304 (A-492542); 2′-OMe and PS (A-1700637) did not show the formation of a 23mer at 24 hours in liver homogenate.

The liver homogenate metabolism summary for the above exemplary single-stranded oligonucleotides and the parent siRNA duplex are shown in FIG. 4.

### In Vivo Activity of Single-Stranded Loop Oligonucleotide Conjugates

The inhibition of mTTR expression by the above exemplary single-stranded oligonucleotides (listed in Table 1) in mouse was conducted at a single dosage of 0.2 mg/kg, 0.4 mg/kg, and 1 mg/kg.

Female C57BL/6 mice approximately 8 weeks of age were obtained from Charles River Laboratories and randomly assigned to each group. Mice were acclimated in-house for 48 hours before the study started. Animals were dosed subcutaneously at 10 μL/g with loopmer siRNA, siRNA, or with PBS saline control. The doses used in this study were 0.2, 0.4, and 1 mg/kg. The test compounds were diluted into phosphate buffered saline (PBS, pH 7.4). All solutions were stored at 4° C. until the time of injection. Blood was collected utilizing the retro-orbital eye bleed procedure as per the IACUC-approved protocol. The sample was collected in Becton Dickinson serum separator tubes (Fisher Scientific, Cat #BD365967).

For analysis of TTR, serum samples were kept at room temperature for 1 hour and then spun in a microcentrifuge at 21,000×g at room temperature for 10 minutes. Serum was transferred into 1.5 mL microcentrifuge tubes for storage at −80° C. until the time of assay. Serum samples were diluted at 1:4,000 and assayed using a commercially available kit from ALPCO specific for the detection of mouse prealbumin (Cat #41-PALMS-E01). Protein concentrations (μg/mL) were determined by comparison to a purified TTR standard and the manufacturer's instructions were followed.

The results indicate that these single-stranded oligonucleotides (with GalNAc conjugates) showed efficient silencing in mice. The single-stranded oligonucleotides having a loop region (a linking group L) containing a triplet of 2′-F and a triplet of 2′-deoxy (DNA) showed a comparable or enhanced potency as compared to the parent duplex siRNA, whereas the single-stranded oligonucleotides having a more stable loop region showed a delayed onset of potency.

### Additional Single-Stranded Oligonucleotides

Additional single-stranded loop oligonucleotides targeting SOD1 are shown in Schemes 3.1-3.3. The indications for the chemical modifications are the same as in Table 1. (Uhd) is 2′-O-hexadecyl-uridine-3′-phosphate. Q304 is

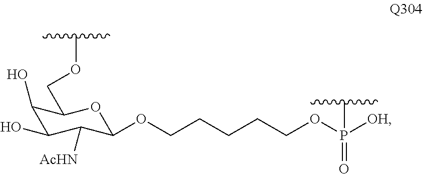 [SMILES: *OCC1OC(OCCCCCOP(*)(=O)O)C(NC(C)=O)C(O)C1O]

### and Y211 is

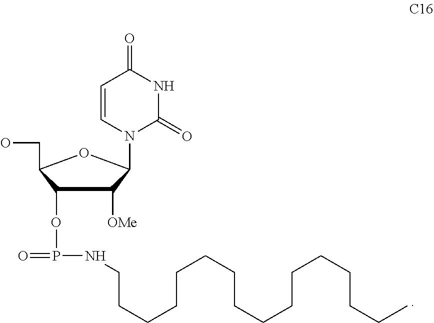

Additional single-stranded loop oligonucleotides targeting β-cat are shown in Schemes 4.1-4.4. The indications for the chemical modifications are the same as in Table 1. L10 is

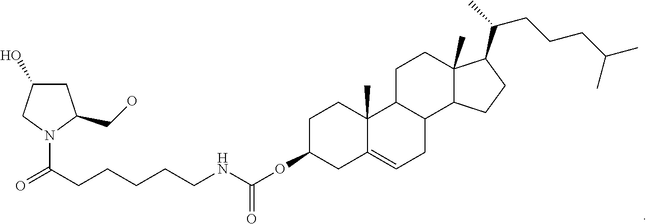 [SMILES: CC(C)CCC[C@@H](C)[C@H]1CCC2C3CC=C4C[C@@H](OC(=O)NCCCCCC(=O)N5C[C@H](O)C[C@H]5CO)CC[C@]4(C)C3CC[C@@]21C]

Additional single-stranded loop oligonucleotides targeting oc-mTTR are shown in Schemes 5.1-5.2. The indications for the chemical modifications are the same as in Table 1.

Additional single-stranded loop oligonucleotides targeting h/cTTR are shown in Schemes 6.1-6.2. The indications for the chemical modifications are the same as in Table 1.

### Example 2: Oligonucleotide Constructs Comprising Two Single-Stranded Loop Oligonucleotides

Various designs for oligonucleotide constructs comprising two single-stranded loop oligonucleotides are shown in Schemes 7.1-7.3. In these schemes, two single-stranded loop oligonucleotides are connected at the loop region by a linker to form a gemini style structure. The linkers connecting the two single-stranded loop oligonucleotides can be any one disclosed in this disclosure. Examples of the linkers are shown in Schemes 7.1-7.3 below. The nucleotide(s) in the loop region(s) of the two single-stranded loop oligonucleotides where the oligonucleotides are connected can take on any chemical modification disclosed in this disclosure to facilitate the linkers to connect the oligonucleotides. Exemplary modifications of the nucleotide(s) in the loop region(s) where the oligonucleotides are connected are shown in Schemes 7.1-7.3 below.

The linkers connecting the two single-stranded loop oligonucleotides may be formed by various precursor molecules such as

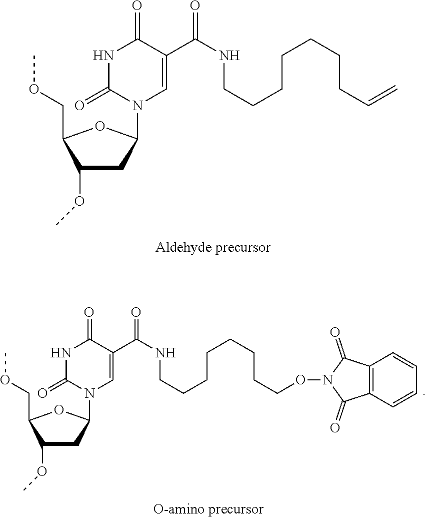

For instance, an exemplary process to form an oligonucleotide construct having a gemini style structure from a single-stranded loop oligonucleotide are shown in Scheme 7.4, wherein the linker connecting the two single-stranded loop oligonucleotides is an oxime linker or an aminooxy linker.

### Example 3: Exemplary Single-Stranded Oligonucleotides

### Chemical Synthesis of GalNAc Conjugated Single-Stranded Oligonucleotide

In this example, single-stranded loop oligonucleotide constructs have been designed where the sense strand and antisense strand were connected by a linker group.

The synthesis procedures for the single-stranded loop oligonucleotides are the same as described above in Example 1. The synthetic scheme of the single-stranded loop oligonucleotide is shown in Scheme 8 below.

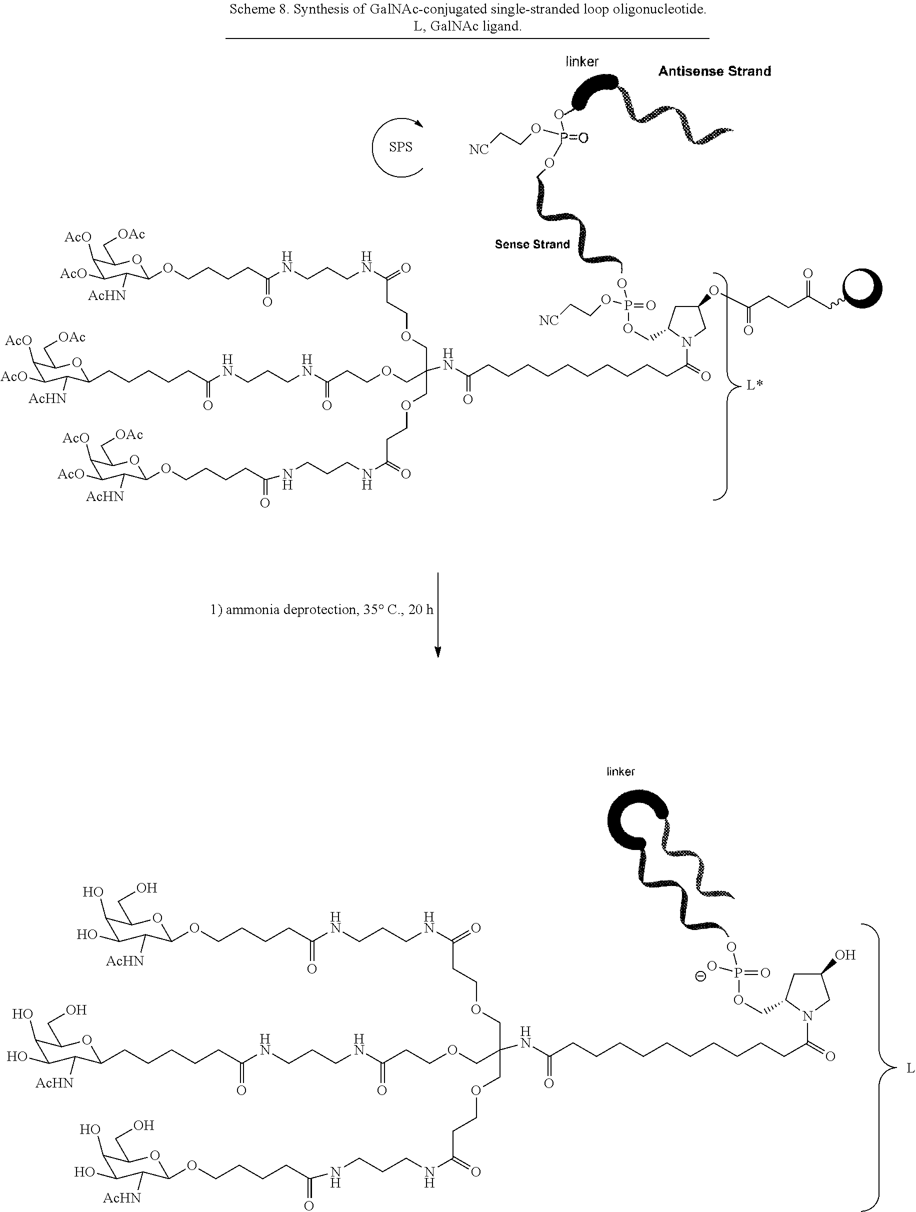

The synthesis of the single-stranded loop oligonucleotides involved only single strand synthesis which can avoid the two-step synthesis and annealing for the synthesis of a typical duplex siRNA. Additionally, the connected structure can enhance metabolic stability without reducing the duplex thermal stability.

### Design of Single-Stranded Loop Oligonucleotide Conjugates

The design of single-stranded chemically modified RNA that can be efficiently loaded into RISC machinery and subsequently knocks down the target gene through the RNAi pathway has been designed in this example. An siRNA duplex containing triantennary GalNAc attached to a 5′ sense strand targeting rodent TTR was used as parent (ON-1) for the design of the single-stranded loop oligonucleotides. A loop region connecting the 5′ end of the sense strand to the 3′ end of the antisense strand was designed with multiple chemistries to to result in loopmers that are stable in circulation and can be cleaved by nucleases after internalization in the liver. The effect of modifying chemistry in the loop region was illustrated by comparing various single-stranded loop oligonucleotides with different loop region stabilities (Table 4).

For instance, one oligonucleotide contains a loop region containing 2′-OMe modified nucleotides (On-2), then three of the nucleotides in the loop region were replaced with less stable 2′-F modified nucleotides (On-3), then the loop was further destabilized by introducing unmodified nucleotides by using either combinations of RNA and DNA bases (On-4, On-5) or complete DNA bases (On-6 and On-7) with 2′-OMe designs. Also, to provide maximal stability phosphorothioates were used in the 2′-OMe modified region (On-8). Additionally, 5′-(E)-vinyl phosphonate was included in the single-stranded loop oligonucleotide to understand the effect on the efficacy in the single-stranded loop oligonucleotide designs for 2′-O Me- and 2′-F—containing single-stranded loop oligonucleotide (On-9, On-10, On-11).

### In Vivo Activity of Single-Stranded Loop Oligonucleotide Conjugates

The efficacy of the single-stranded loop oligonucleotides to knock down TTR in C57BL/6 mice was evaluated. The exemplary single-stranded loop oligonucleotides, along with double-stranded siRNA as a positive control, were administered in a single dose to mice at three different dose levels (1 mg/kg, 0.4 mg/kg, and 0.2 mg/kg).

The experimental protocol for the in vivo activity study in mice are the same as described above in Example 1.

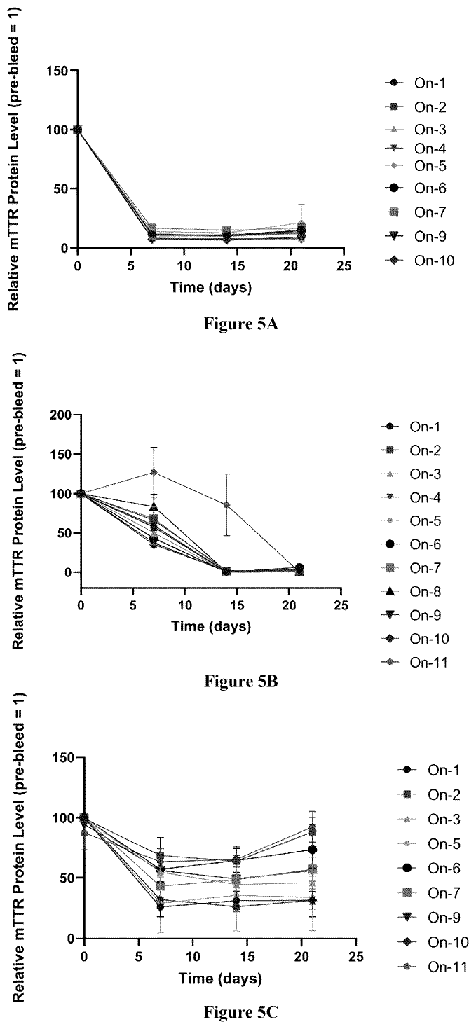

At the highest dose of 1 mg/kg (FIG. 5A), significant knockdown of TTR was achieved by all exemplary single-stranded loop oligonucleotides, but differences in knockdown were minimal to rank order the designs of these exemplary single-stranded loop oligonucleotides. At the next dose level of 0.4 mg/kg (FIG. 5B), ON-11 showed delayed knock-down of TTR protein reaching nadir at 21 days post-dosing, whereas other exemplary single-stranded loop oligonucleotides reached a nadir at day 14 with comparable potency.

At higher doses (1 mg/kg and 0.4 mg/kg), single-stranded loop oligonucleotides demonstrated comparable efficacy to the parent double stranded siRNA.

At the lowest dose of 0.2 mg/mL (FIG. 5C), differences in the potency in these single-stranded loop oligonucleotides were observed. On-5 containing hybrid RNA/DNA loop design and On-13 containing 2′F loop design with 5′-VP showed the highest potency comparable to parent ds-siRNA (80% knockdown of mTTR, FIG. 5C). The single-stranded loop oligonucleotide containing DNA loop design (On-7) showed the next highest potency with ˜60% knockdown followed by On-3 (˜55%), On-6, and On-9 (˜50% KD). Single-stranded loop oligonucleotides containing 2′-OMe (On-2) and 2′-OMe (On-11) with phosphorothioates showed a relative lower potency of ˜30% (On-2) and 20% (On-11), respectively. A delayed activity by On-11 observed at a higher 0.4 mg/kg dose was not observed at 0.2 mg/kg dose.

### In Vitro Stability/Metabolism of the Oligonucleotide-Conjugates in Rat Plasma

The in vitro metabolic stability of the exemplary single-stranded loop oligonucleotides and linear siRNA controls in rat plasma were studied and compared. Rat plasma and liver homogenates were chosen as surrogates for mice plasma and liver homogenates as rat matrices are better at predicting long-term in vivo clearance compared to mice.

The experimental protocol for the in vitro metabolic stability study in rat plasma are the same as described above in Example 1.

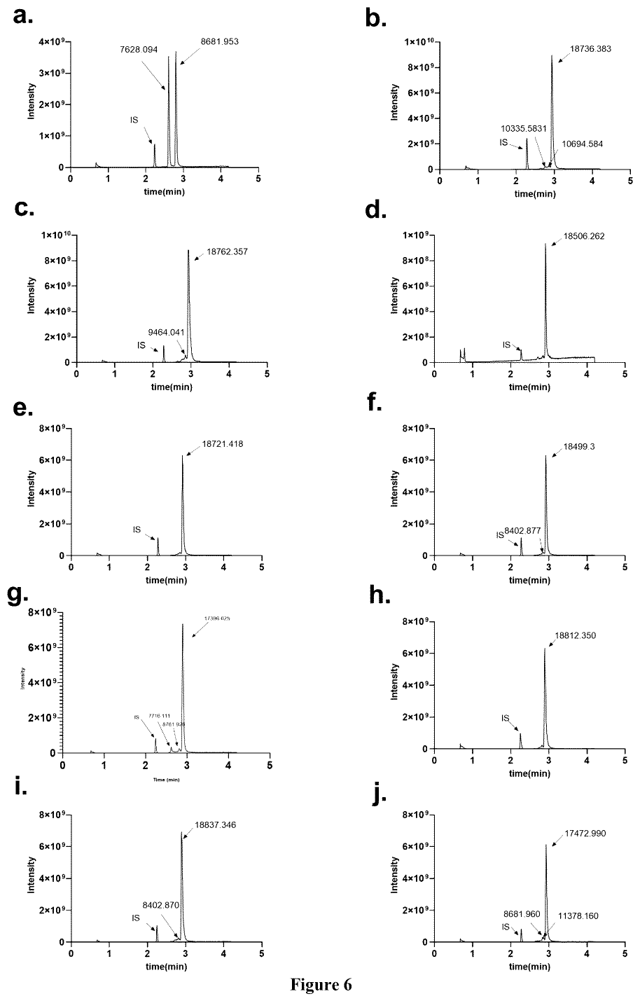

The oligonucleotides were incubated in rat plasma for 24 hours at 37° C. and metabolite profiling was performed using high resolution liquid chromatography and mass spectrometry. The results are summarized in FIG. 6 and Table 5.

No significant metabolism was observed in plasma for all the exemplary single-stranded loop oligonucleotides and the controls, the majority of the parent remained intact after 24 hours (>99%).

### In Vitro Stability/Metabolism of the Oligonucleotide-Conjugates in Rat Liver Homogenate

The in vitro metabolic stability of the exemplary single-stranded loop oligonucleotides and linear siRNA controls in rat liver homogenates were studied and compared.

The experimental protocol for the in vitro metabolic stability study in rat liver homogenates are the same as described above in Example 1.

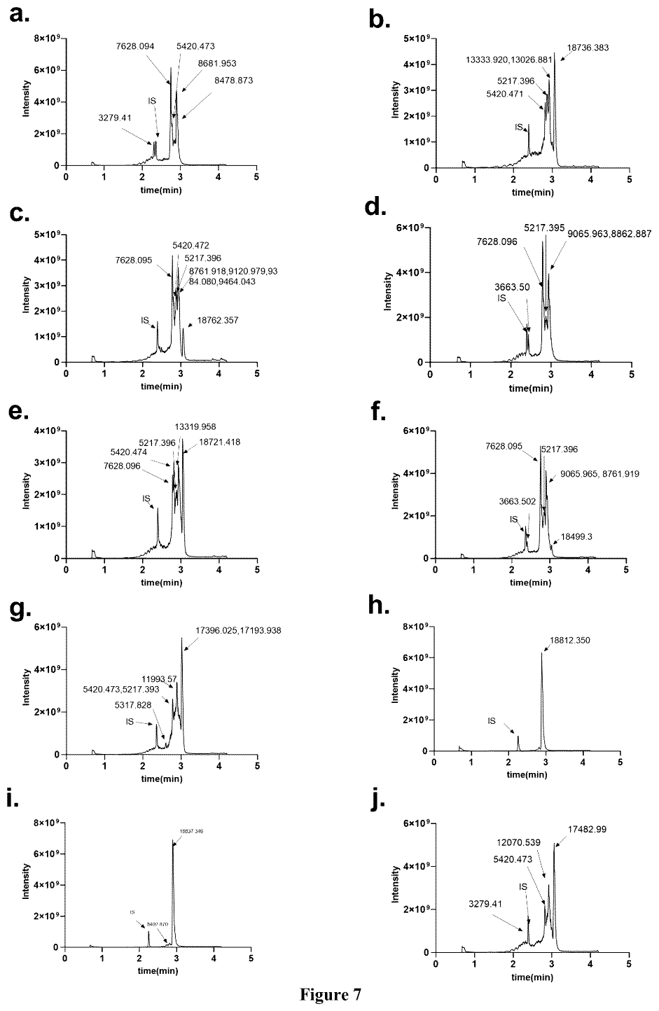

The oligonucleotides were incubated in rat liver homogenates for 24 hours at 37° C. and metabolite profiling was performed using high resolution liquid chromatography and mass spectrometry. The results are summarized in FIG. 7 and Table 6.

No significant metabolism was observed for parent ds-siRNA (On-1) antisense strand. Loss of one GalNAc was a major antisense metabolite observed for On-1 (FIG. 7a). No metabolism was observed in the loop region of On-2. Major metabolites observed for On-2 are with cleavages near the 39-41 region of the single-stranded loop oligonucleotide; this region corresponds to the sense strand fluoro triplet region nucleotide 9-11 in On-1 (ds-siRNA control). The major metabolite observed for On-3 (FIG. 7b) is nucleotide 23, corresponding to the antisense strand in control On-1 (7628.094 Da). Multiple metabolites were observed in the loop region at nucleotides 29, 30, 31, and 32 releasing ds-siRNA as the major product. The major metabolite for On-4 (FIG. 7c) was again 23 mer antisense strand (7628.094 Da). The single-stranded loop oligonucleotide was cleaved at position 30 releasing 22 mer corresponding sense as the major metabolite. On-5 (FIG. 7d) containing a hybrid RNA-DNA loop design released 23 mer as the metabolite but the cleavage on the sense strand was not observed at position 31; instead, cleavage was observed at position 30 (8862.887), leaving additional dT on the sense strand of the released product. On-6 (FIG. 7e) with DNA triplet also released 23 mer corresponding antisense strand as the metabolite, but the cleavage on sense strand was not observed. On-7 containing multiple deoxythymidine (dT) loop region (FIG. 7f) showed clear cleavage at position 23 (mass 7628.094 Da) and position 31 (8761.919 Da) corresponding to 21 mer sense strand in On-1. No cleavage in the loop region was observed for On-8 (FIG. 7g) containing phosphorothioates in the loop region indicating a stable loop, and multiple minor metabolites were observed in non-loop regions of siRNA next to fluoro nucleotides. On-9 (FIG. 7h), 5′-(E)-vinyl phosphonate containing counterpart of On-2, showed a similar metabolism pattern as On-2 with no metabolism in 2′-OMe containing loop region. On-10 (FIG. 7i), 5′-(E)-vinyl phosphonate containing counterpart of On-3, showed a similar metabolism pattern as On-3, releasing 23 (7704.066) and 21 mer (8761.919 Da) antisense and sense strands, respectively, and multiple cleavages at 29, 30, and 32 nucleotides. On-11 (FIG. 7j), 5′-(E)-vinyl phosphonate containing counterpart of On-8, similarly showed no metabolism in 2′-OMe and phosphorothioate containing loop region.

Correlation of LC-MS Intensity to mTTR Knockdown.

Correlation analysis was performed using GraphPad Prism 8.4.3 (GraphPad Software, San Diego, California USA). Pearson's correlation was performed to correlate either intensity of parent loopmerRNA or antisense 22/23 mer formed with mTTR knockdown and two-tailed P value was calculated. P value of less than 0.05 was considered statistically significant.

The single-stranded loop oligonucleotide On-5 containing RNA-DNA hybrid loop region showed most potency in vivo comparable to parent ds-siRNA control ˜75% KD; in vitro metabolism showed the complete metabolism of loop region (no full-length parent was observed) releasing 23mer antisense and 22mer sense strand. On-7, with complete DNA-containing loop region was ranked second in potency ˜₆₀% KD, also demonstrated complete break down into 23 mer and 21mer antisense and sense strands in liver homogenates, respectively. On-3 (2′-F triplet with 2′-OMe) and On-9 (DNA triplet 2′-O Me) showed 50% potency and correlated well with the liver homogenate, showing a significant amount of the single-stranded loop oligonucleotides stable after 24 hours. The single-stranded loop oligonucleotides showing a minimal release of 23mer antisense (On-3, 2′O Me loop; On-8, 2′OMe and phosphorothioate loop) in liver homogenate has relatively low in vivo efficacy (<25% knockdown).

The intensity of the extracted ion chromatographic peaks from the in vitro liver homogenate metabolism stability experiments discussed above was plotted against the in vivo mTTR knockdown data. To differentiate and understand the structure-activity relationship of the single-stranded loop oligonucleotides, a lower 0.2 mg/kg dose group was used.

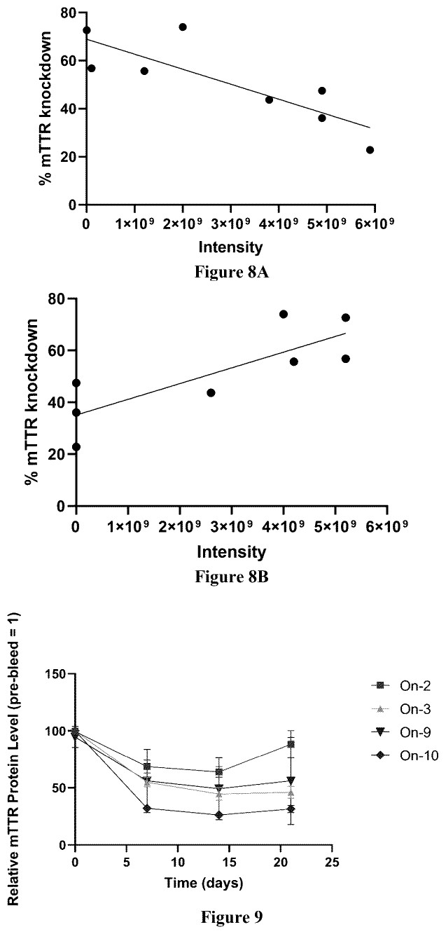

In the first condition, loss of parent RNA was followed and the intensity correlated to mean mTTR knockdown at nadir from mice at 0.2 mg/kg dose. A statistically significant negative correlation was observed with pearson r of −0.8300 (P=0.01) (FIG. 8A) between the intact single-stranded loop oligonucleotide intensity and % mTTR knockdown, indicating that the presence of a higher concentration of intact single-stranded loop oligonucleotide limits the availability for RISC loading, thereby showing decreased knockdown in vivo.

In the second condition, the formation of 22/23mer metabolite corresponding to antisense strand in the ds-siRNA was correlated with % KD. A statistically significant positive correlation was observed with pearson r of 0.8118 (P=0.04) (FIG. 8B) between the formation of the antisense metabolite and % mTTR knockdown, indicating that the formation of an antisense 22/23 mer metabolite is essential for in vivo efficacy. The figure also showed that the concentration of the metabolite formation correlated well with the knockdown efficacy.

These results indicate that the nuclease cleavage of the loop region of the single-stranded loop oligonucleotide to release double standard siRNA is essential for efficient target knockdown.

Addition of 5′-VP enhanced the potency in both 2′OMe-containing On-9 and 2′F containing-On-10 (FIG. 9B). No metabolism differences for 2′OMe-containing sequences On-2 and On-9 were observed; in particular, the release of the 23 mer antisense was not observed. An increase in the number of cleavage sites was observed in 2′-F-containing sequence On-10, as compared to the version without the 5′-VP modification (On-3). In both single-stranded loop oligonucleotides, the 23mer was released with or without the 5′-VP modification.

In summary, the results indicate that introducing natural DNA and RNA nucleobases or 2′-F nucleobases in the loop region of the single-stranded loop oligonucleotides destabilized the loop leading to an efficient nuclease cleavage and a release of double-stranded siRNA, which was further loaded into RISC and caused gene silencing. This increase in the metabolism of the loop region therefore releases the siRNA duplex for an efficient gene knockdown. Using modifications such as 2′-OMe and phosphorothioate made the loop region stable and less susceptible to nuclease cleavage, thereby reducing the activity of the single-stranded loop oligonucleotides. Addition of 5′-VP enhanced the activity of the single-stranded loop oligonucleotides.

### Example 4. Exemplary Single-Stranded Oligonucleotides

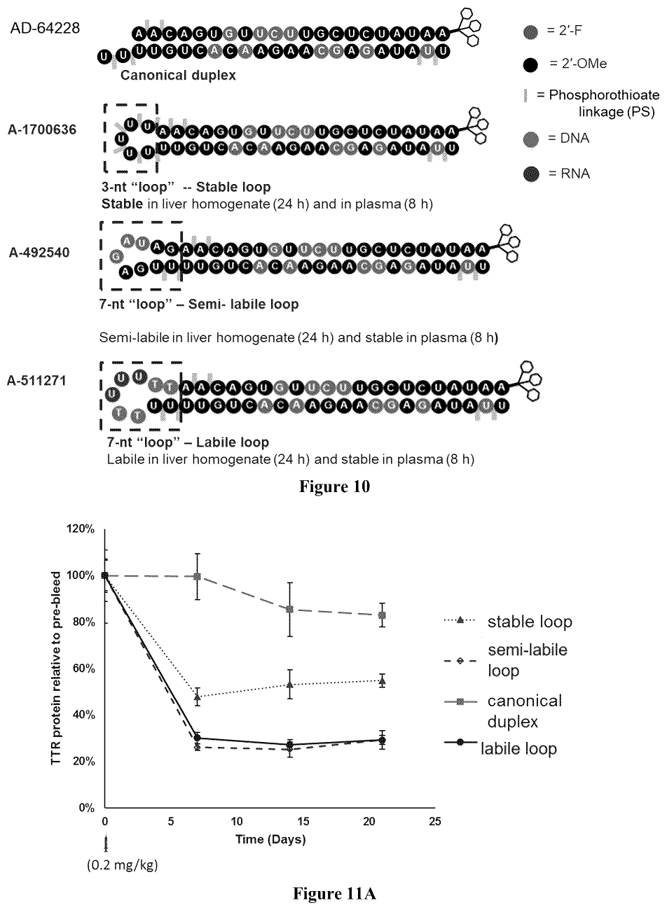

In this example, different sizes and chemistry in the “loop” linkers are illustrated to impart different properties to the single-stranded loop oligonucleotides. The oligonucleotides used in this example are shown in FIG. 10. Their sequences are listed and characterized in Table 1 above. The sequences of the parent siRNA duplex are shown in Table 3 above. The synthesis procedures for the single-stranded loop oligonucleotides are the same as described above in Example 1.

The in vitro metabolism of all above exemplary single-stranded oligonucleotides were assessed in mouse plasma and liver homogenates, and compared to the parent siRNA duplex. The experimental protocol for the in vitro metabolism study are the same as described above in Example 1.

The inhibition of mTTR expression by the above exemplary single-stranded oligonucleotides in a mouse was conducted at a single dosage of 0.2 mg/kg over 20 days. The experimental protocol for the in vivo knockdown study are the same as described above in Example 1.

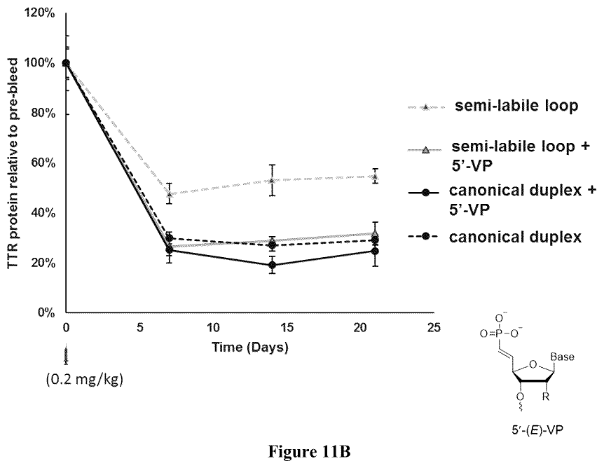

The results are shown in FIGS. 11A-11B. FIG. 11A shows the inhibition of mTTR expression by the above exemplary single-stranded oligonucleotides (listed in FIG. 10) in a mouse at a single dosage of 0.2 mg/kg. FIG. 11B shows the inhibition of the mTTR expression by certain exemplary single-stranded oligonucleotides (listed in FIG. 10) in a mouse at a single dosage of 0.2 mg/kg, as compared to the same oligonucleotide but with a 5′-(E)-vinylphosphonate (VP) modification. The results indicate that the 5′-(E)-(VP) modification can help recover the loss in potency in mice when the loop of the single-stranded oligonucleotide is semi-labile. These results also indicate that the intracellular loop cleavage in the single-stranded oligonucleotides supports the in vivo knockdown activities.

### Example 5. Exemplary Single-Stranded Oligonucleotides

In this example, exemplary single-stranded loop oligonucleotides were constructed having a cleavable linker connecting a sense and antisense strand at 5′ and 3′ end, respectively. The cleavable linker can act as a prodrug: when the linker is cleaved in tissue, it can release the parent double-stranded siRNA. The sequences of the single-stranded loop oligonucleotides used in this example are shown in Table 7 below; the sequences of the parent siRNA duplex are also shown in Table 7 below. The synthesis procedures for the single-stranded loop oligonucleotides are similar as those described above in Example 1.

The inhibition of mTTR expression by the above exemplary single-stranded oligonucleotides to knock down TTR in C57BL/6 mice was evaluated, according to the in vivo evaluation protocol and dosage regime according to Table 8. The experimental protocol for the in vivo activity study in mice are similar to those as described above in Example 1.

Briefly, the exemplary single-stranded loop oligonucleotides were administered intravitreally in a single dose to female C57BL/6 mice at the dose level of 2.5 mg/ml. The parent double-stranded siRNA duplex (with no loop) was administered as a control. Two non-target double-stranded siRNA duplexes (with no loop) were also administered as controls. The tissues from whole eye were collected and flash frozen on D14. The results of the TTR mRNA knockdown in mouse whole eye tissue on D14 by these exemplary single-stranded loop oligonucleotides and siRNA duplex controls were analyzed by qPCR

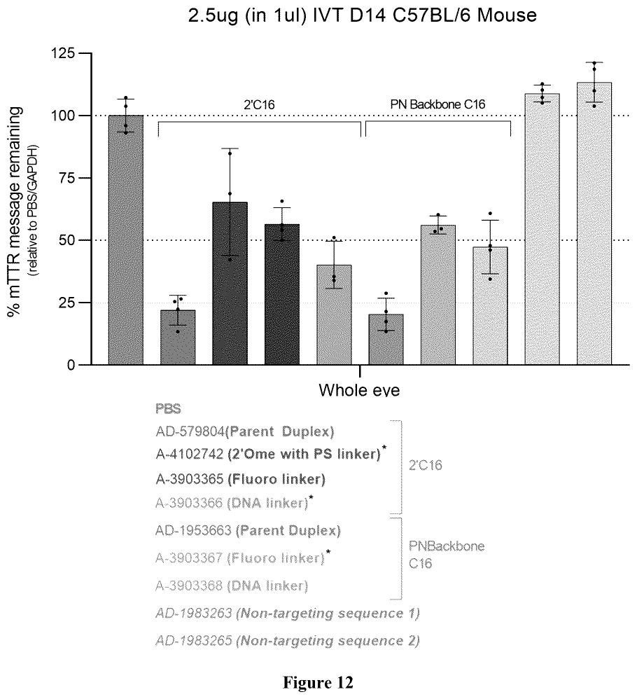

The results are shown in FIG. 12. For the oligonucleotides having the 2′ C16 backbone design, the single-stranded loop oligonucleotide containing a DNA loop region showed most potency in vivo activity (˜60% KD), although not as good as the parent ds-siRNA control (˜75% KD). The oligonucleotides having PN backbone C16 design showed the similar trend. The overall trend of the in vivo activity by the exemplary single-stranded loop oligonucleotides containing different loop regions were: DNA loop region >2′-F triplet with 2′-OMe>2′-OMe with PS.

The single-stranded loop oligonucleotide containing 2′-OMe with PS loop region showed relatively low in vivo efficacy (e.g., A-3903366, ˜30% KD), perhaps due to its “non-cleavable” nature.

TABLE 8
Protocol and dosage regime for in vivo knockdown of TTR in mice the parent siRNA duplex
targeting mTTR, non-target control duplex, and single-stranded loop RNAs targeting mTTR

| | | | | | | | Dose | | |
|---|---|---|---|---|---|---|---|---|---|
| | Treatment | | | | N (20 | | Route/ | Time | Tissues/ |
| Group | (TTR) | Conjugate | Loop | Dose | mice) | Animal | Regimen | point | Analysis |
| 1 | PBS | — | | — | 2 (4 | Female | D 0 | D 14 | Flash |
| 2 | AD-579804.29 | 2′ C16 | None | 2.5 ug | eyes) | C57BL6 | Single | | frozen |
| | | (Uhd) | (parent) | in 1 ul | | Mouse | IVT | | (qPCR): |
| 3 | A-4102742.1 | | 2′Ome | (2.5 | | | Dose | | Whole |
| | | | with PS | mg/ml) | | | | | Eye (40 |
| 4 | A-3903365.1 | | Fluoro | | | | | | samples) |
| 5 | A-3903366.1 | | DNA | | | | | | |
| 6 | AD-1953663.2 | PN- | None | | | | | | |
| | | Backbone | (parent) | | | | | | |
| 7 | A-3903367.1 | C16 (Y211) | Fluoro | | | | | | |
| 8 | A-3903368.1 | | DNA | | | | | | |
| | AD-1983263.2 | 2′ C16 | N/A, non- | | | | | | |
| | AD-1983265.2 | (Uhd) | targeting | | | | | | |
| | | | control | | | | | | |
| | | | duplex | | | | | | |

1. A single-stranded oligonucleotide according to formula (I): 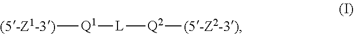
wherein:
Z¹ is a first oligonucleotide, comprising 10-100 optionally modified nucleotides that is substantially complementary to a target gene;
Z² is a second oligonucleotide, comprising 10-100 optionally modified nucleotides that is substantially complementary to Z¹;
Z¹ and Z² are capable of forming an intra-strand duplexed region comprising 3 or more consecutive base pairs;
L is a linking group;
Q¹ and Q² each independently represent 0 to 12 optionally modified nucleotides; and
at least one nucleotide in formula (I) is a modified nucleotide.

2-4. (canceled)

5. The single-stranded oligonucleotide of claim 1, wherein L contains a linking moiety represented by a formula: #—(N)ₙ—**,
wherein:
# is the bond to Q¹ and ** is the bond to Q²;
n is 3 to 12; and
each N is independently a linking monomer having a chain length of 3 or more atoms.

6. The single-stranded oligonucleotide of claim 5, wherein one or more N is an optionally modified nucleotide.

7. The single-stranded oligonucleotide of claim 6, wherein one or more N is independently selected from the group consisting of a 2′-deoxynucleotide (dN), a 2′-deoxy-2′-fluoronucleotide (fN), a ribonucleotide (rN), 2′-O-methylnucleotide (mN), and 2′-aranucleotide (aN).

8-9. (canceled)

10. The single-stranded oligonucleotide of claim 5, wherein one or more N is independently selected from the group consisting of:

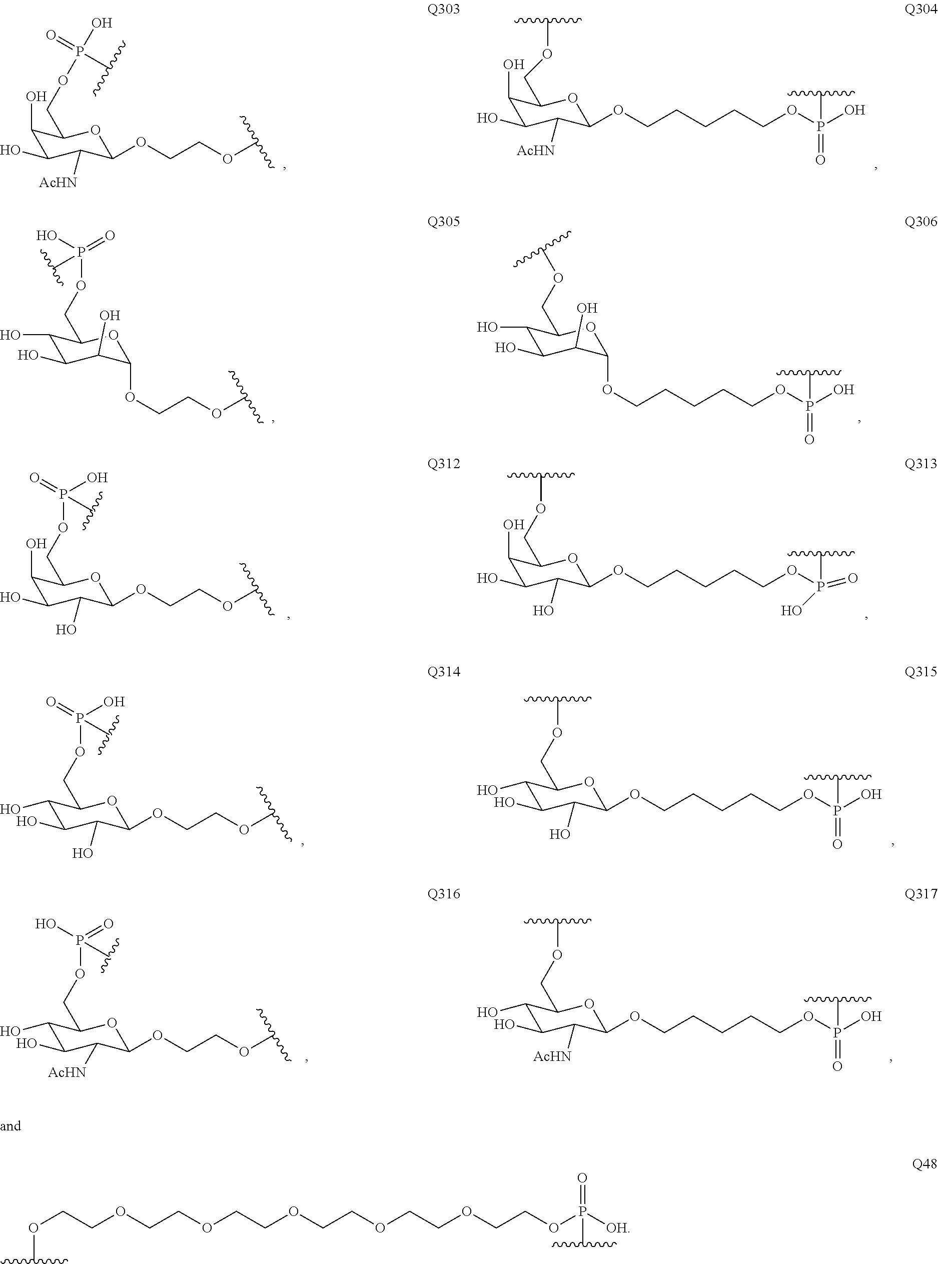

11. The single-stranded oligonucleotide of claim 5, wherein one or more N is independently selected from the group consisting of: 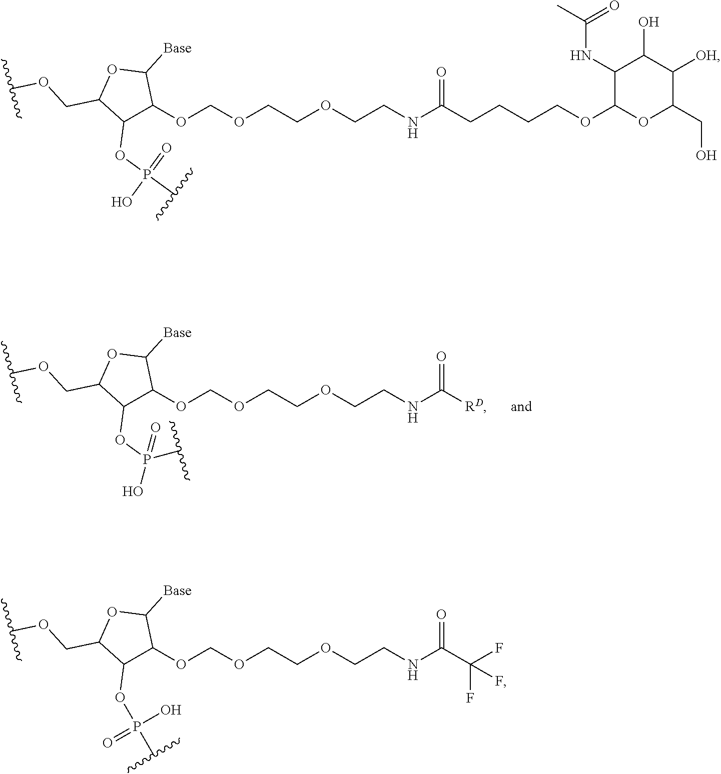
wherein:
Base is an optionally modified nucleobase, and
R^{D} is a C₄₋₃₀ alkyl, C₄₋₃₀ alkyenyl, or C₄₋₃₀ alkynyl.

12. The single-stranded oligonucleotide of claim 5, wherein one or more N comprises a mono-, di-, tri-, tetra-, penta- or poly-prolinol, optionally conjugated with a ligand; a mono-, di-, tri-, tetra-, penta- or poly-hydroxyprolinol, optionally conjugated with a ligand; an optionally modified nucleotide; or combinations thereof.

13. The single-stranded oligonucleotide of claim 12, wherein one or more N comprises a moiety selected from the group consisting of:

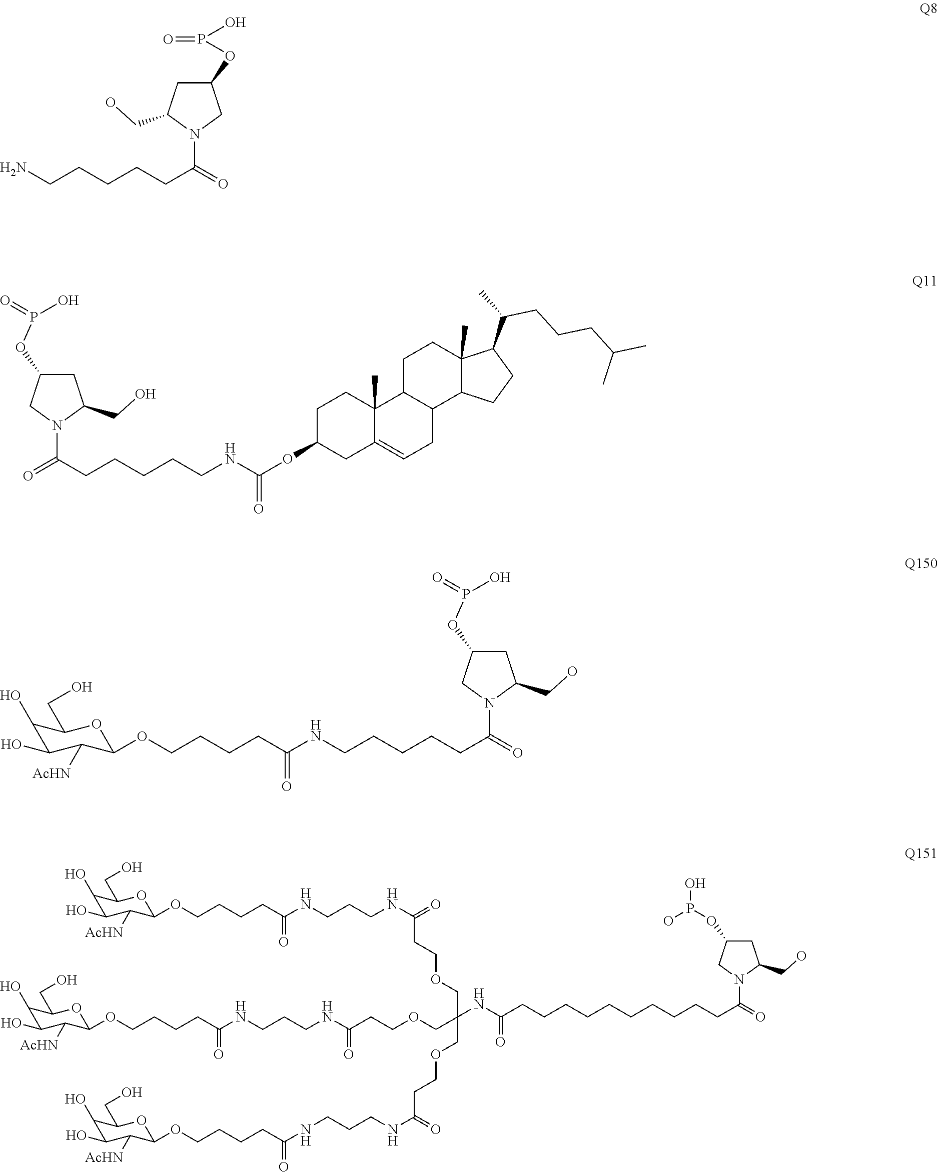

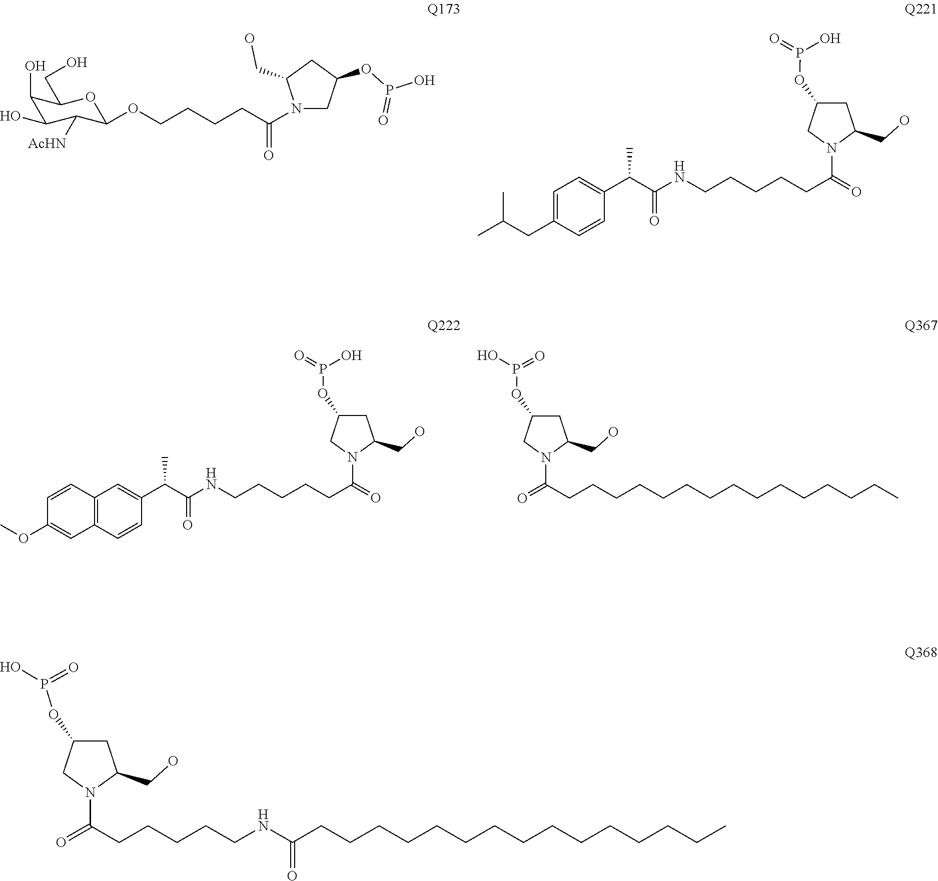

14. The single-stranded oligonucleotide of claim 1, wherein L contains a linking moiety represented by a formula: #—(N)ₙ—**,
wherein:
# is the bond to Q¹ and ** is the bond to Q²;
n is 3 to 10; and
each N is independently an optionally modified nucleotide, Y16, Y34, Q48, Q303, Q304, Q305, Q306, Q312, Q313, Q314, Q315, Q316, Q317, Q8, Q11, Q150, Q151, Q173, Q221, Q222, Q367, or Q368.

15. The single-stranded oligonucleotide of claim 14, wherein n is 4-8.

16. The single-stranded oligonucleotide of claim 14, wherein n is 5.

17. The single-stranded oligonucleotide of claim 14, wherein L contains 3-5 of 2′-deoxy nucleotides, a triplet of 2′-deoxy-2′-fluoro nucleotides, a triplet of ribonucleotides, a triplet of 2′-O-methyl nucleotides, or a triplet of Q304.

18. The single-stranded oligonucleotide of claim 14, wherein L is selected from the group consisting of:
#-mN-mN-mN-mN-mN-**
#-rN-rN-rN-rN-rN-**,
#-rN-rN-fN-fN-fN-**,
#-dN-dN-fN-fN-fN-**
#-dN-rN-rN-rN-dN-**,
#-dN-dN-dN-dN-dN-**,
#-mN-mN-dN-dN-dN-**,
#-mN-mN-rN-dN-dN-**,
#-mN-mN-rN-rN-rN-**, and
#-mN-mN-fN-fN-fN-**,
wherein:
dN represents a 2′-deoxy nucleotide,
fN represents a 2′-deoxy-2′-fluoro nucleotide,
rN represents a ribonucleotide, and
mN represents a 2′-O-methyl nucleotide.

19. The single-stranded oligonucleotide of claim 14, wherein L is selected from the group consisting of:
#---mN-mN-Q304-Q304-Q304---**
#---dN-dN-Q304-Q304-Q304---**
#---dN-rN-Q304-Q304-Q304---**
#---rN-dN-Q304-Q304-Q304---**, and
#---dN-rN-Q304-Q304-Q304---**
wherein:
dN represents a 2′-deoxy nucleotide,
fN represents a 2′-deoxy-2′-fluoro nucleotide,
rN represents a ribonucleotide, and
mN represents a 2′-O-methyl nucleotide.

20. The single-stranded oligonucleotide of claim 18, wherein one or more internucleotide linkages between the nucleotides in L are modified internucleotide linkages independently selected from the group consisting of a phosphodiester, phosphotriester (optionally comprising the linkage phosphorus atom in either Rp configuration or Sp configuration), hydrogen phosphonate, alkyl or aryl phosphonate, phosphoramidate (optionally comprising the linkage phosphorus atom in either Rp configuration or Sp configuration), phosphorothioate (optionally comprising the linkage phosphorus atom in either Rp configuration or Sp configuration), and a nitrogen-modified phosphorous-containing linkage (PN-linkage) (optionally comprising the linkage phosphorus atom in either Rp configuration or Sp configuration).

21. The single-stranded oligonucleotide of claim 1, further comprising one or more ligands.

22-24. (canceled)

25. The single-stranded oligonucleotide of claim 1, wherein one or more of the five internucleotide linkages among the six 3′-terminal nucleotides, or among the six 5′-terminal nucleotides is a modified internucleotide linkage.

26. (canceled)

27. The single-stranded oligonucleotide of claim 1, wherein one or more of the five internucleotide linkages among the six 5′-terminal nucleotides of Z² or among the six 5′-terminal nucleotides of Z¹ is a modified internucleotide linkage.

28. (canceled)

29. The single-stranded oligonucleotide of any one of claims 1-16, further comprising one or more modified internucleotide linkage between the 3′-terminal nucleotide of Z¹ and the first nucleotide of Q¹, and/or one or more modified internucleotide linkage between the nucleotides of Q¹.

30. (canceled)

31. The single-stranded oligonucleotide of claim 1, characterized by one or more of:
(a) Z¹ and Z² each independently contain 19-23 optionally modified nucleotides;
(b) Q¹ and Q² each independently contain 0 to 2 optionally modified nucleotides;
(c) the duplexed region formed by Z¹ and Z² contains no more than 3 mismatched base pairs;
(d) the duplexed region formed by Z¹ and Z² forms a blunt end;
(e) at least one nucleotide in Z² is a modified nucleotide;
(f) at least one nucleotide in Z¹ is a modified nucleotide;
(g) Z² comprises at least one modified internucleotide linkage;
(h) Z¹ comprises at least one modified internucleotide linkage;
(i) the 5′-terminal nucleotide comprises a 5′-phosphate or 5′-phosphate mimic modification;
(j) the 3′-terminal nucleotide is conjugated to a ligand, optionally through a linker;
(k) Z¹ contains no more than 3 mismatches to the target gene; and
(l) L contains a linking moiety represented by a formula: #—(N)ₙ—**, wherein n is 3 to 5, and each N is independently an optionally modified nucleotide, Q48, Q303, Q304, Q305, Q306, Q312, Q313, Q314, Q315, Q316, Q317, Q8, Q11, Q150, Q151, Q173, Q221, Q222, Q367, or Q368.

32. The single-stranded oligonucleotide of claim 31, characterized by one or more of:
(a) Z¹ and Z² each independently contain 21 optionally modified nucleotides;
(b) Q¹ and Q² each independently contain 2 optionally modified nucleotides;
(c) the duplexed region formed by Z¹ and Z² contains no more than 3 mismatched base pairs;
(d) the duplexed region formed by Z¹ and Z² forms a blunt end;
(e) all the nucleotides in Z² are modified nucleotides;
(f) all the nucleotides in Z¹ are modified nucleotides;
(g) Z² comprises at least two consecutive modified internucleotide linkages;
(h) Z¹ comprises at least two consecutive modified internucleotide linkages;
(i) the 5′-terminal nucleotide of Z¹ comprises a 5′-phosphate or 5′-phosphate mimic modification;
(j) the 3′-terminal nucleotide of Z² is conjugated to a ligand, optionally through a linker; and
(k) Z¹ contains no more than 3 mismatches to the target gene; and
(l) L contains a linking moiety represented by a formula: #—(N)ₙ—**, wherein n is 5, and each N is independently an optionally modified nucleotide, or Q304.

33. The single-stranded oligonucleotide of claim 1, wherein Z¹ and Z² each independently comprise 15-40 optionally modified nucleotides.

34-36. (canceled)

37. The single-stranded oligonucleotide of claim 1, wherein Q¹ and Q² are each independently 1 to 6 optionally modified nucleotides.

38-40. (canceled)


SEQUENCE LISTING (136 sequences)

SEQ ID NO: 1 - protein, 8 aa, unidentified
GRGDSPKC

SEQ ID NO: 2 - protein, 5 aa, unidentified
  SITE 4: note = D-phenylalanine
  REGION 1..5: note = circular
RGDFC

SEQ ID NO: 3 - protein, 29 aa, unidentified
AALEALAEALEALAEALEALAEAAAAGGC

SEQ ID NO: 4 - protein, 30 aa, unidentified
AALAEALAEALAEALAEALAEALAAAAGGC

SEQ ID NO: 5 - protein, 15 aa, unidentified
ALEALAEALEALAEA

SEQ ID NO: 6 - protein, 22 aa, unidentified
GLFEAIEGFIENGWEGMIWDYG

SEQ ID NO: 7 - protein, 23 aa, unidentified
GLFGAIAGFIENGWEGMIDGWYG

SEQ ID NO: 8 - protein, 48 aa, Una virus
GLFEAIEGFIENGWEGMIDGWYGCGLFEAIEGFIENGWEGMIDGWYGC

SEQ ID NO: 9 - protein, 44 aa, unidentified
GLFEAIEGFIENGWEGMIDGGCGLFEAIEGFIENGWEGMIDGGC

SEQ ID NO: 10 - protein, 35 aa, unidentified
GLFGALAEALAEALAEHLAEALAEALEALAAGGSC

SEQ ID NO: 11 - protein, 34 aa, unidentified
GLFEAIEGFIENGWEGLAEALAEALEALAAGGSC

SEQ ID NO: 12 - protein, 41 aa, unidentified
  SITE 17: note = Nle
  SITE 38: note = Nle
GLFEAIEGFIENGWEGXIDGKGLFEAIEGFIENGWEGXIDG

SEQ ID NO: 13 - protein, 19 aa, unidentified
LFEALLELLESLWELLLEA

SEQ ID NO: 14 - protein, 20 aa, unidentified
GLFKALLKLLKSLWKLLLKA

SEQ ID NO: 15 - protein, 20 aa, unidentified
GLFRALLRLLRSLWRLLLRA

SEQ ID NO: 16 - protein, 30 aa, unidentified
WEAKLAKALAKALAKHLAKALAKALKACEA

SEQ ID NO: 17 - protein, 22 aa, unidentified
GLFFEAIAEFIEGGWEGLIEGC

SEQ ID NO: 18 - protein, 26 aa, unidentified
GIGAVLKVLTTGLPALISWIKRKRQQ

SEQ ID NO: 19 - protein, 8 aa, unidentified
HHHHHWYG

SEQ ID NO: 20 - protein, 10 aa, unidentified
CHKKKKKKHC

SEQ ID NO: 21 - protein, 16 aa, unidentified
RQIKIWFQNRRMKWKK

SEQ ID NO: 22 - protein, 14 aa, unidentified
GRKKRRQRRRPPQC

SEQ ID NO: 23 - protein, 27 aa, unidentified
GALFLGWLGAAGSTMGAWSQPKKKRKV

SEQ ID NO: 24 - protein, 18 aa, unidentified
LLIILRRRIRKQAHAHSK

SEQ ID NO: 25 - protein, 26 aa, unidentified
GWTLNSAGYLLKINLKALAALAKKIL

SEQ ID NO: 26 - protein, 18 aa, unidentified
KLALKLALKALKAALKLA

SEQ ID NO: 27 - protein, 9 aa, unidentified
RRRRRRRRR

SEQ ID NO: 28 - protein, 10 aa, unidentified
KFFKFFKFFK

SEQ ID NO: 29 - protein, 37 aa, unidentified
LLGDFFRKSKEKIGKEFKRIVQRIKDFLRNLVPRTES

SEQ ID NO: 30 - protein, 31 aa, unidentified
SWLSKTAKKLENSAKKRISEGIAIAIQGGPR

SEQ ID NO: 31 - protein, 30 aa, unidentified
ACYCRIPACIAGERRYGTCIYQGRLWAFCC

SEQ ID NO: 32 - protein, 36 aa, unidentified
DHYNCVSSGGQCLYSACPIFTKIQGTCYRGKAKCCK

SEQ ID NO: 33 - protein, 42 aa, unidentified
  SITE 42: note = C-terminal -NH2
RRRPRPPYLPRPRPPPFFPPRLPPRIPPGFPPRFPPRFPGKR

SEQ ID NO: 34 - protein, 13 aa, unidentified
  SITE 13: note = C-terminal -NH2
ILPWKWPWWPWRR

SEQ ID NO: 35 - protein, 16 aa, unidentified
AAVALLPAVLLALLAP

SEQ ID NO: 36 - protein, 11 aa, unidentified
AALLPVLLAAP

SEQ ID NO: 37 - protein, 12 aa, unidentified
RKCRIVVIRVCR

SEQ ID NO: 38 - RNA, 51 nt, synthetic construct
  source 1..51: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate
  modified_base 26: mod_base = OTHER; note = 2'-O-methyluridine-3'-phosphate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-methyluridine-3'-phosphate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine-3'-phosphate
  modified_base 40: mod_base = OTHER; note = 2'-deoxy-2'-fluorocytidine-3'-phosphate
  modified_base 41: mod_base = OTHER; note = 2'-deoxy-2'-fluorouridine-3'-phosphate
  modified_base 42: mod_base = OTHER; note = 2'-O-methyluridine-3'-phosphate
  modified_base 43: mod_base = OTHER; note = 2'-O-methylguanosine-3'-phosphate
  modified_base 44: mod_base = OTHER; note = 2'-O-methylcytidine-3'-phosphate
  modified_base 45: mod_base = OTHER; note = 2'-O-methyluridine-3'-phosphate
  modified_base 46: mod_base = OTHER; note = 2'-O-methylcytidine-3'-phosphate
  modified_base 47: mod_base = OTHER; note = 2'-O-methyluridine-3'-phosphate
  modified_base 48: mod_base = OTHER; note = 2'-O-methyladenosine-3'-phosphate
  modified_base 49: mod_base = OTHER; note = 2'-O-methyluridine-3'-phosphate
  modified_base 50: mod_base = OTHER; note = 2'-O-methyladenosine-3'-phosphate
  modified_base 51: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
ttatagagcaagaacactgttttgatttagaacagtgttcttgctctataa

SEQ ID NO: 39 - RNA, 51 nt, synthetic construct; identical to SEQ ID NO: 38
  source 1..51: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate
  modified_base 26: mod_base = OTHER; note = 2'-O-methyluridine-3'-phosphate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-methyluridine-3'-phosphate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine-3'-phosphate
  modified_base 40: mod_base = OTHER; note = 2'-deoxy-2'-fluorocytidine-3'-phosphate
  modified_base 41: mod_base = OTHER; note = 2'-deoxy-2'-fluorouridine-3'-phosphate
  modified_base 42: mod_base = OTHER; note = 2'-O-methyluridine-3'-phosphate
  modified_base 43: mod_base = OTHER; note = 2'-O-methylguanosine-3'-phosphate
  modified_base 44: mod_base = OTHER; note = 2'-O-methylcytidine-3'-phosphate
  modified_base 45: mod_base = OTHER; note = 2'-O-methyluridine-3'-phosphate
  modified_base 46: mod_base = OTHER; note = 2'-O-methylcytidine-3'-phosphate
  modified_base 47: mod_base = OTHER; note = 2'-O-methyluridine-3'-phosphate
  modified_base 48: mod_base = OTHER; note = 2'-O-methyladenosine-3'-phosphate
  modified_base 49: mod_base = OTHER; note = 2'-O-methyluridine-3'-phosphate
  modified_base 50: mod_base = OTHER; note = 2'-O-methyladenosine-3'-phosphate
  modified_base 51: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester

SEQ ID NO: 40 - RNA, 51 nt, synthetic construct
  source 1..51: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate
  modified_base 26: mod_base = OTHER; note = 2'-deoxy-2'-fluoroguanosine-3'-phosphate
  modified_base 27: mod_base = OTHER; note = 2'-deoxy-2'-fluoroadenosine-3'-phosphate
  modified_base 28: mod_base = OTHER; note = 2'-deoxy-2'-fluorouridine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-methyluridine-3'-phosphate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine-3'-phosphate
  modified_base 40: mod_base = OTHER; note = 2'-deoxy-2'-fluorocytidine-3'-phosphate
  modified_base 41: mod_base = OTHER; note = 2'-deoxy-2'-fluorouridine-3'-phosphate
  modified_base 42: mod_base = OTHER; note = 2'-O-methyluridine-3'-phosphate
  modified_base 43: mod_base = OTHER; note = 2'-O-methylguanosine-3'-phosphate
  modified_base 44: mod_base = OTHER; note = 2'-O-methylcytidine-3'-phosphate
  modified_base 45: mod_base = OTHER; note = 2'-O-methyluridine-3'-phosphate
  modified_base 46: mod_base = OTHER; note = 2'-O-methylcytidine-3'-phosphate
  modified_base 47: mod_base = OTHER; note = 2'-O-methyluridine-3'-phosphate
  modified_base 48: mod_base = OTHER; note = 2'-O-methyladenosine-3'-phosphate
  modified_base 49: mod_base = OTHER; note = 2'-O-methyluridine-3'-phosphate
  modified_base 50: mod_base = OTHER; note = 2'-O-methyladenosine-3'-phosphate
  modified_base 51: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
ttatagagcaagaacactgttttgagatagaacagtgttcttgctctataa

SEQ ID NO: 41 - RNA, 51 nt, synthetic construct; identical to SEQ ID NO: 40
  source 1..51: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate
  modified_base 26: mod_base = OTHER; note = 2'-deoxy-2'-fluoroguanosine-3'-phosphate
  modified_base 27: mod_base = OTHER; note = 2'-deoxy-2'-fluoroadenosine-3'-phosphate
  modified_base 28: mod_base = OTHER; note = 2'-deoxy-2'-fluorouridine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-methyluridine-3'-phosphate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine-3'-phosphate
  modified_base 40: mod_base = OTHER; note = 2'-deoxy-2'-fluorocytidine-3'-phosphate
  modified_base 41: mod_base = OTHER; note = 2'-deoxy-2'-fluorouridine-3'-phosphate
  modified_base 42: mod_base = OTHER; note = 2'-O-methyluridine-3'-phosphate
  modified_base 43: mod_base = OTHER; note = 2'-O-methylguanosine-3'-phosphate
  modified_base 44: mod_base = OTHER; note = 2'-O-methylcytidine-3'-phosphate
  modified_base 45: mod_base = OTHER; note = 2'-O-methyluridine-3'-phosphate
  modified_base 46: mod_base = OTHER; note = 2'-O-methylcytidine-3'-phosphate
  modified_base 47: mod_base = OTHER; note = 2'-O-methyluridine-3'-phosphate
  modified_base 48: mod_base = OTHER; note = 2'-O-methyladenosine-3'-phosphate
  modified_base 49: mod_base = OTHER; note = 2'-O-methyluridine-3'-phosphate
  modified_base 50: mod_base = OTHER; note = 2'-O-methyladenosine-3'-phosphate
  modified_base 51: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester

SEQ ID NO: 42 - RNA, 25 nt, synthetic construct
  source 1..25: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate connected at the 3'-end via a phosphodiester linkage to Q304Q304Q304
ttatagagcaagaacactgttttga

SEQ ID NO: 43 - RNA, 25 nt, synthetic construct
  source 1..25: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate connected at the 3'-end via a phosphodiester linkage to Q304Q304Q304
ttatagagcaagaacactgttttga

SEQ ID NO: 44 - DNA, 51 bp, synthetic construct
  source 1..51: mol_type = other DNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate
  modified_base 26: mod_base = OTHER; note = guanosine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-methyluridine-3'-phosphate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine-3'-phosphate
  modified_base 40: mod_base = OTHER; note = 2'-deoxy-2'-fluorocytidine-3'-phosphate
  modified_base 41: mod_base = OTHER; note = 2'-deoxy-2'-fluorouridine-3'-phosphate
  modified_base 42: mod_base = OTHER; note = 2'-O-methyluridine-3'-phosphate
  modified_base 43: mod_base = OTHER; note = 2'-O-methylguanosine-3'-phosphate
  modified_base 44: mod_base = OTHER; note = 2'-O-methylcytidine-3'-phosphate
  modified_base 45: mod_base = OTHER; note = 2'-O-methyluridine-3'-phosphate
  modified_base 46: mod_base = OTHER; note = 2'-O-methylcytidine-3'-phosphate
  modified_base 47: mod_base = OTHER; note = 2'-O-methyluridine-3'-phosphate
  modified_base 48: mod_base = OTHER; note = 2'-O-methyladenosine-3'-phosphate
  modified_base 49: mod_base = OTHER; note = 2'-O-methyluridine-3'-phosphate
  modified_base 50: mod_base = OTHER; note = 2'-O-methyladenosine-3'-phosphate
  modified_base 51: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
  misc_feature 1..26: note = RNA
  misc_feature 29..51: note = RNA
  misc_feature 27..28: note = DNA
ttatagagcaagaacactgttttgagatagaacagtgttcttgctctataa

SEQ ID NO: 45 - DNA, 51 bp, synthetic construct; identical to SEQ ID NO: 44
  source 1..51: mol_type = other DNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-methyluridine-3'-phosphate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine-3'-phosphate
  modified_base 40: mod_base = OTHER; note = 2'-deoxy-2'-fluorocytidine-3'-phosphate
  modified_base 41: mod_base = OTHER; note = 2'-deoxy-2'-fluorouridine-3'-phosphate
  modified_base 42: mod_base = OTHER; note = 2'-O-methyluridine-3'-phosphate
  modified_base 43: mod_base = OTHER; note = 2'-O-methylguanosine-3'-phosphate
  modified_base 44: mod_base = OTHER; note = 2'-O-methylcytidine-3'-phosphate
  modified_base 45: mod_base = OTHER; note = 2'-O-methyluridine-3'-phosphate
  modified_base 46: mod_base = OTHER; note = 2'-O-methylcytidine-3'-phosphate
  modified_base 47: mod_base = OTHER; note = 2'-O-methyluridine-3'-phosphate
  modified_base 48: mod_base = OTHER; note = 2'-O-methyladenosine-3'-phosphate
  modified_base 49: mod_base = OTHER; note = 2'-O-methyluridine-3'-phosphate
  modified_base 50: mod_base = OTHER; note = 2'-O-methyladenosine-3'-phosphate
  modified_base 51: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
  misc_feature 1..25: note = RNA
  misc_feature 29..51: note = RNA
  misc_feature 26..28: note = DNA

SEQ ID NO: 46 - DNA, 51 bp, synthetic construct
  source 1..51: mol_type = other DNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-methyluridine-3'-phosphate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine-3'-phosphate
  modified_base 40: mod_base = OTHER; note = 2'-deoxy-2'-fluorocytidine-3'-phosphate
  modified_base 41: mod_base = OTHER; note = 2'-deoxy-2'-fluorouridine-3'-phosphate
  modified_base 42: mod_base = OTHER; note = 2'-O-methyluridine-3'-phosphate
  modified_base 43: mod_base = OTHER; note = 2'-O-methylguanosine-3'-phosphate
  modified_base 44: mod_base = OTHER; note = 2'-O-methylcytidine-3'-phosphate
  modified_base 45: mod_base = OTHER; note = 2'-O-methyluridine-3'-phosphate
  modified_base 46: mod_base = OTHER; note = 2'-O-methylcytidine-3'-phosphate
  modified_base 47: mod_base = OTHER; note = 2'-O-methyluridine-3'-phosphate
  modified_base 48: mod_base = OTHER; note = 2'-O-methyladenosine-3'-phosphate
  modified_base 49: mod_base = OTHER; note = 2'-O-methyluridine-3'-phosphate
  modified_base 50: mod_base = OTHER; note = 2'-O-methyladenosine-3'-phosphate
  modified_base 51: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
  misc_feature 1..23: note = RNA
  misc_feature 31..51: note = RNA
  misc_feature 24..30: note = DNA
ttatagagcaagaacactgtttttttttttaacagtgttcttgctctataa

SEQ ID NO: 47 - DNA, 51 bp, synthetic construct; identical to SEQ ID NO: 46
  source 1..51: mol_type = other DNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 26: mod_base = OTHER; note = uridine-3'-phosphate
  modified_base 27: mod_base = OTHER; note = uridine-3'-phosphate
  modified_base 28: mod_base = OTHER; note = uridine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-methyluridine-3'-phosphate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine-3'-phosphate
  modified_base 40: mod_base = OTHER; note = 2'-deoxy-2'-fluorocytidine-3'-phosphate
  modified_base 41: mod_base = OTHER; note = 2'-deoxy-2'-fluorouridine-3'-phosphate
  modified_base 42: mod_base = OTHER; note = 2'-O-methyluridine-3'-phosphate
  modified_base 43: mod_base = OTHER; note = 2'-O-methylguanosine-3'-phosphate
  modified_base 44: mod_base = OTHER; note = 2'-O-methylcytidine-3'-phosphate
  modified_base 45: mod_base = OTHER; note = 2'-O-methyluridine-3'-phosphate
  modified_base 46: mod_base = OTHER; note = 2'-O-methylcytidine-3'-phosphate
  modified_base 47: mod_base = OTHER; note = 2'-O-methyluridine-3'-phosphate
  modified_base 48: mod_base = OTHER; note = 2'-O-methyladenosine-3'-phosphate
  modified_base 49: mod_base = OTHER; note = 2'-O-methyluridine-3'-phosphate
  modified_base 50: mod_base = OTHER; note = 2'-O-methyladenosine-3'-phosphate
  modified_base 51: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
  misc_feature 1..23: note = RNA
  misc_feature 26..28: note = RNA
  misc_feature 31..51: note = RNA
  misc_feature 24..25: note = DNA
  misc_feature 29..30: note = DNA

SEQ ID NO: 48 - RNA, 47 nt, synthetic construct
  source 1..47: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphorothioate
  modified_base 24: mod_base = OTHER; note = 2'-O-methyluridine-3'-phosphorothioate
  modified_base 25: mod_base = OTHER; note = 2'-O-methyluridine-3'-phosphorothioate
  modified_base 26: mod_base = OTHER; note = 2'-O-methyluridine-3'-phosphorothioate
  modified_base 27: mod_base = OTHER; note = 2'-O-methyladenosine-3'-phosphorothioate
  modified_base 28: mod_base = OTHER; note = 2'-O-methyladenosine-3'-phosphorothioate
  modified_base 29: mod_base = OTHER; note = 2'-O-methylcytidine-3'-phosphate
  modified_base 30: mod_base = OTHER; note = 2'-O-methyladenosine-3'-phosphate
  modified_base 31: mod_base = OTHER; note = 2'-O-methylguanosine-3'-phosphate
  modified_base 32: mod_base = OTHER; note = 2'-O-methyluridine-3'-phosphate
  modified_base 33: mod_base = OTHER; note = 2'-deoxy-2'-fluoroguanosine-3'-phosphate
  modified_base 34: mod_base = OTHER; note = 2'-O-methyluridine-3'-phosphate
  modified_base 35: mod_base = OTHER; note = 2'-deoxy-2'-fluorouridine-3'-phosphate
  modified_base 36: mod_base = OTHER; note = 2'-deoxy-2'-fluorocytidine-3'-phosphate
  modified_base 37: mod_base = OTHER; note = 2'-deoxy-2'-fluorouridine-3'-phosphate
  modified_base 38: mod_base = OTHER; note = 2'-O-methyluridine-3'-phosphate
  modified_base 39: mod_base = OTHER; note = 2'-O-methylguanosine-3'-phosphate
  modified_base 40: mod_base = OTHER; note = 2'-O-methylcytidine-3'-phosphate
  modified_base 41: mod_base = OTHER; note = 2'-O-methyluridine-3'-phosphate
  modified_base 42: mod_base = OTHER; note = 2'-O-methylcytidine-3'-phosphate
  modified_base 43: mod_base = OTHER; note = 2'-O-methyluridine-3'-phosphate
  modified_base 44: mod_base = OTHER; note = 2'-O-methyladenosine-3'-phosphate
  modified_base 45: mod_base = OTHER; note = 2'-O-methyluridine-3'-phosphate
  modified_base 46: mod_base = OTHER; note = 2'-O-methyladenosine-3'-phosphate
  modified_base 47: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
ttatagagcaagaacactgtttttttaacagtgttcttgctctataa

SEQ ID NO: 49 - RNA, 47 nt, synthetic construct; identical to SEQ ID NO: 48
  source 1..47: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphorothioate
  modified_base 24: mod_base = OTHER; note = 2'-O-methyluridine-3'-phosphorothioate
  modified_base 25: mod_base = OTHER; note = 2'-O-methyluridine-3'-phosphorothioate
  modified_base 26: mod_base = OTHER; note = 2'-O-methyluridine-3'-phosphorothioate
  modified_base 27: mod_base = OTHER; note = 2'-O-methyladenosine-3'-phosphorothioate
  modified_base 28: mod_base = OTHER; note = 2'-O-methyladenosine-3'-phosphorothioate
  modified_base 29: mod_base = OTHER; note = 2'-O-methylcytidine-3'-phosphate
  modified_base 30: mod_base = OTHER; note = 2'-O-methyladenosine-3'-phosphate
  modified_base 31: mod_base = OTHER; note = 2'-O-methylguanosine-3'-phosphate
  modified_base 32: mod_base = OTHER; note = 2'-O-methyluridine-3'-phosphate
  modified_base 33: mod_base = OTHER; note = 2'-deoxy-2'-fluoroguanosine-3'-phosphate
  modified_base 34: mod_base = OTHER; note = 2'-O-methyluridine-3'-phosphate
  modified_base 35: mod_base = OTHER; note = 2'-deoxy-2'-fluorouridine-3'-phosphate
  modified_base 36: mod_base = OTHER; note = 2'-deoxy-2'-fluorocytidine-3'-phosphate
  modified_base 37: mod_base = OTHER; note = 2'-deoxy-2'-fluorouridine-3'-phosphate
  modified_base 38: mod_base = OTHER; note = 2'-O-methyluridine-3'-phosphate
  modified_base 39: mod_base = OTHER; note = 2'-O-methylguanosine-3'-phosphate
  modified_base 40: mod_base = OTHER; note = 2'-O-methylcytidine-3'-phosphate
  modified_base 41: mod_base = OTHER; note = 2'-O-methyluridine-3'-phosphate
  modified_base 42: mod_base = OTHER; note = 2'-O-methylcytidine-3'-phosphate
  modified_base 43: mod_base = OTHER; note = 2'-O-methyluridine-3'-phosphate
  modified_base 44: mod_base = OTHER; note = 2'-O-methyladenosine-3'-phosphate
  modified_base 45: mod_base = OTHER; note = 2'-O-methyluridine-3'-phosphate
  modified_base 46: mod_base = OTHER; note = 2'-O-methyladenosine-3'-phosphate
  modified_base 47: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester

SEQ ID NO: 50 - RNA, 51 nt, synthetic construct; identical to SEQ ID NO: 38
  source 1..51: mol_type = other RNA

SEQ ID NO: 51 - RNA, 51 nt, synthetic construct; identical to SEQ ID NO: 38
  source 1..51: mol_type = other RNA

SEQ ID NO: 52 - RNA, 51 nt, synthetic construct; identical to SEQ ID NO: 40
  source 1..51: mol_type = other RNA

SEQ ID NO: 53 - RNA, 51 nt, synthetic construct; identical to SEQ ID NO: 40
  source 1..51: mol_type = other RNA

SEQ ID NO: 54 - RNA, 25 nt, synthetic construct
  source 1..25: mol_type = other RNA
  modified_base 25: mod_base = OTHER; note = adenosine-3'-phosphate connected at the 3'-end via a phosphodiester linkage to Q304Q304Q304
ttatagagcaagaacactgttttga

SEQ ID NO: 55 - RNA, 23 nt, synthetic construct
  source 1..23: mol_type = other RNA
  modified_base 1: mod_base = OTHER; note = adenosine-3'-phosphate connected at the 5'-end via a phosphodiester linkage to Q304Q304Q304
agaacagtgttcttgctctataa

SEQ ID NO: 56 - RNA, 25 nt, synthetic construct
  source 1..25: mol_type = other RNA
  modified_base 25: mod_base = OTHER; note = adenosine-3'-phosphate connected at the 3'-end via a phosphodiester linkage to Q304Q304Q304
ttatagagcaagaacactgttttga

SEQ ID NO: 57 - RNA, 23 nt, synthetic construct
  source 1..23: mol_type = other RNA
  modified_base 1: mod_base = OTHER; note = adenosine-3'-phosphate connected at the 5'-end via a phosphodiester linkage to Q304Q304Q304
agaacagtgttcttgctctataa

SEQ ID NO: 58 - RNA, 51 nt, synthetic construct; identical to SEQ ID NO: 40
  source 1..51: mol_type = other RNA
  modified_base 28: mod_base = OTHER; note = 5'-methyluridine-3'-phosphate

SEQ ID NO: 59 - RNA, 51 nt, synthetic construct; identical to SEQ ID NO: 40
  source 1..51: mol_type = other RNA
  modified_base 28: mod_base = OTHER; note = 5'-methyluridine-3'-phosphate

SEQ ID NO: 60 - RNA, 51 nt, synthetic construct
  source 1..51: mol_type = other RNA
  modified_base 24: mod_base = OTHER; note = 5'-methyluridine-3'-phosphate
  modified_base 25: mod_base = OTHER; note = 5'-methyluridine-3'-phosphate
  modified_base 26: mod_base = OTHER; note = 5'-methyluridine-3'-phosphate
  modified_base 27: mod_base = OTHER; note = 5'-methyluridine-3'-phosphate
  modified_base 28: mod_base = OTHER; note = 5'-methyluridine-3'-phosphate
  modified_base 29: mod_base = OTHER; note = 5'-methyluridine-3'-phosphate
  modified_base 30: mod_base = OTHER; note = 5'-methyluridine-3'-phosphate
ttatagagcaagaacactgtttttttttttaacagtgttcttgctctataa

SEQ ID NO: 61 - RNA, 51 nt, synthetic construct; identical to SEQ ID NO: 60
  source 1..51: mol_type = other RNA
  modified_base 24: mod_base = OTHER; note = 5'-methyluridine-3'-phosphate
  modified_base 25: mod_base = OTHER; note = 5'-methyluridine-3'-phosphate
  modified_base 29: mod_base = OTHER; note = 5'-methyluridine-3'-phosphate
  modified_base 30: mod_base = OTHER; note = 5'-methyluridine-3'-phosphate

SEQ ID NO: 62 - RNA, 47 nt, synthetic construct; identical to SEQ ID NO: 48
  source 1..47: mol_type = other RNA

SEQ ID NO: 63 - RNA, 47 nt, synthetic construct; identical to SEQ ID NO: 48
  source 1..47: mol_type = other RNA

SEQ ID NO: 64 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
aacagtgttcttgctctataa

SEQ ID NO: 65 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 66 - RNA, 31 nt, synthetic construct
  source 1..31: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate
  modified_base 26: mod_base = OTHER; note = 2'-O-methyluridine-3'-phosphate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine
ttatagagcaagaacactgttttgatttaga

SEQ ID NO: 67 - RNA, 32 nt, synthetic construct
  source 1..32: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate
  modified_base 26: mod_base = OTHER; note = 2'-O-methyluridine-3'-phosphate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine
ttatagagcaagaacactgttttgatttagaa

SEQ ID NO: 68 - RNA, 23 nt, synthetic construct
  source 1..23: mol_type = other RNA
  modified_base 1: mod_base = OTHER; note = 2'-O-methyladenosine-3'-phosphate 5'phosphate
  modified_base 2: mod_base = OTHER; note = 2'-O-methylguanosine-3'-phosphate
  modified_base 3: mod_base = OTHER; note = 2'-O-methyladenosine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphate
  modified_base 6: mod_base = OTHER; note = 2'-O-methyladenosine-3'-phosphate
  modified_base 7: mod_base = OTHER; note = 2'-O-methylguanosine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-O-methyluridine-3'-phosphate
  modified_base 11: mod_base = OTHER; note = 2'-deoxy-2'-fluorouridine-3'-phosphate
  modified_base 12: mod_base = OTHER; note = 2'-deoxy-2'-fluorocytidine-3'-phosphate
  modified_base 13: mod_base = OTHER; note = 2'-deoxy-2'-fluorouridine-3'-phosphate
  modified_base 14: mod_base = OTHER; note = 2'-O-methyluridine-3'-phosphate
  modified_base 15: mod_base = OTHER; note = 2'-O-methylguanosine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyluridine-3'-phosphate
  modified_base 22: mod_base = OTHER; note = 2'-O-methyladenosine-3'-phosphate
  modified_base 23: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
agaacagtgttcttgctctataa

SEQ ID NO: 69 - RNA, 20 nt, synthetic construct
  source 1..20: mol_type = other RNA
  modified_base 1: mod_base = OTHER; note = 2'-O-methyladenosine-3'-phosphorothioate 5'phosphate
  modified_base 2: mod_base = OTHER; note = 2'-O-methylcytidine-3'-phosphate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methyluridine-3'-phosphate
  modified_base 6: mod_base = OTHER; note = 2'-deoxy-2'-fluoroguanosine-3'-phosphate
  modified_base 7: mod_base = OTHER; note = 2'-O-methyluridine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorouridine-3'-phosphate
  modified_base 11: mod_base = OTHER; note = 2'-O-methyluridine-3'-phosphate
  modified_base 12: mod_base = OTHER; note = 2'-O-methylguanosine-3'-phosphate
  modified_base 13: mod_base = OTHER; note = 2'-O-methylcytidine-3'-phosphate
  modified_base 14: mod_base = OTHER; note = 2'-O-methyluridine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyladenosine-3'-phosphate
  modified_base 20: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
acagtgttcttgctctataa

SEQ ID NO: 70 - RNA, 21 nt, synthetic construct
  source 1..21: mol_type = other RNA
  modified_base 1: mod_base = OTHER; note = 2'-O-methyladenosine-3'-phosphorothioate 5'phosphate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
aacagtgttcttgctctataa

SEQ ID NO: 71 - RNA, 36 nt, synthetic construct
  source 1..36: mol_type = other RNA
  modified_base 1: mod_base = OTHER; note = 2'-O-methyladenosine-3'-phosphate 5'phosphate
  modified_base 2: mod_base = OTHER; note = 2'-O-methyladenosine-3'-phosphate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-deoxy-2'-fluoroadenosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methylcytidine-3'-phosphate
  modified_base 9: mod_base = OTHER; note = 2'-O-methyluridine-3'-phosphate
  modified_base 10: mod_base = OTHER; note = 2'-O-methylguanosine-3'-phosphate
  modified_base 11: mod_base = OTHER; note = 2'-O-methyluridine-3'-phosphate
  modified_base 12: mod_base = OTHER; note = 2'-O-methyluridine-3'-phosphorothioate
  modified_base 13: mod_base = OTHER; note = 2'-O-methyluridine-3'-phosphorothioate
  modified_base 14: mod_base = OTHER; note = 2'-O-methyluridine-3'-phosphorothioate
  modified_base 15: mod_base = OTHER; note = 2'-O-methyluridine-3'-phosphorothioate
  modified_base 16: mod_base = OTHER; note = 2'-O-methyluridine-3'-phosphorothioate
  modified_base 17: mod_base = OTHER; note = 2'-O-methyluridine-3'-phosphorothioate
  modified_base 18: mod_base = OTHER; note = 2'-O-methyladenosine-3'-phosphorothioate
  modified_base 19: mod_base = OTHER; note = 2'-O-methyladenosine-3'-phosphorothioate
  modified_base 20: mod_base = OTHER; note = 2'-O-methylcytidine-3'-phosphate
  modified_base 21: mod_base = OTHER; note = 2'-O-methyladenosine-3'-phosphate
  modified_base 22: mod_base = OTHER; note = 2'-O-methylguanosine-3'-phosphate
  modified_base 23: mod_base = OTHER; note = 2'-O-methyluridine-3'-phosphate
  modified_base 24: mod_base = OTHER; note = 2'-deoxy-2'-fluoroguanosine-3'-phosphate
  modified_base 25: mod_base = OTHER; note = 2'-O-methyluridine-3'-phosphate
  modified_base 26: mod_base = OTHER; note = 2'-deoxy-2'-fluorouridine-3'-phosphate
  modified_base 27: mod_base = OTHER; note = 2'-deoxy-2'-fluorocytidine-3'-phosphate
  modified_base 28: mod_base = OTHER; note = 2'-deoxy-2'-fluorouridine-3'-phosphate
  modified_base 29: mod_base = OTHER; note = 2'-O-methyluridine-3'-phosphate
  modified_base 30: mod_base = OTHER; note = 2'-O-methylguanosine-3'-phosphate
  modified_base 31: mod_base = OTHER; note = 2'-O-methylcytidine-3'-phosphate
  modified_base 32: mod_base = OTHER; note = 2'-O-methyluridine-3'-phosphate
  modified_base 33: mod_base = OTHER; note = 2'-O-methylcytidine-3'-phosphate
  modified_base 34: mod_base = OTHER; note = 2'-O-methyluridine-3'-phosphate
  modified_base 35: mod_base = OTHER; note = 2'-O-methyladenosine-3'-phosphate
  modified_base 36: mod_base = OTHER; note = 2'-O-methyluridine
aagaacactgtttttttaacagtgttcttgctctat

SEQ ID NO: 72 - RNA, 23 nt, synthetic construct
  source 1..23: mol_type = other RNA
  modified_base 1: mod_base = OTHER; note = 2'-O-methyladenosine-3'-phosphate 5'phosphate
  modified_base 2: mod_base = OTHER; note = 2'-O-methylguanosine-3'-phosphate
  modified_base 3: mod_base = OTHER; note = 2'-O-methyladenosine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphate
  modified_base 6: mod_base = OTHER; note = 2'-O-methyladenosine-3'-phosphate
  modified_base 7: mod_base = OTHER; note = 2'-O-methylguanosine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-O-methyluridine-3'-phosphate
  modified_base 11: mod_base = OTHER; note = 2'-deoxy-2'-fluorouridine-3'-phosphate
  modified_base 12: mod_base = OTHER; note = 2'-deoxy-2'-fluorocytidine-3'-phosphate
  modified_base 13: mod_base = OTHER; note = 2'-deoxy-2'-fluorouridine-3'-phosphate
  modified_base 14: mod_base = OTHER; note = 2'-O-methyluridine-3'-phosphate
  modified_base 15: mod_base = OTHER; note = 2'-O-methylguanosine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyluridine-3'-phosphate
  modified_base 22: mod_base = OTHER; note = 2'-O-methyladenosine-3'-phosphate
  modified_base 23: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
agaacagtgttcttgctctataa

SEQ ID NO: 73 - RNA, 34 nt, synthetic construct
  source 1..34: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphorothioate
  modified_base 24: mod_base = OTHER; note = 2'-O-methyluridine-3'-phosphorothioate
  modified_base 25: mod_base = OTHER; note = 2'-O-methyluridine-3'-phosphorothioate
  modified_base 26: mod_base = OTHER; note = 2'-O-methyluridine-3'-phosphorothioate
  modified_base 27: mod_base = OTHER; note = 2'-O-methyladenosine-3'-phosphorothioate
  modified_base 28: mod_base = OTHER; note = 2'-O-methyladenosine-3'-phosphorothioate
  modified_base 29: mod_base = OTHER; note = 2'-O-methylcytidine-3'-phosphate
  modified_base 30: mod_base = OTHER; note = 2'-O-methyladenosine-3'-phosphate
  modified_base 31: mod_base = OTHER; note = 2'-O-methylguanosine-3'-phosphate
  modified_base 32: mod_base = OTHER; note = 2'-O-methyluridine-3'-phosphate
  modified_base 33: mod_base = OTHER; note = 2'-deoxy-2'-fluoroguanosine-3'-phosphate
  modified_base 34: mod_base = OTHER; note = 2'-O-methyluridine
ttatagagcaagaacactgtttttttaacagtgt

SEQ ID NO: 74 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
aacagtgttcttgctctataa

SEQ ID NO: 75 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester having lost one N-acetylgalactosamine (minus one GalNAc)
aacagtgttcttgctctataa

SEQ ID NO: 76 - RNA, 11 nt, synthetic construct
  source 1..11: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyluridine-3'-phosphate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
ttgctctataa

SEQ ID NO: 77 - RNA, 40 nt, synthetic construct
  source 1..40: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate
  modified_base 26: mod_base = OTHER; note = 2'-O-methyluridine-3'-phosphate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-methyluridine-3'-phosphate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine-3'-phosphate
  modified_base 40: mod_base = OTHER; note = 2'-deoxy-2'-fluorocytidine
ttatagagcaagaacactgttttgatttagaacagtgttc

SEQ ID NO: 78 - RNA, 39 nt, synthetic construct
  source 1..39: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate
  modified_base 26: mod_base = OTHER; note = 2'-O-methyluridine-3'-phosphate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-methyluridine-3'-phosphate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine
ttatagagcaagaacactgttttgatttagaacagtgtt

SEQ ID NO: 79 - RNA, 11 nt, synthetic construct
  source 1..11: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyluridine-3'-phosphate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
ttgctctataa

SEQ ID NO: 80 - RNA, 11 nt, synthetic construct
  source 1..11: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyluridine-3'-phosphate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester having lost one N-acetylgalactosamine (minus one GalNAc)
ttgctctataa

SEQ ID NO: 81 - RNA, 21 nt, synthetic construct
  source 1..21: mol_type = other RNA
  modified_base 1: mod_base = OTHER; note = 2'-O-methyladenosine-3'-phosphorothioate 5'phosphate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
aacagtgttcttgctctataa

SEQ ID NO: 82 - RNA, 22 nt, synthetic construct
  source 1..22: mol_type = other RNA
  modified_base 1: mod_base = OTHER; note = 2'-O-methylguanosine-3'-phosphate 5'phosphate
  modified_base 2: mod_base = OTHER; note = 2'-O-methyladenosine-3'-phosphorothioate
  modified_base 3: mod_base = OTHER; note = 2'-O-methyladenosine-3'-phosphorothioate
  modified_base 4: mod_base = OTHER; note = 2'-O-methylcytidine-3'-phosphate
  modified_base 5: mod_base = OTHER; note = 2'-O-methyladenosine-3'-phosphate
  modified_base 6: mod_base = OTHER; note = 2'-O-methylguanosine-3'-phosphate
  modified_base 7: mod_base = OTHER; note = 2'-O-methyluridine-3'-phosphate
  modified_base 8: mod_base = OTHER; note = 2'-deoxy-2'-fluoroguanosine-3'-phosphate
  modified_base 9: mod_base = OTHER; note = 2'-O-methyluridine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorocytidine-3'-phosphate
  modified_base 12: mod_base = OTHER; note = 2'-deoxy-2'-fluorouridine-3'-phosphate
  modified_base 13: mod_base = OTHER; note = 2'-O-methyluridine-3'-phosphate
  modified_base 14: mod_base = OTHER; note = 2'-O-methylguanosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methyladenosine-3'-phosphate
  modified_base 22: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
gaacagtgttcttgctctataa

SEQ ID NO: 83 - RNA, 28 nt, synthetic construct
  source 1..28: mol_type = other RNA
  modified_base 1: mod_base = OTHER; note = 2'-O-methyladenosine-3'-phosphate 5'phosphate
  modified_base 2: mod_base = OTHER; note = 2'-deoxy-2'-fluoroadenosine-3'-phosphate
  modified_base 3: mod_base = OTHER; note = 2'-O-methylcytidine-3'-phosphate
  modified_base 4: mod_base = OTHER; note = 2'-deoxy-2'-fluoroadenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = 2'-O-methylguanosine-3'-phosphate
  modified_base 8: mod_base = OTHER; note = 2'-O-methyluridine-3'-phosphate
  modified_base 9: mod_base = OTHER; note = 2'-O-methyluridine-3'-phosphorothioate
  modified_base 10: mod_base = OTHER; note = 2'-O-methyluridine-3'-phosphorothioate
  modified_base 11: mod_base = OTHER; note = 2'-O-methyluridine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroguanosine-3'-phosphate
  modified_base 15: mod_base = OTHER; note = 2'-deoxy-2'-fluoroadenosine-3'-phosphate
  modified_base 16: mod_base = OTHER; note = 2'-deoxy-2'-fluorouridine-3'-phosphate
  modified_base 17: mod_base = OTHER; note = 2'-O-methyladenosine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methylcytidine-3'-phosphate
  modified_base 22: mod_base = OTHER; note = 2'-O-methyladenosine-3'-phosphate
  modified_base 23: mod_base = OTHER; note = 2'-O-methylguanosine-3'-phosphate
  modified_base 24: mod_base = OTHER; note = 2'-O-methyluridine-3'-phosphate
  modified_base 25: mod_base = OTHER; note = 2'-deoxy-2'-fluoroguanosine-3'-phosphate
  modified_base 26: mod_base = OTHER; note = 2'-O-methyluridine-3'-phosphate
  modified_base 27: mod_base = OTHER; note = 2'-deoxy-2'-fluorouridine-3'-phosphate
  modified_base 28: mod_base = OTHER; note = 2'-deoxy-2'-fluorocytidine
aacactgttttgagatagaacagtgttc

SEQ ID NO: 84 - RNA, 23 nt, synthetic construct
  source 1..23: mol_type = other RNA
  modified_base 1: mod_base = OTHER; note = 2'-O-methyladenosine-3'-phosphate 5'phosphate
  modified_base 2: mod_base = OTHER; note = 2'-O-methylguanosine-3'-phosphate
  modified_base 3: mod_base = OTHER; note = 2'-O-methyladenosine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphate
  modified_base 6: mod_base = OTHER; note = 2'-O-methyladenosine-3'-phosphate
  modified_base 7: mod_base = OTHER; note = 2'-O-methylguanosine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-O-methyluridine-3'-phosphate
  modified_base 11: mod_base = OTHER; note = 2'-deoxy-2'-fluorouridine-3'-phosphate
  modified_base 12: mod_base = OTHER; note = 2'-deoxy-2'-fluorocytidine-3'-phosphate
  modified_base 13: mod_base = OTHER; note = 2'-deoxy-2'-fluorouridine-3'-phosphate
  modified_base 14: mod_base = OTHER; note = 2'-O-methyluridine-3'-phosphate
  modified_base 15: mod_base = OTHER; note = 2'-O-methylguanosine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyluridine-3'-phosphate
  modified_base 22: mod_base = OTHER; note = 2'-O-methyladenosine-3'-phosphate
  modified_base 23: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
agaacagtgttcttgctctataa

SEQ ID NO: 85 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 86 - RNA, 11 nt, synthetic construct
  source 1..11: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyluridine-3'-phosphate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
ttgctctataa

SEQ ID NO: 87 - RNA, 11 nt, synthetic construct
  source 1..11: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyluridine-3'-phosphate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester having lost one N-acetylgalactosamine (minus one GalNAc)
ttgctctataa

SEQ ID NO: 88 - DNA, 22 bp, synthetic construct
  source 1..22: mol_type = other DNA
  modified_base 1: mod_base = OTHER; note = 2'-deoxythymidine-3'-phosphate 5'phosphate
  modified_base 2: mod_base = OTHER; note = 2'-O-methyladenosine-3'-phosphorothioate
  modified_base 3: mod_base = OTHER; note = 2'-O-methyladenosine-3'-phosphorothioate
  modified_base 4: mod_base = OTHER; note = 2'-O-methylcytidine-3'-phosphate
  modified_base 5: mod_base = OTHER; note = 2'-O-methyladenosine-3'-phosphate
  modified_base 6: mod_base = OTHER; note = 2'-O-methylguanosine-3'-phosphate
  modified_base 7: mod_base = OTHER; note = 2'-O-methyluridine-3'-phosphate
  modified_base 8: mod_base = OTHER; note = 2'-deoxy-2'-fluoroguanosine-3'-phosphate
  modified_base 9: mod_base = OTHER; note = 2'-O-methyluridine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorocytidine-3'-phosphate
  modified_base 12: mod_base = OTHER; note = 2'-deoxy-2'-fluorouridine-3'-phosphate
  modified_base 13: mod_base = OTHER; note = 2'-O-methyluridine-3'-phosphate
  modified_base 14: mod_base = OTHER; note = 2'-O-methylguanosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methyladenosine-3'-phosphate
  modified_base 22: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
  misc_feature 2..22: note = RNA
taacagtgttcttgctctataa

SEQ ID NO: 89 - DNA, 22 bp, synthetic construct
  source 1..22: mol_type = other DNA
  modified_base 1: mod_base = OTHER; note = 2'-deoxythymidine-3'-phosphate 5'phosphate
  modified_base 2: mod_base = OTHER; note = 2'-O-methyladenosine-3'-phosphorothioate
  modified_base 3: mod_base = OTHER; note = 2'-O-methyladenosine-3'-phosphorothioate
  modified_base 4: mod_base = OTHER; note = 2'-O-methylcytidine-3'-phosphate
  modified_base 5: mod_base = OTHER; note = 2'-O-methyladenosine-3'-phosphate
  modified_base 6: mod_base = OTHER; note = 2'-O-methylguanosine-3'-phosphate
  modified_base 7: mod_base = OTHER; note = 2'-O-methyluridine-3'-phosphate
  modified_base 8: mod_base = OTHER; note = 2'-deoxy-2'-fluoroguanosine-3'-phosphate
  modified_base 9: mod_base = OTHER; note = 2'-O-methyluridine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorocytidine-3'-phosphate
  modified_base 12: mod_base = OTHER; note = 2'-deoxy-2'-fluorouridine-3'-phosphate
  modified_base 13: mod_base = OTHER; note = 2'-O-methyluridine-3'-phosphate
  modified_base 14: mod_base = OTHER; note = 2'-O-methylguanosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methyladenosine-3'-phosphate
  modified_base 22: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester having lost one N-acetylgalactosamine (minus one GalNAc)
  misc_feature 2..22: note = RNA
taacagtgttcttgctctataa

SEQ ID NO: 90 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 91 - RNA, 11 nt, synthetic construct
  source 1..11: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyluridine-3'-phosphate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester having lost one N-acetylgalactosamine (minus one GalNAc)
ttgctctataa

SEQ ID NO: 92 - RNA, 11 nt, synthetic construct
  source 1..11: mol_type = other RNA
  modified_base 1: mod_base = OTHER; note = 2'-O-methyladenosine-3'-phosphate
  modified_base 2: mod_base = OTHER; note = 2'-deoxy-2'-fluoroguanosine-3'-phosphate
  modified_base 3: mod_base = OTHER; note = 2'-O-methyladenosine-3'-phosphate
  modified_base 4: mod_base = OTHER; note = 2'-deoxy-2'-fluoroguanosine-3'-phosphate
  modified_base 5: mod_base = OTHER; note = 2'-deoxy-2'-fluorocytidine-3'-phosphate
  modified_base 6: mod_base = OTHER; note = 2'-O-methyladenosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methyladenosine-3'-phosphate
  modified_base 10: mod_base = OTHER; note = 2'-deoxy-2'-fluoroadenosine-3'-phosphate
  modified_base 11: mod_base = OTHER; note = 2'-O-methylcytidine
agagcaagaac

SEQ ID NO: 93 - DNA, 40 bp, synthetic construct
  source 1..40: mol_type = other DNA
  modified_base 1: mod_base = OTHER; note = 2'-O-methyladenosine-3'-phosphate 5'phosphate
  modified_base 2: mod_base = OTHER; note = 2'-O-methyladenosine-3'-phosphate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-deoxy-2'-fluoroadenosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methylcytidine-3'-phosphate
  modified_base 9: mod_base = OTHER; note = 2'-O-methyluridine-3'-phosphate
  modified_base 10: mod_base = OTHER; note = 2'-O-methylguanosine-3'-phosphate
  modified_base 11: mod_base = OTHER; note = 2'-O-methyluridine-3'-phosphate
  modified_base 12: mod_base = OTHER; note = 2'-O-methyluridine-3'-phosphorothioate
  modified_base 13: mod_base = OTHER; note = 2'-O-methyluridine-3'-phosphorothioate
  modified_base 14: mod_base = OTHER; note = 2'-O-methyluridine-3'-phosphate
  modified_base 15: mod_base = OTHER; note = 2'-O-methylguanosine-3'-phosphate
  modified_base 16: mod_base = OTHER; note = 2'-O-methyladenosine-3'-phosphate
  modified_base 20: mod_base = OTHER; note = 2'-O-methyladenosine-3'-phosphate
  modified_base 21: mod_base = OTHER; note = 2'-O-methylguanosine-3'-phosphate
  modified_base 22: mod_base = OTHER; note = 2'-O-methyladenosine-3'-phosphorothioate
  modified_base 23: mod_base = OTHER; note = 2'-O-methyladenosine-3'-phosphorothioate
  modified_base 24: mod_base = OTHER; note = 2'-O-methylcytidine-3'-phosphate
  modified_base 25: mod_base = OTHER; note = 2'-O-methyladenosine-3'-phosphate
  modified_base 26: mod_base = OTHER; note = 2'-O-methylguanosine-3'-phosphate
  modified_base 27: mod_base = OTHER; note = 2'-O-methyluridine-3'-phosphate
  modified_base 28: mod_base = OTHER; note = 2'-deoxy-2'-fluoroguanosine-3'-phosphate
  modified_base 29: mod_base = OTHER; note = 2'-O-methyluridine-3'-phosphate
  modified_base 30: mod_base = OTHER; note = 2'-deoxy-2'-fluorouridine-3'-phosphate
  modified_base 31: mod_base = OTHER; note = 2'-deoxy-2'-fluorocytidine-3'-phosphate
  modified_base 32: mod_base = OTHER; note = 2'-deoxy-2'-fluorouridine-3'-phosphate
  modified_base 33: mod_base = OTHER; note = 2'-O-methyluridine-3'-phosphate
  modified_base 34: mod_base = OTHER; note = 2'-O-methylguanosine-3'-phosphate
  modified_base 35: mod_base = OTHER; note = 2'-O-methylcytidine-3'-phosphate
  modified_base 36: mod_base = OTHER; note = 2'-O-methyluridine-3'-phosphate
  modified_base 37: mod_base = OTHER; note = 2'-O-methylcytidine-3'-phosphate
  modified_base 38: mod_base = OTHER; note = 2'-O-methyluridine-3'-phosphate
  modified_base 39: mod_base = OTHER; note = 2'-O-methyladenosine-3'-phosphate
  modified_base 40: mod_base = OTHER; note = 2'-O-methyluridine
  misc_feature 1..16: note = RNA
  misc_feature 20..40: note = RNA
  misc_feature 17..19: note = DNA
aagaacactgttttgagatagaacagtgttcttgctctat

SEQ ID NO: 94 - RNA, 11 nt, synthetic construct
  source 1..11: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyluridine-3'-phosphate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
ttgctctataa

SEQ ID NO: 95 - RNA, 11 nt, synthetic construct
  source 1..11: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyluridine-3'-phosphate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester having lost one N-acetylgalactosamine (minus one GalNAc)
ttgctctataa

SEQ ID NO: 96 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 97 - DNA, 28 bp, synthetic construct
  source 1..28: mol_type = other DNA
  modified_base 1: mod_base = OTHER; note = 2'-O-methyluridine-3'-phosphate 5'phosphate
  modified_base 2: mod_base = OTHER; note = 2'-O-methyluridine-3'-phosphorothioate
  modified_base 3: mod_base = OTHER; note = 2'-O-methyluridine-3'-phosphorothioate
  modified_base 4: mod_base = OTHER; note = 2'-O-methyluridine-3'-phosphate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methylguanosine-3'-phosphate
  modified_base 17: mod_base = OTHER; note = 2'-O-methyluridine-3'-phosphate
  modified_base 18: mod_base = OTHER; note = 2'-deoxy-2'-fluoroguanosine-3'-phosphate
  modified_base 19: mod_base = OTHER; note = 2'-O-methyluridine-3'-phosphate
  modified_base 20: mod_base = OTHER; note = 2'-deoxy-2'-fluorouridine-3'-phosphate
  modified_base 21: mod_base = OTHER; note = 2'-deoxy-2'-fluorocytidine-3'-phosphate
  modified_base 22: mod_base = OTHER; note = 2'-deoxy-2'-fluorouridine-3'-phosphate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methylcytidine-3'-phosphate
  modified_base 26: mod_base = OTHER; note = 2'-O-methyluridine-3'-phosphate
  modified_base 27: mod_base = OTHER; note = 2'-O-methylcytidine-3'-phosphate
  modified_base 28: mod_base = OTHER; note = 2'-O-methyluridine
  misc_feature 1..4: note = RNA
  misc_feature 12..28: note = RNA
  misc_feature 5..11: note = DNA
tttttttttttaacagtgttcttgctct

SEQ ID NO: 98 - RNA, 21 nt, synthetic construct
  source 1..21: mol_type = other RNA
  modified_base 1: mod_base = OTHER; note = 2'-O-methyladenosine-3'-phosphorothioate 5'phosphate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
aacagtgttcttgctctataa

SEQ ID NO: 99 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 100 - RNA, 11 nt, synthetic construct
  source 1..11: mol_type = other RNA
  modified_base 1: mod_base = OTHER; note = 2'-O-methyladenosine-3'-phosphate
  modified_base 2: mod_base = OTHER; note = 2'-deoxy-2'-fluoroguanosine-3'-phosphate
  modified_base 3: mod_base = OTHER; note = 2'-O-methyladenosine-3'-phosphate
  modified_base 4: mod_base = OTHER; note = 2'-deoxy-2'-fluoroguanosine-3'-phosphate
  modified_base 5: mod_base = OTHER; note = 2'-deoxy-2'-fluorocytidine-3'-phosphate
  modified_base 6: mod_base = OTHER; note = 2'-O-methyladenosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methyladenosine-3'-phosphate
  modified_base 10: mod_base = OTHER; note = 2'-deoxy-2'-fluoroadenosine-3'-phosphate
  modified_base 11: mod_base = OTHER; note = 2'-O-methylcytidine
agagcaagaac

SEQ ID NO: 101 - RNA, 47 nt, synthetic construct; identical to SEQ ID NO: 48
  source 1..47: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphorothioate
  modified_base 24: mod_base = OTHER; note = 2'-O-methyluridine-3'-phosphorothioate
  modified_base 25: mod_base = OTHER; note = 2'-O-methyluridine-3'-phosphorothioate
  modified_base 26: mod_base = OTHER; note = 2'-O-methyluridine-3'-phosphorothioate
  modified_base 27: mod_base = OTHER; note = 2'-O-methyladenosine-3'-phosphorothioate
  modified_base 28: mod_base = OTHER; note = 2'-O-methyladenosine-3'-phosphorothioate
  modified_base 29: mod_base = OTHER; note = 2'-O-methylcytidine-3'-phosphate
  modified_base 30: mod_base = OTHER; note = 2'-O-methyladenosine-3'-phosphate
  modified_base 31: mod_base = OTHER; note = 2'-O-methylguanosine-3'-phosphate
  modified_base 32: mod_base = OTHER; note = 2'-O-methyluridine-3'-phosphate
  modified_base 33: mod_base = OTHER; note = 2'-deoxy-2'-fluoroguanosine-3'-phosphate
  modified_base 34: mod_base = OTHER; note = 2'-O-methyluridine-3'-phosphate
  modified_base 35: mod_base = OTHER; note = 2'-deoxy-2'-fluorouridine-3'-phosphate
  modified_base 36: mod_base = OTHER; note = 2'-deoxy-2'-fluorocytidine-3'-phosphate
  modified_base 37: mod_base = OTHER; note = 2'-deoxy-2'-fluorouridine-3'-phosphate
  modified_base 38: mod_base = OTHER; note = 2'-O-methyluridine-3'-phosphate
  modified_base 39: mod_base = OTHER; note = 2'-O-methylguanosine-3'-phosphate
  modified_base 40: mod_base = OTHER; note = 2'-O-methylcytidine-3'-phosphate
  modified_base 41: mod_base = OTHER; note = 2'-O-methyluridine-3'-phosphate
  modified_base 42: mod_base = OTHER; note = 2'-O-methylcytidine-3'-phosphate
  modified_base 43: mod_base = OTHER; note = 2'-O-methyluridine-3'-phosphate
  modified_base 44: mod_base = OTHER; note = 2'-O-methyladenosine-3'-phosphate
  modified_base 45: mod_base = OTHER; note = 2'-O-methyluridine-3'-phosphate
  modified_base 46: mod_base = OTHER; note = 2'-O-methyladenosine-3'-phosphate
  modified_base 47: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester having lost one N-acetylgalactosamine (minus one GalNAc)

SEQ ID NO: 102 - RNA, 36 nt, synthetic construct; identical to SEQ ID NO: 71
  source 1..36: mol_type = other RNA
  modified_base 1: mod_base = OTHER; note = 2'-O-methyladenosine-3'-phosphate 5'phosphate
  modified_base 2: mod_base = OTHER; note = 2'-O-methyladenosine-3'-phosphate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-deoxy-2'-fluoroadenosine-3'-phosphate
  modified_base 6: mod_base = OTHER; note = 2'-O-methylcytidine-3'-phosphate
  modified_base 7: mod_base = OTHER; note = 2'-deoxy-2'-fluoroadenosine-3'-phosphate
  modified_base 8: mod_base = OTHER; note = 2'-O-methylcytidine-3'-phosphate
  modified_base 9: mod_base = OTHER; note = 2'-O-methyluridine-3'-phosphate
  modified_base 10: mod_base = OTHER; note = 2'-O-methylguanosine-3'-phosphate
  modified_base 11: mod_base = OTHER; note = 2'-O-methyluridine-3'-phosphate
  modified_base 12: mod_base = OTHER; note = 2'-O-methyluridine-3'-phosphorothioate
  modified_base 13: mod_base = OTHER; note = 2'-O-methyluridine-3'-phosphorothioate
  modified_base 14: mod_base = OTHER; note = 2'-O-methyluridine-3'-phosphorothioate
  modified_base 15: mod_base = OTHER; note = 2'-O-methyluridine-3'-phosphorothioate
  modified_base 16: mod_base = OTHER; note = 2'-O-methyluridine-3'-phosphorothioate
  modified_base 17: mod_base = OTHER; note = 2'-O-methyluridine-3'-phosphorothioate
  modified_base 18: mod_base = OTHER; note = 2'-O-methyladenosine-3'-phosphorothioate
  modified_base 19: mod_base = OTHER; note = 2'-O-methyladenosine-3'-phosphorothioate
  modified_base 20: mod_base = OTHER; note = 2'-O-methylcytidine-3'-phosphate
  modified_base 21: mod_base = OTHER; note = 2'-O-methyladenosine-3'-phosphate
  modified_base 22: mod_base = OTHER; note = 2'-O-methylguanosine-3'-phosphate
  modified_base 23: mod_base = OTHER; note = 2'-O-methyluridine-3'-phosphate
  modified_base 24: mod_base = OTHER; note = 2'-deoxy-2'-fluoroguanosine-3'-phosphate
  modified_base 25: mod_base = OTHER; note = 2'-O-methyluridine-3'-phosphate
  modified_base 26: mod_base = OTHER; note = 2'-deoxy-2'-fluorouridine-3'-phosphate
  modified_base 27: mod_base = OTHER; note = 2'-deoxy-2'-fluorocytidine-3'-phosphate
  modified_base 28: mod_base = OTHER; note = 2'-deoxy-2'-fluorouridine-3'-phosphate
  modified_base 29: mod_base = OTHER; note = 2'-O-methyluridine-3'-phosphate
  modified_base 30: mod_base = OTHER; note = 2'-O-methylguanosine-3'-phosphate
  modified_base 31: mod_base = OTHER; note = 2'-O-methylcytidine-3'-phosphate
  modified_base 32: mod_base = OTHER; note = 2'-O-methyluridine-3'-phosphate
  modified_base 33: mod_base = OTHER; note = 2'-O-methylcytidine-3'-phosphate
  modified_base 34: mod_base = OTHER; note = 2'-O-methyluridine-3'-phosphate
  modified_base 35: mod_base = OTHER; note = 2'-O-methyladenosine-3'-phosphate
  modified_base 36: mod_base = OTHER; note = 2'-O-methyluridine

SEQ ID NO: 103 - RNA, 11 nt, synthetic construct
  source 1..11: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyluridine-3'-phosphate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
ttgctctataa

SEQ ID NO: 104 - RNA, 11 nt, synthetic construct
  source 1..11: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyluridine-3'-phosphate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester having lost one N-acetylgalactosamine (minus one GalNAc)
ttgctctataa

SEQ ID NO: 105 - RNA, 16 nt, synthetic construct
  source 1..16: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine
ttatagagcaagaaca

SEQ ID NO: 106 - RNA, 51 nt, synthetic construct; identical to SEQ ID NO: 38
  source 1..51: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate
  modified_base 26: mod_base = OTHER; note = 2'-O-methyluridine-3'-phosphate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-methyluridine-3'-phosphate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine-3'-phosphate
  modified_base 40: mod_base = OTHER; note = 2'-deoxy-2'-fluorocytidine-3'-phosphate
  modified_base 41: mod_base = OTHER; note = 2'-deoxy-2'-fluorouridine-3'-phosphate
  modified_base 42: mod_base = OTHER; note = 2'-O-methyluridine-3'-phosphate
  modified_base 43: mod_base = OTHER; note = 2'-O-methylguanosine-3'-phosphate
  modified_base 44: mod_base = OTHER; note = 2'-O-methylcytidine-3'-phosphate
  modified_base 45: mod_base = OTHER; note = 2'-O-methyluridine-3'-phosphate
  modified_base 46: mod_base = OTHER; note = 2'-O-methylcytidine-3'-phosphate
  modified_base 47: mod_base = OTHER; note = 2'-O-methyluridine-3'-phosphate
  modified_base 48: mod_base = OTHER; note = 2'-O-methyladenosine-3'-phosphate
  modified_base 49: mod_base = OTHER; note = 2'-O-methyluridine-3'-phosphate
  modified_base 50: mod_base = OTHER; note = 2'-O-methyladenosine-3'-phosphate
  modified_base 51: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester having lost one N-acetylgalactosamine (minus one GalNAc)

SEQ ID NO: 107 - RNA, 40 nt, synthetic construct; identical to SEQ ID NO: 77
  source 1..40: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate
  modified_base 26: mod_base = OTHER; note = 2'-O-methyluridine-3'-phosphate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-methyluridine-3'-phosphate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine-3'-phosphate
  modified_base 40: mod_base = OTHER; note = 2'-deoxy-2'-fluorocytidine

SEQ ID NO: 108 - RNA, 39 nt, synthetic construct; identical to SEQ ID NO: 78
  source 1..39: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate
  modified_base 26: mod_base = OTHER; note = 2'-O-methyluridine-3'-phosphate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-methyluridine-3'-phosphate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine

SEQ ID NO: 109 - RNA, 11 nt, synthetic construct
  source 1..11: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyluridine-3'-phosphate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
ttgctctataa

SEQ ID NO: 110 - RNA, 11 nt, synthetic construct
  source 1..11: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyluridine-3'-phosphate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester having lost one N-acetylgalactosamine (minus one GalNAc)
ttgctctataa

SEQ ID NO: 111 - RNA, 23 nt, synthetic construct
  source 1..23: mol_type = other RNA
  modified_base 1: mod_base = OTHER; note = 2'-O-methyladenosine-3'-phosphate
  modified_base 2: mod_base = OTHER; note = 2'-O-methylguanosine-3'-phosphate
  modified_base 3: mod_base = OTHER; note = 2'-O-methyladenosine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphate
  modified_base 6: mod_base = OTHER; note = 2'-O-methyladenosine-3'-phosphate
  modified_base 7: mod_base = OTHER; note = 2'-O-methylguanosine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-O-methyluridine-3'-phosphate
  modified_base 11: mod_base = OTHER; note = 2'-deoxy-2'-fluorouridine-3'-phosphate
  modified_base 12: mod_base = OTHER; note = 2'-deoxy-2'-fluorocytidine-3'-phosphate
  modified_base 13: mod_base = OTHER; note = 2'-deoxy-2'-fluorouridine-3'-phosphate
  modified_base 14: mod_base = OTHER; note = 2'-O-methyluridine-3'-phosphate
  modified_base 15: mod_base = OTHER; note = 2'-O-methylguanosine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyluridine-3'-phosphate
  modified_base 22: mod_base = OTHER; note = 2'-O-methyladenosine-3'-phosphate
  modified_base 23: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
agaacagtgttcttgctctataa

SEQ ID NO: 112 - RNA, 22 nt, synthetic construct
  source 1..22: mol_type = other RNA
  modified_base 1: mod_base = OTHER; note = 2'-O-methylguanosine-3'-phosphate 5'phosphate
  modified_base 2: mod_base = OTHER; note = 2'-O-methyladenosine-3'-phosphorothioate
  modified_base 3: mod_base = OTHER; note = 2'-O-methyladenosine-3'-phosphorothioate
  modified_base 4: mod_base = OTHER; note = 2'-O-methylcytidine-3'-phosphate
  modified_base 5: mod_base = OTHER; note = 2'-O-methyladenosine-3'-phosphate
  modified_base 6: mod_base = OTHER; note = 2'-O-methylguanosine-3'-phosphate
  modified_base 7: mod_base = OTHER; note = 2'-O-methyluridine-3'-phosphate
  modified_base 8: mod_base = OTHER; note = 2'-deoxy-2'-fluoroguanosine-3'-phosphate
  modified_base 9: mod_base = OTHER; note = 2'-O-methyluridine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorocytidine-3'-phosphate
  modified_base 12: mod_base = OTHER; note = 2'-deoxy-2'-fluorouridine-3'-phosphate
  modified_base 13: mod_base = OTHER; note = 2'-O-methyluridine-3'-phosphate
  modified_base 14: mod_base = OTHER; note = 2'-O-methylguanosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methyladenosine-3'-phosphate
  modified_base 22: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
gaacagtgttcttgctctataa

SEQ ID NO: 113 - RNA, 21 nt, synthetic construct
  source 1..21: mol_type = other RNA
  modified_base 1: mod_base = OTHER; note = 2'-O-methyladenosine-3'-phosphorothioate 5'phosphate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
aacagtgttcttgctctataa

SEQ ID NO: 114 - RNA, 23 nt, synthetic construct
  source 1..23: mol_type = other RNA
  modified_base 1: mod_base = OTHER; note = 2'-O-methyladenosine-3'-phosphate 5'phosphate
  modified_base 2: mod_base = OTHER; note = 2'-O-methylguanosine-3'-phosphate
  modified_base 3: mod_base = OTHER; note = 2'-O-methyladenosine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphate
  modified_base 6: mod_base = OTHER; note = 2'-O-methyladenosine-3'-phosphate
  modified_base 7: mod_base = OTHER; note = 2'-O-methylguanosine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-O-methyluridine-3'-phosphate
  modified_base 11: mod_base = OTHER; note = 2'-deoxy-2'-fluorouridine-3'-phosphate
  modified_base 12: mod_base = OTHER; note = 2'-deoxy-2'-fluorocytidine-3'-phosphate
  modified_base 13: mod_base = OTHER; note = 2'-deoxy-2'-fluorouridine-3'-phosphate
  modified_base 14: mod_base = OTHER; note = 2'-O-methyluridine-3'-phosphate
  modified_base 15: mod_base = OTHER; note = 2'-O-methylguanosine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyluridine-3'-phosphate
  modified_base 22: mod_base = OTHER; note = 2'-O-methyladenosine-3'-phosphate
  modified_base 23: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
agaacagtgttcttgctctataa

SEQ ID NO: 115 - RNA, 24 nt, synthetic construct
  source 1..24: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyladenosine-3'-phosphate
  modified_base 3: mod_base = OTHER; note = 2'-O-methylguanosine-3'-phosphate
  modified_base 4: mod_base = OTHER; note = 2'-O-methyladenosine-3'-phosphorothioate
  modified_base 5: mod_base = OTHER; note = 2'-O-methyladenosine-3'-phosphorothioate
  modified_base 6: mod_base = OTHER; note = 2'-O-methylcytidine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-deoxy-2'-fluoroguanosine-3'-phosphate
  modified_base 11: mod_base = OTHER; note = 2'-O-methyluridine-3'-phosphate
  modified_base 12: mod_base = OTHER; note = 2'-deoxy-2'-fluorouridine-3'-phosphate
  modified_base 13: mod_base = OTHER; note = 2'-deoxy-2'-fluorocytidine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-O-methylguanosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylcytidine-3'-phosphate
  modified_base 20: mod_base = OTHER; note = 2'-O-methyluridine-3'-phosphate
  modified_base 21: mod_base = OTHER; note = 2'-O-methyladenosine-3'-phosphate
  modified_base 22: mod_base = OTHER; note = 2'-O-methyluridine-3'-phosphate
  modified_base 23: mod_base = OTHER; note = 2'-O-methyladenosine-3'-phosphate
  modified_base 24: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
tagaacagtgttcttgctctataa

SEQ ID NO: 116 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 117 - RNA, 11 nt, synthetic construct
  source 1..11: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyluridine-3'-phosphate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester having lost one N-acetylgalactosamine (minus one GalNAc)
ttgctctataa

SEQ ID NO: 118 - RNA, 11 nt, synthetic construct
  source 1..11: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyluridine-3'-phosphate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester having lost two N-acetylgalactosamine (minus two GalNAc)
ttgctctataa

SEQ ID NO: 119 - RNA, 36 nt, synthetic construct
  source 1..36: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphorothioate
  modified_base 24: mod_base = OTHER; note = 2'-O-methyluridine-3'-phosphorothioate
  modified_base 25: mod_base = OTHER; note = 2'-O-methyluridine-3'-phosphorothioate
  modified_base 26: mod_base = OTHER; note = 2'-O-methyluridine-3'-phosphorothioate
  modified_base 27: mod_base = OTHER; note = 2'-O-methyladenosine-3'-phosphorothioate
  modified_base 28: mod_base = OTHER; note = 2'-O-methyladenosine-3'-phosphorothioate
  modified_base 29: mod_base = OTHER; note = 2'-O-methylcytidine-3'-phosphate
  modified_base 30: mod_base = OTHER; note = 2'-O-methyladenosine-3'-phosphate
  modified_base 31: mod_base = OTHER; note = 2'-O-methylguanosine-3'-phosphate
  modified_base 32: mod_base = OTHER; note = 2'-O-methyluridine-3'-phosphate
  modified_base 33: mod_base = OTHER; note = 2'-deoxy-2'-fluoroguanosine-3'-phosphate
  modified_base 34: mod_base = OTHER; note = 2'-O-methyluridine-3'-phosphate
  modified_base 35: mod_base = OTHER; note = 2'-deoxy-2'-fluorouridine-3'-phosphate
  modified_base 36: mod_base = OTHER; note = 2'-deoxy-2'-fluorocytidine
ttatagagcaagaacactgtttttttaacagtgttc

SEQ ID NO: 120 - RNA, 34 nt, synthetic construct; identical to SEQ ID NO: 73
  source 1..34: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphorothioate
  modified_base 24: mod_base = OTHER; note = 2'-O-methyluridine-3'-phosphorothioate
  modified_base 25: mod_base = OTHER; note = 2'-O-methyluridine-3'-phosphorothioate
  modified_base 26: mod_base = OTHER; note = 2'-O-methyluridine-3'-phosphorothioate
  modified_base 27: mod_base = OTHER; note = 2'-O-methyladenosine-3'-phosphorothioate
  modified_base 28: mod_base = OTHER; note = 2'-O-methyladenosine-3'-phosphorothioate
  modified_base 29: mod_base = OTHER; note = 2'-O-methylcytidine-3'-phosphate
  modified_base 30: mod_base = OTHER; note = 2'-O-methyladenosine-3'-phosphate
  modified_base 31: mod_base = OTHER; note = 2'-O-methylguanosine-3'-phosphate
  modified_base 32: mod_base = OTHER; note = 2'-O-methyluridine-3'-phosphate
  modified_base 33: mod_base = OTHER; note = 2'-deoxy-2'-fluoroguanosine-3'-phosphate
  modified_base 34: mod_base = OTHER; note = 2'-O-methyluridine

SEQ ID NO: 121 - RNA, 11 nt, synthetic construct
  source 1..11: mol_type = other RNA
  modified_base 1: mod_base = OTHER; note = 2'-deoxy-2'-fluorouridine-3'-phosphate 5'phosphate
  modified_base 2: mod_base = OTHER; note = 2'-O-methyluridine-3'-phosphate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
ttgctctataa

SEQ ID NO: 122 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-hexadecyl-uridine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
aacagtgttcttgctctataa

SEQ ID NO: 123 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 124 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphate
  modified_base 4: mod_base = OTHER; note = 2'-O-methylcytidine-3'-phosphate
  modified_base 5: mod_base = OTHER; note = 2'-O-methyladenosine-3'-phosphate
  modified_base 6: mod_base = OTHER; note = 2'-O-hexadecyl-guanosine-3'-phosphate
  modified_base 7: mod_base = OTHER; note = 2'-deoxy-2'-fluoroadenosine-3'-phosphate
  modified_base 8: mod_base = OTHER; note = 2'-O-methyluridine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-O-methyluridine-3'-phosphate
  modified_base 15: mod_base = OTHER; note = 2'-O-methyladenosine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methylcytidine-3'-phosphorothioate
  modified_base 20: mod_base = OTHER; note = 2'-O-methylguanosine-3'-phosphorothioate
  modified_base 21: mod_base = OTHER; note = 2'-O-methyladenosine
acccagatatttttatcgcga

SEQ ID NO: 125 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorocytidine-3'-phosphorothioate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methyluridine-3'-phosphate
  modified_base 8: mod_base = OTHER; note = 2'-deoxy-2'-fluoroadenosine-3'-phosphate
  modified_base 9: mod_base = OTHER; note = 2'-deoxy-2'-fluoroadenosine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methyladenosine-3'-phosphate
  modified_base 13: mod_base = OTHER; note = 2'-O-methyluridine-3'-phosphate
  modified_base 14: mod_base = OTHER; note = 2'-deoxy-2'-fluoroadenosine-3'-phosphate
  modified_base 15: mod_base = OTHER; note = 2'-O-methyluridine-3'-phosphate
  modified_base 16: mod_base = OTHER; note = 2'-deoxy-2'-fluorocytidine-3'-phosphate
  modified_base 17: mod_base = OTHER; note = 2'-O-methyluridine-3'-phosphate
  modified_base 18: mod_base = OTHER; note = 2'-O-methylguanosine-3'-phosphate
  modified_base 19: mod_base = OTHER; note = 2'-O-methylguanosine-3'-phosphate
  modified_base 20: mod_base = OTHER; note = 2'-O-methylguanosine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tcgcgataaaaatatctgggtcg

SEQ ID NO: 126 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methylguanosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-O-hexadecyl-cytidine-3'-phosphate
  modified_base 7: mod_base = OTHER; note = 2'-deoxy-2'-fluoroguanosine-3'-phosphate
  modified_base 8: mod_base = OTHER; note = 2'-O-methyladenosine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-O-methyluridine-3'-phosphate
  modified_base 15: mod_base = OTHER; note = 2'-O-methyladenosine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methylcytidine-3'-phosphorothioate
  modified_base 20: mod_base = OTHER; note = 2'-O-methylguanosine-3'-phosphorothioate
  modified_base 21: mod_base = OTHER; note = 2'-O-methyladenosine
tagtacgactatttatcgcga

SEQ ID NO: 127 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorocytidine-3'-phosphorothioate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methyluridine-3'-phosphate
  modified_base 8: mod_base = OTHER; note = 2'-deoxy-2'-fluoroadenosine-3'-phosphate
  modified_base 9: mod_base = OTHER; note = 2'-deoxy-2'-fluoroadenosine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methyladenosine-3'-phosphate
  modified_base 13: mod_base = OTHER; note = 2'-O-methylguanosine-3'-phosphate
  modified_base 14: mod_base = OTHER; note = 2'-deoxy-2'-fluorouridine-3'-phosphate
  modified_base 15: mod_base = OTHER; note = 2'-O-methylcytidine-3'-phosphate
  modified_base 16: mod_base = OTHER; note = 2'-deoxy-2'-fluoroguanosine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methylcytidine-3'-phosphate
  modified_base 20: mod_base = OTHER; note = 2'-O-methyluridine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
tcgcgataaatagtcgtactagt

SEQ ID NO: 128 - RNA, 47 nt, synthetic construct; identical to SEQ ID NO: 48
  source 1..47: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphorothioate
  modified_base 24: mod_base = OTHER; note = 2'-O-methyluridine-3'-phosphorothioate
  modified_base 25: mod_base = OTHER; note = 2'-O-methyluridine-3'-phosphorothioate
  modified_base 26: mod_base = OTHER; note = 2'-O-methyluridine-3'-phosphorothioate
  modified_base 27: mod_base = OTHER; note = 2'-O-methyladenosine-3'-phosphorothioate
  modified_base 28: mod_base = OTHER; note = 2'-O-methyladenosine-3'-phosphorothioate
  modified_base 29: mod_base = OTHER; note = 2'-O-methylcytidine-3'-phosphate
  modified_base 30: mod_base = OTHER; note = 2'-O-methyladenosine-3'-phosphate
  modified_base 31: mod_base = OTHER; note = 2'-O-methylguanosine-3'-phosphate
  modified_base 32: mod_base = OTHER; note = 2'-O-hexadecyl-uridine-3'-phosphate
  modified_base 33: mod_base = OTHER; note = 2'-deoxy-2'-fluoroguanosine-3'-phosphate
  modified_base 34: mod_base = OTHER; note = 2'-O-methyluridine-3'-phosphate
  modified_base 35: mod_base = OTHER; note = 2'-deoxy-2'-fluorouridine-3'-phosphate
  modified_base 36: mod_base = OTHER; note = 2'-deoxy-2'-fluorocytidine-3'-phosphate
  modified_base 37: mod_base = OTHER; note = 2'-deoxy-2'-fluorouridine-3'-phosphate
  modified_base 38: mod_base = OTHER; note = 2'-O-methyluridine-3'-phosphate
  modified_base 39: mod_base = OTHER; note = 2'-O-methylguanosine-3'-phosphate
  modified_base 40: mod_base = OTHER; note = 2'-O-methylcytidine-3'-phosphate
  modified_base 41: mod_base = OTHER; note = 2'-O-methyluridine-3'-phosphate
  modified_base 42: mod_base = OTHER; note = 2'-O-methylcytidine-3'-phosphate
  modified_base 43: mod_base = OTHER; note = 2'-O-methyluridine-3'-phosphate
  modified_base 44: mod_base = OTHER; note = 2'-O-methyladenosine-3'-phosphate
  modified_base 45: mod_base = OTHER; note = 2'-O-methyluridine-3'-phosphorothioate
  modified_base 46: mod_base = OTHER; note = 2'-O-methyladenosine-3'-phosphorothioate
  modified_base 47: mod_base = OTHER; note = 2'-O-methyladenosine

SEQ ID NO: 129 - RNA, 51 nt, synthetic construct; identical to SEQ ID NO: 40
  source 1..51: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate
  modified_base 26: mod_base = OTHER; note = 2'-deoxy-2'-fluoroguanosine-3'-phosphate
  modified_base 27: mod_base = OTHER; note = 2'-deoxy-2'-fluoroadenosine-3'-phosphate
  modified_base 28: mod_base = OTHER; note = 2'-deoxy-2'-fluorouridine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-hexadecyl-uridine-3'-phosphate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine-3'-phosphate
  modified_base 40: mod_base = OTHER; note = 2'-deoxy-2'-fluorocytidine-3'-phosphate
  modified_base 41: mod_base = OTHER; note = 2'-deoxy-2'-fluorouridine-3'-phosphate
  modified_base 42: mod_base = OTHER; note = 2'-O-methyluridine-3'-phosphate
  modified_base 43: mod_base = OTHER; note = 2'-O-methylguanosine-3'-phosphate
  modified_base 44: mod_base = OTHER; note = 2'-O-methylcytidine-3'-phosphate
  modified_base 45: mod_base = OTHER; note = 2'-O-methyluridine-3'-phosphate
  modified_base 46: mod_base = OTHER; note = 2'-O-methylcytidine-3'-phosphate
  modified_base 47: mod_base = OTHER; note = 2'-O-methyluridine-3'-phosphate
  modified_base 48: mod_base = OTHER; note = 2'-O-methyladenosine-3'-phosphate
  modified_base 49: mod_base = OTHER; note = 2'-O-methyluridine-3'-phosphorothioate
  modified_base 50: mod_base = OTHER; note = 2'-O-methyladenosine-3'-phosphorothioate
  modified_base 51: mod_base = OTHER; note = 2'-O-methyladenosine

SEQ ID NO: 130 - DNA, 51 bp, synthetic construct
  source 1..51: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-hexadecyl-uridine-3'-phosphate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine-3'-phosphate
  modified_base 40: mod_base = OTHER; note = 2'-deoxy-2'-fluorocytidine-3'-phosphate
  modified_base 41: mod_base = OTHER; note = 2'-deoxy-2'-fluorouridine-3'-phosphate
  modified_base 42: mod_base = OTHER; note = 2'-O-methyluridine-3'-phosphate
  modified_base 43: mod_base = OTHER; note = 2'-O-methylguanosine-3'-phosphate
  modified_base 44: mod_base = OTHER; note = 2'-O-methylcytidine-3'-phosphate
  modified_base 45: mod_base = OTHER; note = 2'-O-methyluridine-3'-phosphate
  modified_base 46: mod_base = OTHER; note = 2'-O-methylcytidine-3'-phosphate
  modified_base 47: mod_base = OTHER; note = 2'-O-methyluridine-3'-phosphate
  modified_base 48: mod_base = OTHER; note = 2'-O-methyladenosine-3'-phosphate
  modified_base 49: mod_base = OTHER; note = 2'-O-methyluridine-3'-phosphorothioate
  modified_base 50: mod_base = OTHER; note = 2'-O-methyladenosine-3'-phosphorothioate
  modified_base 51: mod_base = OTHER; note = 2'-O-methyladenosine
  misc_feature 1..23: note = RNA
  misc_feature 29..51: note = RNA
  misc_feature 24..28: note = DNA
ttatagagcaagaacactgtttttttttagaacagtgttcttgctctataa

SEQ ID NO: 131 - RNA, 23 nt, synthetic construct
  source 1..23: mol_type = other RNA
  modified_base 1: mod_base = OTHER; note = 2'-O-methyladenosine-3'-phosphate connected at the 5'-end via a phosphodiester linkage to Q304Q304Q304
  modified_base 2: mod_base = OTHER; note = 2'-O-methylguanosine-3'-phosphate
  modified_base 3: mod_base = OTHER; note = 2'-O-methyladenosine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphate
  modified_base 6: mod_base = OTHER; note = 2'-O-methyladenosine-3'-phosphate
  modified_base 7: mod_base = OTHER; note = 2'-O-methylguanosine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-O-methyluridine-3'-phosphate
  modified_base 11: mod_base = OTHER; note = 2'-deoxy-2'-fluorouridine-3'-phosphate
  modified_base 12: mod_base = OTHER; note = 2'-deoxy-2'-fluorocytidine-3'-phosphate
  modified_base 13: mod_base = OTHER; note = 2'-deoxy-2'-fluorouridine-3'-phosphate
  modified_base 14: mod_base = OTHER; note = 2'-O-methyluridine-3'-phosphate
  modified_base 15: mod_base = OTHER; note = 2'-O-methylguanosine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyluridine-3'-phosphate
  modified_base 22: mod_base = OTHER; note = 2'-O-methyladenosine-3'-phosphate
  modified_base 23: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
agaacagtgttcttgctctataa

SEQ ID NO: 132 - RNA, 23 nt, synthetic construct
  source 1..23: mol_type = other RNA
  modified_base 1: mod_base = OTHER; note = 2'-O-methyladenosine-3'-phosphate connected at the 5'-end via a phosphodiester linkage to Q304Q304Q304
  modified_base 2: mod_base = OTHER; note = 2'-O-methylguanosine-3'-phosphate
  modified_base 3: mod_base = OTHER; note = 2'-O-methyladenosine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphate
  modified_base 6: mod_base = OTHER; note = 2'-O-methyladenosine-3'-phosphate
  modified_base 7: mod_base = OTHER; note = 2'-O-methylguanosine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-O-methyluridine-3'-phosphate
  modified_base 11: mod_base = OTHER; note = 2'-deoxy-2'-fluorouridine-3'-phosphate
  modified_base 12: mod_base = OTHER; note = 2'-deoxy-2'-fluorocytidine-3'-phosphate
  modified_base 13: mod_base = OTHER; note = 2'-deoxy-2'-fluorouridine-3'-phosphate
  modified_base 14: mod_base = OTHER; note = 2'-O-methyluridine-3'-phosphate
  modified_base 15: mod_base = OTHER; note = 2'-O-methylguanosine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methyladenosine-3'-phosphate
  modified_base 21: mod_base = OTHER; note = 2'-O-methyluridine-3'-phosphate
  modified_base 22: mod_base = OTHER; note = 2'-O-methyladenosine-3'-phosphate
  modified_base 23: mod_base = OTHER; note = 2'-O-methyladenosine-3'-phosphate ((2S,4R)-1-[29-[[ 2-(acetylamino)-2-deoxy-beta-D-galactopyranosyl]oxy]-14,14 -bis[[3-[[3-[[5-[[2-(acetylamino)-2-deoxy-beta-D-galactopy ranosyl]oxy]-1-oxopentyl]amino]propyl]amino]-3-oxopropoxy] methyl]-1,12,19,25-tetraoxo-16-oxa-13,20,24-triazanonacos- 1-yl]-4-hydroxy-2-pyrrolidinyl)methyl ester
agaacagtgttcttgctctataa

SEQ ID NO: 133 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-methyluridine-3'-(n-hexadecyl)phosphoramidate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
aacagtgttcttgctctataa

SEQ ID NO: 134 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
ttatagagcaagaacactgtttt

SEQ ID NO: 135 - RNA, 51 nt, synthetic construct; identical to SEQ ID NO: 40
  source 1..51: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 24: mod_base = OTHER; note = 2'-O-methylguanosine-3'-phosphate
  modified_base 25: mod_base = OTHER; note = 2'-O-methyladenosine-3'-phosphate
  modified_base 26: mod_base = OTHER; note = 2'-deoxy-2'-fluoroguanosine-3'-phosphate
  modified_base 27: mod_base = OTHER; note = 2'-deoxy-2'-fluoroadenosine-3'-phosphate
  modified_base 28: mod_base = OTHER; note = 2'-deoxy-2'-fluorouridine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-methyluridine-3'-(n-hexadecyl)phosphoramidate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine-3'-phosphate
  modified_base 40: mod_base = OTHER; note = 2'-deoxy-2'-fluorocytidine-3'-phosphate
  modified_base 41: mod_base = OTHER; note = 2'-deoxy-2'-fluorouridine-3'-phosphate
  modified_base 42: mod_base = OTHER; note = 2'-O-methyluridine-3'-phosphate
  modified_base 43: mod_base = OTHER; note = 2'-O-methylguanosine-3'-phosphate
  modified_base 44: mod_base = OTHER; note = 2'-O-methylcytidine-3'-phosphate
  modified_base 45: mod_base = OTHER; note = 2'-O-methyluridine-3'-phosphate
  modified_base 46: mod_base = OTHER; note = 2'-O-methylcytidine-3'-phosphate
  modified_base 47: mod_base = OTHER; note = 2'-O-methyluridine-3'-phosphate
  modified_base 48: mod_base = OTHER; note = 2'-O-methyladenosine-3'-phosphate
  modified_base 49: mod_base = OTHER; note = 2'-O-methyluridine-3'-phosphorothioate
  modified_base 50: mod_base = OTHER; note = 2'-O-methyladenosine-3'-phosphorothioate
  modified_base 51: mod_base = OTHER; note = 2'-O-methyladenosine-3'-phosphate

SEQ ID NO: 136 - DNA, 51 bp, synthetic construct; identical to SEQ ID NO: 130
  source 1..51: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine-3'-phosphate
  modified_base 29: mod_base = OTHER; note = 2'-O-methyladenosine-3'-phosphate
  modified_base 30: mod_base = OTHER; note = 2'-O-methylguanosine-3'-phosphate
  modified_base 31: mod_base = OTHER; note = 2'-O-methyladenosine-3'-phosphorothioate
  modified_base 32: mod_base = OTHER; note = 2'-O-methyladenosine-3'-phosphorothioate
  modified_base 33: mod_base = OTHER; note = 2'-O-methylcytidine-3'-phosphate
  modified_base 34: mod_base = OTHER; note = 2'-O-methyladenosine-3'-phosphate
  modified_base 35: mod_base = OTHER; note = 2'-O-methylguanosine-3'-phosphate
  modified_base 36: mod_base = OTHER; note = 2'-O-methyluridine-3'-(n-hexadecyl)phosphoramidate
  modified_base 37: mod_base = OTHER; note = 2'-deoxy-2'-fluoroguanosine-3'-phosphate
  modified_base 38: mod_base = OTHER; note = 2'-O-methyluridine-3'-phosphate
  modified_base 39: mod_base = OTHER; note = 2'-deoxy-2'-fluorouridine-3'-phosphate
  modified_base 40: mod_base = OTHER; note = 2'-deoxy-2'-fluorocytidine-3'-phosphate
  modified_base 41: mod_base = OTHER; note = 2'-deoxy-2'-fluorouridine-3'-phosphate
  modified_base 42: mod_base = OTHER; note = 2'-O-methyluridine-3'-phosphate
  modified_base 43: mod_base = OTHER; note = 2'-O-methylguanosine-3'-phosphate
  modified_base 44: mod_base = OTHER; note = 2'-O-methylcytidine-3'-phosphate
  modified_base 45: mod_base = OTHER; note = 2'-O-methyluridine-3'-phosphate
  modified_base 46: mod_base = OTHER; note = 2'-O-methylcytidine-3'-phosphate
  modified_base 47: mod_base = OTHER; note = 2'-O-methyluridine-3'-phosphate
  modified_base 48: mod_base = OTHER; note = 2'-O-methyladenosine-3'-phosphate
  modified_base 49: mod_base = OTHER; note = 2'-O-methyluridine-3'-phosphorothioate
  modified_base 50: mod_base = OTHER; note = 2'-O-methyladenosine-3'-phosphorothioate
  modified_base 51: mod_base = OTHER; note = 2'-O-methyladenosine-3'-phosphate
  misc_feature 1..23: note = RNA
  misc_feature 29..51: note = RNA
  misc_feature 24..28: note = DNA